(19) United States
(12) Patent Application Publication
(10) Pub. No.: US 2025/0171460 A1
(43) Pub. Date: May 29, 2025

(54) INDUCERS OF KLF2 AND METHODS OF USE THEREOF

(71) Applicant: Riparian Pharmaceuticals, Inc., Watertown, MA (US)

(72) Inventor: Michael Serrano-Wu, Belmont, MA (US)

(73) Assignee: Riparian Pharmaceuticals, Inc., Watertown, MA (US)

(21) Appl. No.: 18/836,034

(22) PCT Filed: Feb. 7, 2023
(86) PCT No.: PCT/US2023/012487

§ 371 Date: Aug. 6, 2024

Related U.S. Application Data
(60) Provisional application No. 63/307,416, filed on Feb. 7, 2022.

(51) Int. Cl. C07D 498/04; A61K 31/553; C07D 498/14

(52) U.S. Cl. CPC C07D 498/04; A61K 31/553; C07D 498/14

(57) ABSTRACT

The present disclosure provides compounds that are inducers of KLF2 and pharmaceutical compositions comprising the same. The present disclosure further provides method of treating an inflammatory disease or endothelial dysfunction comprising administering a therapeutically effective amount of the compounds disclosed herein.

### CROSS-REFERENCE TO RELATED APPLICATIONS

This application claims the benefit of priority to U.S. Provisional Application No. 63/307,416, filed on Feb. 7, 2022, the contents of which are hereby incorporated by reference in their entirety.

### STATEMENT OF GOVERNMENT SUPPORT

This invention was made with government support under Grant Number R44HL118826 awarded by the National Institutes of Health National Heart, Lung and Blood Institute.

### BACKGROUND OF THE INVENTION

The vascular endothelium is an important regulator of vascular integrity and vascular homeostasis. The vascular endothelium is a dynamic interface that regulates vasotone, inflammation, hemostasis and vascular remodeling. Dysfunction of the vascular endothelium, including vasoconstriction, impaired vasoreactivity, inflammation, thrombosis, altered barrier permeability and loss of vascular quiescence, is a key driver of many vascular diseases. As such, the vascular endothelium is important for maintaining vascular and cardiovascular health.

Kruppel-like Factor 2 (KLF2) is a shear stress-induced transcription factor that may confer anti-inflammatory and/or anti-thrombotic properties to vascular endothelial cells. In endothelial cells, KLF2 may be involved in transcriptional processes for regulating inflammation, thrombosis hemostasis, vascular tone, and blood vessel development. KLF2 is a key regulator of activation, differentiation, and migration processes in various immune cell types including monocytes, macrophages, neutrophils, T lymphocytes, B lymphocytes and natural killer cells.

Accordingly, compounds that induce KLF2 may be useful for maintaining vascular health or for treating vascular or inflammatory conditions.

### SUMMARY

In some embodiments, the invention provides compounds represented by formula I.

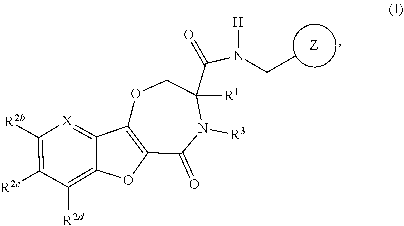

or a pharmaceutically acceptable salt thereof, wherein:
R¹ represents lower alkyl;
X represents C—R^{2a} or N;
R^{2a}, R^{2b}, R^{2c}, and R^{2d} each independently represent hydrogen, alkyl, alkenyl, alkynyl, halo, aryl, heteroaryl, cycloalkyl, heterocyclyl, cyano, acyl, carboxy, ester, or amido;
R³ represents alkyl, alkenyl, alkynyl, aryl, heteroaryl, cycloalkyl, heterocyclyl, arylalkyl, heteroaralkyl, (cycloalkyl)alkyl, heterocyclylalkyl, amidoalkyl, alkoxyalkyl, or acylalkyl; and
Z represents a substituted or unsubstituted aryl or heteroaryl ring, e.g., optionally substituted with one or more groups chosen from alkyl, alkenyl, alkynyl, cyano, acyl, carboxy, ester, amido, alkoxy, and halo.

In certain embodiments, the present invention is for treating an inflammatory disease or endothelial dysfunction, wherein the method comprises administering a therapeutically effective amount of a compound or a composition described herein.

In certain embodiments, the present invention provides a pharmaceutical composition for treating an inflammatory disease or endothelial dysfunction, the composition comprising an effective amount of any of the compounds described herein (e.g., a compound of the invention, such as a compound of formula (I) or a pharmaceutically acceptable salt thereof).

In certain embodiments, the pharmaceutical preparations may be for use in treating or preventing a condition or disease as described herein.

In certain embodiments, the compound of formula (I) is chosen from:

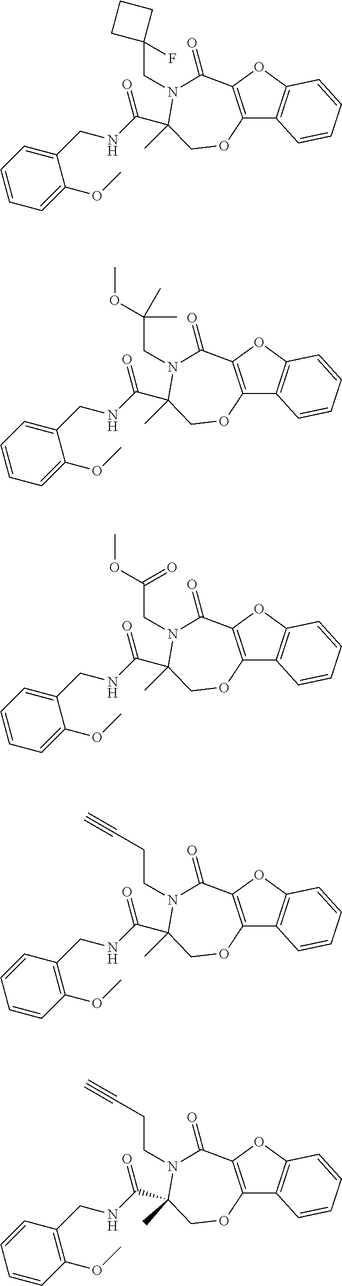 [SMILES: COc1ccccc1CNC(=O)C1(C)COc2c(oc3ccccc23)C(=O)N1CC1(F)CCC1 ; COc1ccccc1CNC(=O)C1(C)COc2c(oc3ccccc23)C(=O)N1CC(C)(C)OC ; COC(=O)CN1C(=O)c2oc3ccccc3c2OCC1(C)C(=O)NCc1ccccc1OC ; C#CCCN1C(=O)c2oc3ccccc3c2OCC1(C)C(=O)NCc1ccccc1OC ; C#CCCN1C(=O)c2oc3ccccc3c2OC[C@]1(C)C(=O)NCc1ccccc1OC]

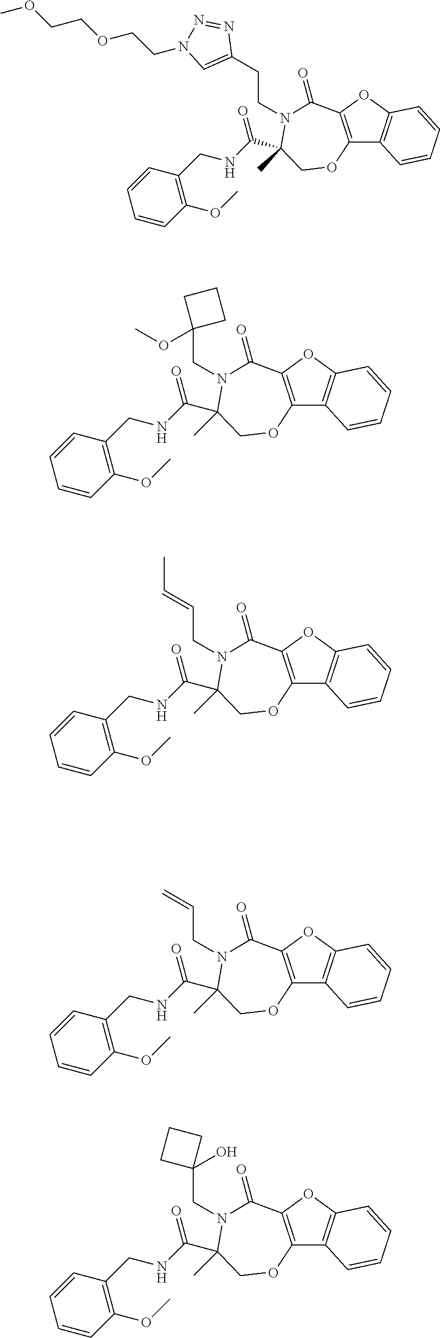 [SMILES: COCCOCCn1cc(CCN2C(=O)c3oc4ccccc4c3OC[C@]2(C)C(=O)NCc2ccccc2OC)nn1 ; COc1ccccc1CNC(=O)C1(C)COc2c(oc3ccccc23)C(=O)N1CC1(OC)CCC1 ; C/C=C/CN1C(=O)c2oc3ccccc3c2OCC1(C)C(=O)NCc1ccccc1OC ; C=CCN1C(=O)c2oc3ccccc3c2OCC1(C)C(=O)NCc1ccccc1OC ; COc1ccccc1CNC(=O)C1(C)COc2c(oc3ccccc23)C(=O)N1CC1(O)CCC1]

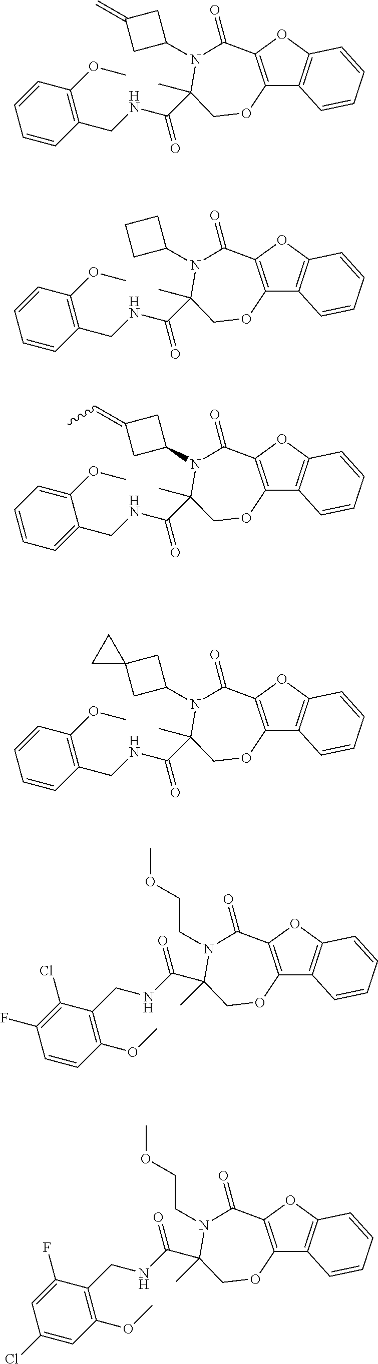

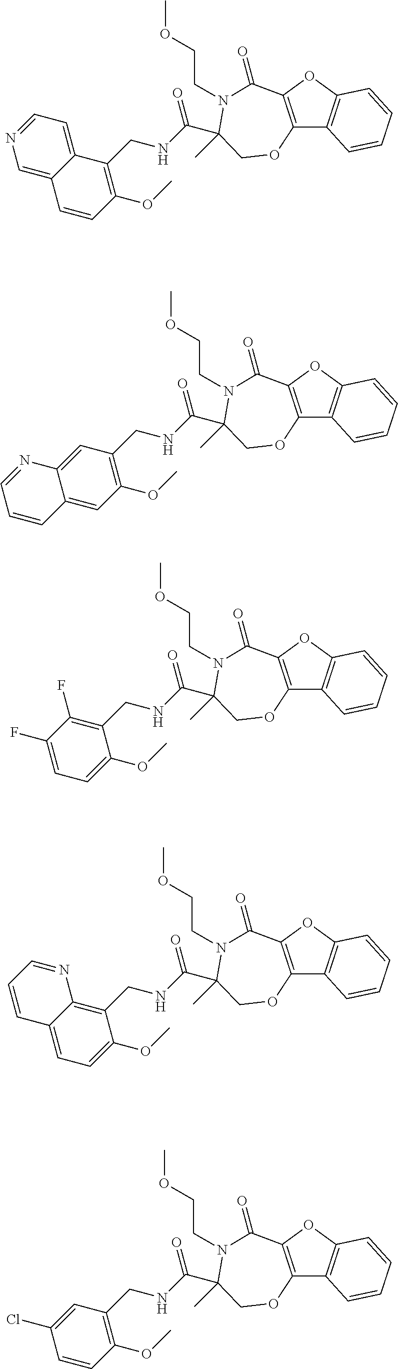 [SMILES: COCCN1C(=O)c2oc3ccccc3c2OCC1(C)C(=O)NCc1c(OC)ccc2cnccc12 ; COCCN1C(=O)c2oc3ccccc3c2OCC1(C)C(=O)NCc1cc2ncccc2cc1OC ; COCCN1C(=O)c2oc3ccccc3c2OCC1(C)C(=O)NCc1c(OC)ccc(F)c1F ; COCCN1C(=O)c2oc3ccccc3c2OCC1(C)C(=O)NCc1c(OC)ccc2cccnc12 ; COCCN1C(=O)c2oc3ccccc3c2OCC1(C)C(=O)NCc1cc(Cl)ccc1OC]

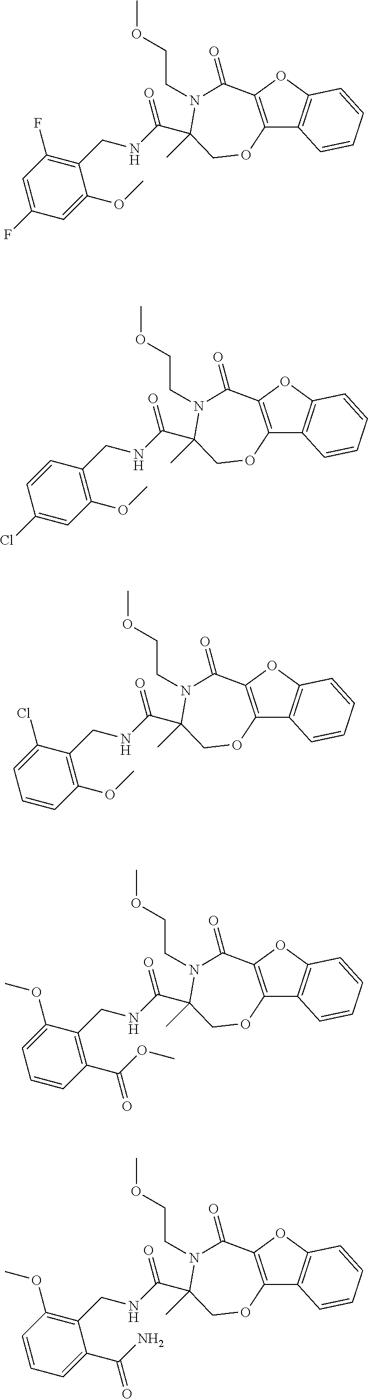 [SMILES: COCCN1C(=O)c2oc3ccccc3c2OCC1(C)C(=O)NCc1c(F)cc(F)cc1OC ; COCCN1C(=O)c2oc3ccccc3c2OCC1(C)C(=O)NCc1ccc(Cl)cc1OC ; COCCN1C(=O)c2oc3ccccc3c2OCC1(C)C(=O)NCc1c(Cl)cccc1OC ; COCCN1C(=O)c2oc3ccccc3c2OCC1(C)C(=O)NCc1c(OC)cccc1C(=O)OC ; COCCN1C(=O)c2oc3ccccc3c2OCC1(C)C(=O)NCc1c(OC)cccc1C(N)=O]

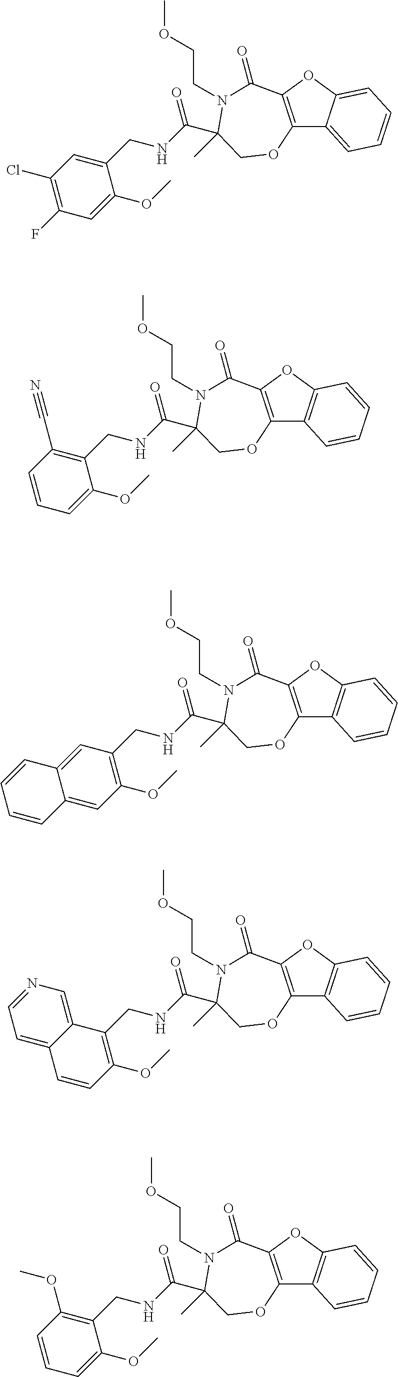 [SMILES: COCCN1C(=O)c2oc3ccccc3c2OCC1(C)C(=O)NCc1cc(Cl)c(F)cc1OC ; COCCN1C(=O)c2oc3ccccc3c2OCC1(C)C(=O)NCc1c(C#N)cccc1OC ; COCCN1C(=O)c2oc3ccccc3c2OCC1(C)C(=O)NCc1cc2ccccc2cc1OC ; COCCN1C(=O)c2oc3ccccc3c2OCC1(C)C(=O)NCc1c(OC)ccc2ccncc12 ; COCCN1C(=O)c2oc3ccccc3c2OCC1(C)C(=O)NCc1c(OC)cccc1OC]

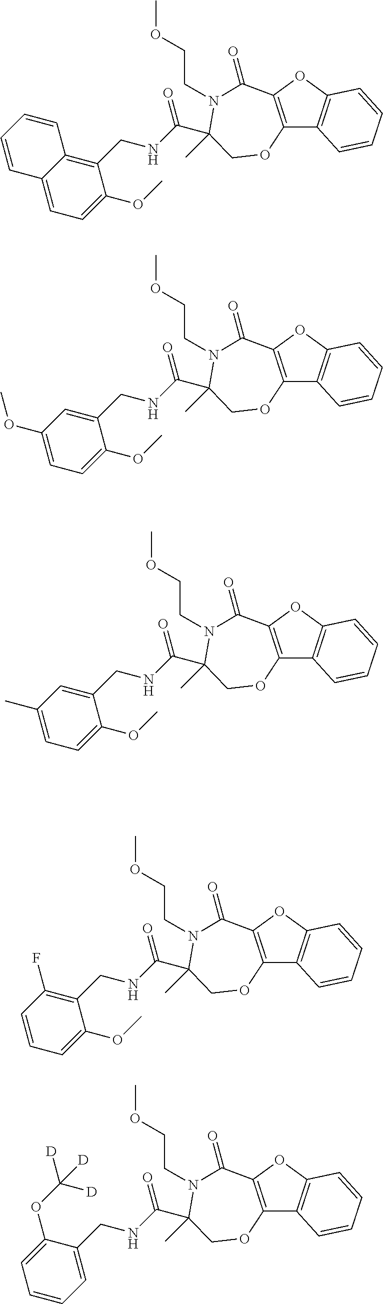 [SMILES: COCCN1C(=O)c2oc3ccccc3c2OCC1(C)C(=O)NCc1c(OC)ccc2ccccc12 ; COCCN1C(=O)c2oc3ccccc3c2OCC1(C)C(=O)NCc1cc(OC)ccc1OC ; COCCN1C(=O)c2oc3ccccc3c2OCC1(C)C(=O)NCc1cc(C)ccc1OC ; COCCN1C(=O)c2oc3ccccc3c2OCC1(C)C(=O)NCc1c(F)cccc1OC ; [2H]C([2H])([2H])Oc1ccccc1CNC(=O)C1(C)COc2c(oc3ccccc23)C(=O)N1CCOC]

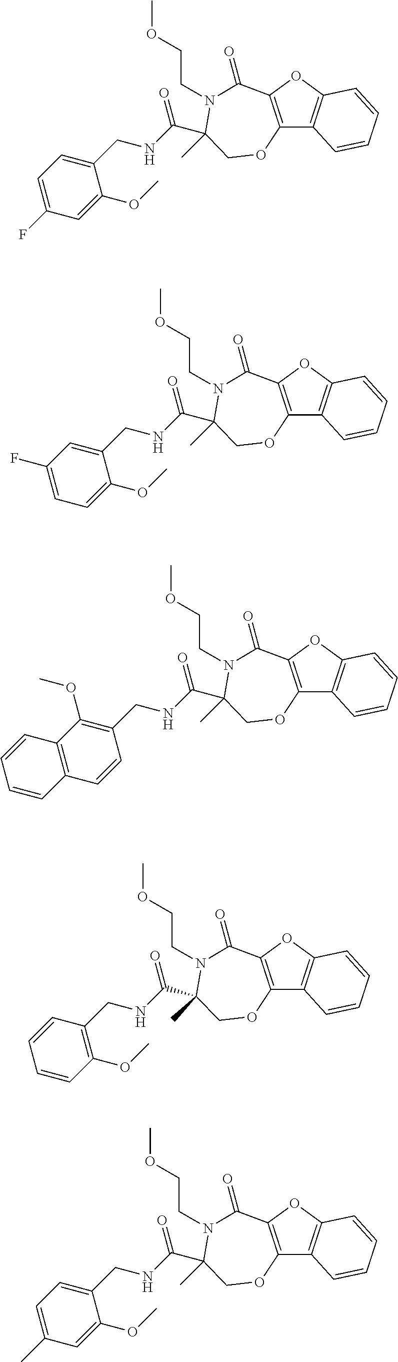 [SMILES: COCCN1C(=O)c2oc3ccccc3c2OCC1(C)C(=O)NCc1ccc(F)cc1OC ; COCCN1C(=O)c2oc3ccccc3c2OCC1(C)C(=O)NCc1cc(F)ccc1OC ; COCCN1C(=O)c2oc3ccccc3c2OCC1(C)C(=O)NCc1ccc2ccccc2c1OC ; COCCN1C(=O)c2oc3ccccc3c2OC[C@]1(C)C(=O)NCc1ccccc1OC ; COCCN1C(=O)c2oc3ccccc3c2OCC1(C)C(=O)NCc1ccc(C)cc1OC]

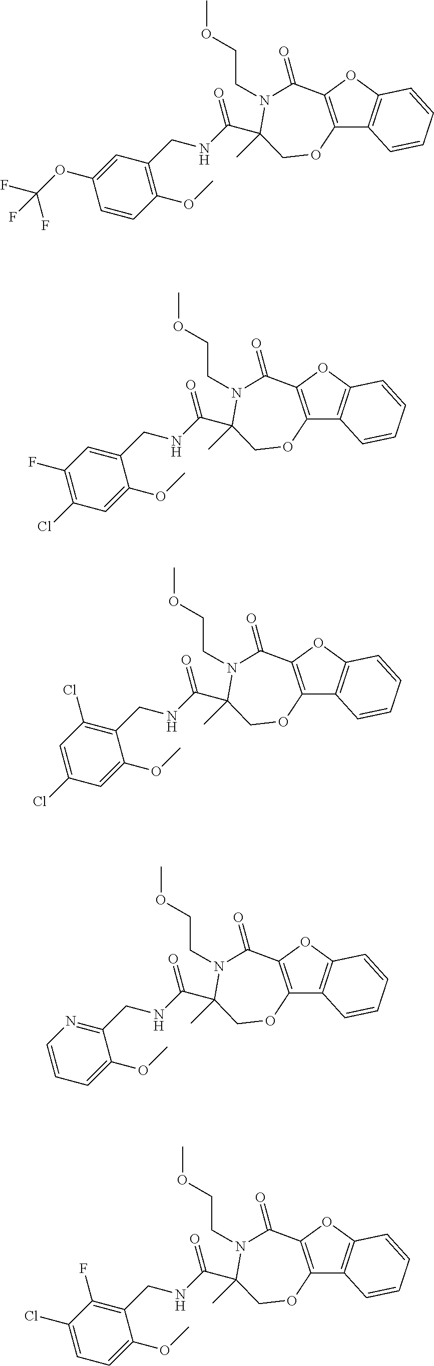 [SMILES: COCCN1C(=O)c2oc3ccccc3c2OCC1(C)C(=O)NCc1cc(OC(F)(F)F)ccc1OC ; COCCN1C(=O)c2oc3ccccc3c2OCC1(C)C(=O)NCc1cc(F)c(Cl)cc1OC ; COCCN1C(=O)c2oc3ccccc3c2OCC1(C)C(=O)NCc1c(Cl)cc(Cl)cc1OC ; COCCN1C(=O)c2oc3ccccc3c2OCC1(C)C(=O)NCc1ncccc1OC ; COCCN1C(=O)c2oc3ccccc3c2OCC1(C)C(=O)NCc1c(OC)ccc(Cl)c1F]

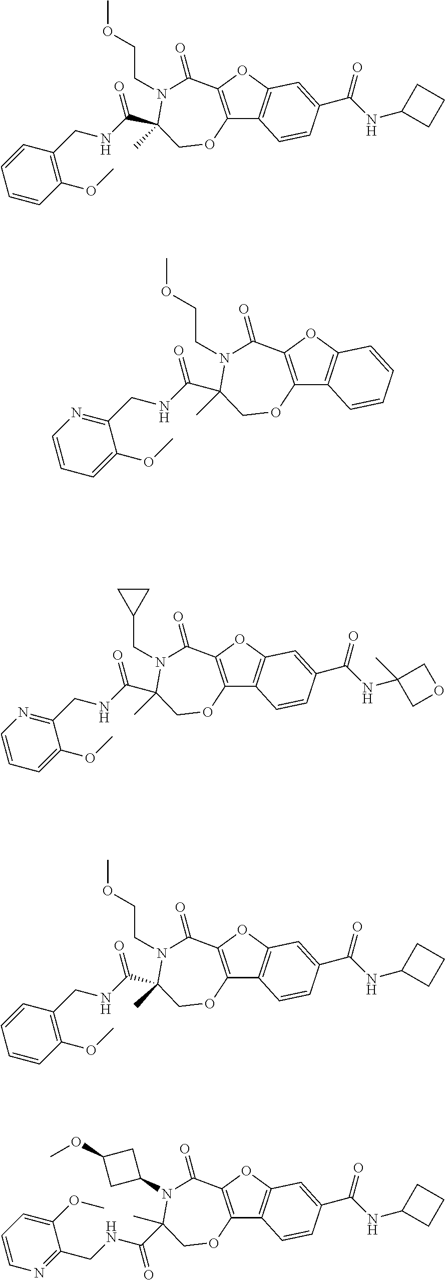 [SMILES: COCCN1C(=O)c2oc3cc(C(=O)NC4CCC4)ccc3c2OC[C@@]1(C)C(=O)NCc1ccccc1OC ; COCCN1C(=O)c2oc3ccccc3c2OCC1(C)C(=O)NCc1ncccc1OC ; COc1cccnc1CNC(=O)C1(C)COc2c(oc3cc(C(=O)NC4(C)COC4)ccc23)C(=O)N1CC1CC1 ; COCCN1C(=O)c2oc3cc(C(=O)NC4CCC4)ccc3c2OC[C@]1(C)C(=O)NCc1ccccc1OC ; COc1cccnc1CNC(=O)C1(C)COc2c(oc3cc(C(=O)NC4CCC4)ccc23)C(=O)N1[C@H]1C[C@@H](OC)C1]

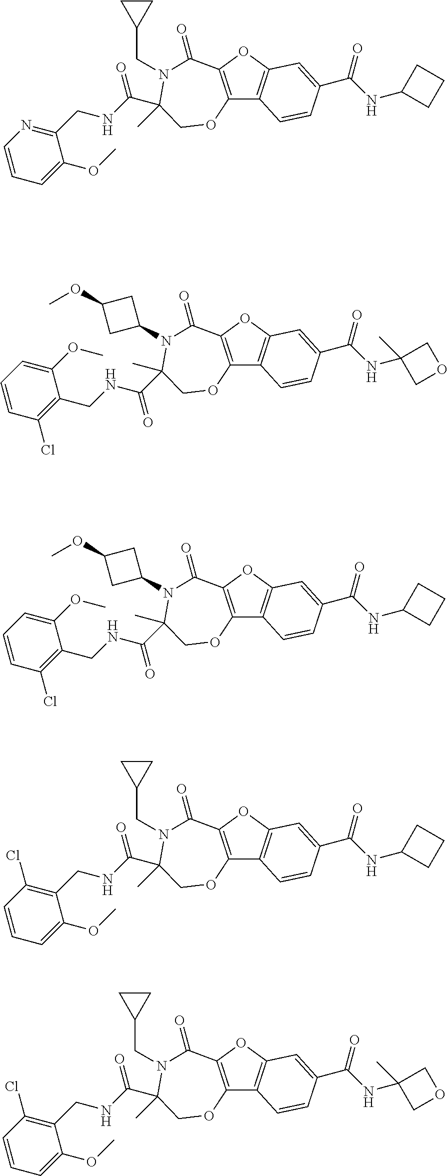 [SMILES: COc1cccnc1CNC(=O)C1(C)COc2c(oc3cc(C(=O)NC4CCC4)ccc23)C(=O)N1CC1CC1 ; COc1cccc(Cl)c1CNC(=O)C1(C)COc2c(oc3cc(C(=O)NC4(C)COC4)ccc23)C(=O)N1[C@H]1C[C@@H](OC)C1 ; COc1cccc(Cl)c1CNC(=O)C1(C)COc2c(oc3cc(C(=O)NC4CCC4)ccc23)C(=O)N1[C@H]1C[C@@H](OC)C1 ; COc1cccc(Cl)c1CNC(=O)C1(C)COc2c(oc3cc(C(=O)NC4CCC4)ccc23)C(=O)N1CC1CC1 ; COc1cccc(Cl)c1CNC(=O)C1(C)COc2c(oc3cc(C(=O)NC4(C)COC4)ccc23)C(=O)N1CC1CC1]

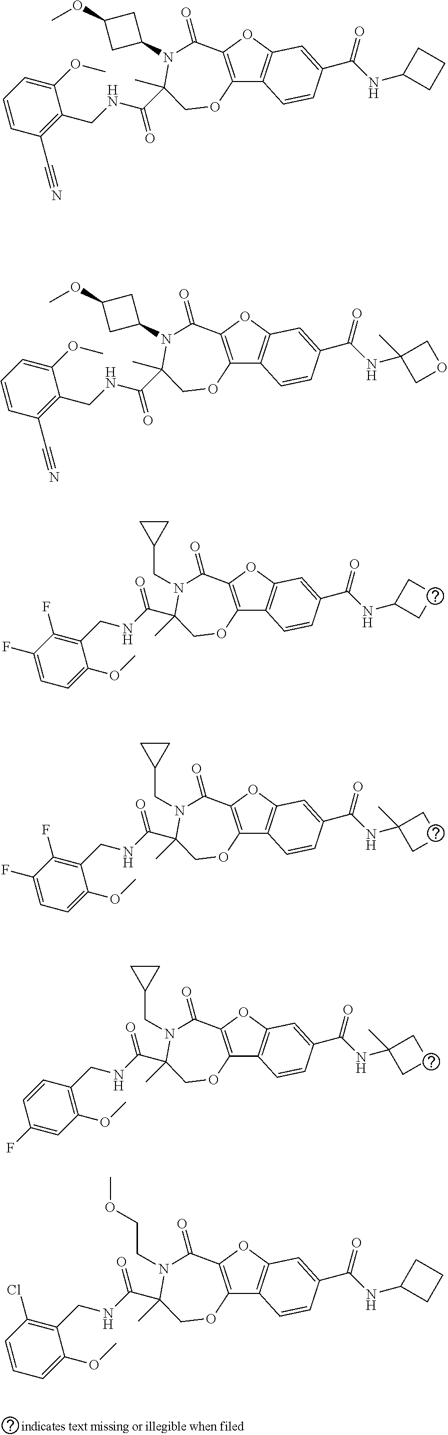 [SMILES: COc1cccc(C#N)c1CNC(=O)C1(C)COc2c(oc3cc(C(=O)NC4CCC4)ccc23)C(=O)N1[C@H]1C[C@@H](OC)C1 ; COc1cccc(C#N)c1CNC(=O)C1(C)COc2c(oc3cc(C(=O)NC4(C)COC4)ccc23)C(=O)N1[C@H]1C[C@@H](OC)C1 ; COc1ccc(F)c(F)c1CNC(=O)C1(C)COc2c(oc3cc(C(=O)NC4CCC4)ccc23)C(=O)N1CC1CC1 ; COc1ccc(F)c(F)c1CNC(=O)C1(C)COc2c(oc3cc(C(=O)NC4(C)CCC4)ccc23)C(=O)N1CC1CC1 ; COc1cc(F)ccc1CNC(=O)C1(C)COc2c(oc3cc(C(=O)NC4(C)CCC4)ccc23)C(=O)N1CC1CC1 ; COCCN1C(=O)c2oc3cc(C(=O)NC4CCC4)ccc3c2OCC1(C)C(=O)NCc1c(Cl)cccc1OC]

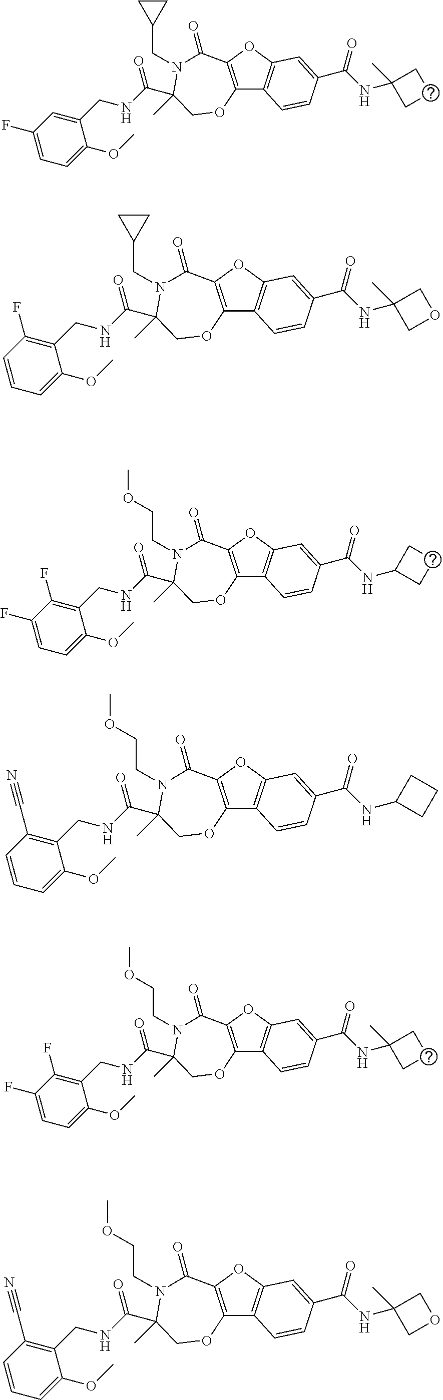 [SMILES: COc1ccc(F)cc1CNC(=O)C1(C)COc2c(oc3cc(C(=O)NC4(C)CCC4)ccc23)C(=O)N1CC1CC1 ; COc1cccc(F)c1CNC(=O)C1(C)COc2c(oc3cc(C(=O)NC4(C)COC4)ccc23)C(=O)N1CC1CC1 ; COCCN1C(=O)c2oc3cc(C(=O)NC4CCC4)ccc3c2OCC1(C)C(=O)NCc1c(OC)ccc(F)c1F ; COCCN1C(=O)c2oc3cc(C(=O)NC4CCC4)ccc3c2OCC1(C)C(=O)NCc1c(C#N)cccc1OC ; COCCN1C(=O)c2oc3cc(C(=O)NC4(C)CCC4)ccc3c2OCC1(C)C(=O)NCc1c(OC)ccc(F)c1F ; COCCN1C(=O)c2oc3cc(C(=O)NC4(C)COC4)ccc3c2OCC1(C)C(=O)NCc1c(C#N)cccc1OC]

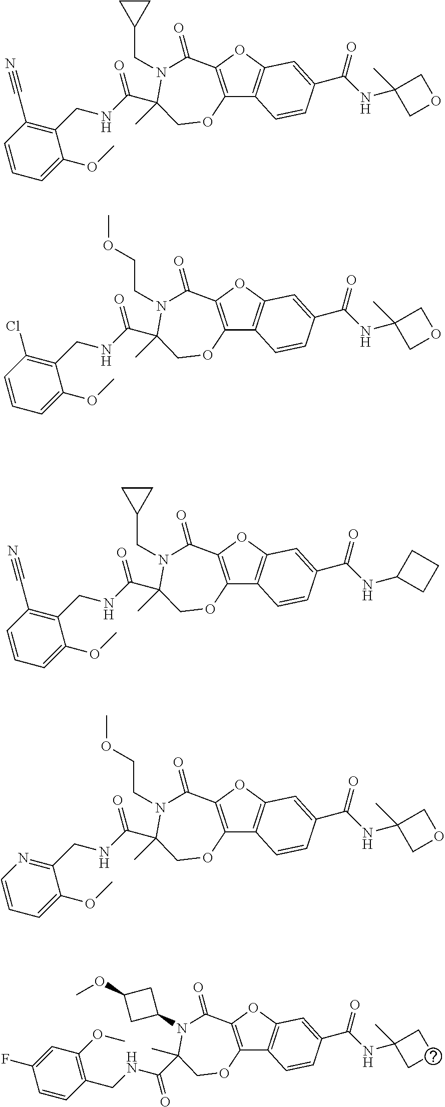 [SMILES: COc1cccc(C#N)c1CNC(=O)C1(C)COc2c(oc3cc(C(=O)NC4(C)COC4)ccc23)C(=O)N1CC1CC1 ; COCCN1C(=O)c2oc3cc(C(=O)NC4(C)COC4)ccc3c2OCC1(C)C(=O)NCc1c(Cl)cccc1OC ; COc1cccc(C#N)c1CNC(=O)C1(C)COc2c(oc3cc(C(=O)NC4CCC4)ccc23)C(=O)N1CC1CC1 ; COCCN1C(=O)c2oc3cc(C(=O)NC4(C)COC4)ccc3c2OCC1(C)C(=O)NCc1ncccc1OC ; COc1cc(F)ccc1CNC(=O)C1(C)COc2c(oc3cc(C(=O)NC4(C)CCC4)ccc23)C(=O)N1[C@H]1C[C@@H](OC)C1]

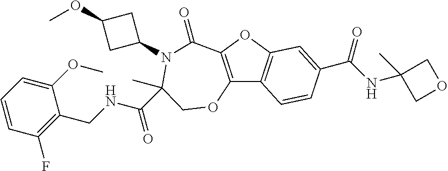 [SMILES: COc1cccc(F)c1CNC(=O)C1(C)COc2c(oc3cc(C(=O)NC4(C)COC4)ccc23)C(=O)N1[C@H]1C[C@@H](OC)C1]

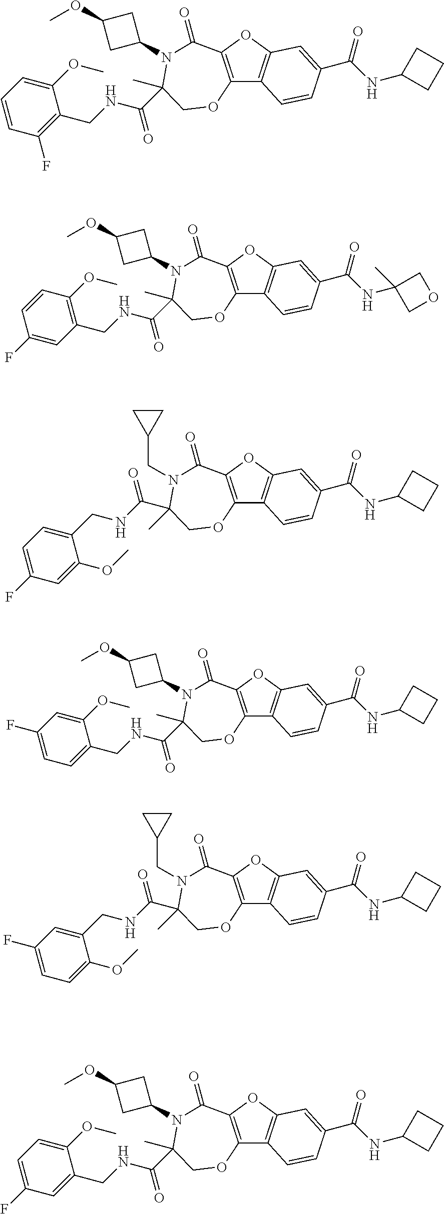 [SMILES: COc1cccc(F)c1CNC(=O)C1(C)COc2c(oc3cc(C(=O)NC4CCC4)ccc23)C(=O)N1[C@H]1C[C@@H](OC)C1 ; COc1ccc(F)cc1CNC(=O)C1(C)COc2c(oc3cc(C(=O)NC4(C)COC4)ccc23)C(=O)N1[C@H]1C[C@@H](OC)C1 ; COc1cc(F)ccc1CNC(=O)C1(C)COc2c(oc3cc(C(=O)NC4CCC4)ccc23)C(=O)N1CC1CC1 ; COc1cc(F)ccc1CNC(=O)C1(C)COc2c(oc3cc(C(=O)NC4CCC4)ccc23)C(=O)N1[C@H]1C[C@@H](OC)C1 ; COc1ccc(F)cc1CNC(=O)C1(C)COc2c(oc3cc(C(=O)NC4CCC4)ccc23)C(=O)N1CC1CC1 ; COc1ccc(F)cc1CNC(=O)C1(C)COc2c(oc3cc(C(=O)NC4CCC4)ccc23)C(=O)N1[C@H]1C[C@@H](OC)C1]

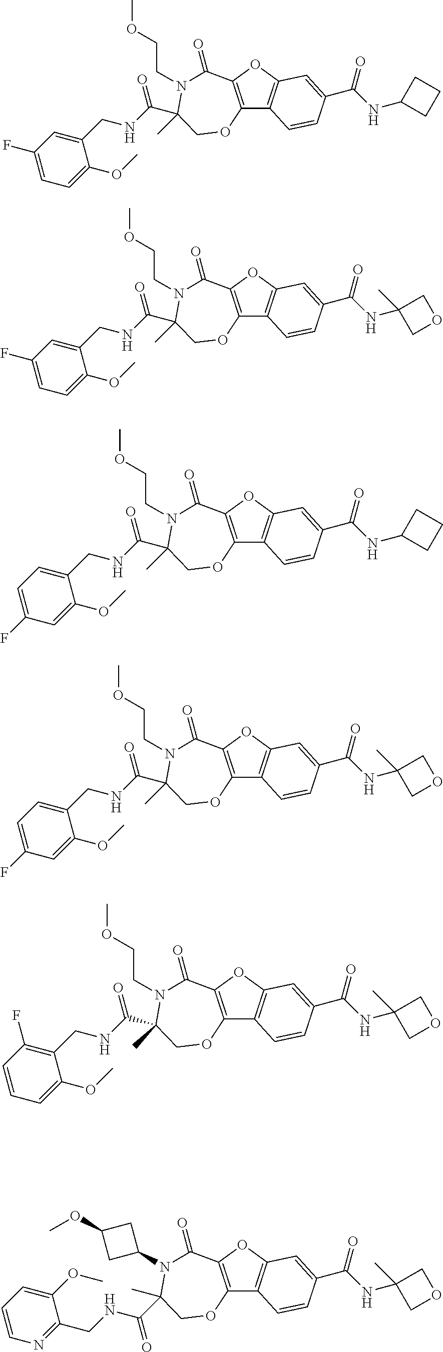

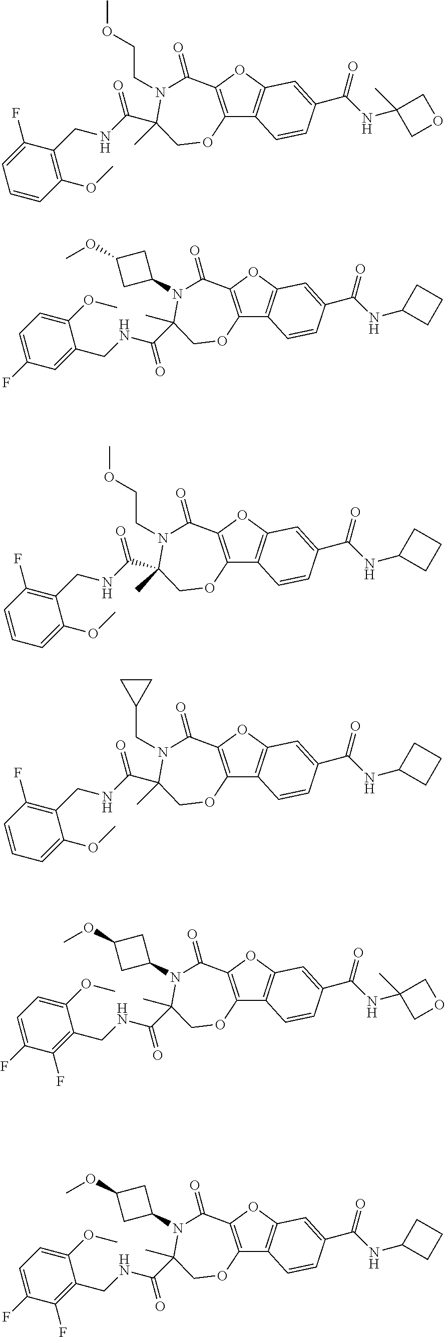 [SMILES: COCCN1C(=O)c2oc3cc(C(=O)NC4(C)COC4)ccc3c2OCC1(C)C(=O)NCc1c(F)cccc1OC ; COc1ccc(F)cc1CNC(=O)C1(C)COc2c(oc3cc(C(=O)NC4CCC4)ccc23)C(=O)N1[C@H]1C[C@H](OC)C1 ; COCCN1C(=O)c2oc3cc(C(=O)NC4CCC4)ccc3c2OC[C@]1(C)C(=O)NCc1c(F)cccc1OC ; COc1cccc(F)c1CNC(=O)C1(C)COc2c(oc3cc(C(=O)NC4CCC4)ccc23)C(=O)N1CC1CC1 ; COc1ccc(F)c(F)c1CNC(=O)C1(C)COc2c(oc3cc(C(=O)NC4(C)COC4)ccc23)C(=O)N1[C@H]1C[C@@H](OC)C1 ; COc1ccc(F)c(F)c1CNC(=O)C1(C)COc2c(oc3cc(C(=O)NC4CCC4)ccc23)C(=O)N1[C@H]1C[C@@H](OC)C1]

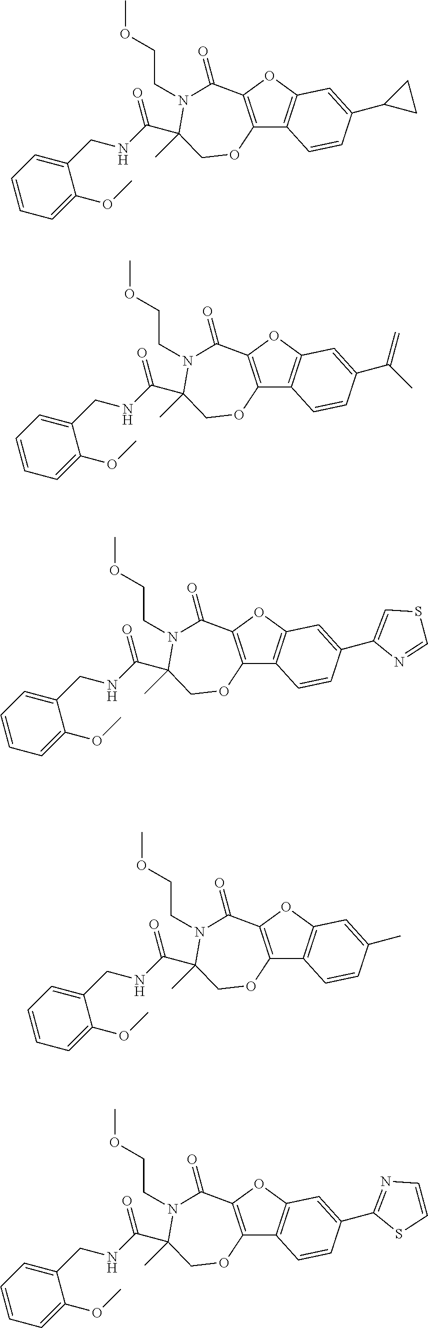 [SMILES: COCCN1C(=O)c2oc3cc(C4CC4)ccc3c2OCC1(C)C(=O)NCc1ccccc1OC ; C=C(C)c1ccc2c3c(oc2c1)C(=O)N(CCOC)C(C)(C(=O)NCc1ccccc1OC)CO3 ; COCCN1C(=O)c2oc3cc(-c4cscn4)ccc3c2OCC1(C)C(=O)NCc1ccccc1OC ; COCCN1C(=O)c2oc3cc(C)ccc3c2OCC1(C)C(=O)NCc1ccccc1OC ; COCCN1C(=O)c2oc3cc(-c4nccs4)ccc3c2OCC1(C)C(=O)NCc1ccccc1OC]

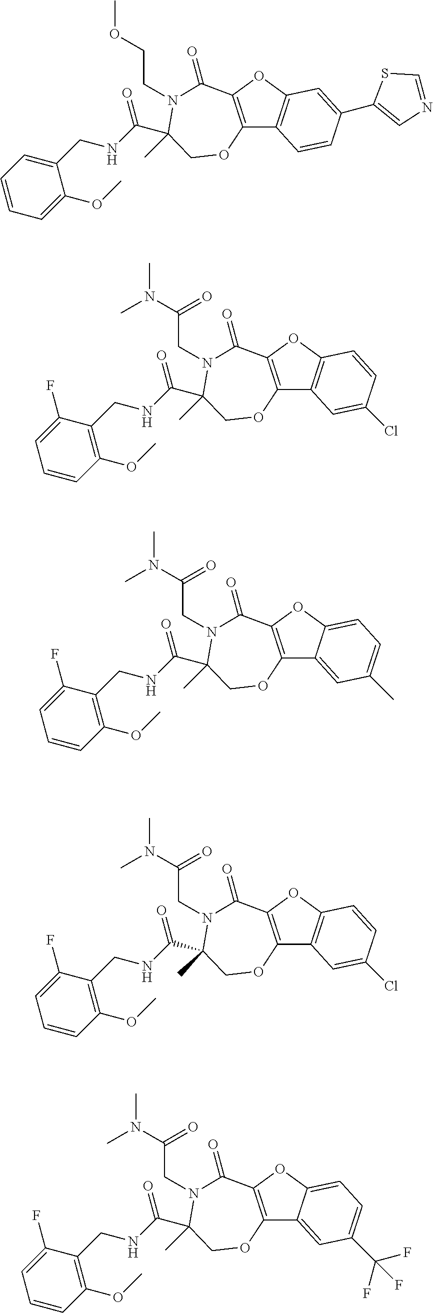 [SMILES: COCCN1C(=O)c2oc3cc(-c4cncs4)ccc3c2OCC1(C)C(=O)NCc1ccccc1OC ; COc1cccc(F)c1CNC(=O)C1(C)COc2c(oc3ccc(Cl)cc23)C(=O)N1CC(=O)N(C)C ; COc1cccc(F)c1CNC(=O)C1(C)COc2c(oc3ccc(C)cc23)C(=O)N1CC(=O)N(C)C ; COc1cccc(F)c1CNC(=O)[C@@]1(C)COc2c(oc3ccc(Cl)cc23)C(=O)N1CC(=O)N(C)C ; COc1cccc(F)c1CNC(=O)C1(C)COc2c(oc3ccc(C(F)(F)F)cc23)C(=O)N1CC(=O)N(C)C]

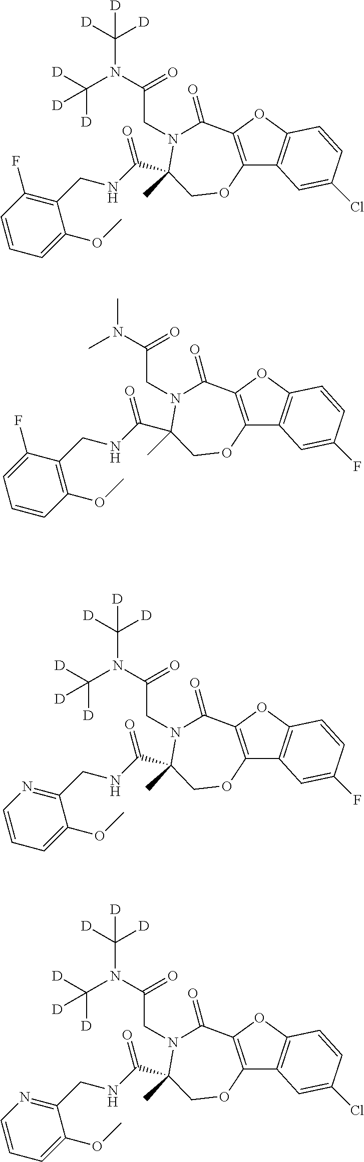 [SMILES: [2H]C([2H])([2H])N(C(=O)CN1C(=O)c2oc3ccc(Cl)cc3c2OC[C@]1(C)C(=O)NCc1c(F)cccc1OC)C([2H])([2H])[2H] ; COc1cccc(F)c1CNC(=O)C1(C)COc2c(oc3ccc(F)cc23)C(=O)N1CC(=O)N(C)C ; [2H]C([2H])([2H])N(C(=O)CN1C(=O)c2oc3ccc(F)cc3c2OC[C@]1(C)C(=O)NCc1ncccc1OC)C([2H])([2H])[2H] ; [2H]C([2H])([2H])N(C(=O)CN1C(=O)c2oc3ccc(Cl)cc3c2OC[C@]1(C)C(=O)NCc1ncccc1OC)C([2H])([2H])[2H]]

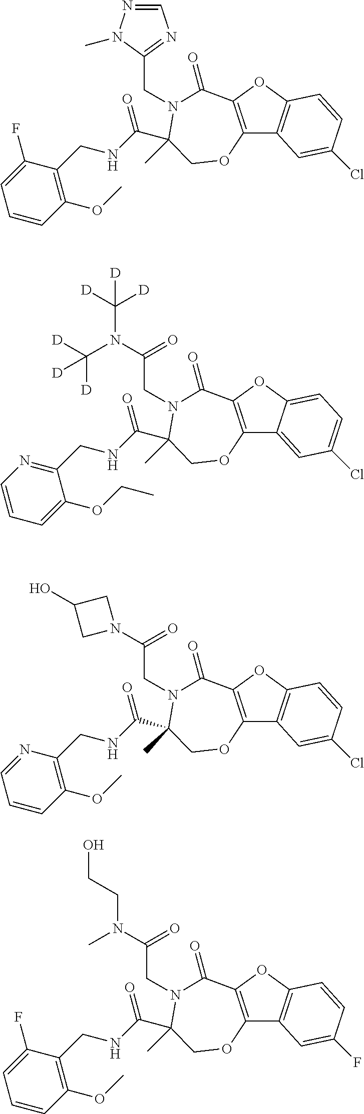 [SMILES: COc1cccc(F)c1CNC(=O)C1(C)COc2c(oc3ccc(Cl)cc23)C(=O)N1Cc1ncnn1C ; [2H]C([2H])([2H])N(C(=O)CN1C(=O)c2oc3ccc(Cl)cc3c2OCC1(C)C(=O)NCc1ncccc1OCC)C([2H])([2H])[2H] ; COc1cccnc1CNC(=O)[C@@]1(C)COc2c(oc3ccc(Cl)cc23)C(=O)N1CC(=O)N1CC(O)C1 ; COc1cccc(F)c1CNC(=O)C1(C)COc2c(oc3ccc(F)cc23)C(=O)N1CC(=O)N(C)CCO]

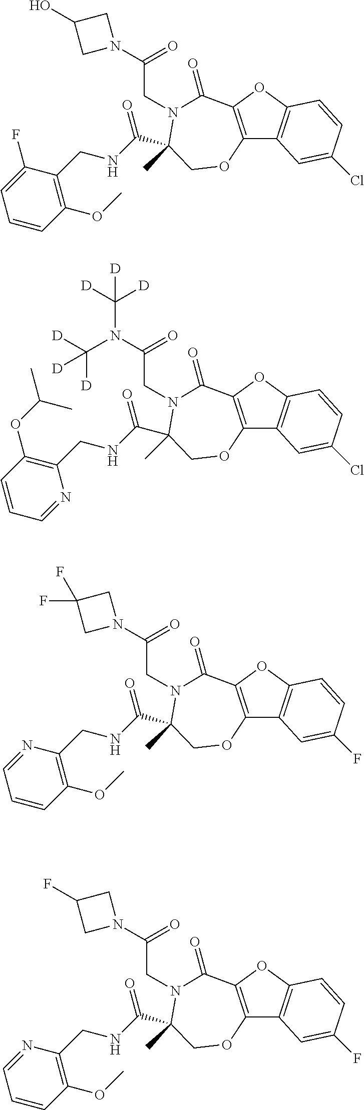 [SMILES: COc1cccc(F)c1CNC(=O)[C@@]1(C)COc2c(oc3ccc(Cl)cc23)C(=O)N1CC(=O)N1CC(O)C1 ; [2H]C([2H])([2H])N(C(=O)CN1C(=O)c2oc3ccc(Cl)cc3c2OCC1(C)C(=O)NCc1ncccc1OC(C)C)C([2H])([2H])[2H] ; COc1cccnc1CNC(=O)[C@@]1(C)COc2c(oc3ccc(F)cc23)C(=O)N1CC(=O)N1CC(F)(F)C1 ; COc1cccnc1CNC(=O)[C@@]1(C)COc2c(oc3ccc(F)cc23)C(=O)N1CC(=O)N1CC(F)C1]

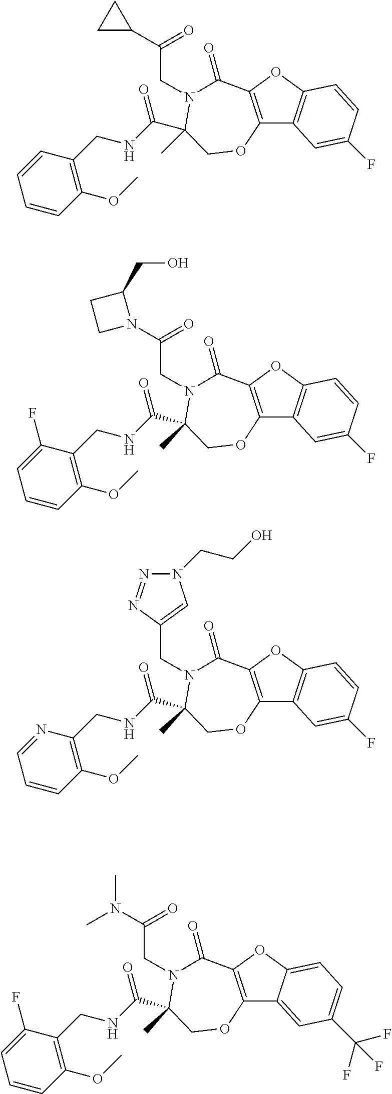 [SMILES: COc1ccccc1CNC(=O)C1(C)COc2c(oc3ccc(F)cc23)C(=O)N1CC(=O)C1CC1 ; COc1cccc(F)c1CNC(=O)[C@@]1(C)COc2c(oc3ccc(F)cc23)C(=O)N1CC(=O)N1CC[C@H]1CO ; COc1cccnc1CNC(=O)[C@@]1(C)COc2c(oc3ccc(F)cc23)C(=O)N1Cc1cn(CCO)nn1 ; COc1cccc(F)c1CNC(=O)[C@@]1(C)COc2c(oc3ccc(C(F)(F)F)cc23)C(=O)N1CC(=O)N(C)C]

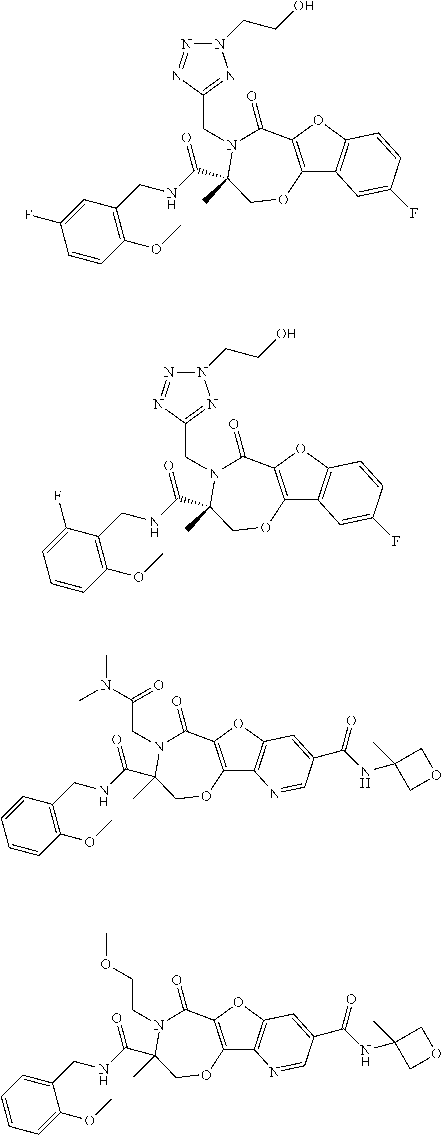 [SMILES: COc1ccc(F)cc1CNC(=O)[C@@]1(C)COc2c(oc3ccc(F)cc23)C(=O)N1Cc1nnn(CCO)n1 ; COc1cccc(F)c1CNC(=O)[C@@]1(C)COc2c(oc3ccc(F)cc23)C(=O)N1Cc1nnn(CCO)n1 ; COc1ccccc1CNC(=O)C1(C)COc2c(oc3cc(C(=O)NC4(C)COC4)cnc23)C(=O)N1CC(=O)N(C)C ; COCCN1C(=O)c2oc3cc(C(=O)NC4(C)COC4)cnc3c2OCC1(C)C(=O)NCc1ccccc1OC]

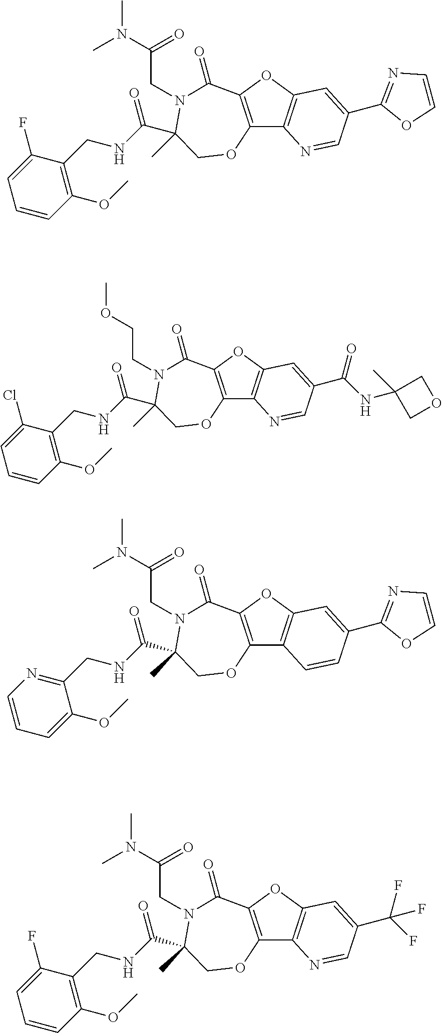 [SMILES: COc1cccc(F)c1CNC(=O)C1(C)COc2c(oc3cc(-c4ncco4)cnc23)C(=O)N1CC(=O)N(C)C ; COCCN1C(=O)c2oc3cc(C(=O)NC4(C)COC4)cnc3c2OCC1(C)C(=O)NCc1c(Cl)cccc1OC ; COc1cccnc1CNC(=O)[C@@]1(C)COc2c(oc3cc(-c4ncco4)ccc23)C(=O)N1CC(=O)N(C)C ; COc1cccc(F)c1CNC(=O)[C@@]1(C)COc2c(oc3cc(C(F)(F)F)cnc23)C(=O)N1CC(=O)N(C)C]

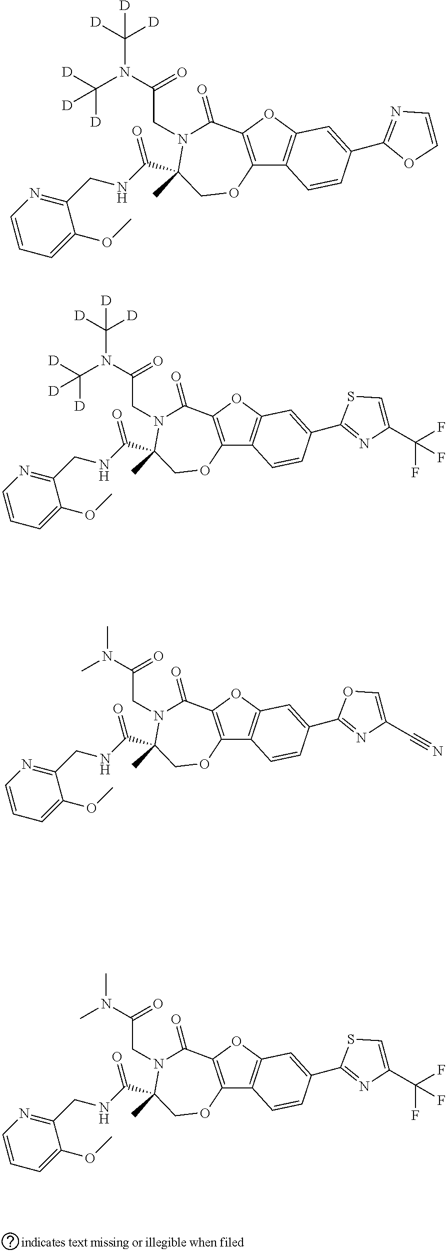 [SMILES: [2H]C([2H])([2H])N(C(=O)CN1C(=O)c2oc3cc(-c4ncco4)ccc3c2OC[C@]1(C)C(=O)NCc1ncccc1OC)C([2H])([2H])[2H] ; [2H]C([2H])([2H])N(C(=O)CN1C(=O)c2oc3cc(-c4nc(C(F)(F)F)cs4)ccc3c2OC[C@]1(C)C(=O)NCc1ncccc1OC)C([2H])([2H])[2H] ; COc1cccnc1CNC(=O)[C@@]1(C)COc2c(oc3cc(-c4nc(C#N)co4)ccc23)C(=O)N1CC(=O)N(C)C ; COc1cccnc1CNC(=O)[C@@]1(C)COc2c(oc3cc(-c4nc(C(F)(F)F)cs4)ccc23)C(=O)N1CC(=O)N(C)C]

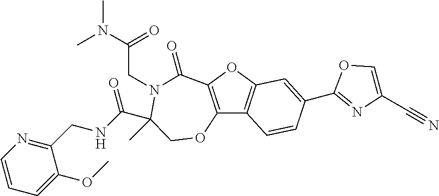 [SMILES: COc1cccnc1CNC(=O)C1(C)COc2c(oc3cc(-c4nc(C#N)co4)ccc23)C(=O)N1CC(=O)N(C)C]

or a pharmaceutically acceptable salt thereof.

### BRIEF DESCRIPTION OF THE DRAWINGS

FIG. 1 is a Molecular Stereoscopic Structure Ellipsoid Diagram for an exemplary compound of the invention.

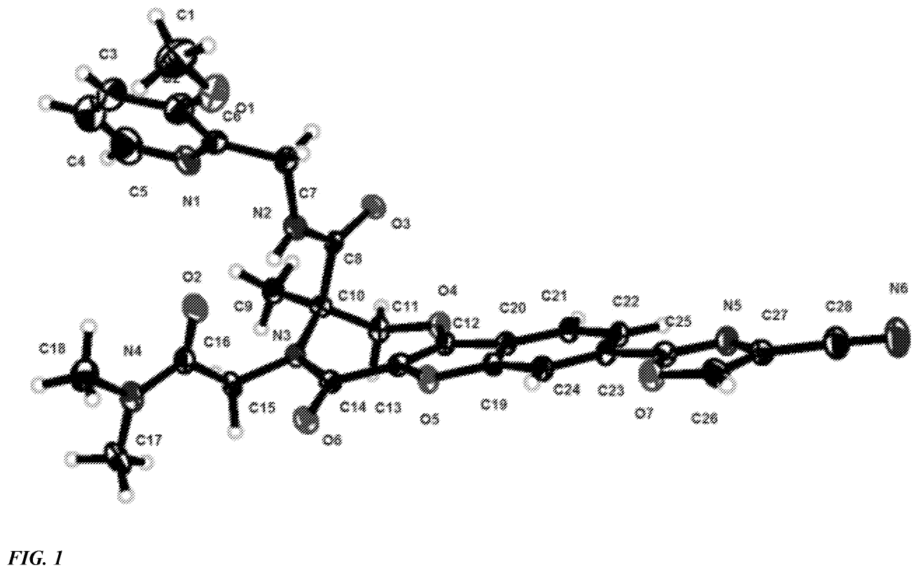

### DETAILED DESCRIPTION

In some embodiments, the invention provides a compound of formula I:

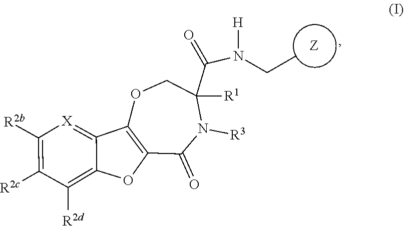

or a pharmaceutically acceptable salt thereof, wherein:
R¹ represents lower alkyl;
X represents C—R^{2a} or N;
R^{2a}, R^{2b}, R^{2c}, and R^{2d} each independently represent hydrogen, alkyl, alkenyl, alkynyl, halo, aryl, heteroaryl, cycloalkyl, heterocyclyl, cyano, acyl, carboxy, ester, or amido;
R³ represents alkyl, alkenyl, alkynyl, aryl, heteroaryl, cycloalkyl, heterocyclyl, arylalkyl, heteroaralkyl, (cycloalkyl)alkyl, heterocyclylalkyl, amidoalkyl, alkoxyalkyl, or acylalkyl; and
Z represents a substituted or unsubstituted aryl or heteroaryl ring, e.g., optionally substituted with one or more groups chosen from alkyl, alkenyl, alkynyl, cyano, acyl, carboxy, ester, amido, alkoxy, and halo.

In certain embodiments, R¹ is methyl.

In certain embodiments, X is N. In other embodiments, X is C—R^{2a}.

In certain embodiments, R^{2a}, R^{2b}, R^{2c}, and R^{2d} independently represent hydrogen, methyl, propenyl, chloro, fluoro, haloalkyl (e.g., trifluoromethyl), a five-membered heteroaryl, cyclopropyl, or amido having the structure:

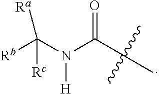

In certain such embodiments, R^{a} is hydrogen or alkyl, and R^{b} and R^{c} taken together form a cycloalkyl or heterocyclyl, e.g., cyclobutyl. In certain embodiments, R^{a} is hydrogen or methyl, preferably methyl. In certain embodiments, R^{b} and R^{c} taken together form oxetane.

In certain embodiments, at least one of R^{2a}, R^{2b}, R^{2c}, and R^{2d} is the 5-membered heteroaryl, such as thiazolyl or oxazolyl, optionally substituted with trifluoromethyl, chloro, or cyano. In certain such embodiments, the 5-membered heteroaryl is oxazol-2-yl, e.g., 4-cyanooxazol-2-yl.

In certain embodiments, R^{2a} is hydrogen.

In certain embodiments, R^{2b} and R^{2d} are each hydrogen.

In certain embodiments, R^{2c} and R^{2d} are each hydrogen

In certain embodiments, R^{2b}, R^{2c}, and R^{2d} are each hydrogen.

In certain preferred embodiments, if X is C—R^{2a}, at least one of R^{2a}, R^{2b}, R^{2c}, and R^{2d} is not hydrogen, such that the benzofuran ring has at least one non-hydrogen substitution. For example in certain embodiments, if X is C—H, then preferably one of R^{2b}, R^{2c}, and R^{2d} is not hydrogen. In certain of these preferred embodiments, R^{2c} is the non-hydrogen substitution and is preferably a 5-membered heteroaryl such as oxazolyl, which is optionally substituted.

In certain embodiments, if X is C—R^{2a}, then R^{2a}, R^{2b}, R^{2c}, and R^{2d} are each hydrogen.

In certain preferred embodiments, R³ is amidoalkyl, such as an amidoalkyl having the structure:

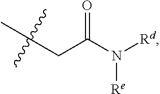

wherein R^{d} and R^{e} are independently chosen from alkyl or hydroxyalkyl, preferably alkyl (e.g., methyl), or R^{d} and R^{e} taken together form a heterocyclic ring. In certain such embodiments, R^{d} and R^{e} are each methyl. In certain embodiments, R^{d} and R^{e} are independently enriched for deuterium at one or more hydrogen-bearing sites. For example, R^{d} and/or R^{e} may contain a non-natural abundance of deuterium, preferably wherein a hydrogen position is at least 15%, at least 25%, at least 50%, at least 60%, at least 75%, or at least 80% deuterium. In certain embodiments, R^{d} is methyl and R^{e} is —(CH₂)₂OH.

In certain embodiments, R^{d} and R^{e} taken together with the nitrogen to which they are attached form an azetidine optionally substituted with one or more halo, hydroxyl, or hydroxyalkyl, for example:

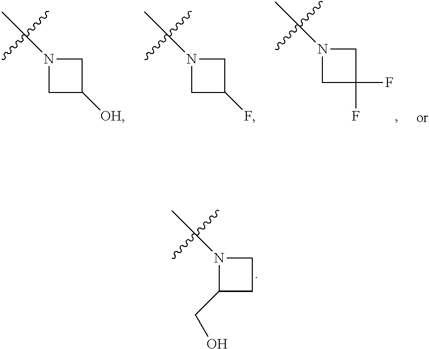 [SMILES: *N1CC(O)C1 ; *N1CC(F)C1 ; *N1CC(F)(F)C1 ; *N1CCC1CO]

In certain embodiments, R³ is C₃-C₆ cycloalkyl, for example:

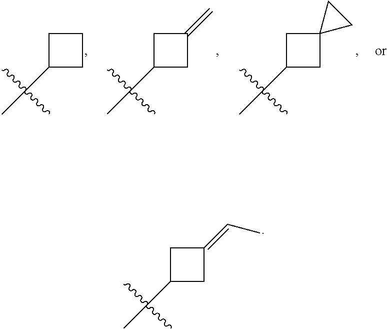

In certain embodiments, R³ is C₁-C₆ alkyl (including, for example, C₁-C₂ alkyl or C₃-C₆ alkyl), C₂-C₆ alkenyl, or C₂-C₆ alkynyl, optionally substituted with alkoxy, for example:

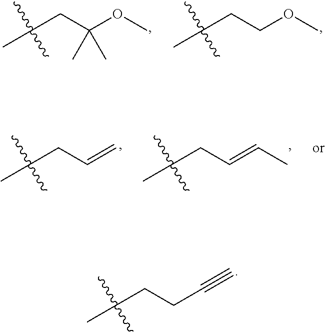

In certain embodiments, R³ is —CH₂-cycloalkyl optionally substituted with halo, alkoxy, or hydroxyl, for example:

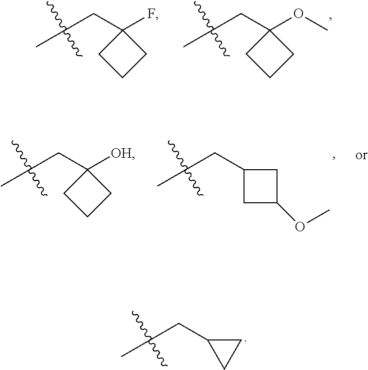

In certain embodiments, R³ is acylalkyl having the structure:

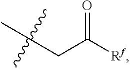

wherein R^{f} represents alkyl or cycloalkyl, e.g., ethyl or cyclopropyl.

In certain embodiments, R³ represents —(CH₂)₁₋₃-heteroaryl, optionally substituted with alkyl, hydroxyalkyl or alkoxyalkoxyalkyl. In certain embodiments, the heteroaryl is tetrazole, 1,2,3-triazole, or 1,2,4-triazole. In certain embodiments, R³ is:

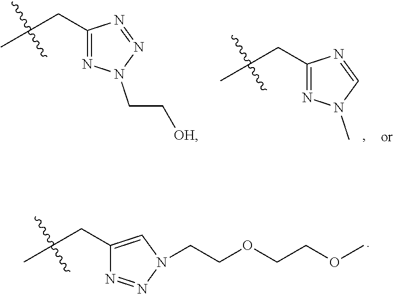

In certain embodiments, Z is phenyl, pyridinyl, naphthyl, isoquinolinyl, or quinolinyl, preferably pyridyl, each of which is optionally substituted with one or more groups chosen from lower alkyl, lower alkoxy, halo, haloalkoxy, amido, and cyano. In certain embodiments, Z is substituted with one or more groups, or for example at least two groups, chosen from methoxy, isopropyloxy, chloro, fluoro, trifluoromethoxy, cyano, and carbamoyl. In certain embodiments, Z is phenyl substituted with methoxy and at least one additional substituent.

In certain preferred embodiments, Z is pyridyl optionally substituted with an alkoxy (e.g., methoxy). In certain embodiments, Z is mono-, di-, or trisubstituted. In certain embodiments, Z is:

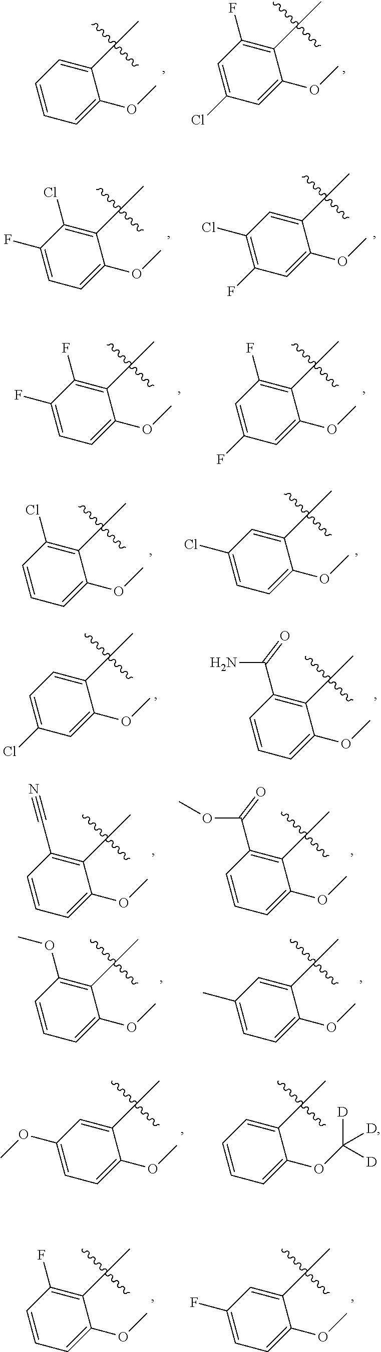

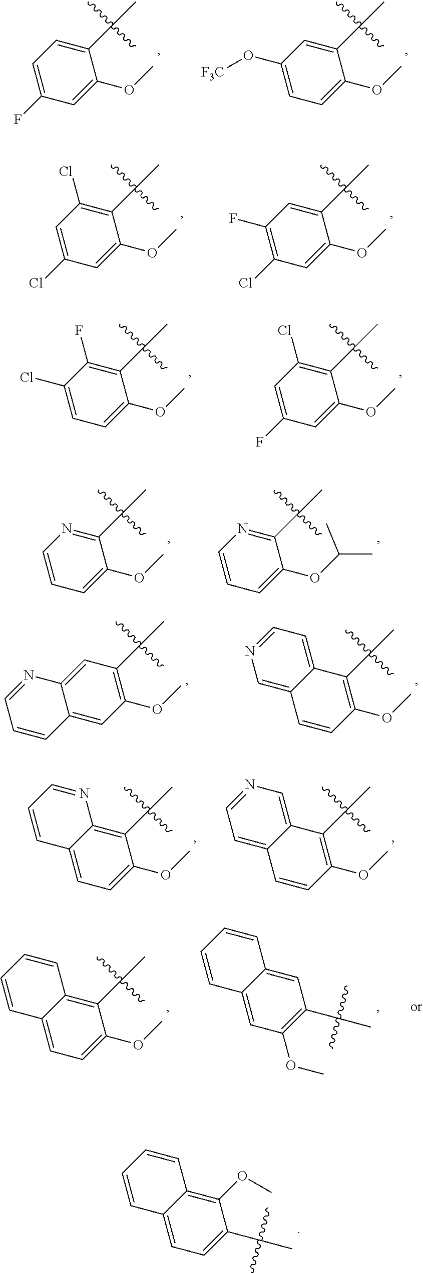

In certain embodiments, the compound of formula (I) is not:

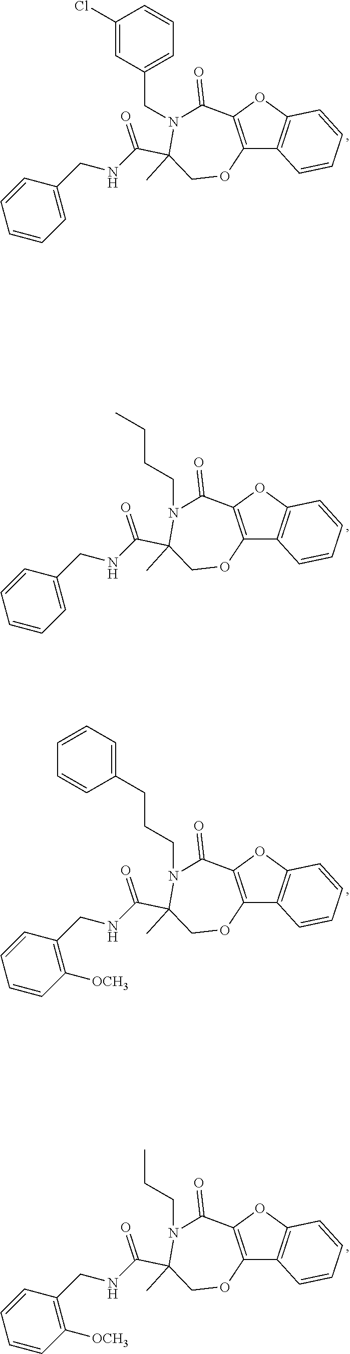 [SMILES: CC1(C(=O)NCc2ccccc2)COc2c(oc3ccccc23)C(=O)N1Cc1cccc(Cl)c1 ; CCCCN1C(=O)c2oc3ccccc3c2OCC1(C)C(=O)NCc1ccccc1 ; COc1ccccc1CNC(=O)C1(C)COc2c(oc3ccccc23)C(=O)N1CCCc1ccccc1 ; CCCN1C(=O)c2oc3ccccc3c2OCC1(C)C(=O)NCc1ccccc1OC]

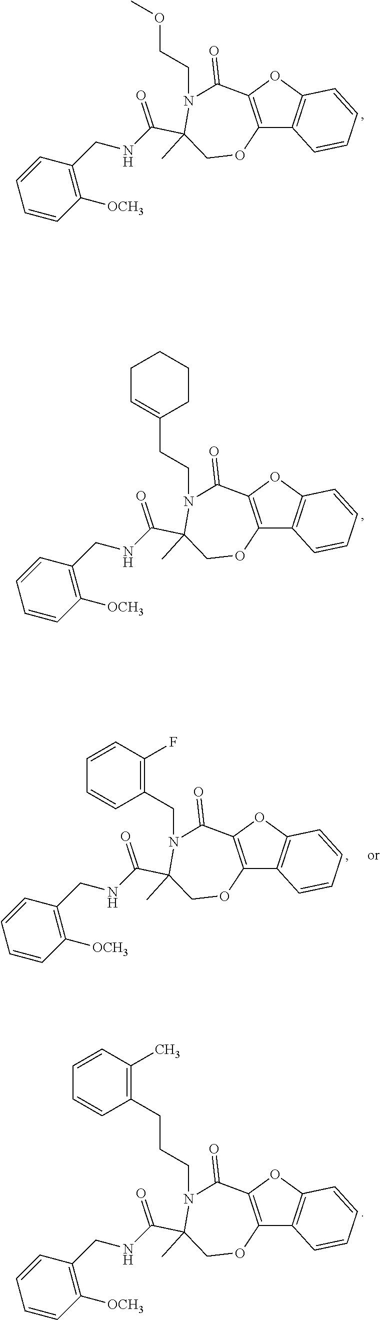 [SMILES: COCCN1C(=O)c2oc3ccccc3c2OCC1(C)C(=O)NCc1ccccc1OC ; COc1ccccc1CNC(=O)C1(C)COc2c(oc3ccccc23)C(=O)N1CCC1=CCCCC1 ; COc1ccccc1CNC(=O)C1(C)COc2c(oc3ccccc23)C(=O)N1Cc1ccccc1F ; COc1ccccc1CNC(=O)C1(C)COc2c(oc3ccccc23)C(=O)N1CCCc1ccccc1C]

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known. The ability of such agents to induce KLF2 and/or vasoprotection may render them suitable as “therapeutic agents” in the methods and compositions of this disclosure.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

The present disclosure further includes isotopically-labeled compounds of the disclosure. An “isotopically” or “radio-labeled” compound is a compound of the disclosure where one or more atoms are replaced or substituted by an atom having an atomic mass or mass number different from the atomic mass or mass number typically found in nature (i.e., naturally occurring). Suitable radionuclides that may be incorporated in compounds of the present disclosure include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. For example, one or more protium (H) atoms in a compound of the present disclosure can be replaced by deuterium atoms (e.g., one or more hydrogen atoms of a C₁₋₆ alkyl group of Formula (I) can be enriched with deuterium atoms, e.g., enriched for —CD₃ in place of a more naturally abundant —C(¹H)₃ methyl group).

In certain embodiments of the compounds disclosed herein, certain atoms may be isotopically enriched, e.g., for radioisotopic labelling or for a metabolically beneficial isotope effect (e.g., by isotopically enriching for deuterium at a hydrogen substituent). In such embodiments, the compound may be isotopically enriched for the desired isotope such that at least 15%, at least 25%, at least 50%, at least 60%, at least 75%, at least 80%, or even at least 90% more of the molecules of the compound in the composition have the desired isotope at the indicated position.

In some embodiments, the compound is enriched for two or more deuterium atoms. In some embodiments, the compound includes 1, 2, 3, 4, 5, 6, 7, 8, 9, 10, 11, 12, 13, 14, 15, 16, 17, 18, 19, 20, 21, 22, 23, or 24 deuterium atoms. In some embodiments, all of the hydrogen atoms in a compound can be enriched for deuterium atoms instead of protium atoms.

Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can used in various studies such as NMR spectroscopy, metabolism experiments, and/or assays.

Substitution with heavier isotopes, such as deuterium for protium, may afford certain therapeutic advantages resulting from greater metabolic stability, for example, increased in vivo half-life or reduced dosage requirements, and hence may be preferred in some circumstances. (see, e.g., A. Kerekes et.al. J. Med. Chem. 2011, 54, 201-210; R. Xu et.al. J. Label Compd. Radiopharm. 2015, 58, 308-312).

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present invention can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, heteroaryl, cycloalkyl, heterocyclyl, amino, aminoalkyl, cyano, haloalkyl, haloalkoxy, —OCO—CH₂—O-alkyl, —OP(O)(O-alkyl)₂ or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

As used herein, the term “alkyl” refers to saturated aliphatic groups, including but not limited to C₁-C₁₀ straight-chain alkyl groups or C₁-C₁₀ branched-chain alkyl groups. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x toy carbons in the chain. C₀alkyl indicates a hydrogen where the group is in a terminal position, a bond if internal. A C₁₋₆ alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amide”, as used herein, refers to a group

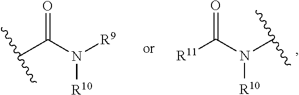

wherein R⁹, R¹⁰, and R¹¹, each independently represent a hydrogen or hydrocarbyl group, or R⁹ and R¹⁰ taken together with the N atom to which they are attached complete a heterocycle having from 4 to 8 atoms in the ring structure, or R¹⁰ and R¹¹ taken together with the N atom to which they are attached complete a heterocycle having from 4 to 8 atoms in the ring structure.

The term “amidino”, as used herein, refers to a group

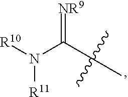

wherein R⁹, R¹⁰, and R¹¹, each independently represent a hydrogen or hydrocarbyl group, or R⁹ and R¹⁰ taken together with the N atom to which they are attached complete a heterocycle having from 4 to 8 atoms in the ring structure, or R¹⁰ and R¹¹ taken together with the N atom to which they are attached complete a heterocycle having from 4 to 8 atoms in the ring structure.

The term “amido”, as used herein, refers to a group

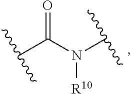

wherein R¹⁰ represents a hydrogen or hydrocarbyl group.

The terms “amine” and “amino” are art-recognized and refer to both unsubstituted and substituted amines and salts thereof, e.g., a moiety that can be represented by

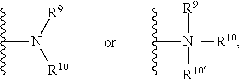

wherein R⁹, R¹⁰, and R¹⁰′ each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “amidoalkyl”, as used herein, refers to an alkyl group substituted with an amido group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term“azido” is art-recognized and refers to the group —N₃.

The term “carbamate” is art-recognized and refers to a group

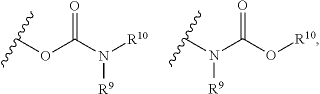

wherein R⁹ and R¹⁰ independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbocyclylalkyl”, as used herein, refers to an alkyl group substituted with a carbocycle group.

The term “carbonate” is art-recognized and refers to a group —OCO₂—.

The term “carboxy”, as used herein, refers to a group represented by the formula —CO₂H.

A “cycloalkyl” group is a cyclic hydrocarbon which is completely saturated. “Cycloalkyl” includes monocyclic and bicyclic rings. Typically, a monocyclic cycloalkyl group has from 3 to about 10 carbon atoms, more typically 3 to 8 carbon atoms unless otherwise defined. The second ring of a bicyclic cycloalkyl may be selected from saturated, unsaturated and aromatic rings. Cycloalkyl includes bicyclic molecules in which one, two or three or more atoms are shared between the two rings. The term “fused cycloalkyl” refers to a bicyclic cycloalkyl in which each of the rings shares two adjacent atoms with the other ring. The second ring of a fused bicyclic cycloalkyl may be selected from saturated, unsaturated and aromatic rings. A “(cycloalkyl)alkyl” group is a cycloalkyl attached to an alkyl group.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical.

Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The term “haloalkyl” as used herein refers to an alkyl group wherein one or more hydrogens is replaced with a halogen.

The term “haloalkoxy” as used herein refers to an alkoxy group in which one or more hydrogen atoms is replaced with a halogen atom.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, tetrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms.

Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “imine” is art-recognized and refers to a group

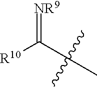

wherein R⁹ is a hydrogen or a hydrocarbyl group, and R¹⁰ represents a hydrocarbyl group, or R⁹ and R¹⁰ taken together with the N atom to which R⁹ is attached complete a heterocycle having from 4 to 8 atoms in the ring structure.

The term “lower” when used in conjunction with a chemical moiety, such as, acyl, acyloxy, alkyl, alkenyl, alkynyl, or alkoxy is meant to include groups where there are ten or fewer atoms in the substituent, exclusive of hydrogen atoms, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The term “oxime” is art recognized and refers to the group

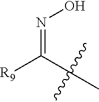

wherein R⁹ represents hydrogen or a hydrocarbyl group.

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”.

Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamide” is art-recognized and refers to the group represented by the general formulae

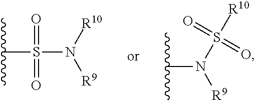

wherein R⁹ and R¹⁰ independently represents hydrogen or hydrocarbyl.

The term “sulfoxide” is art-recognized and refers to the group-S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds.

The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR⁹ or —SC(O)R⁹ wherein R⁹ represents a hydrocarbyl.

The term “thioether”, as used herein, is equivalent to an ether, wherein the oxygen is replaced with a sulfur.

The term “urea” is art-recognized and may be represented by the general formula

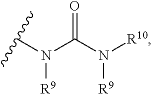

wherein R⁹ and R¹⁰ independently represent hydrogen or a hydrocarbyl.

The term “modulate” as used herein includes the inhibition or suppression of a function or activity (such as cell proliferation) as well as the enhancement of a function or activity.

The term “induce” as used herein includes the promotion of a function, activity, or expression of a particular protein or enzyme. In certain embodiments, a compound disclosed herein induces KLF2, e.g., increases the expression of KLF2. KLF2 induction may be measured according to techniques known to those skilled in the art, such as a cell-based assay.

For example, KLF2 induction can be measured according to the Example described hereinbelow.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds represented by Formula I. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of Formula I are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of Formula I for laboratory use, or for subsequent conversion to a pharmaceutically acceptable acid addition salt.

The term “pharmaceutically acceptable basic addition salt” as used herein means any non-toxic organic or inorganic base addition salt of any acid compounds represented by Formula I or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixture and separate individual isomers.

Some of the compounds may also exist in tautomeric forms. Such forms, although not explicitly indicated in the formulae described herein, are intended to be included within the scope of the present disclosure.

“Prodrug” or “pharmaceutically acceptable prodrug” refers to a compound that is metabolized, for example hydrolyzed or oxidized, in the host after administration to form the compound of the present disclosure (e.g., compounds of formula I). Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. Nos. 6,875,751, 7,585,851, and 7,964,580, the disclosures of which are incorporated herein by reference. The prodrugs of this disclosure are metabolized to produce a compound of Formula I. The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

The term “Log of solubility”, “Log S” or “log S” as used herein is used in the art to quantify the aqueous solubility of a compound. The aqueous solubility of a compound significantly affects its absorption and distribution characteristics. A low solubility often goes along with a poor absorption. Log S value is a unit stripped logarithm (base 10) of the solubility measured in mol/liter.

### Pharmaceutical Compositions

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment. A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a selfemulsifying drug delivery system or a selfmicroemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

In some instances, the pharmaceutical composition may be a solid dispersion. The term “solid dispersion” refers to a system in a solid state comprising at least two components, wherein one component is dispersed throughout the other component or components. For example, the solid dispersion can be an amorphous solid dispersion. The term “amorphous solid dispersion” as used herein, refers to stable solid dispersions comprising an amorphous drug substance and a polymer. By “amorphous drug substance,” it is meant that the amorphous solid dispersion contains drug substance in a substantially amorphous solid state form.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof, (10) complexing agents, such as, modified and unmodified cyclodextrins; (11) a biocompatible polymer, such as those used to make amorphous solid dispersions, and (12) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in microencapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

In certain embodiments, compounds of the invention may be used alone or conjointly administered with another type of therapeutic agent.

The present disclosure includes the use of pharmaceutically acceptable salts (see Berge et al. (1977) “Pharmaceutical Salts”, J. Pharm. Sci. 66: 1-19.) of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benethamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine

### Methods of Treatment

Provided herein are methods of treating an inflammatory disease or endothelial dysfunction comprising administering a therapeutically effective amount of a compound of the invention, such as a compound of formula (I), a pharmaceutically acceptable salt thereof, or the composition comprising a compound of formula (I).

In certain embodiments, the inflammatory disease or endothelial dysfunction is atherosclerosis, coronary artery disease, stroke, peripheral arterial disease, coronary microvascular diseases, angina, systemic hypertension, pulmonary arterial hypertension, heart failure, diabetic microvascular diseases, such as diabetic nephropathy, diabetic retinopathy or diabetic neuropathy, or autoimmune, inflammatory or infectious diseases.

### EXAMPLES

TABLE
Abbreviations

| Abbreviation | Meaning |
|---|---|
| Ac | acetate |
| ACN | acetonitrile |
| br. | broad signal (NMR) |
| Boc | tert-butyloxycarbonyl |
| d | doublet (NMR) |
| dba | dibenzylideneacetone |
| DCM | dichloromethane |
| dd | doublet of doublet (NMR) |
| ddd | doublet of doublet of doublet (NMR) |
| dt | doublet of triplet (NMR) |
| DEA | diethylamine |
| DIPEA | diisopropylethylamine |
| DMA | dimethylamine |
| DMAP | dimethylaminopyridine |
| DMF | N,N-dimethylformamide |
| DMP | Dess-Martin Periodinane |
| DMSO | dimethylsulfoxide |
| DPPF | 1,1′-Bis(diphenylphosphino)ferrocene |
| EA, EtOAc | ethyl acetate |
| ee | enatiomeric excess |
| eq | equilivents |
| ESI | electrospray (ES) ionization |
| g | grams |
| h, hr (hrs) | hour(s) |
| HATU | 1-[Bis(dimethylamino)methylene]-1H-1,2,3-triazolo[4,5- |
| | b]pyridinium 3-oxide hexafluorophosphate |
| Ac | acetate |
| ACN | acetonitrile |
| HCl | hydrogen chloride, hydrochloric acid |
| HPLC | high performance liquid chromatography |
| LC-MS | liquid chromatography-mass spectrometry |
| m | multiplet (NMR) |
| M | molar |
| min | minute(s) |
| MS | mass spectrometry |
| N | normal |
| NMR | nuclear magnetic resonance spectroscopy: chemical shifts |
| | (δ) are given in ppm. |
| PE | Petroleum ether |
| Ph | phenyl |
| q | quartet (NMR) |
| qd | quartet of doublet (NMR) |
| rt | room temperature |
| Rₜ, Rt | retention time |
| s | singlet (NMR) |
| SFC | Supercritical Fluid Chromatography |
| t | triplet (NMR) |
| TEA | triethylamine |
| td | triplet of doublet (NMR) |
| TFAA | trifluoroacetic anhydride |
| THF | tetrahydrofuran |
| TLC | thin layer chromatography |
| δ | chemical shift |

### Intermediate BB-1

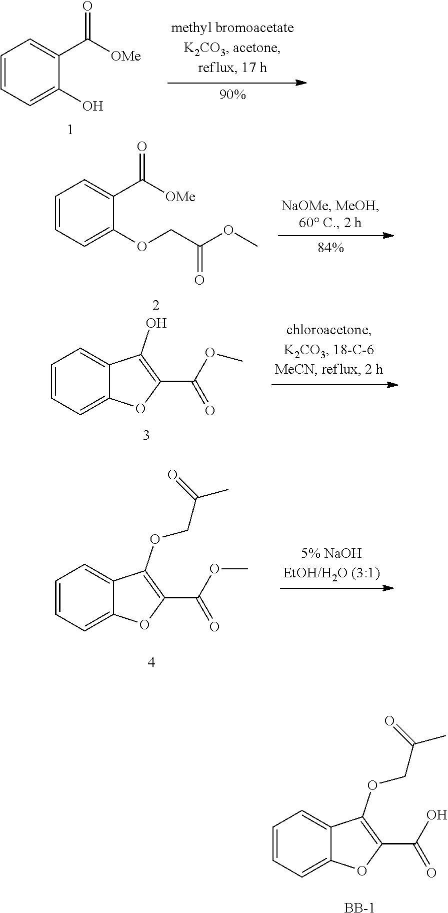

Methyl bromoacetate (11 g, 7.23 mmol, 1.1 eq) was added dropwise over 15 min to a stirred mixture of methyl 2-hydroxybenzoate (10.0 g, 6.57 mmol, 1.0 eq) and potassium carbonate (13.6 g, 9.85 mmol, 1.5 eq) in dry acetone (100 mL). The mixture was heated and stirred under reflux for 17 h. The reaction was monitored by LCMS. The reaction was cooled, and the salts were separated by filtration and washed with acetone. The filtrate was concentrated to give a residue, which was diluted with diethyl ether (200 mL), and the solution was washed successively with water, aqueous sodium carbonate, water, and brine. The crude product was distilled under reduced pressure to give methyl 2-(2-methoxy-2-oxoethoxy)benzoate (13.3 g, 90%). LC-MS: 225.1 [M+H]+

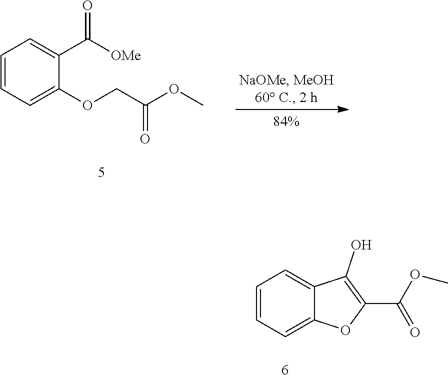

A mixture of methyl 2-(2-methoxy-2-oxoethoxy)benzoate (13.2 g, 58.9 mmol, 1.0 eq) in MeOH (100 mL) was added NaOMe (5 N in methanol, 17.7 mL) and the mixture was stirred at 60° C. for 2 h. The reaction was monitored by LCMS. The resulting mixture was cooled, poured into ice water and acidified with 5% hydrochloric acid to pH 2. The solid was collected by filtration, washed with water and recrystallized from methanol to give methyl 3-hydroxybenzofuran-2-carboxylate (9.5 g, 84%) as white solid. LC-MS: 193.1 [M+H]+

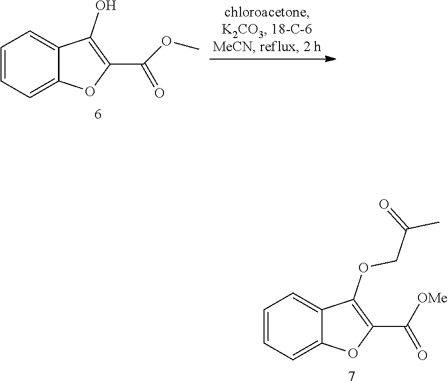

A mixture of methyl 3-hydroxybenzofuran-2-carboxylate (7.2 g, 37.5 mmol, 1.0 eq) in acetonitrile (100 mL) was added K₂CO₃ (6.21 g, 45 mmol, 1.2 eq), chloroacetone (3.82 g, 41.25 mmol, 1.1 eq) and 1,4,7,10,13,16-hexaoxacyclooctadecane (99 mg, 3.75 mmol, 0.1 eq) and the mixture was refluxed for 2 h. The reaction was monitored by LCMS. The resulting mixture was filtered, concentrated, extracted with ethyl acetate to give crude methyl 3-(2-oxopropoxy)benzofuran-2-carboxylate (9.5 g, 100%) as brown solid. LC-MS: 249.1 [M+H]+

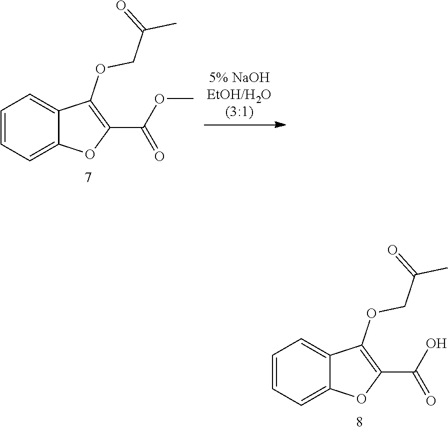

To a solution of crude methyl 3-(2-oxopropoxy)benzofuran-2-carboxylate (9.5 g, 38.3 mmol, 1.0 eq.) in EtOH (30 mL) and 5% NaOH in water (10 mL) was stirred at rt for 2 h. The reaction was monitored by LCMS. The resulting mixture was concentrated, added 1 N HCl to adjust to pH 2, extracted with ethyl acetate. The combined organic layers were washed with brine, dried over Na₂SO₄. The solvent was removed under reduced pressure and the residue was purified by flash chromatography on silica (PE:EA=2:1) to give a crude 3-(2-oxopropoxy)benzofuran-2-carboxylic acid (2.0 g, 22%) as red solid. LC-MS: 235.1 [M+H]+

### Example 1

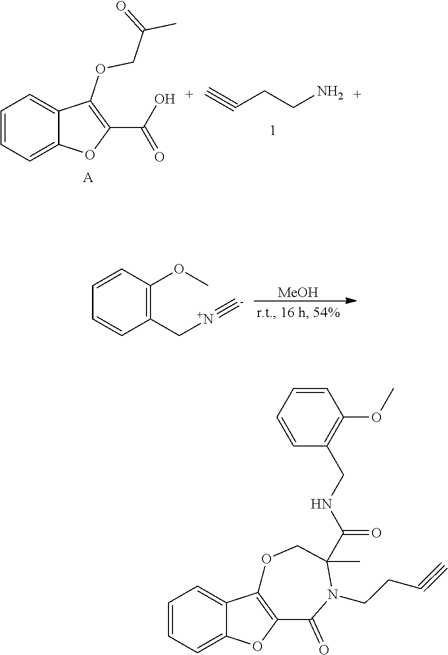

A mixture of compound 3-(2-oxopropoxy)benzofuran-2-carboxylic acid (1.0 g, 4.27 mmol, 1.0 eq), amine but-3-yn-1-amine (295 mg, 4.27 mmol, 1.0 eq), 2-methoxybenzyl isocyanide (628 mg, 4.27 mmol, 1.0 eq) in MeOH (3 mL) was stirred at room temperature for 16 h. The reaction mixture was concentrated under reduced pressure. The residue was purified by column chromatography on silica gel (Hex/EtOAc=1:1) to give example 1 (1.01 g, 54%) as a light yellow solid. LC-MS: 433.3 [M+1]⁺ ¹H NMR (400 MHz, DMSO-d₆) δ (ppm): 8.37 (s, 1H), 7.68 (ddd, J=7.8, 1.3, 0.7 Hz, 1H), 7.60 (dt, J=8.4, 0.9 Hz, 1H), 7.52 (ddd, J=8.4, 7.1, 1.3 Hz, 1H), 7.34 (ddd, J=7.9, 7.1, 0.9 Hz, 1H), 7.04 (d, J=7.8 Hz, 1H), 6.84 (d, J=8.1 Hz, 1H), 6.74 (s, 1H), 6.33 (s, 1H), 4.92 (d, J=12.6 Hz, 1H), 4.40 (d, J=12.5 Hz, 1H), 4.18 (d, J=5.7 Hz, 2H), 3.71 (s, 5H), 3.48 (brs, 1H), 2.90 (s, 1H), 1.70 (s, 4H).

### (1-fluorocyclobutyl)methanamine

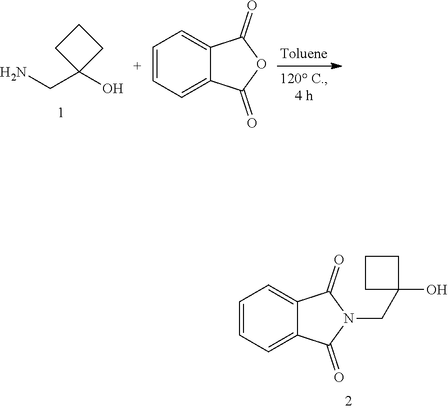

(10 mL) was added phthalic anhydride (1.5 g, 10.1 mmol, 1.02 eq). The reaction mixture was heated to 120° C. and stirred for 4 h. The reaction mixture was concentrated under reduced pressure and purified by column chromatography on silica gel (PE/EA, 50:1 to 4:1) to give compound 2-((1-hydroxycyclobutyl)methyl)isoindoline-1,3-dione (2.0 g, 87%) as a white solid. TLC: PE/EA=4:1, UV; R_{f} (compound 2)=0.40; LC-MS: 254.1 [M+Na]⁺; ¹H NMR (400 MHz, DMSO-d₆) δ (ppm): 7.96-7.81 (m, 4H), 5.09 (s, 1H), 3.68 (s, 2H), 2.21-2.11 (m, 2H), 1.96-1.85 (m, 2H), 1.75-1.62 (m, 1H), 1.58-1.43 (m, 1H).

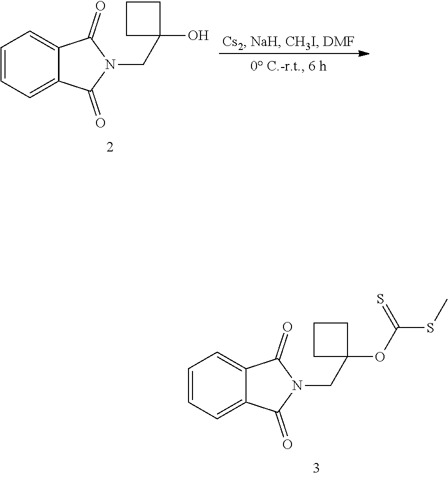 [SMILES: O=C1c2ccccc2C(=O)N1CC1(O)CCC1 ; CSC(=S)OC1(CN2C(=O)c3ccccc3C2=O)CCC1]

To a solution of 2-((1-hydroxycyclobutyl)methyl)isoindoline-1,3-dione (2.0 g, 8.6 mmol, 1.0 eq) in DMF (20 mL) was added NaH (60%, 520 mg, 12.9 mmol, 1.5 eq) at 0° C. The reaction mixture was stirred for 1 h. CS₂ (1.3 g, 17.2 mmol, 2.0 eq) was added to the mixture at 0° C. and the reaction mixture was stirred for 2 h. The mixture was added CH₃I (1.8 g, 12.9 mmol, 1.5 eq) 0° C. and was stirred at room temperature for 3 h. The mixture was diluted with 1N HCl (50 mL) extracted with EtOAc (3×50 mL). The combined organic layers were washed with brine (50 mL), dried over Na₂SO₄, filtered, and concentrated under reduced pressure. The crude product was purified by column chromatography on silica gel (PE/EtOAc=50:1 to 10:1) to give O-(1-((1,3-dioxoisoindolin-2-yl)methyl)cyclobutyl) S-methyl carbonodithioate (1.7 g, 60%) as a yellow solid. TLC: PE/EA=10:1, UV; R_{f} (compound 2)=0.20; R_{f} (compound 3)=0.60.

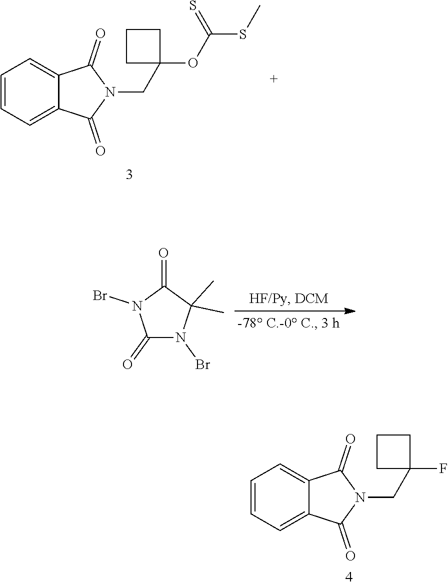 [SMILES: CSC(=S)OC1(CN2C(=O)c3ccccc3C2=O)CCC1 ; CC1(C)C(=O)N(Br)C(=O)N1Br ; O=C1c2ccccc2C(=O)N1CC1(F)CCC1]

To a solution of 1,3-dibromo-5,5-dimethylimidazolidine-2,4-dione (4.4 g, 15.5 mmol, 5.0 eq) in DCM (30 mL) was added HF/Py (65%-70%, 3 mL) in dry ice-acetone bath. The reaction mixture was added O-(1-((1,3-dioxoisoindolin-2-yl)methyl)cyclobutyl) S-methyl carbonodithioate (1.0 g, 3.1 mmol, 1.0 eq) in DCM (20 mL) at this temperature. The reaction mixture was stirred for 3 h at 0° C. The reaction mixture was quenched with NaOH (aq. 2N, 100 mL), extracted with DCM (100 mL×2), dried over anhydrous Na₂SO₄, filtered, and concentrated under reduced pressure. The crude product was purified by column chromatography on silica gel (PE/EtOAc=50:1 to 10:1) to give compound 2-((1-fluorocyclobutyl)methyl) isoindoline-1,3-dione (600 mg, 82%) as a yellow solid. TLC: PE/EA=20:1, UV; R_{f} (compound 3)=0.40; R_{f} (compound 4)=0.60; ¹H NMR (400 MHz, DMSO-d₆) δ (ppm): 7.93-7.85 (m, 4H), 3.92 (d, J=21.8 Hz, 2H), 2.34-2.09 (m, 4H), 1.85-1.72 (m, 1H), 1.66-1.53 (m, 1H).

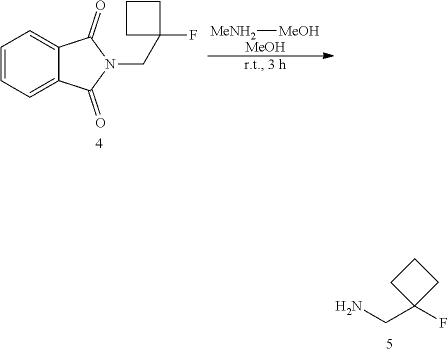

To a solution of 2-((1-fluorocyclobutyl)methyl)isoindoline-1,3-dione (600 mg, 2.6 mmol, 1.0 eq) in MeOH (5 mL) was added methylamine in methanol (10 M, 5 mL). The reaction mixture was stirred at r.t. for 3 h. The reaction mixture was concentrated under reduced pressure and purified by column chromatography on silica gel (DCM/MeOH, 50:1 to 10:1) to give (1-fluorocyclobutyl)methanamine (30 mg, 11%) as a colorless oil. TLC: DCM/MeOH=10:1, UV and stained with indenhydrin; R_{f} (compound 4)=0.95; R_{f} (compound 5)=0.30; LC-MS: 104.1 [M+H]⁺.

Using the above procedures and intermediates, the following compounds were prepared:

| Example | Structure | Analytical Data |
|---|---|---|
| 2 | 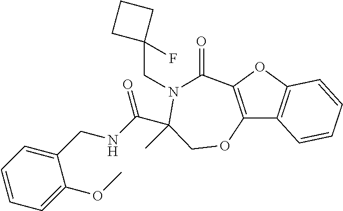 [SMILES: COc1ccccc1CNC(=O)C1(C)COc2c(oc3ccccc23)C(=O)N1CC1(F)CCC1] | 1H NMR (400 MHZ, CDCl3) δ 7.50 (d, J = 7.9 Hz, 1H), 7.45- 7.38 (m, 2H), 7.22-7.15 (m, 1H), 6.96-6.82 (m, 2H), 6.63-6.56 (m, 1H), 6.49 (d, J = 7.0 Hz, 2H), 4.88 (d, J = 11.8 Hz, 1H), 4.43- 4.21 (m, 2H), 4.17-3.96 (m, 3H), 3.71 (s, 3H), 2.39-2.11 (m, 4H), 1.89-1.77 (m, 5H). LC-MS: 467.2[M + 1]+ |
| 3 | 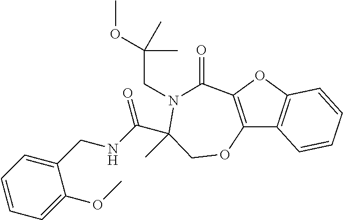 [SMILES: COc1ccccc1CNC(=O)C1(C)COc2c(oc3ccccc23)C(=O)N1CC(C)(C)OC] | 1H NMR (400 MHZ, DMSO) δ 8.17 (s, 1H), 7.67 (d, J = 7.7 Hz, 1H), 7.60 (d, J = 8.4 Hz, 1H), 7.56- 7.46 (m, 1H), 7.38-7.27 (m, 1H), 7.03 (t, J = 7.2 Hz, 1H), 6.82 (d, J = 8.0 Hz, 1H), 6.61 (s, 1H), 6.30 (s, 1H), 5.00 (d, J = 12.2 Hz, 1H), 4.44 (d, J = 12.2 Hz, 1H), 4.30-3.94 (m, 3H), 3.70 (s, 3H), 3.50-3.38 (m, 1H), 3.07 (s, 3H), 1.80 (s, 3H), 1.17 (s, 3H), 1.11 (s, 3H). LC-MS: 467.2[M + 1]+ |
| 4 | 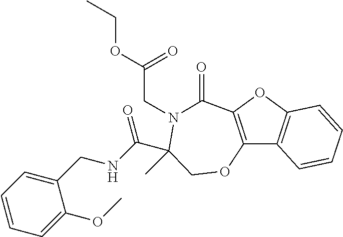 [SMILES: CCOC(=O)CN1C(=O)c2oc3ccccc3c2OCC1(C)C(=O)NCc1ccccc1OC] | 1H NMR (400 MHZ, DMSO) δ 8.59 (t, J = 5.8 Hz, 1H), 7.70 (d, J = 7.8 Hz, 1H), 7.63 (d, J = 8.4 Hz, 1H), 7.59-7.52 (m, 1H), 7.36 (t, J = 7.5 Hz, 1H), 7.01 (t, J = 6.7 Hz, 1H), 6.82 (d, J = 8.1 Hz, 1H), 6.54 (s, 1H), 6.17 (s, 1H), 4.98 (d, J = 11.7 Hz, 1H), 4.57 (d, J = 16.4 Hz, 1H), 4.39 (d, J = 12.2 Hz, 1H), 4.31-4.02 (m, 5H), 3.70 (s, 3H), 1.65 (s, 3H), 1.22 (t, J = 7.1 Hz, 3H). LC-MS: 467.1[M + 1]+ |
| 5 | 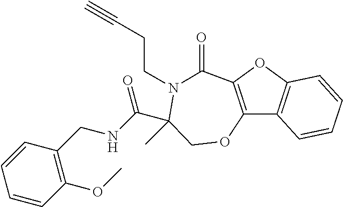 [SMILES: C#CCCN1C(=O)c2oc3ccccc3c2OCC1(C)C(=O)NCc1ccccc1OC] | 1H NMR (400 MHZ, CDCl3) δ 7.52 (d, J = 7.9 Hz, 1H), 7.45- 7.40 (m, 2H), 7.21 (ddd, J = 8.0, 5.2, 2.9 Hz, 1H), 6.99-6.95 (m, 2H), 6.72 (t, J = 4 Hz, 1H), 6.5-6.57 (m, 2H), 4.80 (d, J = 11.9 Hz, 1H), 4.37 (dd, J = 14.2, 6.8 Hz, 1H), 4.23 (dd, J = 14.3, 5.1 Hz, 1H), 4.17 (d, J = 12.2 Hz, 1H), 3.86-3.84 (m, 1H), 3.72 (s, 3H), 3.73-3.58 (m, 1H), 2.73-3.69 (m, 1H), 2.66-2.49 (m, 1H), 2.00 (t, J = 2.7 Hz, 1H), 1.79 (s, 3H). LC-MS: 433.2[M + 1]+ |
| 6 | 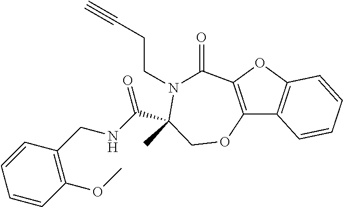 [SMILES: C#CCCN1C(=O)c2oc3ccccc3c2OC[C@]1(C)C(=O)NCc1ccccc1OC] | 1H NMR (400 MHZ, CDCl3) δ 7.52 (d, J = 7.9 Hz, 1H), 7.45- 7.40 (m, 2H), 7.21 (ddd, J = 8.0, 5.2, 2.9 Hz, 1H), 6.99-6.95 (m, 2H), 6.72 (t, J = 4 Hz, 1H), 6.5-6.57 (m, 2H), 4.80 (d, J = 11.9 Hz, 1H), 4.37 (dd, J = 14.2, 6.8 Hz, 1H), 4.23 (dd, J = 14.3, 5.1 Hz, 1H), 4.17 (d, J = 12.2 Hz, 1H), 3.86-3.84 (m, 1H), 3.72 (s, 3H), 3.73-3.58 (m, 1H), 2.73-3.69 (m, 1H), 2.66-2.49 (m, 1H), 2.00 (t, J = 2.7 Hz, 1H), 1.79 (s, 3H). LC-MS: 433.2[M + 1]+ |
| 7 | 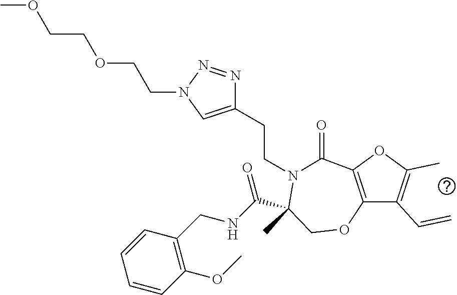 | 1H NMR (400 MHZ, CDCl3) δ 7.61 (s, 1H), 7.51 (d, J = 7.9 Hz, 1H), 7.46-7.37 (m, 2H), 7.20 (ddd, J = 8.0, 5.1, 3.0 Hz, 1H), 7.04 (t, J = 5.8 Hz, 1H), 6.98 (d, J = 7.2 Hz, 1H), 6.93 (t, J = 7.7 Hz, 1H), 6.54 (dd, J = 12.2, 7.8 Hz, 2H), 4.81 (d, J = 12.1 Hz, 1H), 4.62- 4.45 (m, 2H), 4.40 (dd, J = 14.3, 6.8 Hz, 1H), 4.21 (dd, J = 14.2, 5.0 Hz, 1H), 4.08 (d, J = 12.1 Hz, 1H), 4.05-3.94 (m, 1H), 3.94-3.78 (m, 3H), 3.70 (s, 3H), 3.64-3.55 (m, 2H), 3.52-3.48 (m, 2H), 3.36 (s, 3H), 3.23-3.16 (m, 1H), 3.11-3.01 (m, 1H), 1.79 (s, 3H). LC-MS: 478.2[M + 1] |
| 9 | 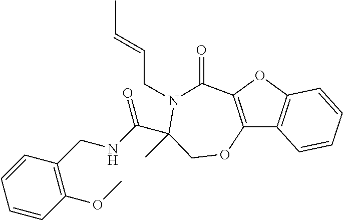 [SMILES: C/C=C/CN1C(=O)c2oc3ccccc3c2OCC1(C)C(=O)NCc1ccccc1OC] | 1H NMR (400 MHZ, CDCl3) δ 7.53 (d, J = 7.9 Hz, 1H), 7.48- 7.37 (m, 2H), 7.24-7.16 (m, 1H), 7.05-6.92 (m, 2H), 6.67 (t, J = 5.5 Hz, 1H), 6.63-6.51 (m, 2H), 5.78- 5.64 (m, 1H), 5.65-5.49 (m, 1H), 4.78 (d, J = 11.8 Hz, 1H), 4.41- 4.33 (m, 1H), 4.32-4.25 (m, 1H), 4.25-4.18 (m, 1H), 4.17-4.05 (m, 2H), 3.73 (s, 3H), 1.73 (s, 3H), 1.62 (dd, J = 6.1, 1.0 Hz, 3H). LC-MS: 435.20[M + 1]+ |
| 10 | 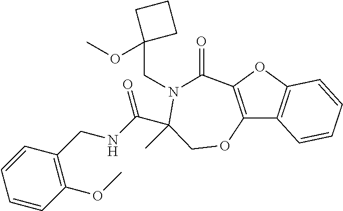 [SMILES: COc1ccccc1CNC(=O)C1(C)COc2c(oc3ccccc23)C(=O)N1CC1(OC)CCC1] | 1H NMR (400 MHZ, DMSO) δ 8.20-8.05 (m, 1H), 7.66 (d, J = 7.8 Hz, 1H), 7.59 (d, J = 8.4 Hz, 1H), 7.56-7.45 (m, 1H), 7.33 (t, J = 7.5 Hz, 1H), 7.04 (t, J = 7.5 Hz, 1H), 6.82 (d, J = 8.1 Hz, 1H), 6.68 (s, 1H), 6.43-6.25 (m, 1H), 4.96 (d, J = 12.1 Hz, 1H), 4.35 (d, J = 12.3 Hz, 1H), 4.25 (d, J = 14.5 Hz, 1H), 4.20- 4.03 (m, 2H), 3.74-3.71 (m, 1H), 3.70 (s, 3H), 3.10 (s, 3H), 2.31- 1.99 (m, 2H), 1.94-1.79 (m, 2H), 1.75 (s, 3H), 1.72-1.52 (m, 2H). |
| | | LC-MS: 479.20[M + 1]+ |
| 11 | 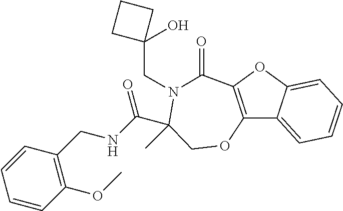 [SMILES: COc1ccccc1CNC(=O)C1(C)COc2c(oc3ccccc23)C(=O)N1CC1(O)CCC1] | 1H NMR (400 MHZ, DMSO) δ 8.23 (t, J = 5.8 Hz, 1H), 7.65 (d, J = 7.8 Hz, 1H), 7.59 (d, J = 8.4 Hz, 1H), 7.57-7.48 (m, 1H), 7.33 (t, J = 7.2 Hz, 1H), 6.99 (t, J = 7.3 Hz, 1H), 6.80 (d, J = 8.1 Hz, 1H), 6.53 (d, J = 7.0 Hz, 1H), 6.21 (t, J = 7.2 Hz, 1H), 5.38 (s, 1H), 4.99 (d, J = 12.2 Hz, 1H), 4.45 (d, J = 12.2 Hz, 1H), 4.25-4.01 (m, 3H), 3.69 (s, 3H), 3.60 (d, J = 15.0 Hz, 1H), 2.25-2.05 (m, 2H), 1.99-1.84 (m, 2H), 1.82 (s, 3H), 1.74-1.49 (m, |
| | | 2H). LC-MS: 465.20[M + 1]+ |
| 12 | 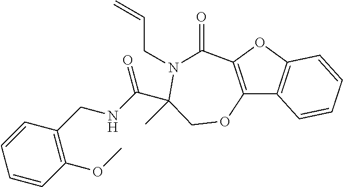 [SMILES: C=CCN1C(=O)c2oc3ccccc3c2OCC1(C)C(=O)NCc1ccccc1OC] | 1H NMR (400 MHZ, CDCl3) δ 7.53 (d, J = 7.9 Hz, 1H), 7.48- 7.38 (m, 2H), 7.24-7.16 (m, 1H), 7.06-6.92 (m, 2H), 6.69 (t, J = 5.4 Hz, 1H), 6.65-6.55 (m, 2H), 6.04- 5.86 (m, 1H), 5.25-5.13 (m, 2H), 4.78 (d, J = 12.0 Hz, 1H), 4.52-4.42 (m, 1H), 4.42-4.33 (m, 1H), 4.28- 4.20 (m, 1H), 4.19-4.12 (m, 1H), 4.12-4.04 (m, 1H), 3.72 (s, 3H), 1.72 (s, 3H). LC-MS: 421.20[M + 1]+ |
| 13 | 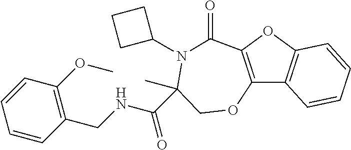 [SMILES: COc1ccccc1CNC(=O)C1(C)COc2c(oc3ccccc23)C(=O)N1C1CCC1] | 1H NMR (400 MHZ, DMSO) δ 8.46 (s, 1H), 7.71 (d, J = 7.7 Hz, 1H), 7.62 (d, J = 8.4 Hz, 1H), 7.56- 7.46 (m, 1H), 7.40-7.29 (m, 1H), 7.27-7.12 (m, 2H), 6.96 (d, J = 8.1 Hz, 1H), 6.91-6.78 (m, 1H), 4.62 (d, J = 12.8 Hz, 1H), 4.40 (d, J = 12.7 Hz, 1H), 4.34-4.19 (m, 2H), 3.81 (d, J = 14.3 Hz, 3H), 3.75- 3.62 (m, 1H), 2.97-2.78 (m, 2H), |
| | | 2.05-1.86 (m, 2H), 1.78-1.62 (m, |
| | | 1H), 1.59-1.45 (m, 1H), 1.42 (s, 3H). |
| | | LC-MS: 435.20[M + 1]+ |
| 14 | 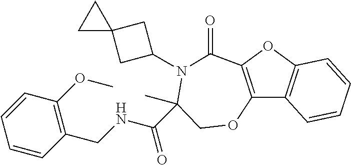 [SMILES: COc1ccccc1CNC(=O)C1(C)COc2c(oc3ccccc23)C(=O)N1C1CC2(CC2)C1] | 1H NMR (400 MHZ, CDCl3) δ 7.59 (d, J = 7.8 Hz, 1H), 7.53-7.47 (m, 1H), 7.47-7.40 (m, 1H), 7.26-7.16 (m, 3H), 6.87 (t, J = 7.5 Hz, 1H), 6.76 (d, J = 7.9 Hz, 1H), 6.68-6.54 (m, 1H), 4.45 (d, J = 5.8 Hz, 2H), 4.35 (s, 2H), 4.14-3.96 (m, 1H), 3.74 (s, 3H), 3.22- 3.04 (m, 2H), 2.09-1.95 (m, 1H), 1.91 (t, J = 9.3 Hz, 1H), 1.59 (s, 3H), 0.47-0.28 (m, 4H). LC-MS: 461.20[M + 1]+ |
| 15 | 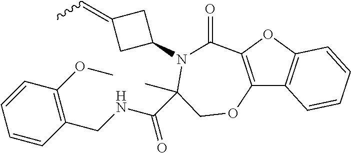 | 1H NMR (400 MHZ, CDCl3) δ 7.58 (d, J = 7.9 Hz, 1H), 7.52-7.40 (m, 2H), 7.25-7.19 (m, 3H), 6.87 (t, J = 7.3 Hz, 1H), 6.77 (d, J = 7.1 Hz, 1H), 6.68-6.55 (m, 1H), 5.29-5.07 (m, 1H), 4.60-4.24 (m, 4H), 3.92-3.80 (m, 1H), 3.76 (s, 3H), 3.58-3.30 (m, 2H), 2.84-2.46 (m, 2H), 1.58 (d, J = 4.3 Hz, 3H), 1.51-1.41(m, 3H). LC-MS: 461.20[M + 1]+ |
| 51 | 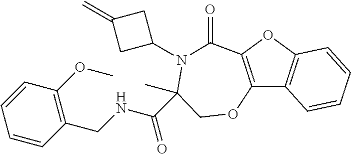 [SMILES: C=C1CC(N2C(=O)c3oc4ccccc4c3OCC2(C)C(=O)NCc2ccccc2OC)C1] | 1H NMR (400 MHZ, CDCl3) δ 7.59 (d, J = 7.9 Hz, 1H), 7.52-7.41 (m, 2H), 7.29-7.26 (m, 1H), 7.25-7.19 (m, 2H), 6.87 (t, J = 7.4 Hz, 1H), 6.77 (d, J = 7.5 Hz, 1H), 6.56-6.54 (m, 1H), 4.80 (d, J = 10.5 Hz, 2H), 4.45 (d, J = 5.8 Hz, 2H), 4.41-4.26 (m, 2H), 3.88-3.79 (m, 1H), 3.76 (s, 3H), 3.68-3.48 (m, 2H), 2.84- 2.68 (m, 1H), 2.68-2.53 (m, 1H), 1.58 (s, 3H). LC-MS: 447.20[M + 1]+ |

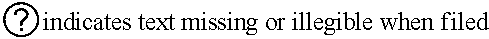

### Intermediate BB-2

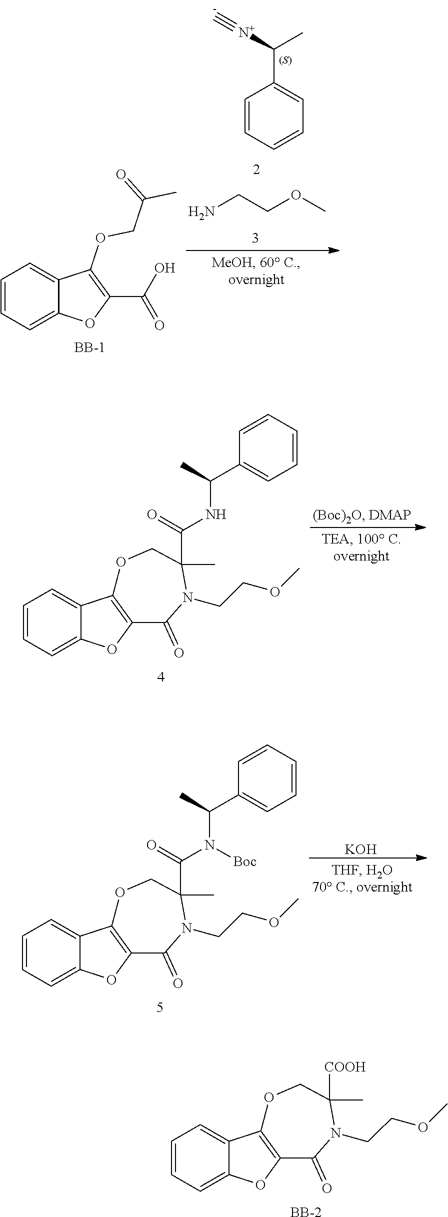

A mixture of 3-(2-oxopropoxy)benzofuran-2-carboxylic acid (220 g, 0.94 mol, 1.0 eq), 2-methoxyethylamine (90 g, 1.13 mol, 1.2 eq), and (S)-(1-isocyanoethyl)benzene (160 g, 1.22 mol, 1.3 eq) in MeOH (1 L) was stirred at 60° C. overnight and concentrated under reduced pressure. The crude product was by column chromatography on silica gel (Hex/EtOAc, 100:1 to 50:1) to give 4-(2-methoxyethyl)-3-methyl-5-oxo-N—((S)-1-phenylethyl)-2,3,4,5-tetrahydrobenzofuro[2,3-f][1,4]oxazepine-3-carboxamide (240 g, 60%) as a light-yellow solid. LC-MS: 423.1 [M+H]⁺; TLC: PE/EA=1:1, UV; R_{f}(compound 1)=0.70; R_{f} (compound 4)=0.40.

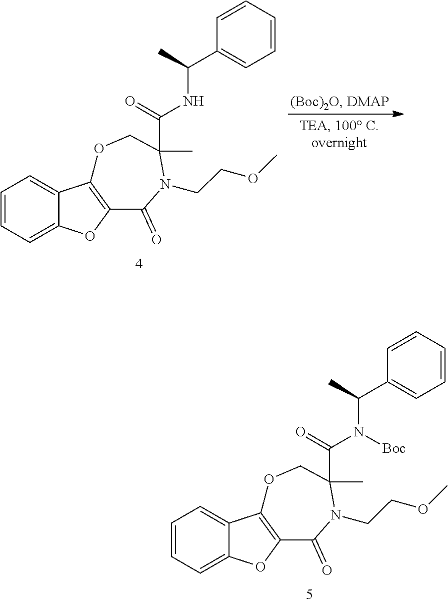 [SMILES: COCCN1C(=O)c2oc3ccccc3c2OCC1(C)C(=O)N[C@@H](C)c1ccccc1 ; COCCN1C(=O)c2oc3ccccc3c2OCC1(C)C(=O)N(C(=O)OC(C)(C)C)[C@@H](C)c1ccccc1]

A mixture of 4-(2-methoxyethyl)-3-methyl-5-oxo-N—((S)-1-phenylethyl)-2,3,4,5-tetrahydrobenzofuro[2,3-f][1,4]oxazepine-3-carboxamide (240 g, 0.57 mol, 1.0 eq), (Boc)₂O (496 g, 2.28 mol, 4.0 eq), and DMAP (69 g, 0.57 mol, 1.0 eq) in TEA (500 mL) was stirred at 110° C. overnight. The mixture was diluted with EtOAc (500 mL). The organic layer was washed with brine, dried over anhydrous Na₂SO₄, filtered, and concentrated under reduced pressure. The residue was purified by column chromatography on silica gel (PE:EtOAc=1:1) to give tert-butyl (4-(2-methoxyethyl)-3-methyl-5-oxo-2,3,4,5-tetrahydrobenzofuro[2,3-f][1,4]oxazepine-3-carbonyl)((S)-1-phenylethyl)carbamate (160 g, 54%) as a light-yellow solid. LC-MS: 523.1 [M+H]⁺; TLC: PE/EA=1:1, UV; R_{f} (compound 4)=0.40; R_{f} (compound 5)=0.70.

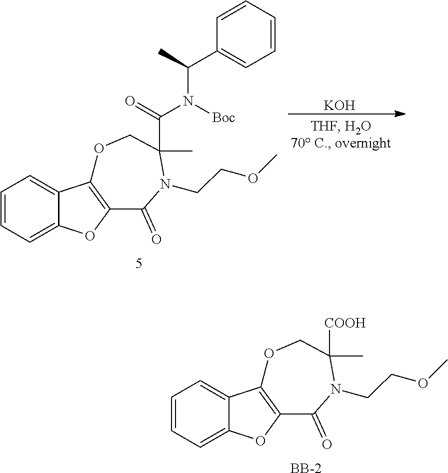

To a solution of tert-butyl (4-(2-methoxyethyl)-3-methyl-5-oxo-2,3,4,5-tetrahydrobenzofuro[2,3-f][1,4]oxazepine-3-carbonyl)((S)-1-phenylethyl)carbamate (160 g, 0.31 mol, 1.0 eq) in EtOH (500 mL), THE (250 mL) was added KOH solution (10% w/w in H₂O, 500 mL). The reaction mixture was stirred at 70° C. overnight, added 6 N HCl to adjust to pH 1-2. The mixture was filtered and the cake was washed with water (200 mL). The solid was dried at 25° C. under reduced pressure to give the desired product (76 g, 23%). LC-MS: 320.1[M+1]+; 1H NMR (400 MHz, DMSO) δ 13.28 (s, 1H), 7.68 (d, J=7.6 Hz, 1H), 7.61 (d, J=8.4 Hz, 1H), 7.51 (ddd, J=8.4, 7.2, 1.3 Hz, 1H), 7.38-7.24 (m, 1H), 4.83 (d, J=12.3 Hz, 1H), 4.38 (d, J=12.3 Hz, 1H), 3.83-3.72 (m, 1H), 3.64 (dt, J=14.5, 6.1 Hz, 1H), 3.54-3.41 (m, 2H), 3.23 (s, 3H), 1.74 (s, 3H).

### (4-chloro-5-fluoro-2-methoxyphenyl)methanamine

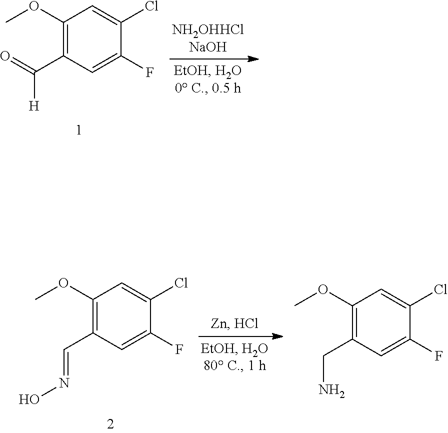

A mixture of NaOH (192 mg, 4.8 mmol, 3.0 eq.) and hydroxylamine hydrochloride (167 mg, 2.4 mmol, 1.5 eq.) was dissolved in 1 mL of water and 5 mL of ethanol at 0° C. for 5 min. To the above mixture, 4-chloro-5-fluoro-2-methoxybenzaldehyde (300 mg, 1.6 mmol, 1.0 eq.) was added. The reaction mixture was warmed to room temperature and stirred for 1 h. At the end of the reaction (confirmed by TLC) the reaction was diluted with water (10 mL) and the aqueous phase was extracted with EA (3×10 mL). The organic phases were washed with brine (10 mL), dried over anhydrous Na₂SO₄ and concentrated under reduced pressure to yield the crude product (E)-4-chloro-5-fluoro-2-methoxybenzaldehyde oxime (300 mg, 92%) as a white solid. LC-MS: 204.1[M+H]⁺ (V1638-085); TLC: PE/EA=10:1, UV; R_{f} (compound 1)=0.70; R_{f} (compound 2)=0.50.

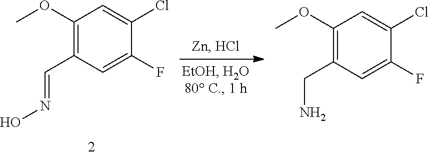

(E)-4-chloro-5-fluoro-2-methoxybenzaldehyde oxime (300 mg, 1.5 mmol) was dissolved in EtOH (6 mL) and water (1 mL). concentrated HCl (2 mL) was added dropwise at 0° C. and the mixture was stirred 10 min before Zn powder (780 mg, 12 mmol, 8 eq.) was added. The mixture was stirred 1 h at 80° C. The mixture was cooled to room temperature and filtered. The filtrate was concentrated under reduced pressure to give a white solid (4-chloro-5-fluoro-2-methoxyphenyl)methanamine (280 mg, 98%). The crude product was used directly in the next step without further purification. TLC: PE/EA=10:1, UV; R_{f}(compound 2)=0.50; R_{f} (compound 3)=0.00; LC-MS: 190.1 [M+H]⁺.

### (3-chloro-2-fluoro-6-methoxyphenyl)methanamine

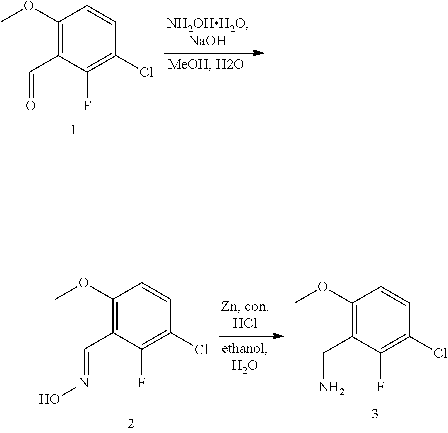

To a solution of 3-chloro-2-fluoro-6-methoxybenzaldehyde (2.0 g, 10.61 mmol) in ethanol (20 mL) was added NaOH (1.3 g, 32.50 mmol, 3.0 eq.) and hydroxylamine hydrochloride (2.2 g, 31.66 mmol, 1.5 eq.) at 0° C. The mixture was allowed to warm to room temperature and stirred for 3 h. water (20 mL) was added and the aqueous phase was extracted with EA (3×20 mL). The combined organic phases were washed with brine (20 mL), dried over NaSO₄, filtered and concentrated in vacuo to give the crude products, which was used directly in the next step. LC-MS: 204.1 [M+H]⁺.

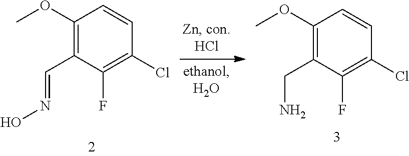

To a solution of 3-chloro-2-fluoro-6-methoxybenzaldehyde oxime (200 mg, 0.98 mmol, 1.0 eq) in ethanol (3 mL) was added con. HCl (1 mL) and water (0.5 mL) at 0° C. The mixture was stirred for 15 min. Zn powder (383 mg, 5.89 mmol, 6.0 eq) was added. The reaction mixture was heated to 80° C. for 1 h, cooled to room temperature, and filtered. The filtrate was concentrated under reduced pressure to give the hydrochloride salt of (3-chloro-2-fluoro-6-methoxyphenyl)methanamine (190 mg, 100%) as a yellow solid. LC-MS: 190.1 [M+H]⁺.

Using the above procedures and intermediates, the following compounds were prepared:

| Example | Structure | Analytical Data |
|---|---|---|
| 16 | 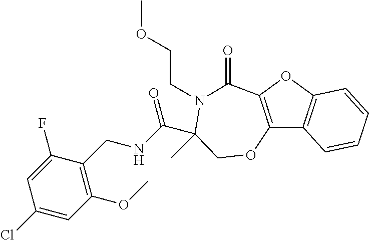 [SMILES: COCCN1C(=O)c2oc3ccccc3c2OCC1(C)C(=O)NCc1c(F)cc(Cl)cc1OC] | 1H NMR (400 MHZ, CDCl3) δ 7.52 (d, J = 7.8 Hz, 1H), 7.46- 7.33 (m, 3H), 7.20 (ddd, J = 8.0, 6.9, 1.2 Hz, 1H), 6.31-6.28 (m, 2H), 4.87 (d, J = 12.0 Hz, 1H), 4.66 (dd, J = 14.1, 7.6 Hz, 1H), 4.37-4.31 (m, 1H), 4.07 (d, J = 11.8 Hz, 1H), 3.94-3.86 (m, 1H), 3.79 (ddd, J = 9.8, 5.6, 4.1 Hz, 1H), 3.69 (s, 3H), 3.64- 3.48 (m, 2H), 3.32 (s, 3H), 1.77 (s, 3H). LC-MS: 491.1[M + 1]+ |
| 17 | 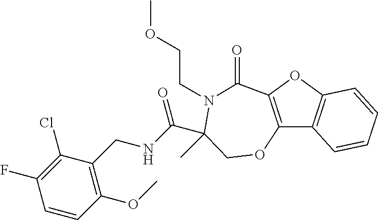 [SMILES: COCCN1C(=O)c2oc3ccccc3c2OCC1(C)C(=O)NCc1c(OC)ccc(F)c1Cl] | 1H NMR (400 MHZ, CDCl3) δ 7.53 (d, J = 7.9 Hz, 1H), 7.43- 7.34 (m, 3H), 7.20 (ddd, J = 8.0, 6.3, 1.7 Hz, 1H), 6.64 (t, J = 8.7 Hz, 1H), 6.38 (dd, J = 9.0, 3.8 Hz, 1H), 4.95-4.75 (m, 2H), 4.36-4.30 (m, 1H), 4.19-4.03 (m, 2H), 3.81-3.75 (m, 1H), 3.70 (s, 3H), 3.62-3.48 (m, 2H), 3.26 (s, 3H), 1.78 (s, 3H). LC-MS: 491.1[M + 1]+ |
| 18 | 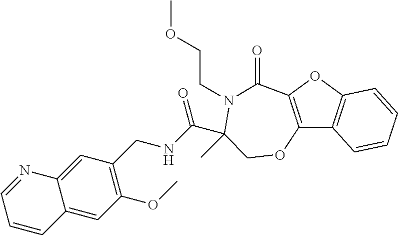 [SMILES: COCCN1C(=O)c2oc3ccccc3c2OCC1(C)C(=O)NCc1cc2ncccc2cc1OC] | 1H NMR (400 MHZ, CDCl3) δ 8.66 (dd, J = 4.1, 1.3 Hz, 1H), 7.80 (d, J = 8.1 Hz, 1H), 7.72 (s, 1H), 7.66-7.57 (m, 1H), 7.29- 7.16 (m, 4H), 6.87 (t, J = 7.3 Hz, 1H), 6.72 (s, 1H), 4.87 (d, J = 11.8 Hz, 1H), 4.60 (dd, J = 14.4, 7.3 Hz, 1H), 4.41-4.22 (m, 2H), 4.07 (d, J = 11.8 Hz, 1H), 3.88 (s, 3H), 3.85-3.78 (m, 1H), 3.66-3.53 (m, 2H), 3.30 (s, 3H), 1.81 (s, 3H). LC-MS: 490.1[M + 1]+ |
| 19 | 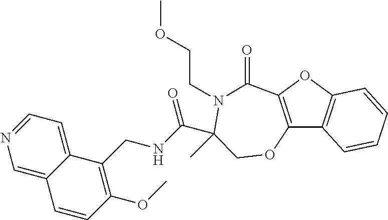 [SMILES: COCCN1C(=O)c2oc3ccccc3c2OCC1(C)C(=O)NCc1c(OC)ccc2cnccc12] | 1H NMR (400 MHZ, CDCl3) δ 8.82 (s, 1H), 8.11 (d, J = 6.0 Hz, 1H), 7.71 (d, J = 6.2 Hz, 1H), 7.59 (d, J = 9.0 Hz, 1H), 7.52- (m, 1H), 7.41-7.33 (m, 1H), 7.26-7.18 (m, 2H), 7.16-7.06 (m, 2H), 4.98 (dd, J = 14.1, 7.7 Hz, 1H), 4.88 (d, J = 11.6 Hz, 1H), 4.47-4.25 (m, 2H), 4.02 (d, J = 11.9 Hz, 1H), 3.96 (s, 3H), 3.81-3.72 (m, 1H), 3.59-3.45 (m, 2H), 3.22 (s, 3H), 1.79 (s, 3H). LC-MS: 490.2[M + 1]+ |
| 20 | 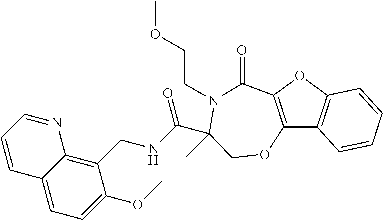 [SMILES: COCCN1C(=O)c2oc3ccccc3c2OCC1(C)C(=O)NCc1c(OC)ccc2cccnc12] | 1H NMR (400 MHZ, CDCl3) δ 8.83 (d, J = 2.8 Hz, 1H), 8.48 (s, 1H), 7.93 (d, J = 7.0 Hz, 1H), 7.46 (d, J = 8.9 Hz, 1H), 7.42-7.30 (m, 2H), 7.26-7.22 (m, 2H), 7.11 (t, J = 7.5 Hz, 1H), 7.02 (d, J = 8.7 Hz, 1H), 5.22 (dd, J = 14.3, 7.0 Hz, 1H), 4.81 (d, J = 12.2 Hz, 1H), 4.69 (d, J = 11.3 Hz, 1H), 4.07 (d, J = 11.8 Hz, 1H), 3.97-3.86 (m, 1H), 3.86- 3.73 (m, 4H), 3.71-3.56 (m, 2H), 3.23 (s, 3H), 1.77 (s, 3H). LC-MS: 490.1[M + 1]+ |
| 21 | 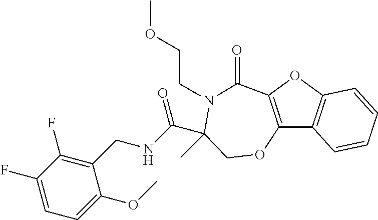 [SMILES: COCCN1C(=O)c2oc3ccccc3c2OCC1(C)C(=O)NCc1c(OC)ccc(F)c1F] | 1H NMR (400 MHZ, CDCl3) δ 7.54 (d, J = 7.9 Hz, 1H), 7.47- 7.36 (m, 3H), 7.20 (ddd, J = 8.0, 5.9, 2.1 Hz, 1H), 6.61 (dd, J = 18.5, 9.3 Hz, 1H), 6.16 (d, J = 8.6 Hz, 1H), 4.87 (d, J = 11.8 Hz, 1H), 4.66 (dd, J = 14.0, 7.2 Hz, 1H), 4.41-4.27 (m, 1H), 4.15-3.95 (m, 2H), 3.84-3.75 (m, 1H), 3.67 (s, 3H), 3.63- 3.47 (m, 2H), 3.30 (s, 3H), 1.77 (s, 3H). LC-MS: 475.1[M + 1]+ |
| 22 | 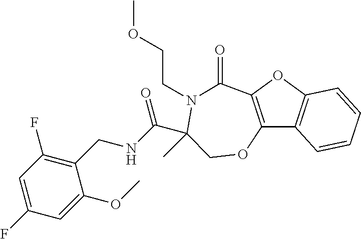 [SMILES: COCCN1C(=O)c2oc3ccccc3c2OCC1(C)C(=O)NCc1c(F)cc(F)cc1OC] | 1H NMR (400 MHZ, CDCl3) δ 7.53 (d, J = 7.9 Hz, 1H), 7.45- 7.32 (m, 3H), 7.19 (ddd, J = 8.0, 6.6, 1.5 Hz, 1H), 6.12-5.97 (m, 2H), 4.87 (d, J = 11.8 Hz, 1H), 4.63 (dd, J = 14.1, 7.5 Hz, 1H), 4.44-4.26 (m, 1H), 4.07 (d, J = 11.9 Hz, 1H), 3.91 (d, J = 11.2 Hz, 1H), 3.84-3.76 (m, 1H), 3.68 (s, 3H), 3.63-3.48 (m, 2H), 3.31 (s, 3H), 1.77 (s, 3H). LC-MS: 475.1[M + 1]+ |
| 23 | 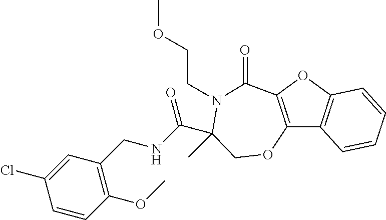 [SMILES: COCCN1C(=O)c2oc3ccccc3c2OCC1(C)C(=O)NCc1cc(Cl)ccc1OC] | 1H NMR (400 MHZ, CDCl3) δ 7.57-7.47 (m, 2H), 7.45-7.35 (m, 2H), 7.20 (ddd, J = 8.0, 6.4, 1.7 Hz, 1H), 6.95 (d, J = 2.5 Hz, 1H), 6.74 (d, J = 6.8 Hz, 1H), 6.41 (d, J = 8.7 Hz, 1H), 4.88 (d, J = 11.8 Hz, 1H), 4.43-4.28 (m, 2H), 4.11-3.99 (m, 2H), 3.84- 3.77 (m, 1H), 3.74 (s, 3H), 3.65- 3.50 (m, 2H), 3.31 (s, 3H), 1.78 (s, 3H). LC-MS: 473.1[M + 1]+ |
| 24 | 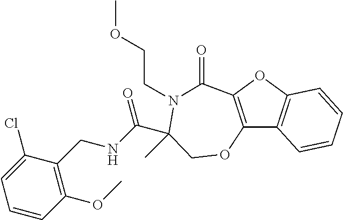 [SMILES: COCCN1C(=O)c2oc3ccccc3c2OCC1(C)C(=O)NCc1c(Cl)cccc1OC] | 1H NMR (400 MHZ, CDCl3) δ 7.55 (d, J = 7.9 Hz, 1H), 7.42- 7.37 (m, 2H), 7.26-7.23 (m, 1H), 7.22-7.16 (m, 1H), 6.85 (t, J = 8.1 Hz, 1H), 6.67 (d, J = 8.0 Hz, 1H), 6.49 (d, J = 8.2 Hz, 1H), 4.85 (d, J = 11.9 Hz, 1H), 4.73 (dd, J = 13.8, 6.6 Hz, 1H), 4.29-4.19 (m, 2H), 4.10 (d, J = 11.9 Hz, 1H), 3.81-3.67 (m, 4H), 3.61-3.50 (m, 2H), 3.19 (s, 3H), 1.76 (s, 3H). LC-MS: 473.1[M + 1]+ |
| 25 | 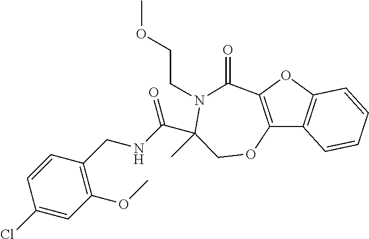 [SMILES: COCCN1C(=O)c2oc3ccccc3c2OCC1(C)C(=O)NCc1ccc(Cl)cc1OC] | 1H NMR (400 MHZ, CDCl3) δ 7.54-7.36 (m, 4H), 7.20 (ddd, J = 8.0, 6.9, 1.2 Hz, 1H), 6.75 (d, J = 8.0 Hz, 1H), 6.50 (d, J = 1.4 Hz, 1H), 6.34 (d, J = 7.6 Hz, 1H), 4.88 (d, J = 11.8 Hz, 1H), 4.46-4.26 (m, 2H), 4.08 (d, J = 11.9 Hz, 1H), 3.98 (dd, J = 14.3, 4.6 Hz, 1H), 3.84-3.71 (m, 4H), 3.65-3.51 (m, 2H), 3.32 (s, 3H), 1.78 (s, 3H). LC-MS: 473.1[M + 1]+ |
| 26 | 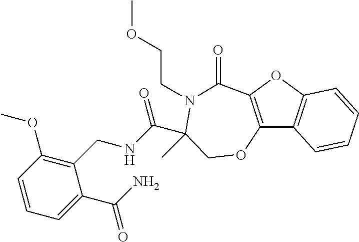 [SMILES: COCCN1C(=O)c2oc3ccccc3c2OCC1(C)C(=O)NCc1c(OC)cccc1C(N)=O] | 1H NMR (400 MHZ, CDCl3) δ 8.61 (s, 1H), 8.03 (s, 1H), 7.42- 7.32 (m, 3H), 7.12 (ddd, J = 8.1, 6.6, 1.4 Hz, 1H), 6.80-6.69 (m, 2H), 6.52-6.42 (m, 1H), 5.94 (s, 1H), 4.88-4.75 (m, 2H), 4.45-4.32 (m, 1H), 4.08-3.96 (m, 2H), 3.88-3.79 (m, 4H), 3.70-3.55 (m, 2H), 3.45 (s, 3H), 1.78 (s, 3H). LC-MS: 482.2[M + 1]+ |
| 27 | 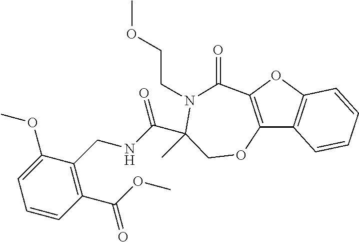 [SMILES: COCCN1C(=O)c2oc3ccccc3c2OCC1(C)C(=O)NCc1c(OC)cccc1C(=O)OC] | 1H NMR (400 MHZ, CDCl3) δ 7.48 (d, J = 7.8 Hz, 1H), 7.46- 7.34 (m, 3H), 7.20-7.15 (m, 1H), 7.12 (d, J = 7.7 Hz, 1H), 6.92 (s, 1H), 6.68 (d, J = 7.5 Hz, 1H), 4.99 (dd, J = 13.6, 7.3 Hz, 1H), 4.80 (d, J = 11.2 Hz, 1H), 4.24 (d, J = 10.6 Hz, 1H), 4.08 (d, J = 11.6 Hz, 2H), 3.89 (s, 3H), 3.75-3.66 (m, 5H), 3.63- 3.56 (m, 1H), 3.26 (s, 3H), 1.76 (s, 3H). LC-MS: 497.2[M + 1]+ |
| 28 | 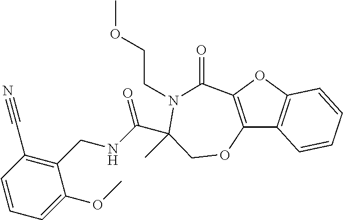 [SMILES: COCCN1C(=O)c2oc3ccccc3c2OCC1(C)C(=O)NCc1c(C#N)cccc1OC] | 1H NMR (400 MHZ, CDCl3) δ 7.59 (s, 1H), 7.48 (d, J = 7.9 Hz, 1H), 7.43-7.35 (m, 2H), 7.21-7.14 (m, 1H), 6.85 (d, J = 4.1 Hz, 2H), 6.70-6.57 (m, 1H), 4.87 (d, J = 11.8 Hz, 1H), 4.80 (dd, J = 14.1, 7.2 Hz, 1H), 4.43- 4.31 (m, 1H), 4.13 (dd, J = 14.0, 4.2 Hz, 1H), 4.05 (d, J = 11.8 Hz, 1H), 3.85-3.78 (m, 1H), 3.71 (s, 3H), 3.65-3.58 (m, 1H), 3.57-3.47 (m, 1H), 3.33 (s, 3H), 1.78 (s, 3H). LC-MS: 464.2[M + 1]+ |
| 29 | 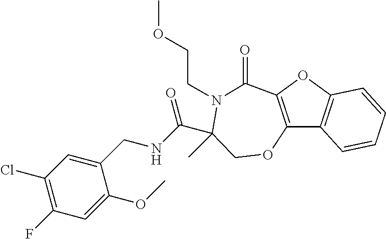 [SMILES: COCCN1C(=O)c2oc3ccccc3c2OCC1(C)C(=O)NCc1cc(Cl)c(F)cc1OC] | 1H NMR (400 MHZ, CDCl3) δ 7.56 (s, 1H), 7.49 (d, J = 7.8 Hz, 1H), 7.42 (ddd, J = 8.4, 7.1, 1.3 Hz, 1H), 7.36 (d, J = 8.4 Hz, 1H), 7.23-7.15 (m, 1H), 6.96 (d, J = 8.3 Hz, 1H), 6.26 (d, J = 10.7 Hz, 1H), 4.88 (d, J = 11.9 Hz, 1H), 4.50-4.30 (m, 2H), 4.06 (d, J = 11.8 Hz, 1H), 3.92 (dd, J = 14.2, 4.4 Hz, 1H), 3.85- 3.74 (m, 4H), 3.66-3.58 (m, 1H), 3.56-3.48 (m, 1H), 3.37 (s, 3H), 1.79 (s, 3H). LC-MS: 491.2[M + 1]+ |
| 30 | 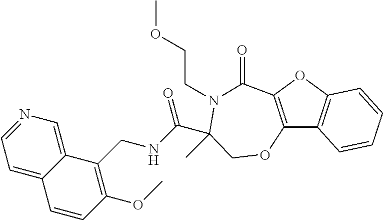 [SMILES: COCCN1C(=O)c2oc3ccccc3c2OCC1(C)C(=O)NCc1c(OC)ccc2ccncc12] | 1H NMR (400 MHZ, CDCl3) δ 9.48 (s, 1H), 8.29 (d, J = 5.6 Hz, 1H), 7.48 (d, J = 9.0 Hz, 2H), 7.39-7.28 (m, 4H), 7.20 (d, J = 9.0 Hz, 1H), 7.10 (t, J = 7.3 Hz, 1H), 5.07 (dd, J = 14.1, 6.8 Hz, 1H), 4.85 (d, J = 11.5 Hz, 1H), 4.55 (dd, J = 14.0, 4.5 Hz, 1H), 4.28 (s, 1H), 4.05 (d, J = 11.8 Hz, 1H), 3.88 (s, 3H), 3.80- 3.71 (m, 1H), 3.56-3.44 (m, 2H), 3.09 (s, 3H), 1.77 (s, 3H). LC-MS: 490.2[M + 1]+ |
| 31 | 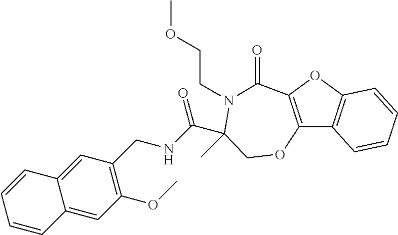 [SMILES: COCCN1C(=O)c2oc3ccccc3c2OCC1(C)C(=O)NCc1cc2ccccc2cc1OC] | 1H NMR (400 MHZ, CDCl3) δ 7.55 (t, J = 12.0, 6.8 Hz, 1H), 7.48 (d, J = 8.1 Hz, 1H), 7.42- 7.34 (m, 2H), 7.30 (ddd, J = 8.5, 6.2, 2.7 Hz, 2H), 7.25-7.13 (m, 3H), 6.87 (t, J = 7.3 Hz, 1H), 6.79 (s, 1H), 4.87 (d, J = 11.8 Hz, 1H), 4.58 (dd, J = 14.3, 7.5 Hz, 1H), 4.32-4.30 (m, 1H), 4.21 (dd, J = 14.3, 4.5 Hz, 1H), 4.07 (d, J = 11.8 Hz, 1H), 3.92 (s, 3H), 3.85-3.72 (m, 1H), 3.65-3.52 (m, 2H), 3.38-3.11 (m, 3H), 1.79 (s, 3H) LC-MS: 489.2[M + 1]+ |
| 32 | 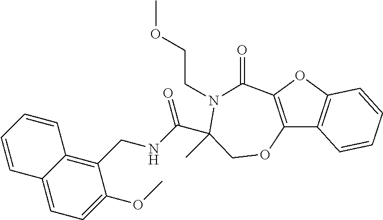 [SMILES: COCCN1C(=O)c2oc3ccccc3c2OCC1(C)C(=O)NCc1c(OC)ccc2ccccc12] | 1H NMR (400 MHZ, CDCl3) δ 7.86-7.76 (m, 1H), 7.59 (dd, J = 8.9, 4.6 Hz, 2H), 7.45 (d, J = 7.8 Hz, 1H), 7.39 (ddd, J = 8.3, 7.0, 1.3 Hz, 1H), 7.33 (d, J = 8.4 Hz, 1H), 7.30 (s, 1H), 7.22-7.11 (m, 3H), 7.08 (d, J = 9.0 Hz, 1H), 4.87 (dd, J = 14.0, 6.2 Hz, 2H), 4.67 (dd, J = 14.0, 4.4 Hz, 1H), 4.24-4.00 (m, 2H), 3.88 (s, 3H), 3.70-3.67 (m, 1H), 3.57-3.37 (m, 2H), 2.96 (s, 3H), 1.75 (s, 3H). LC-MS: 489.2[M + 1]+ |
| 33 | 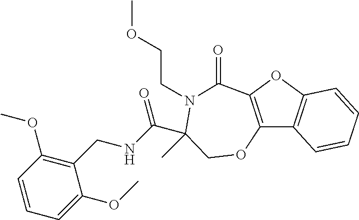 [SMILES: COCCN1C(=O)c2oc3ccccc3c2OCC1(C)C(=O)NCc1c(OC)cccc1OC] | 1H NMR (400 MHZ, CDCl3) δ 7.55 (d, J = 7.9 Hz, 1H), 7.39 (d, J = 3.6 Hz, 2H), 7.19 (dt, J = 8.0, 4.0 Hz, 1H), 7.11 (s, 1H), 6.92 (t, J = 8.3 Hz, 1H), 6.28 (d, J = 8.3 Hz, 2H), 4.83 (d, J = 11.7 Hz, 1H), 4.59 (dd, J = 13.8, 6.5 Hz, 1H), 4.27-4.02 (m, 3H), 3.69 (s, 6H), 3.67-3.50 (m, 3H), 3.19 (s, 3H), 1.75 (s, 3H). LC-MS: 469.2[M + 1]+ |
| 34 | 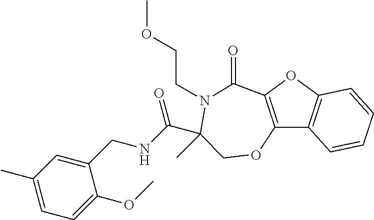 [SMILES: COCCN1C(=O)c2oc3ccccc3c2OCC1(C)C(=O)NCc1cc(C)ccc1OC] | 1H NMR (400 MHZ, CDCl3) δ 7.52 (d, J = 7.9 Hz, 1H), 7.44- 7.32 (m, 3H), 7.20-7.16 (m , 1H), 6.74 (s, 1H), 6.65 (d, J = 7.9 Hz, 1H), 6.45 (d, J = 8.2 Hz, 1H), 4.86 (d, J = 11.9 Hz, 1H), 4.34 (dd, J = 14.1, 7.1 Hz, 1H), 4.28-3.96 (m, 3H), 3.79-3.78 (m, 1H), 3.73 (s, 3H), 3.59-3.57 (m, 2H), 3.26 (s, 3H), 1.97 (s, 3H), 1.77 (s, 3H). LC-MS: 453.2[M + 1]+ |
| 35 | 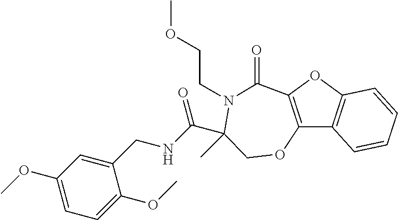 [SMILES: COCCN1C(=O)c2oc3ccccc3c2OCC1(C)C(=O)NCc1cc(OC)ccc1OC] | 1H NMR (400 MHZ, CDCl3) δ 7.54 (d, J = 7.9 Hz, 1H), 7.46- 7.33 (m, 3H), 7.21-7.16 (m, 1H), 6.57 (d, J = 2.8 Hz, 1H), 6.50 (d, J = 8.8 Hz, 1H), 6.42 (dd, J = 8.8, 2.5 Hz, 1H), 4.86 (d, J = 11.8 Hz, 1H), 4.31 (dd, J = 14.2, 6.9 Hz, 1H), 4.26-4.01 (m, 3H), 3.86-3.74 (m, 1H), 3.72 (s, 3H), 3.65-3.58 (m, 2H), 3.56 (s, 3H), 3.25 (s, 3H), 1.77 (s, 3H). LC-MS: 469.0[M + 1]+ |
| 36 | 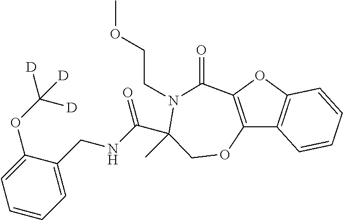 [SMILES: [2H]C([2H])([2H])Oc1ccccc1CNC(=O)C1(C)COc2c(oc3ccccc23)C(=O)N1CCOC] | 1H NMR (400 MHZ, CDCl3) δ 7.54 (d, J = 7.9 Hz, 1H), 7.41- 7.40 (m, 3H), 7.20-7.18 (m, 1H), 6.88 (d, J = 6.9 Hz, 2H), 6.55 (d, J = 7.9 Hz, 1H), 6.46 ((t, J = 4 Hz, 1H), 4.87 (d, J = 12.0 Hz, 1H), 4.46-4.18 (m, 2H), 4.11 (t, J = 13.3 Hz, 2H), 3.77-3.75 (m, 1H), 3.60-3.55 (m, 2H), 3.24 (s, 3H), 1.77 (s, 3H). LC-MS: 442.2[M + 1]+ |
| 37 | 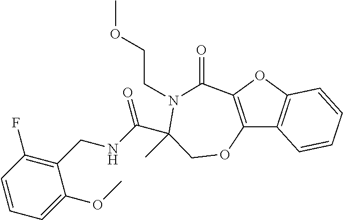 [SMILES: COCCN1C(=O)c2oc3ccccc3c2OCC1(C)C(=O)NCc1c(F)cccc1OC] | 1H NMR (400 MHz, CDCl3) δ 7.54-7.51 (m, 2H), 7.45-7.34 (m, 2H), 7.22-7.18 (m, 1H), 6.67 (dd, J = 8.4, 2.8 Hz, 1H), 6.57- 6.35 (m, 2H), 4.88 (d, J = 11.9 Hz, 1H), 4.37-4.31 (m, 2H), 4.18- 3.94 (m, 2H), 3.89-3.76 (m, 1H), 3.72 (s, 3H), 3.65-3.45 (m, 2H), 3.30 (s, 3H), 1.78 (s, 3H). LC-MS: 457.2[M + 1]+ |
| 38 | 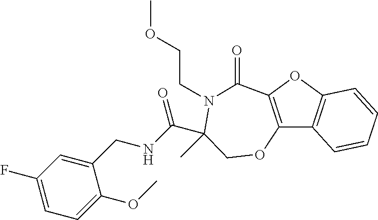 [SMILES: COCCN1C(=O)c2oc3ccccc3c2OCC1(C)C(=O)NCc1cc(F)ccc1OC] | 1H NMR (400 MHZ, CDCl3) δ 7.54-7.51 (m, 2H), 7.45-7.32 (m, 2H), 7.19 (ddd, J = 8.0, 5.5, 2.5 Hz, 1H), 6.67 (dd, J = 8.4, 2.8 Hz, 1H), 6.50 (dt, J = 8.3, 4.3 Hz, 1H), 6.43 (dd, J = 8.8, 4.3 Hz, 1H), 4.88 (d, J = 11.8 Hz, 1H), 4.37-4.31 (m, 2H), 4.17- 4.00 (m, 2H), 3.87-3.76 (m, 1H), 3.72 (s, 3H), 3.64-3.49 (m, 2H), 3.30 (s, 3H), 1.78 (s, 3H). LC-MS: 457.2[M + 1]+ |
| 39 | 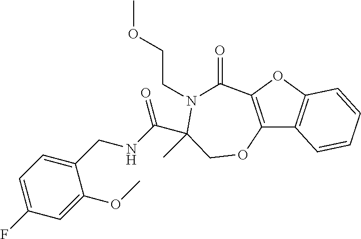 [SMILES: COCCN1C(=O)c2oc3ccccc3c2OCC1(C)C(=O)NCc1ccc(F)cc1OC] | 1H NMR (400 MHZ, CDCl3) δ 7.51 (d, J = 7.9 Hz, 1H), 7.45- 7.38 (m, 3H), 7.19 (ddd, J = 8.0, 6.6, 1.5 Hz, 1H), 6.79 (t, J = 7.4 Hz, 1H), 6.24 (dd, J = 10.8, 2.0 Hz, 1H), 6.05 (t, J = 7.4 Hz, 1H), 4.88 (d, J = 11.8 Hz, 1H), 4.40- 4.34 (m, 2H), 4.08 (d, J = 11.9 Hz, 1H), 3.99 (dd, J = 14.2, 4.5 Hz, 1H), 3.78 (ddd, J = 7.5, 5.8, 3.1 Hz, 1H), 3.74 (s, 3H), 3.62- 3.55 (m, 2H), 3.31 (s, 3H), 1.78 (s, 3H). LC-MS: 457.2[M + 1]+ |
| 40 | 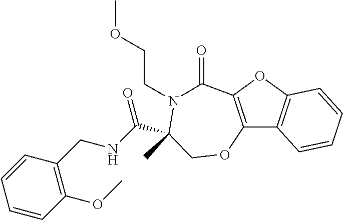 [SMILES: COCCN1C(=O)c2oc3ccccc3c2OC[C@]1(C)C(=O)NCc1ccccc1OC] | 1H NMR (400 MHZ, CDCl3) δ 7.54 (d, J = 7.9 Hz, 1H), 7.47- 7.33 (m, 3H), 7.23-7.14 (m, 1H), 6.91-6.87 (m, 2H), 6.56 (d, J = 8.0 Hz, 1H), 6.46 (t, J = 7.2 Hz, 1H), 4.87 (d, J = 11.8 Hz, 1H), 4.37 (dd, J = 14.3, 6.8 Hz, 1H), 4.30-4.18 (m, 1H), 4.18-4.00 (m, 2H), 3.85-3.74 (m, 1H), 3.74 (s, 3H), 3.65- 3.51 (m, 2H), 3.24 (s, 3H), 1.77 (s, 3H). LC-MS: 438.99[M + 1]+ |
| 41 | 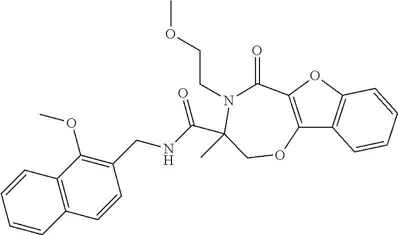 [SMILES: COCCN1C(=O)c2oc3ccccc3c2OCC1(C)C(=O)NCc1ccc2ccccc2c1OC] | 1H NMR (400 MHZ, CDCl3) δ 7.96 (d, J = 8.1 Hz, 1H), 7.65 (d, J = 7.5 Hz, 2H), 7.55 (t, J = 8.0 Hz, 2H), 7.48-7.32 (m, 4H), 7.25 (t, J = 8.0 Hz, 2H), 7.05 (d, J = 8.0 Hz, 1H), 6.91 (d, J = 8.3 Hz, 1H), 4.94 (d, J = 11.5 Hz, 1H), 4.60 (dd, J = 14.6, 6.3 Hz, 1H), 4.40 (dd, J = 14.6, 5.2 Hz, 1H), 4.32-4.21 (m, 1H), 4.18 (d, J = 11.9 Hz, 1H), 3.99-3.81 (m, 1H), 3.81 (s, 3H), 3.58-3.49 (m, 2H), 3.13 (s, 3H), 1.80 (s, 3H). LC-MS: 489.2[M + 1]+ |
| 42 | 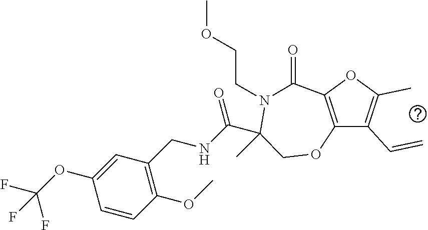 | 1H NMR (400 MHZ, CDCl3) δ 7.55 (t, J = 5.4 Hz, 1H), 7.50 (d, J = 7.9 Hz, 1H), 7.45-7.32 (m, 2H), 7.18 (ddd, J = 8.0, 5.0, 3.0 Hz, 1H), 6.90 (d, J = 2.2 Hz, 1H), 6.71 (d, J = 8.0 Hz, 1H), 6.47 (d, J = 8.9 Hz, 1H), 4.87 (d, J = 11.9 Hz, 1H), 4.33 (dd, J = 14.4, 7.0 Hz, 2H), 4.19-3.98 (m, 2H), 3.83-3.78 (m, 1H), 3.75 (s, 3H), 3.62-3.51 (m, 2H), 3.28 (s, 3H), 1.77 (s, 3H). LC-MS: 523.1[M + 1]+ |
| 43 | 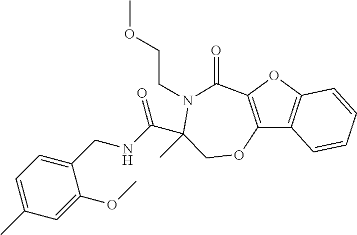 [SMILES: COCCN1C(=O)c2oc3ccccc3c2OCC1(C)C(=O)NCc1ccc(C)cc1OC] | 1H NMR (400 MHZ, CDCl3) δ 7.53 (d, J = 7.9 Hz, 1H), 7.45- 7.37 (m, 2H), 7.34 (s, 1H), 7.18 (ddd, J = 8.0, 4.7, 3.4 Hz, 1H), 6.78 (d, J = 7.5 Hz, 1H), 6.36 (s, 1H), 6.28 (d, J = 7.3 Hz, 1H), 4.86 (d, J = 12.0 Hz, 1H), 4.35 (dd, J = 14.2, 7.0 Hz, 1H), 4.25- 4.21 (m, 1H), 4.11-4.06 (m, 2H), 3.81-3.65 (m, 4H), 3.62- 3.56 (m, 2H), 3.26 (s, 3H), 2.12 (s, 3H), 1.77 (s, 3H). LC-MS: 453.0[M + 1]+ |
| 44 | 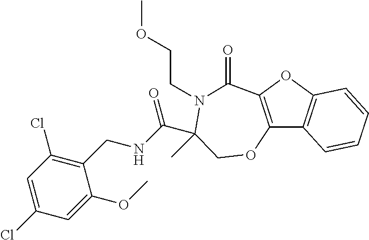 [SMILES: COCCN1C(=O)c2oc3ccccc3c2OCC1(C)C(=O)NCc1c(Cl)cc(Cl)cc1OC] | 1H NMR (400 MHZ, CDCl3) δ 7.53 (d, J = 7.8 Hz, 1H), 7.46- 7.37 (m, 2H), 7.34 (s, 1H), 7.20 (ddd, J = 8.0, 6.9, 1.1 Hz, 1H), 6.59 (s, 1H), 6.43 (s, 1H), 4.98- 4.73 (m, 2H), 4.35-4.31 (m, 1H), 4.08 (d, J = 11.8 Hz, 1H), 4.04-3.91 (m, 1H), 3.85-3.74 (m, 1H), 3.71 (s, 3H), 3.65- 3.45 (m, 2H), 3.29 (s, 3H), 1.78 (s, 3H). LC-MS: 507.0[M + 1]+ |
| 45 | 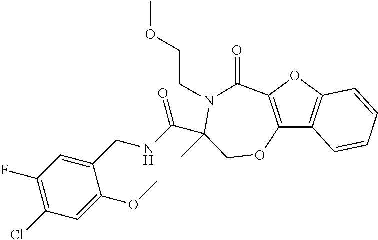 [SMILES: COCCN1C(=O)c2oc3ccccc3c2OCC1(C)C(=O)NCc1cc(F)c(Cl)cc1OC] | 1H NMR (400 MHZ, CDCl3) δ 7.55 (t, J = 5.6 Hz, 1H), 7.50 (d, J = 7.9 Hz, 1H), 7.48-7.41 (m, 1H), 7.38 (d, J = 8.3 Hz, 1H), 7.20 (t, J = 7.4 Hz, 1H), 6.71 (d, J = 8.9 Hz, 1H), 6.48 (d, J = 5.9 Hz, 1H), 4.89 (d, J = 11.9 Hz, 1H), 4.47-4.28 (m, 2H), 4.08 (d, J = 11.9 Hz, 1H), 3.95 (dd, J = 14.4, 4.7 Hz, 1H), 3.87-3.78 (m, 1H), 3.73 (s, 3H), 3.68-3.49 (m, 2H), 3.36 (s, 3H), 1.79 (s, 3H). LC-MS: 491.10[M + 1]+ |
| 46 | 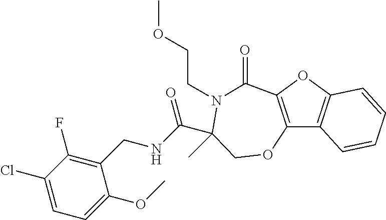 [SMILES: COCCN1C(=O)c2oc3ccccc3c2OCC1(C)C(=O)NCc1c(OC)ccc(Cl)c1F] | 1H NMR (400 MHZ, CDCl3) δ 7.53 (d, J = 7.8 Hz, 1H), 7.49- 7.35 (m, 3H), 7.20 (ddd, J = 8.0, 6.2, 1.9 Hz, 1H), 6.82 (t, J = 8.6 Hz, 1H), 6.23 (d, J = 8.8 Hz, 1H), 4.86 (d, J = 11.8 Hz, 1H), 4.68 (dd, J = 14.0, 7.3 Hz, 1H), 4.42-4.26 (m, 1H), 4.14-3.93 (m, 2H), 3.86-3.74 (m, 1H), 3.69 (s, 3H), 3.65-3.46 (m, 2H), 3.29 (s, 3H), 1.77 (s, 3H). LC-MS: 491.10[M + 1]+ |
| 47 | 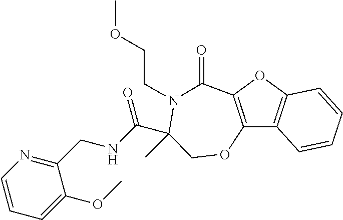 [SMILES: COCCN1C(=O)c2oc3ccccc3c2OCC1(C)C(=O)NCc1ncccc1OC] | 1H NMR (400 MHZ, CDCl3) δ 7.95 (d, J = 4.2 Hz, 1H), 7.83 (s, 1H), 7.63 (d, J = 7.8 Hz, 1H), 7.49-7.37 (m, 2H), 7.25-7.18 (m, 1H), 7.09-6.94 (m, 2H), 4.83 (d, J = 11.8 Hz, 1H), 4.44 (qd, J = 17.1, 4.5 Hz, 2H), 4.27 (d, J = 12.1 Hz, 1H), 4.05-3.92 (m, 1H), 3.86-3.76 (m, 2H), 3.76 (s, 3H), 3.78-3.62 (m, 1H), 3.26 (s, 3H), 1.79 (s, 3H). LC-MS: 440.10[M + 1]+ |
| 48 | 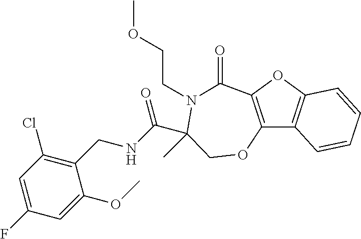 [SMILES: COCCN1C(=O)c2oc3ccccc3c2OCC1(C)C(=O)NCc1c(Cl)cc(F)cc1OC] | 1H NMR (400 MHz, DMSO) δ 7.86 (s, 1H), 7.65-7.53 (m, 2H), 7.53-7.43 (m, 1H), 7.30 (t, J = 7.5 Hz, 1H), 6.93-6.71 (m, 2H), 4.81 (d, J = 11.3 Hz, 1H), 4.40-4.17 (m, 3H), 3.74 (s, 3H), 3.67-3.55 (m, 2H), 3.48 (t, J = 5.9 Hz, 2H), 3.20 (s, 3H), 1.59 (s, 3H). LC-MS: 491.13[M + 1]+ |

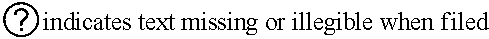

### Intermediate BB-3

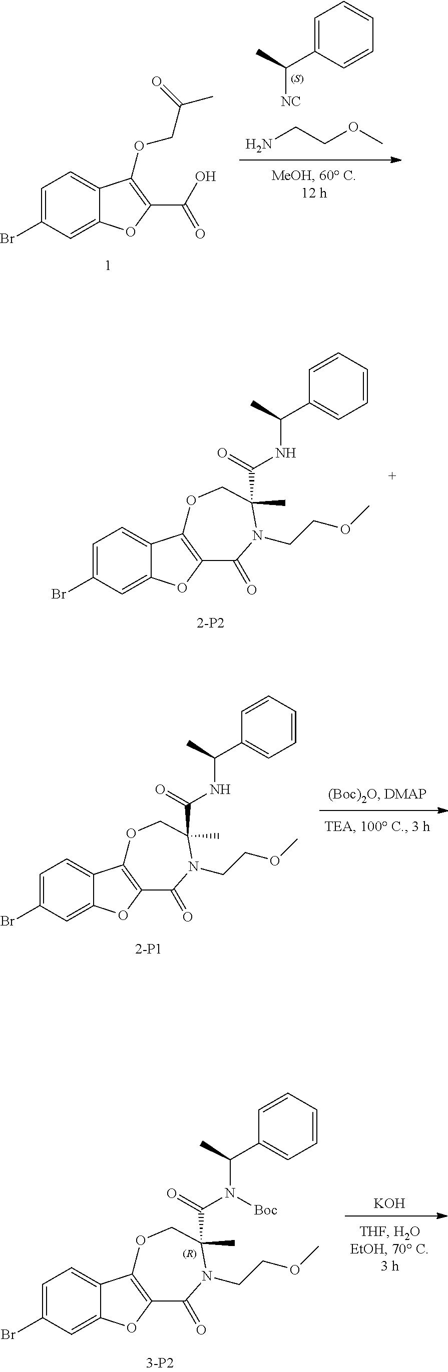

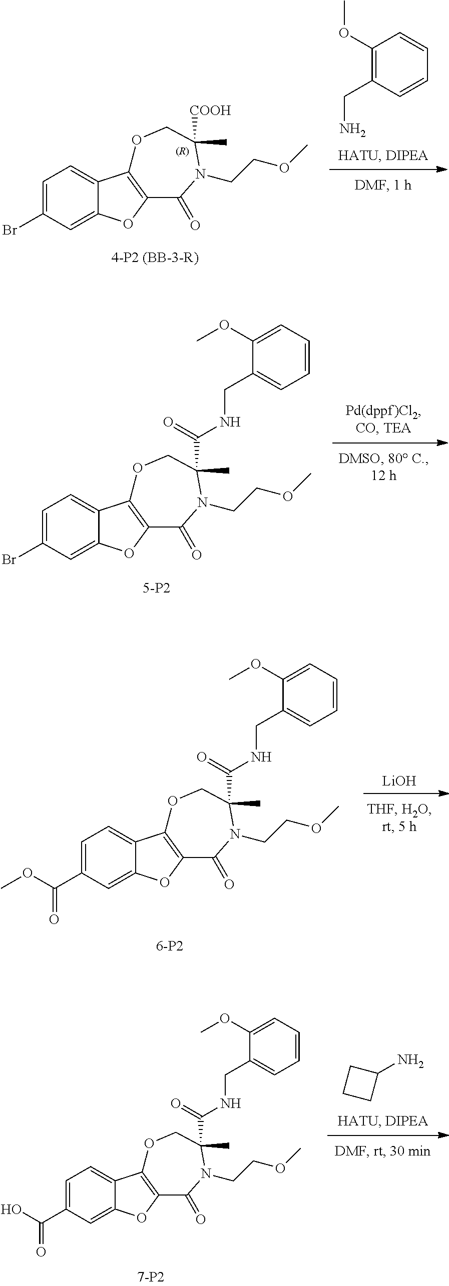

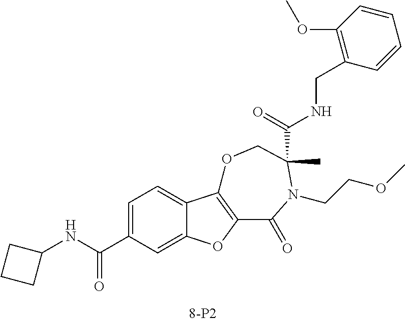 [SMILES: COCCN1C(=O)c2oc3cc(C(=O)NC4CCC4)ccc3c2OC[C@]1(C)C(=O)NCc1ccccc1OC]

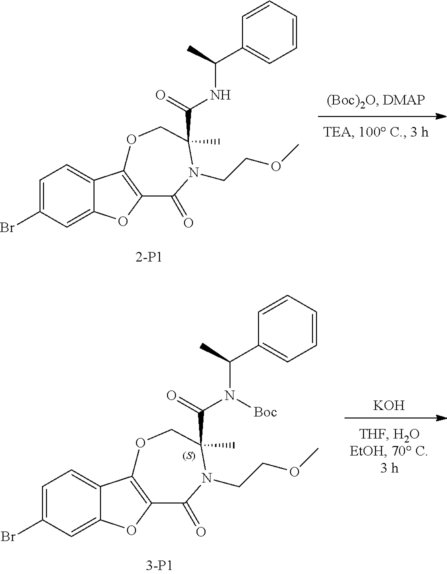

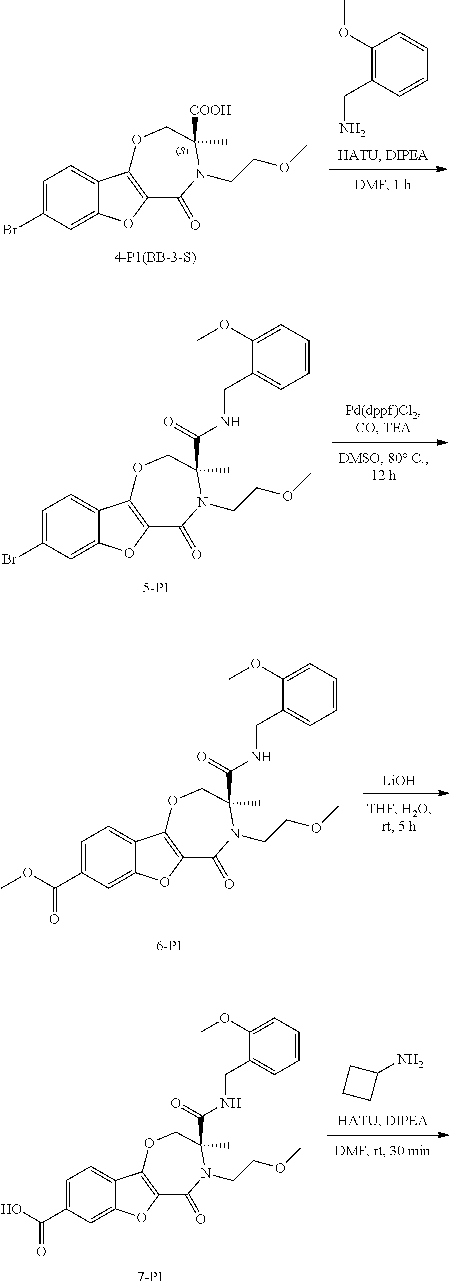

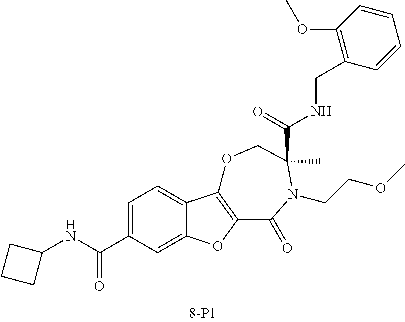 [SMILES: COCCN1C(=O)c2oc3cc(C(=O)NC4CCC4)ccc3c2OC[C@@]1(C)C(=O)NCc1ccccc1OC]

A mixture of 6-bromo-3-(2-oxopropoxy)benzofuran-2-carboxylic acid (4 g, 12.8 mmol, 1.0 eq), (S)-(1-isocyanoethyl)benzene (2.2 g, 16.6 mmol, 1.3 eq), 2-methoxyethanamine (1.4 g, 19.2 mmol, 1.5 eq) in MeOH (50 mL) was stirred at 60° C. overnight. The mixture was concentrated under reduced pressure to afford a residue. The residue was purified by silica gel chromatography (PE:EA=1:1) to afford (R)-8-bromo-4-(2-methoxyethyl)-3-methyl-5-oxo-N—((S)-1-phenylethyl)-2,3,4,5-tetrahydrobenzofuro[2,3-f][1,4]oxazepine-3-carboxamide (2-P2) (670 mg, 10%), (S)-8-bromo-4-(2-methoxyethyl)-3-methyl-5-oxo-N—((S)-1-phenylethyl)-2,3,4,5-tetrahydrobenzofuro[2,3-f][1,4]oxazepine-3-carboxamide(S)-8-bromo-4-(2-methoxyethyl)-3-methyl-5-oxo-N—((S)-1-phenylethyl)-2,3,4,5-tetrahydrobenzofuro[2,3-f][1,4]oxazepine-3-carboxamide (2-P1) (580 mg, 9%) as brown solid. LC-MS: 501.1 [M+1]⁺. The chiral resolution of 2-P1, 2-P2 was carried out using Chiral OD-H column (4.6 mm×252 mm), 40Pre MeOH FA; flow rate at 1.0 mL/min, monitored at 254 nm.

The peak 1 (front peak) was named 2-P1 (6.644 min, 100% ee). LC-MS: 517.1 [M+1]+; 1H NMR (400 MHz, CDCl3) δ 7.66 (d, J=1.3 Hz, 1H), 7.56 (d, J=8.4 Hz, 1H), 7.39 (dd, J=8.4, 1.5 Hz, 1H), 7.34-7.27 (m, 3H), 7.25-7.16 (m, 3H), 5.04-4.70 (m, 2H), 4.33-4.06 (m, 2H), 3.99-3.82 (m, 1H), 3.62-3.34 (m, 2H), 2.95 (s, 3H), 1.71 (s, 3H), 1.05 (d, J=6.4 Hz, 3H).

The peak 2 (2nd peak) was named 2-P2 (7.410 min, 97% ee).

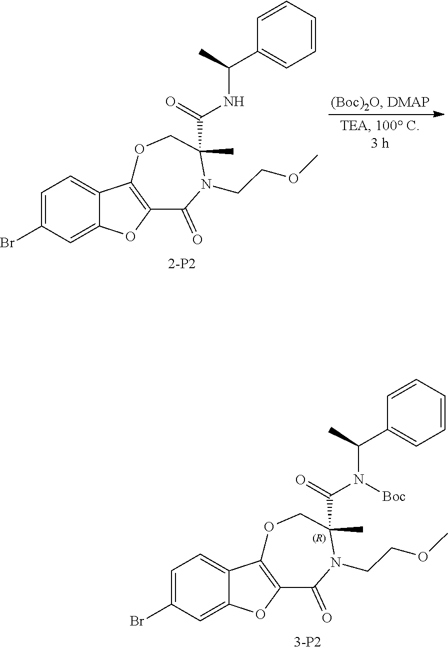

To a solution of (R)-8-bromo-4-(2-methoxyethyl)-3-methyl-5-oxo-N—((S)-1-phenylethyl)-2,3,4,5-tetrahydrobenzofuro[2,3-f][1,4]oxazepine-3-carboxamide (570 mg, 1.14 mmol, 1.0 eq) in TEA (10 mL) was added (Boc)₂O (1 g, 4.54 mmol, 4.0 eq), DMAP (277 mg, 2.27 mmol, 2.0 eq). The mixture was stirred at 100° C. for 3 h. Water (50 mL) was added. The aqueous phase was extracted with EA (3×50 mL). The combined organic solution was washed with brine (50 mL), dried over anhydrous Na₂SO₄, and the solution was concentrated under reduced pressure. The residue was purified by silica gel chromatography (PE:EA=3:1) to give tert-butyl ((R)-8-bromo-4-(2-methoxyethyl)-3-methyl-5-oxo-2,3,4,5-tetrahydrobenzofuro[2,3-f][1,4]oxazepine-3-carbonyl)((S)-1-phenylethyl)carbamate (600 mg, 87%) as brown solid. LC-MS: 603.1 [M+1]⁺.

tert-butyl ((S)-8-bromo-4-(2-methoxyethyl)-3-methyl-5-oxo-2,3,4,5-tetrahydrobenzofuro[2,3-f][1,4]oxazepine-3-carbonyl)((S)-1-phenylethyl)carbamate (3-P1) (284 mg, 80%) was generated as a brown solid in similar fashion. LC-MS: 603.1 [M+1]⁺.

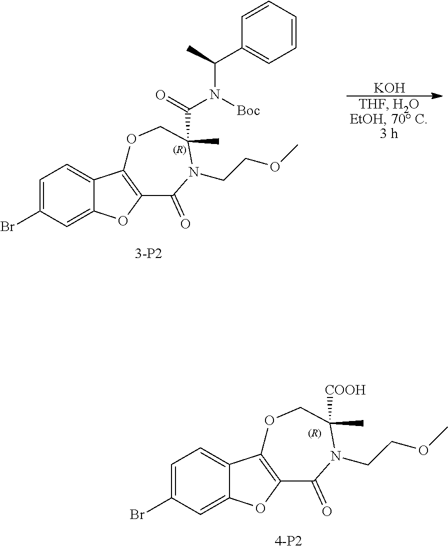

To a solution of tert-butyl ((R)-8-bromo-4-(2-methoxyethyl)-3-methyl-5-oxo-2,3,4,5-tetrahydrobenzofuro[2,3-f][1,4]oxazepine-3-carbonyl)((S)-1-phenylethyl)carbamate (600 mg, 0.99 mmol, 1.0 eq) in THF (5 mL), EtOH (10 mL), H₂O (3 mL) was added KOH (560 mg, 10.0 mmol, 10.0 eq.). The mixture was stirred at 70° C. for 3 h. The reaction mixture was concentrated under reduced pressure and then acidified to pH 4-5 with 1 N HCl. The solid was filtered to give (R)-8-bromo-4-(2-methoxyethyl)-3-methyl-5-oxo-2,3,4,5-tetrahydrobenzofuro[2,3-f][1,4]oxazepine-3-carboxylic acid (440 mg, 110%) as white solid. The crude product used in the next step directly. LC-MS: 398.1 [M+1]⁺.

(S)-8-bromo-4-(2-methoxyethyl)-3-methyl-5-oxo-2,3,4,5-tetrahydrobenzofuro[2,3-f][1,4]oxazepine-3-carboxylic acid (243 mg, 129%) was generated as a white solid in similar fashion; LC-MS: 398.1 [M+1]⁺.

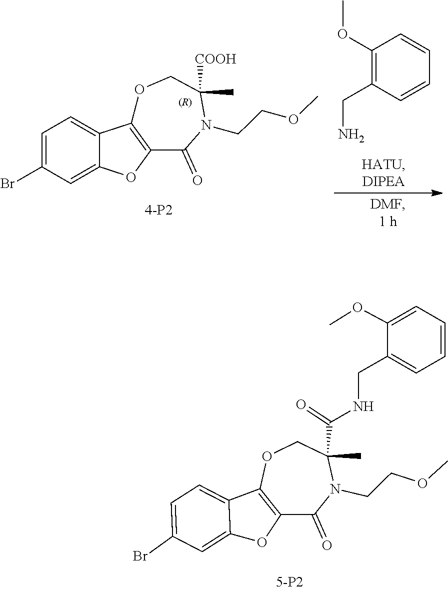

To a solution of (R)-8-bromo-4-(2-methoxyethyl)-3-methyl-5-oxo-2,3,4,5-tetrahydrobenzofuro[2,3-f][1,4]oxazepine-3-carboxylic acid (440 mg, 1.10 mmol, 1.0 eq.) in DMF (10 mL) was added HATU (627 mg, 1.65 mmol, 1.5 eq.), (2-methoxyphenyl)methanamine (226 mg, 1.65 mmol, 1.5 eq.), DIPEA (284 mg, 2.20 mmol, 2.0 eq.) at 0° C. After stirring at room temperature for 1 h, the reaction mixture was diluted with water (30 mL). The aqueous phase was extracted with ethyl acetate (3×30 mL). The combined organic phases were washed with brine (20 mL), and dried over anhydrous magnesium sulfate. The solvent was removed under reduced pressure and the residue was purified by flash column chromatography (EA) to give the title compound as a yellow solid (570 mg, 100%). LCMS: 517.1 [M+1]+.

(S)-8-bromo-N-(2-methoxybenzyl)-4-(2-methoxyethyl)-3-methyl-5-oxo-2,3,4,5-tetrahydrobenzofuro[2,3-f][1,4]oxazepine-3-carboxamide (256 mg, 81%) was generated in similar fashion. LCMS: 517.1 [M+1]+.

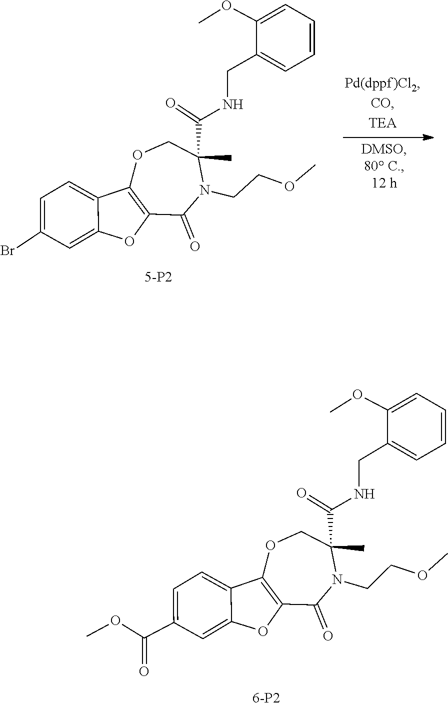

A mixture containing (R)-8-bromo-N-(2-methoxybenzyl)-4-(2-methoxyethyl)-3-methyl-5-oxo-2,3,4,5-tetrahydrobenzofuro[2,3-f][1,4]oxazepine-3-carboxamide (570 mg, 1.10 mmol, 1.0 eq.), Pd(OAc)₂ (25 mg, 0.11 mmol, 0.1 eq.), DPPF (61 mg, 0.11 mmol, 0.1 eq.), TEA (559 mg, 5.50 mmol, 5.0 eq.) in DMSO (10 mL) and MeOH (5 mL) was stirred in a CO gas atmosphere at 85° C. for 12 hours. The reaction mixture was diluted with water (30 mL). The aqueous phase was extracted with ethyl acetate (3×30 mL). The combined organic phases were washed with brine (20 mL), and dried over anhydrous magnesium sulfate. The solvent was removed under reduced pressure and the residue was purified by flash column chromatography (EA) to give the title compound as a light brown solid (400 mg, 73%). LCMS: 497.1 [M+1]⁺.

(S)-methyl 3-((2-methoxybenzyl)carbamoyl)-4-(2-methoxyethyl)-3-methyl-5-oxo-2,3,4,5-tetrahydrobenzofuro[2,3-f][1,4]oxazepine-8-carboxylate (130 mg, 52%) was generated in similar fashion. LCMS: 497.1 [M+1]⁺.

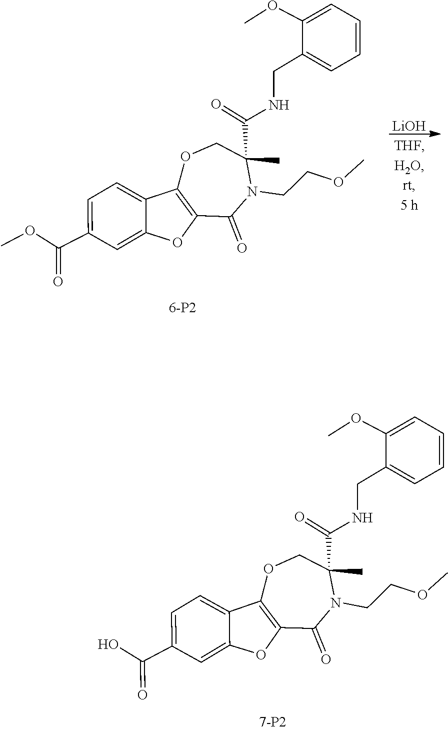

To a solution of (R)-methyl 3-((2-methoxybenzyl)carbamoyl)-4-(2-methoxyethyl)-3-methyl-5-oxo-2,3,4,5-tetrahydrobenzofuro[2,3-f][1,4]oxazepine-8-carboxylate (400 mg, 0.83 mmol, 1.0 eq) in THF (6 mL), H₂O (3 mL) was added LiOH (200 mg, 8.30 mmol, 10.0 eq.). The mixture was stirred at room temperature for 5 h. The reaction mixture was concentrated under reduced pressure and then acidified to pH 4-5 with 1 N HCl. The aqueous phase was extracted with ethyl acetate (3×30 mL). The combined organic phases were washed with brine (20 mL), and dried over anhydrous magnesium sulfate to give crude product (200 mg, 50%). The crude product used in the next step directly. LC-MS: 483.1 [M+1]⁺.

### Example 49

(S)—N₈-cyclobutyl-N₃-(2-methoxybenzyl)-4-(2-methoxyethyl)-3-methyl-5-oxo-2,3,4,5-tetrahydrobenzofuro[2,3-f][1,4]oxazepine-3,8-dicarboxamide (79 mg, 59%). Using (S)-3-((2-methoxybenzyl)carbamoyl)-4-(2-methoxyethyl)-3-methyl-5-oxo-2,3,4,5-tetrahydrobenzofuro[2,3-f][1,4]oxazepine-8-carboxylic acid, the title compound was produced: LC-MS: 536.20[M+1]+; 1H NMR (400 MHz, CDCl3) δ 7.83 (s, 1H), 7.55 (d, J=8.2 Hz, 1H), 7.54-7.49 (m, 1H), 7.49-7.40 (m, 1H), 6.93-6.78 (m, 2H), 6.57 (d, J=8.0 Hz, 1H), 6.44 (t, J=7.2 Hz, 1H), 6.26 (d, J=7.5 Hz, 1H), 4.89 (d, J=11.8 Hz, 1H), 4.70-4.56 (m, 1H), 4.35 (dd, J=14.2, 7.0 Hz, 1H), 4.30-4.21 (m, 1H), 4.18-4.00 (m, 2H), 3.82-3.77 (m, 1H), 3.76 (s, 3H), 3.65-3.52 (m, 2H), 3.25 (s, 3H), 2.54-2.41 (m, 2H), 2.09-1.93 (m, 2H), 1.85-1.80 (m, 2H), 1.78 (s, 3H).

### Example 50

### (R)—N₈-cyclobutyl-N₃-(2-methoxybenzyl)-4-(2-methoxyethyl)-3-methyl-5-oxo-2,3,4,5-tetrahydrobenzofuro[2,3-f][1,4]oxazepine-3,8-dicarboxamide

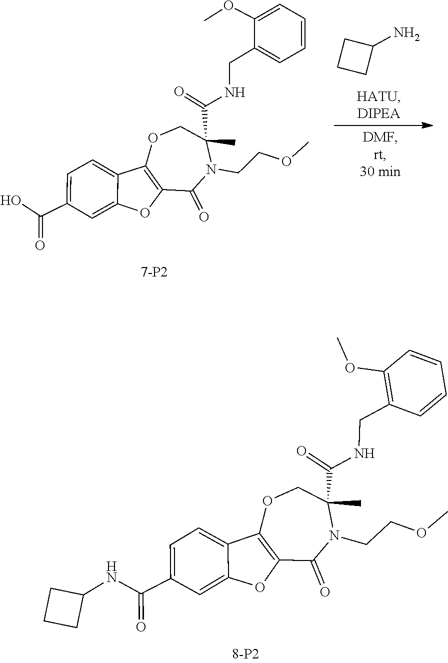

To a solution of (R)-3-((2-methoxybenzyl)carbamoyl)-4-(2-methoxyethyl)-3-methyl-5-oxo-2,3,4,5-tetrahydrobenzofuro[2,3-f][1,4]oxazepine-8-carboxylic acid (200 mg, 0.42 mmol, 1.0 eq.) in DMF (5 mL) was added HATU (239.5 mg, 0.63 mmol, 1.5 eq.), cyclobutanamine (44.8 mg, 0.63 mmol, 1.5 eq.), DIPEA (108.6 mg, 0.84 mmol, 2.0 eq.) at 0° C. After stirring at room temperature for 30 min, the reaction mixture was diluted with water (20 mL). The aqueous phase was extracted with ethyl acetate (3×20 mL). The combined organic phases were washed with brine (20 mL), and dried over anhydrous magnesium sulfate. The solvent was removed under reduced pressure and the residue was purified by flash column chromatography (EA) to give (R)—N₈-cyclobutyl-N₃-(2-methoxybenzyl)-4-(2-methoxyethyl)-3-methyl-5-oxo-2,3,4,5-tetrahydrobenzofuro[2,3-f][1,4]oxazepine-3,8-dicarboxamide as a yellow solid (120 g, 53%).

LC-MS: 536.20[M+1]+. 1H NMR (400 MHz, CDCl3) δ 7.82 (s, 1H), 7.59-7.49 (m, 2H), 7.45 (t, J=5.2 Hz, 1H), 6.92-6.80 (m, 2H), 6.57 (d, J=8.0 Hz, 1H), 6.44 (t, J=7.2 Hz, 1H), 6.28 (d, J=7.4 Hz, 1H), 4.89 (d, J=11.7 Hz, 1H), 4.71-4.52 (m, 1H), 4.35 (dd, J=14.2, 7.0 Hz, 1H), 4.30-4.20 (m, 1H), 4.18-4.01 (m, 2H), 3.84-3.77 (m, 1H), 3.76 (s, 3H), 3.68-3.53 (m, 2H), 3.25 (s, 3H), 2.56-2.38 (m, 2H), 2.15-1.94 (m, 2H), 1.88-1.79 (m, 2H), 1.78 (s, 3H).

### Intermediate BB-4

### 3-((2-methoxybenzyl)carbamoyl)-4-(2-methoxyethyl)-3-methyl-5-oxo-2,3,4,5-tetrahydrobenzofuro[2,3-f][1,4]oxazepine-8-carboxylic acid

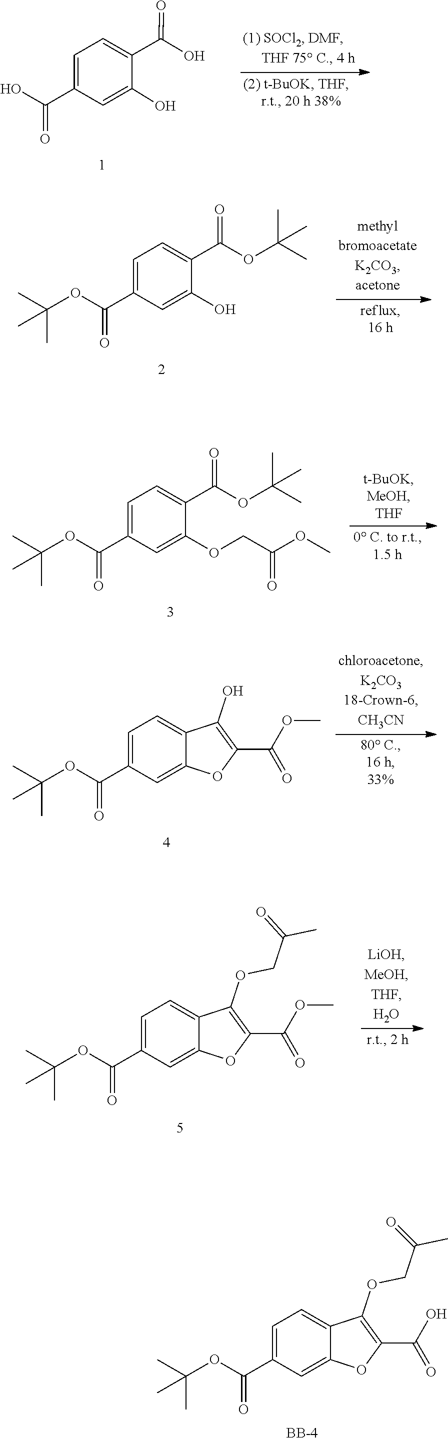

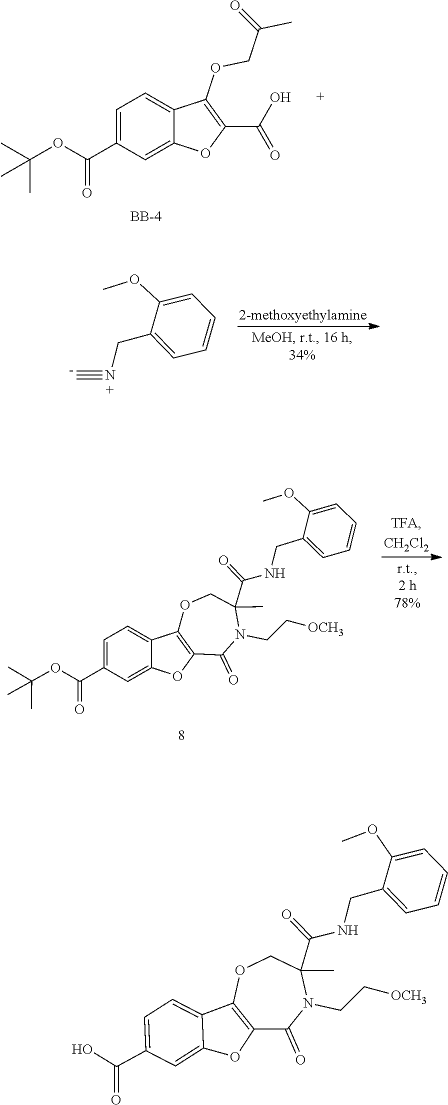

A mixture of 2-hydroxyterephthalic acid (50 g, 274.6 mmol, 1.0 eq), SOCl₂ (300 mL), and DMF (1 mL) in THF (500 mL) was heated at 75° C. and stirred for 4 h. The reaction mixture was cooled to room temperature and concentrated under reduced pressure. The residue was dissolved in THF (100 mL) and added dropwise to a solution of t-BuOK (123 g, 1.1 mol, 4.0 eq) in THF (500 mL). The reaction mixture was stirred at room temperature for 16 h and filtered. The filtrate was concentrated under reduced pressure. The crude product was purified by column chromatography on silica gel (Hex/EtOAc, 100:1 to 50:1) to give di-tert-butyl 2-hydroxyterephthalate (30 g, 38%) as a light-yellow solid. TLC: PE/EA=10:1, UV; R_{f}(compound 1)=0.01; R_{f} (compound 2)=0.80; ¹H NMR (400 MHz, CDCl₃) δ (ppm): 11.00 (d, J=2.1 Hz, 1H), 7.78 (d, J=8.3 Hz, 1H), 7.53 (d, J=1.7 Hz, 1H), 7.42 (dd, J=8.3, 1.7 Hz, 1H), 1.62 (s, 9H), 1.57 (s, 9H).

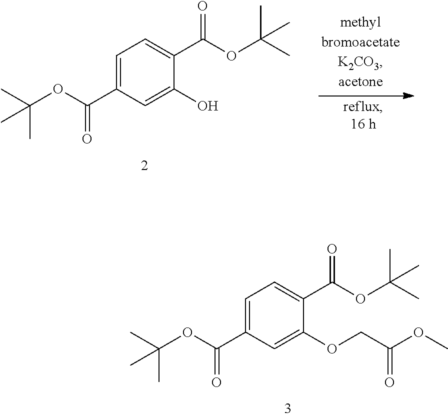

To a solution of di-tert-butyl 2-hydroxyterephthalate (30 g, 96.6 mmol, 1.0 eq) and K₂CO₃ (26.7 g, 193.3 mmol, 2.0 eq) in acetone (300 mL) was added methyl bromoacetate (29.5 g, 193.3 mmol, 2.0 eq). The reaction mixture was heated to reflux for 16 h, cooled to room temperature, and filtered. The filtrate was concentrated under reduced pressure to give crude di-tert-butyl 2-(2-methoxy-2-oxoethoxy)terephthalate (39.9 g) as a yellow solid. TLC: PE/EA=10:1, UV; R_{f}(compound 2)=0.80; R_{f} (compound 3)=0.30; ¹H NMR (400 MHz, CDCl₃) δ (ppm): 7.72-7.68 (m, 1H), 7.59 (dd, J=7.9, 1.5 Hz, 1H), 7.45 (d, J=1.4 Hz, 1H), 4.72 (s, 2H), 3.78 (s, 3H), 1.59 (s, 9H), 1.55 (s, 9H).

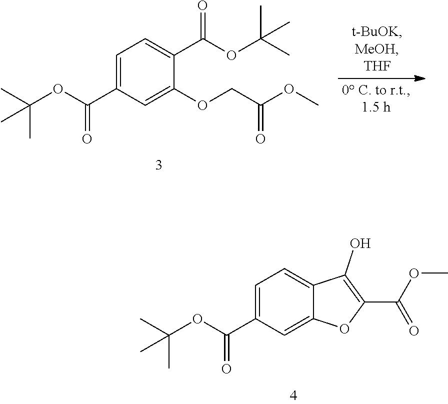

To a solution of di-tert-butyl 2-(2-methoxy-2-oxoethoxy)terephthalate (39.9 g, 108.9 mmol, 1.0 eq) in MeOH (100 mL) at 0° C. was added t-BuOK (18.3 g, 163.4 mmol, 1.5 eq) in THE (200 mL) dropwise. The reaction mixture was stirred at room temperature for 1.5 h. The mixture was quenched by aqueous NH₄Cl solution and extracted with EtOAc. The combined organic layers were dried over anhydrous Na₂SO₄, filtered, and concentrated under reduced pressure to give crude 6-tert-butyl 2-methyl 3-hydroxybenzofuran-2,6-dicarboxylate (27 g) as a light yellow solid. ¹H NMR (400 MHz, CD₃OD): δ (ppm) 7.99 (d, J=1.1 Hz, 1H), 7.86 (dd, J=8.3, 1.4 Hz, 1H), 7.78 (dd, J=8.4, 0.9 Hz, 1H), 3.93 (s, 3H), 1.60 (s, 9H).

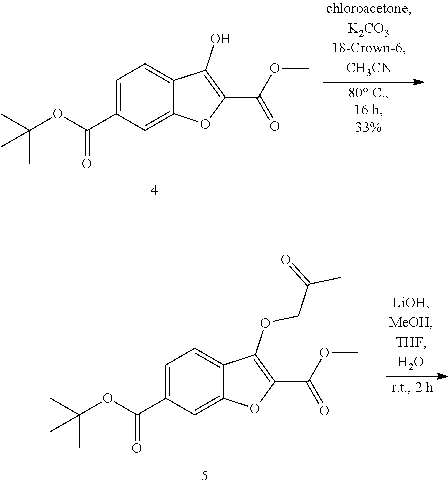

A mixture of 6-tert-butyl 2-methyl 3-hydroxybenzofuran-2,6-dicarboxylate (27 g, 92.4 mmol, 1.0 eq), K₂CO₃ (25.5 g, 184.7 mmol, 2.0 eq), chloroacetone (12.8 g, 138.6 mmol, 1.5 eq), and 18-Crown-6 (2.4 g, 9.2 mmol, 0.1 eq) in CH₃CN (300 mL) was heated to 80° C. and stirred for 16 h. The reaction mixture was cooled to room temperature and filtered. The cake was diluted with EtOAc (3×300 mL). The combined organic layers were washed with brine, dried over anhydrous Na₂SO₄, filtered, and concentrated under reduced pressure. The residue was purified by column chromatography on silica gel (Hex/EtOAc=10:1 to 3:1) to give 6-tert-butyl 2-methyl 3-(2-oxopropoxy)benzofuran-2,6-dicarboxylate (5.3 g, 33%) as a light-yellow solid. TLC: DCM/MeOH=20:1, UV; R_{f}(compound 4)=0.55; R_{f} (compound 5)=0.65; LC-MS: 349.15 [M+H]⁺. ¹H NMR (400 MHz, CDCl₃) δ (ppm): 8.13-8.08 (m, 1H), 7.91 (dt, J=8.3, 1.0 Hz, 1H), 7.74 (dt, J=8.3, 0.7 Hz, 1H), 5.05 (s, 2H), 3.97-3.91 (m, 3H), 2.32-2.25 (m, 3H), 1.65-1.55 (m, 9H).

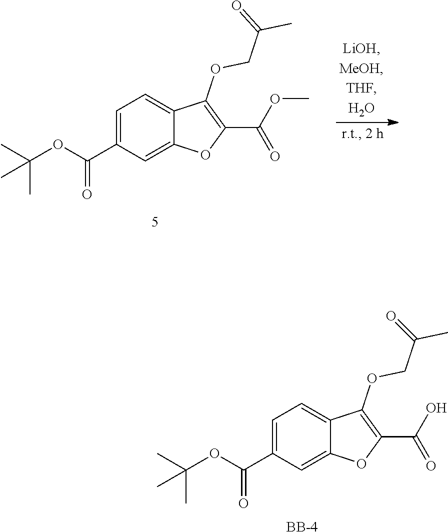

To a solution of 6-tert-butyl 2-methyl 3-(2-oxopropoxy)benzofuran-2,6-dicarboxylate (18.8 g, 53.96 mmol, 1.0 eq) in THF (100 mL) and MeOH (100 mL) was added LiOH solution (5% w/w in H₂O, 50 mL). The reaction mixture was stirred at room temperature for 2 h, added 1 N HCl to adjust to pH 2-3, and extracted with EtOAc (3×200 mL). The combined organic layers were washed with brine, dried over Na₂SO₄, filtered, and concentrated under reduced pressure to give crude 6-(tert-butoxycarbonyl)-3-(2-oxopropoxy)benzofuran-2-carboxylic acid (12 g) as a yellow solid, which was used for the next step directly. LC-MS: 335.05 [M+H]⁺.

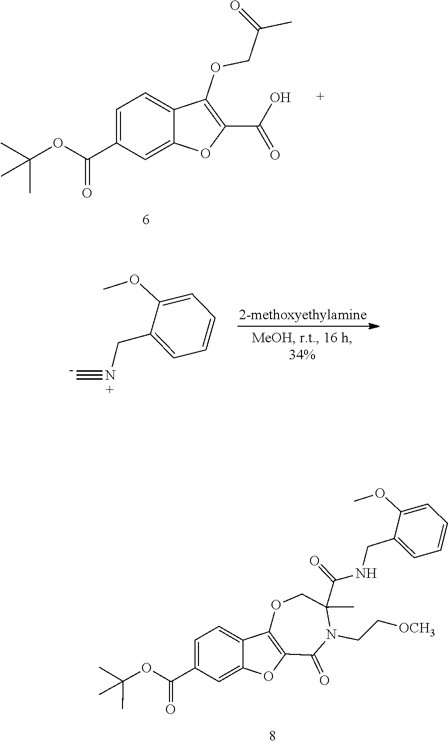

A mixture of 6-(tert-butoxycarbonyl)-3-(2-oxopropoxy)benzofuran-2-carboxylic acid (12.0 g, 35.9 mmol, 1.0 eq), 2-methoxyethylamine (2.7 g, 35.9 mmol, 1.0 eq), and 2-methoxybenzyl isocyanide (5.81 g, 39.5 mmol, 1.1 eq) in MeOH (120 mL) was stirred at room temperature for 16 h and concentrated under reduced pressure. The crude product was recrystallized from MeOH to tert-butyl 3-((2-methoxybenzyl)carbamoyl)-4-(2-methoxyethyl)-3-methyl-5-oxo-2,3,4,5-tetrahydrobenzofuro[2,3-f][1,4]oxazepine-8-carboxylate (6.5 g, 34%) as a white solid. LC-MS: 539.3 [M+H]⁺; ¹H NMR (400 MHz, CDCl₃) δ (ppm): 7.99 (q, J=1.2 Hz, 1H), 7.79 (dt, J=8.3, 1.2 Hz, 1H), 7.56-7.49 (m, 1H), 7.41 (s, 1H), 6.91-6.76 (m, 2H), 6.55 (d, J=8.1 Hz, 1H), 6.42 (s, 1H), 4.86 (d, J=12.0 Hz, 1H), 4.32 (dt, J=23.5, 11.7 Hz, 2H), 4.15-4.02 (m, 2H), 3.81-3.70 (m, 4H), 3.61-3.50 (m, 2H), 3.23 (s, 3H), 1.76 (s, 3H), 1.61 (s, 9H).

### 3-((2-methoxybenzyl)carbamoyl)-4-(2-methoxyethyl)-3-methyl-5-oxo-2,3,4,5-tetrahydrobenzofuro[2,3-f][1,4]oxazepine-8-carboxylic acid

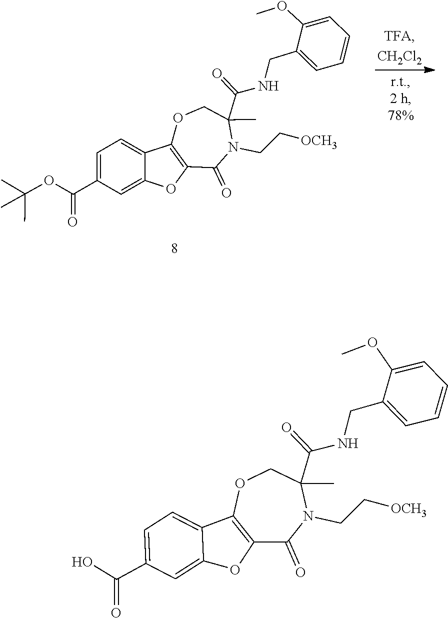 [SMILES: COCCN1C(=O)c2oc3cc(C(=O)OC(C)(C)C)ccc3c2OCC1(C)C(=O)NCc1ccccc1OC ; COCCN1C(=O)c2oc3cc(C(=O)O)ccc3c2OCC1(C)C(=O)NCc1ccccc1OC]

A mixture of tert-butyl 3-((2-methoxybenzyl)carbamoyl)-4-(2-methoxyethyl)-3-methyl-5-oxo-2,3,4,5-tetrahydrobenzofuro[2,3-f][1,4]oxazepine-8-carboxylate (4.0 g, 7.43 mmol, 1.0 eq) and TFA (40 mL) in CH₂Cl₂ (40 mL) was stirred room temperature for 2 h, and concentrated under reduced pressure. The crude product was triturated with MeOH to give compound 3-((2-methoxybenzyl)carbamoyl)-4-(2-methoxyethyl)-3-methyl-5-oxo-2,3,4,5-tetrahydrobenzofuro[2,3-f][1,4]oxazepine-8-carboxylic acid (2.8 g, 78%) as a white solid. LC-MS: 483.2 [M+H]⁺; ¹H NMR (400 MHz, DMSO-d₆) δ (ppm): 8.27 (s, 1H), 8.05 (d, J=1.1 Hz, 1H), 7.91 (d, J=8.0 Hz, 1H), 7.78 (d, J=8.0 Hz, 1H), 7.04 (t, J=7.0 Hz, 1H), 6.84 (d, J=8.0 Hz, 1H), 6.71 (brs, 1H), 6.32 (brs, 1H), 4.93 (d, J=12.5 Hz, 1H), 4.36 (d, J=12.5 Hz, 1H), 4.14 (d, J=5.7 Hz, 2H), 3.95-3.60 (m, 5H), 3.50 (brs, 2H), 3.23 (s, 3H), 1.70 (s, 3H). Two isomers were obtained by chiral HPLC. Chiral IC—H column (0.46 cm ID×15 cm), heptane:ethanol (60:40); flow rate at 0.5 mL/min, monitored at 254 nm. Peak 1: (2.72 min, 100% ee). LC-MS: 483.2 [M+H]⁺; ¹H NMR (400 MHz, DMSO-d₆) δ (ppm): 8.32 (brs, 1H), 8.05 (s, 1H), 7.90 (d, J=8.0 Hz, 1H), 7.74 (d, J=9.7 Hz, 1H), 7.04 (t, J=7.0 Hz, 1H), 6.84 (d, J=8.0 Hz, 1H), 6.73 (brs, 1H), 6.35 (brs, 1H), 4.96 (d, J=12.5 Hz, 1H), 4.38 (d, J=12.5 Hz, 1H), 4.16 (d, J=5.7 Hz, 2H), 3.95-3.60 (m, 5H), 3.50 (brs, 2H), 3.22 (s, 3H), 1.72 (s, 3H). Peak 2 (3.29 min, 99.2% ee).

### General Procedure for Amide Formation

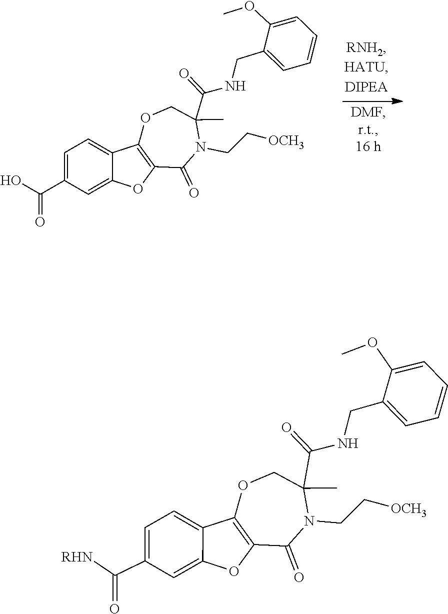

To a solution of the carboxylic acid (100 mg, 0.21 mmol, 1.0 eq) in DMF (2 mL) was added HATU (118 mg, 0.31 mmol, 1.5 e q). The mixture was stirred at room temperature for 10 min. Amine RNH₂ and DIPEA (54 mg, 0.41 mmol, 2.0 eq) were added. The reaction mixture was stirred at room temperature for 16 h. The reaction mixture was poured into H₂O (30 mL) and extracted with EtOAc (3×30 mL). The combined organic layers were washed with brine, dried over Na₂SO₄, filtered, and concentrated under reduced pressure. The crude product was purified by prep-HPLC to give each final product after lyophilization.

### Example 51

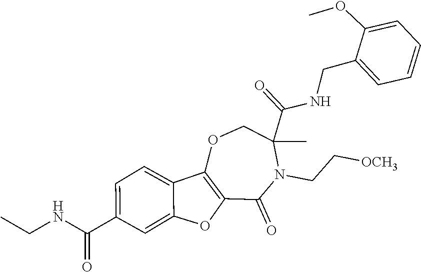 [SMILES: CCNC(=O)c1ccc2c3c(oc2c1)C(=O)N(CCOC)C(C)(C(=O)NCc1ccccc1OC)CO3]

LC-MS: 510.45 [M+H]⁺; ¹H NMR (400 MHz, CDCl₃) δ (ppm): 7.78 (s, 1H), 7.50 (d, J=19.0 Hz, 3H), 6.85 (d, J=6.9 Hz, 2H), 6.56 (s, 1H), 6.41 (s, 1H), 6.23 (s, 1H), 4.94-4.80 (m, 1H), 4.32 (s, 2H), 4.06 (s, 2H), 3.74 (s, 4H), 3.53 (s, 4H), 3.23 (s, 3H), 1.76 (s, 3H), 1.28 (t, J=7.2 Hz, 3H).

### (1r,3r)-3-methoxycyclobutanamine hydrochloride

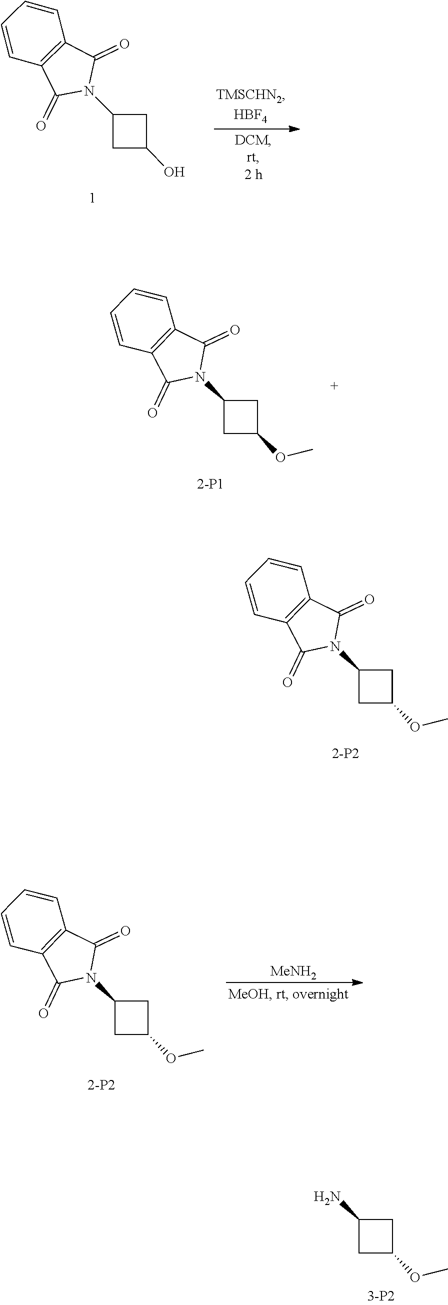

To the mixture of 2-(3-hydroxycyclobutyl)isoindoline-1,3-dione (5.3 g, 24.4 mmol, 1.0 eq), in DCM (50 mL) was added HBF4 (2.4 mL, 48% in water, 24.4 mmol, 1.0 eq.), TMSCHN2 (2 M in hexane, 24.4 mL, 48.8 mmol, 2.0 eq.) at 0° C. The mixture was slowly warmed to room temperature and stirred for 2 h. Water (50 mL) was added. The aqueous phase was extracted with EA (3×50 mL). The combined organic solution was washed with brine (50 mL), dried over anhydrous Na₂SO₄, and the solution was concentrated under reduced pressure. The residue was purified by silica gel chromatography (PE:EA=9:1) to give 2-((1s,3s)-3-methoxycyclobutyl)isoindoline-1,3-dione (2-P1) (300 mg, 5%) as white solid, and 2-((1r,3r)-3-methoxycyclobutyl)isoindoline-1,3-dione (2-P2) (1.8 g, 31%) as white solid. LC-MS of 2-P1: 232.1 [M+1]⁺; 1H NMR of 2-P1 (400 MHz, CDCl3) δ 7.89-7.76 (m, 2H), 7.78-7.61 (m, 2H), 5.11-4.93 (m, 1H), 4.39-4.23 (m, 1H), 3.31 (s, 3H), 3.09-2.90 (m, 2H), 2.55-2.33 (m, 2H). LC-MS of 2-P2: 232.1 [M+1]⁺; 1H NMR of 2-P2 (400 MHz, CDCl3) δ 7.86-7.80 (m, 2H), 7.73-7.68 (m, 2H), 4.39-4.22 (m, 1H), 3.80-3.67 (m, 1H), 3.31 (s, 3H), 2.98-2.81 (m, 2H), 2.72-2.59 (m, 2H).

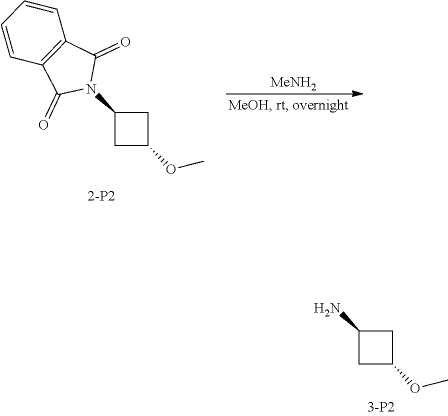

To a solution of 2-((1r,3r)-3-methoxycyclobutyl)isoindoline-1,3-dione (2-P2) (280 mg, 1.21 mmol, 1.0 eq) in MeOH (3 mL) was added MeNH2 (33% in EtOH, 3 mL). The mixture was stirred at room temperature overnight. The insoluble material was filtered off and the mother liquor was concentrated to a low volume and again filtered. The remainder of the volatile material was distilled off and the residue was purified by silica gel chromatography (DCM:MeOH=10:1) and followed by the acidification to pH 3-4 with HCl in EA (80). After concentration under reduced pressure, 110 mg (66) (1r,3r)-3-methoxycyclobutanamine hydrochloride was obtained as white solid. 1H NMR (400 MHz, DMSO) δ 8.36 (s, 3H), 4.15-4.04 (m, 1H), 3.73-3.61 (m, 1H), 3.18-3.11 (s, 3H), 2.39-2.27 (m, 2H), 2.27-2.17 (m, 2H).

Using the above procedures and intermediates, the following compounds were prepared:

| Example | Structure | Analytical Data |
|---|---|---|
| 52 | 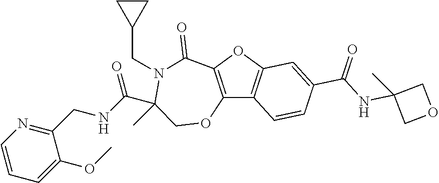 [SMILES: COc1cccnc1CNC(=O)C1(C)COc2c(oc3cc(C(=O)NC4(C)COC4)ccc23)C(=O)N1CC1CC1] | 1H NMR (400 MHz, CDCl3) δ 7.91 (d, J = 3.8 Hz, 1H), 7.85 (s, 1H), 7.75 (s, 1H), 7.67-7.57 (m, 2H), 7.08-6.98 (m, 2H), 6.65 (s, 1H), 4.89 (d, J = 6.5 Hz, 3H), 4.59 (d, J = 6.6 Hz, 2H), 4.43 (d, J = 4.5 Hz, 2H), 4.25 (d, J = 11.7 Hz, 1H), 3.83-3.55 (m, 5H), 1.86 (s, 3H), 1.79 (s, 3H), 1.21-1.10 (m, 1H), 0.62-0.41 (m, 4H). LC-MS: 549.2[M + 1]+ |
| 53 | 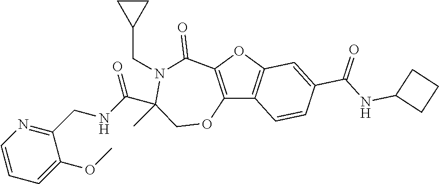 [SMILES: COc1cccnc1CNC(=O)C1(C)COc2c(oc3cc(C(=O)NC4CCC4)ccc23)C(=O)N1CC1CC1] | 1H NMR (400 MHZ, CDCl3) δ 7.90 (d, J = 3.7 Hz, 1H), 7.84 (s, 1H), 7.75 (s, 1H), 7.64 (d, J = 8.2 Hz, 1H), 7.58 (d, J = 8.2 Hz, 1H), 7.08-6.98 (m, 2H), 6.25 (d, J = 7.4 Hz, 1H), 4.88 (d, J = 11.8 Hz, 1H), 4.60 (dd, J = 16.1, 8.2 Hz, 1H), 4.51-4.37 (m, 2H), 4.24 (d, J = 11.9 Hz, 1H), 3.80-3.59 (m, 5H), 2.52-2.39 (m, 2H), 2.04-1.93 (m, 2H), 1.86 (s, 3H), 1.84-1.80 (m, 2H), 1.21-1.08 (m, 1H), 0.60-0.43 (m, 4H). LC-MS: 533.2[M + 1]+ |
| 54 | 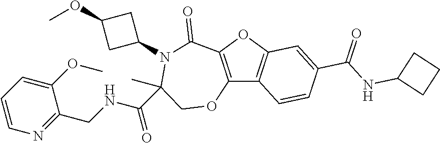 [SMILES: COc1cccnc1CNC(=O)C1(C)COc2c(oc3cc(C(=O)NC4CCC4)ccc23)C(=O)N1[C@H]1C[C@@H](OC)C1] | 1H NMR (400 MHz, CDCl3) δ 8.04 (s, 1H), 7.88 (s, 1H), 7.83 (s, 1H), 7.69-7.59 (m, 2H), 7.23-7.12 (m, 2H), 6.32 (d, J = 7.2 Hz, 1H), 4.68-4.54 (m, 3H), 4.51-4.39 (m, 2H), 3.87 (s, 3H), 3.70-3.52 (m, 2H), 3.21 (s, 3H), 3.03-2.83 (m, 2H), 2.68-2.56 (m, 1H), 2.53-2.35 (m, 3H), 2.08-1.95 (m, 2H), 1.83-1.76 (m, 2H), 1.67 (s, 3H). LC-MS: 563.2[M + 1]+ |
| 55 | 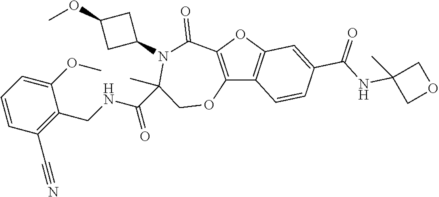 [SMILES: COc1cccc(C#N)c1CNC(=O)C1(C)COc2c(oc3cc(C(=O)NC4(C)COC4)ccc23)C(=O)N1[C@H]1C[C@@H](OC)C1] | 1H NMR (400 MHZ, DMSO) δ 9.02 (s, 1H), 8.46 (s, 1H), 8.05 (s, 1H), 7.85-7.75 (m, 2H), 7.49 (t, J = 8.0 Hz, 1H), 7.42-7.32 (m, 2H), 4.73 (d, J = 6.3 Hz, 2H), 4.53 (d, J = 12.6 Hz, 1H), 4.47-4.33 (m, 5H), 3.85 (s, 3H), 3.54-3.45 (m, 1H), 3.30-3.25 (m, 1H), 3.11 (s, 3H), 2.87-2.67 (m, 2H), 2.38-2.16 (m, 2H), 1.63 (s, 3H), 1.42 (s, 3H). LC-MS: 603.2[M + 1]+ |
| 56 | 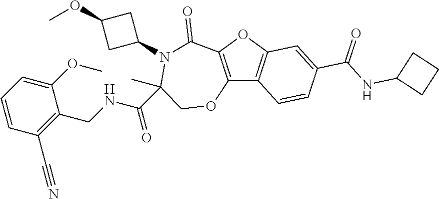 [SMILES: COc1cccc(C#N)c1CNC(=O)C1(C)COc2c(oc3cc(C(=O)NC4CCC4)ccc23)C(=O)N1[C@H]1C[C@@H](OC)C1] | 1H NMR (400 MHZ, DMSO) δ 8.74 (d, J = 7.5 Hz, 1H), 8.46 (s, 1H), 8.04 (s, 1H), 7.85-7.72 (m, 2H), 7.49 (t, J = 8.0 Hz, 1H), 7.42-7.33 (m, 2H), 4.58-4.31 (m, 5H), 3.85 (s, 3H), 3.55-3.45 (m, 1H), 3.30-3.24 (m, 1H), 3.11 (s, 3H), 2.86-2.68 (m, 2H), 2.38-2.18 (m, 4H), 2.15-2.01 (m, 2H), 1.74- 1.62 (m, 2H), 1.42 (s, 3H). LC-MS: 587.2[M + 1]+ |
| 57 | 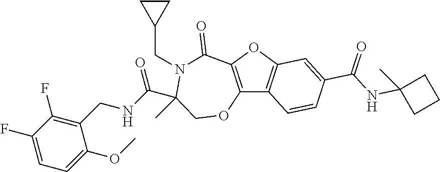 [SMILES: COc1ccc(F)c(F)c1CNC(=O)C1(C)COc2c(oc3cc(C(=O)NC4(C)CCC4)ccc23)C(=O)N1CC1CC1] | 1H NMR (400 MHZ, DMSO) δ 9.02 (s, 1H), 8.19-7.93 (m, 2H), 7.80 (dd, J = 8.3, 1.0 Hz, 1H), 7.69 (d, J = 8.3 Hz, 1H), 7.25-7.06 (m, 1H), 6.71 (d, J = 8.5 Hz, 1H), 4.84- 4.69 (m, 3H), 4.44-4.19 (m, 5H), 3.80-3.62 (m, 4H), 2.97 (s, 1H), 1.76-1.51 (m, 6H), 1.08-0.95 (m, 1H), 0.48-0.38 (m, 2H), 0.34-0.17 (m, 2H). LC-MS: 584.2[M + 1]+ |
| 58 | 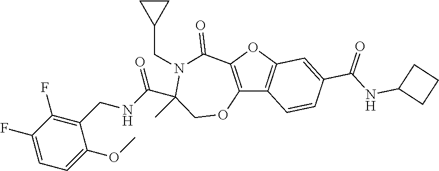 [SMILES: COc1ccc(F)c(F)c1CNC(=O)C1(C)COc2c(oc3cc(C(=O)NC4CCC4)ccc23)C(=O)N1CC1CC1] | 1H NMR (400 MHZ, CDCl3) δ 7.84 (s, 1H), 7.57-7.46 (m, 2H), 6.99 (dd, J = 7.3, 4.1 Hz, 1H), 6.54 (dd, J = 18.6, 9.2 Hz, 1H), 6.32 (d, J = 7.7 Hz, 1H), 6.17 (d, J = 9.0 Hz, 1H), 4.91 (d, J = 11.9 Hz, 1H), 4.76- 4.57 (m, 2H), 4.05 (d, J = 11.9 Hz, 1H), 3.97 (d, J = 12.6 Hz, 1H), 3.91-3.79 (m, 1H), 3.73 (s, 3H), 3.55 (dd, J = 14.9, 5.5 Hz, 1H), 2.54-2.42 (m, 2H), 2.09-1.96 (m, 2H), 1.88- 1.78 (m, 5H), 1.13-1.00 (m, 1H), 0.69-0.58 (m, 2H), 0.54-0.43 (m, 2H). |
| | | LC-MS: 568.2[M + 1]+ |
| 59 | 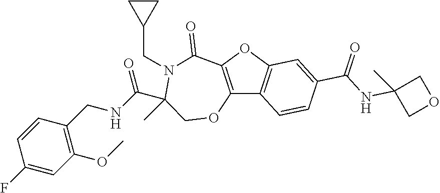 [SMILES: COc1cc(F)ccc1CNC(=O)C1(C)COc2c(oc3cc(C(=O)NC4(C)COC4)ccc23)C(=O)N1CC1CC1] | 1H NMR (400 MHZ, CDCl3) δ 7.75 (s, 1H), 7.52 (dt, J = 15.6, 4.7 Hz, 2H), 7.06 (dd, J = 8.1, 3.9 Hz, 1H), 6.90-6.74 (m, 1H), 6.54 (s, 1H), 6.23 (dd, J = 11.0, 2.3 Hz, 1H), 6.08-5.93 (m, 1H), 4.92 (dd, J = 6.1, 2.2 Hz, 3H), 4.61 (d, J = 6.6 Hz, 2H), 4.39 (dd, J = 14.0, 8.4 Hz, 1H), 4.05 (d, J = 11.9 Hz, 1H), 3.96 (dd, J = 14.0, 3.9 Hz, 1H), 3.92-3.82 (m, 1H), 3.80 (s, 3H), 3.55 (dd, J = 15.0, 5.5 Hz, 1H), 1.87 (s, 3H), 1.82 (s, 3H), 1.16-0.99 (m, 1H), 0.77-0.43 (m, 4H). LC-MS: 566.2[M + 1]+ |
| 60 | 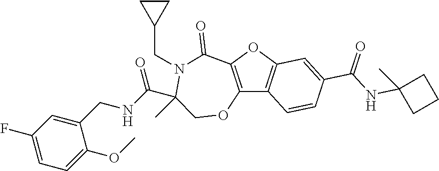 [SMILES: COc1ccc(F)cc1CNC(=O)C1(C)COc2c(oc3cc(C(=O)NC4(C)CCC4)ccc23)C(=O)N1CC1CC1] | 1H NMR (400 MHZ, CDCl3) δ 7.73 (s, 1H), 7.56-7.45 (m, 2H), 7.21 (s, 1H), 6.67 (d, J = 11.8 Hz, 2H), 6.47-6.45 (m, 2H), 4.92 (dd, J = 6.7, 2.9 Hz, 3H), 4.62 (d, J = 6.5 Hz, 2H), 4.35 (dd, J = 14.3, 7.6 Hz, 1H), 4.17-4.03 (m, 2H), 3.81-3.79 (m, 1H), 3.75 (s, 3H), 3.60 (dd, J = 14.9, 5.6 Hz, 1H), 1.86 (s, 3H), 1.82 (s, 3H), 1.13-0.97 (m, 1H), 0.64-0.47 (m, 4H). LC-MS: 566.2[M + 1]+ |
| 61 | 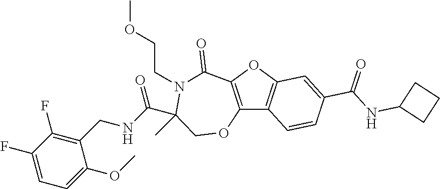 [SMILES: COCCN1C(=O)c2oc3cc(C(=O)NC4CCC4)ccc3c2OCC1(C)C(=O)NCc1c(OC)ccc(F)c1F] | 1H NMR (400 MHz, CDCl3) δ 7.83 (s, 1H), 7.57 (d, J = 8.1 Hz, 1H), 7.52-7.48 (m, 2H), 6.69-6.46 (m, 1H), 6.26 (d, J = 7.6 Hz, 1H), 6.21 (d, J = 9.3 Hz, 1H), 4.89 (d, J = 11.8 Hz, 1H), 4.71-4.60 (m, 2H), 4.37-4.34 (m, 1H), 4.08 (d, J = 11.7 Hz, 1H), 3.95 (d, J = 13.4 Hz, 1H), 3.83-3.76 (m, 1H), 3.71 (s, 3H), 3.62-3.44 (m, 2H), 3.31 (s, 3H), 2.64-2.37 (m, 2H), 2.09-1.92 (m, 2H), 1.83 (dd, J = 9.6, 5.2 Hz, 2H), 1.79 (d, J = 6.5 Hz, 3H). LC-MS: 572.2[M + 1]+ |
| 62 | 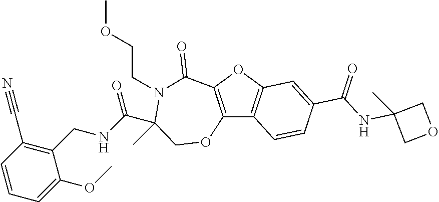 [SMILES: COCCN1C(=O)c2oc3cc(C(=O)NC4(C)COC4)ccc3c2OCC1(C)C(=O)NCc1c(C#N)cccc1OC] | 1H NMR (400 MHZ, CDCl3) δ 7.76 (s, 1H), 7.69 (t, J = 5.1 Hz, 1H), 7.51-7.50 (m, 2H), 6.90 (t, J = 7.8 Hz, 1H), 6.83-6.80 (m, 2H), 6.75 (d, J = 8.2 Hz, 1H), 4.94-4.88 (m, 3H), 4.74 (dd, J = 14.1, 7.0 Hz, 1H), 4.61 (dd, J = 6.4, 1.7 Hz, 2H), 4.32-4.31 (m, 1H), 4.15 (dd, J = 14.1, 3.9 Hz,1H), 4.07 (d, J = 11.9 Hz, 1H), 3.86-3.80 (m, 1H), 3.76 (s, 3H), 3.69-3.51 (m, 2H), 3.33 (s, 3H), 1.82 (s, 3H), 1.79 (s, 3H). LC-MS: 577.2[M + 1]+ |
| 63 | 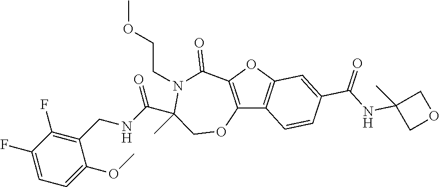 [SMILES: COCCN1C(=O)c2oc3cc(C(=O)NC4(C)COC4)ccc3c2OCC1(C)C(=O)NCc1c(OC)ccc(F)c1F] | 1H NMR (400 MHZ, CDCl3) δ 7.82 (s, 1H), 7.58 (d, J = 8.0 Hz, 1H), 7.51 (dd, J = 8.2, 1.3 Hz, 1H), 7.49-7.44 (m, 1H), 6.68-6.54 (m, 2H), 6.25 (d, J = 8.0 Hz, 1H), 4.93-4.88 (m, 3H), 4.69-4.58 (m, 3H), 4.34-4.31 (m, 1H), 4.10 (d, J = 11.8 Hz, 1H), 3.97 (d, J = 13.1 Hz, 1H), 3.83-3.77 (m, 1H), 3.73 (s, 3H), 3.62-3.52 (m, 2H), 3.31 (s, 3H), 1.82 (s, 3H), 1.78 (s, 3H). LC-MS: 588.2[M + 1]+ |
| 64 | 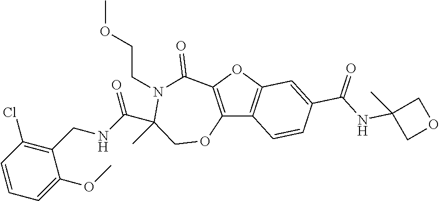 [SMILES: COCCN1C(=O)c2oc3cc(C(=O)NC4(C)COC4)ccc3c2OCC1(C)C(=O)NCc1c(Cl)cccc1OC] | 1H NMR (400 MHZ, CDCl3) δ 7.83 (s, 1H), 7.60 (d, J = 8.2 Hz, 1H), 7.54 (dd, J = 8.2, 1.3 Hz, 1H), 7.30 (t, J = 8.9 Hz, 1H), 6.86 (t, J = 8.2 Hz, 1H), 6.64 (d, J = 8.0 Hz, 1H), 6.58 (s, 1H), 6.53 (d, J = 8.3 Hz, 1H), 4.92-4.87 (m, 3H), 4.70 (dd, J = 13.8, 6.7 Hz, 1H), 4.61 (d, J = 6.7 Hz, 2H), 4.33-4.04 (m, 3H), 3.84-3.76 (m, 1H), 3.73 (s, 3H), 3.61-3.50 (m, 2H), 3.18 (s, 3H), 1.81 (s, 3H), 1.78 (s, 3H). LC-MS: 586.2[M + 1]+ |
| 65 | 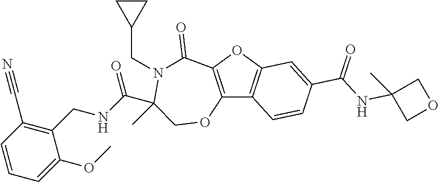 [SMILES: COc1cccc(C#N)c1CNC(=O)C1(C)COc2c(oc3cc(C(=O)NC4(C)COC4)ccc23)C(=O)N1CC1CC1] | 1H NMR (400 MHZ, CDCl3) δ 7.80 (s, 1H), 7.53-7.44 (m, 2H), 7.08 (s, 1H), 6.90-6.82 (m, 1H), 6.80 (d, J = 6.8 Hz, 1H), 6.71 (d, J = 7.6 Hz, 1H), 6.58 (s, 1H), 4.94-4.91 (m, 3H), 4.79 (dd, J = 14.2, 7.6 Hz, 1H), 4.62 (dd, J = 6.4, 1.6 Hz, 2H), 4.17 (dd, J = 14.1, 4.1 Hz, 1H), 4.03 (d, J = 11.9 Hz, 1H), 3.79-3.78 (m, 1H), 3.77 (s, 3H), 3.59 (dd, J = 15.0, 5.6 Hz, 1H), 1.88 (s, 3H), 1.82 (s, 3H), 1.10-1.07 (m, 1H), 0.68-0.59 (m, 2H), 0.52-0.49 (m, 2H). LC-MS: 573.2[M + 1]+ |
| 66 | 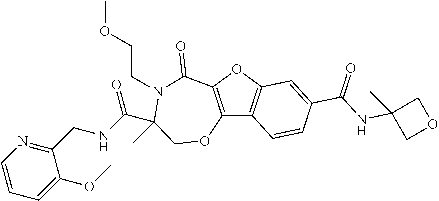 [SMILES: COCCN1C(=O)c2oc3cc(C(=O)NC4(C)COC4)ccc3c2OCC1(C)C(=O)NCc1ncccc1OC] | 1H NMR (400 MHZ, CDCl3) δ 7.94-7.85 (m, 3H), 7.65 (d, J = 8.2 Hz, 1H), 7.60 (d, J = 8.2 Hz, 1H), 7.09-6.98 (m, 2H), 6.70 (s, 1H), 4.90-4.85 (m, 3H), 4.59 (d, J = 6.5 Hz, 2H), 4.48-4.20 (m, 3H), 4.03-3.95 (m, 1H), 3.90-3.78 (m, 2H), 3.76 (s, 3H), 3.71-3.64 (m, 1H), 3.26 (s, 3H), 1.81 (s, 3H), 1.79 (s, 3H). LC-MS: 553.2[M + 1]+ |
| 67 | 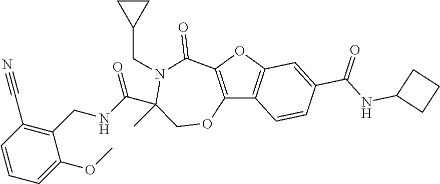 [SMILES: COc1cccc(C#N)c1CNC(=O)C1(C)COc2c(oc3cc(C(=O)NC4CCC4)ccc23)C(=O)N1CC1CC1] | 1H NMR (400 MHZ, CDCl3) δ 7.83 (s, 1H), 7.53-7.41 (m, 2H), 7.06-7.02 (m, 1H), 6.86-6.74 (m, 2H), 6.67 (d, J = 8.0 Hz, 1H), 6.27 (d, J = 7.3 Hz, 1H), 4.92 (d, J = 12.0 Hz, 1H), 4.82 (dd, J = 14.1, 7.9 Hz, 1H), 4.63 (dd, J = 16.1, 8.0 Hz, 1H), 4.11 (dd, J = 14.1, 4.3 Hz, 1H), 4.02 (d, J = 11.9 Hz, 1H), 3.85-3.80 (m, 1H), 3.77 (s, 3H), 3.57 (dd, J = 14.8, 5.5 Hz, 1H), 2.52-2.45 (m, 2H), 2.06-1.96 (m, 2H), 1.88 (s, 3H), 1.86-1.77 (m, 2H), 1.13-1.09 (m, 1H), 0.74-0.59 (m, 2H), 0.54-0.50 (m, 2H). LC-MS: 557.2[M + 1]+ |
| 68 | 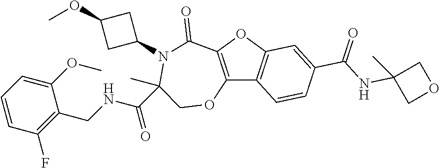 [SMILES: COc1cccc(F)c1CNC(=O)C1(C)COc2c(oc3cc(C(=O)NC4(C)COC4)ccc23)C(=O)N1[C@H]1C[C@@H](OC)C1] | 1H NMR (400 MHZ, DMSO) δ 9.02 (s, 1H), 8.31 (s, 1H), 8.05 (s, 1H), 7.85-7.74 (m, 2H), 7.32 (dd, J = 15.3, 8.2 Hz, 1H), 6.87 (d, J = 8.4 Hz,1H), 6.80 (t, J = 8.8 Hz, 1H), 4.73 (d, J = 6.2 Hz, 2H), 4.56 (d, J = 12.7 Hz, 1H), 4.43-4.22 (m, 5H), 3.82 (s, 3H), 3.55-3.41 (m, 1H), 3.30-3.21 (m, 1H), 3.11 (s, 3H), 2.85-2.65 (m, 2H), 2.36-2.13 (m, 2H), 1.63 (s, 3H), 1.44 (s, 3H). LC-MS: 596.20[M + 1]+ |
| 69 | 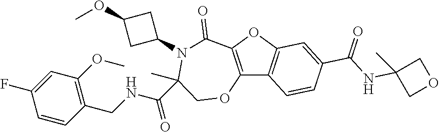 [SMILES: COc1cc(F)ccc1CNC(=O)C1(C)COc2c(oc3cc(C(=O)NC4(C)COC4)ccc23)C(=O)N1[C@H]1C[C@@H](OC)C1] | 1H NMR (400 MHZ, DMSO) δ 9.03 (s, 1H), 8.49 (s, 1H), 8.06 (s, 1H), 7.86-7.76 (m, 2H), 7.20 (s, 1H), 6.89 (d, J = 11.3 Hz, 1H), 6.68 (s, 1H), 4.74 (d, J = 6.3 Hz, 2H), 4.62 (d, J = 12.3 Hz, 1H), 4.47-4.35 (m, 3H), 4.34-4.13 (m, 2H), 3.80 (s, 3H), 3.57-3.41 (m, 1H), 3.31-3.28 (m, 1H), 3.11 (s, 3H), 2.93-2.62 (m, 2H), 2.39-2.14 (m, 2H), 1.63 (s, 3H), 1.45 (s, 3H). |
| | | LC-MS: 596.20[M + 1]+ |
| 70 | 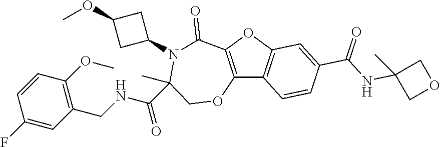 [SMILES: COc1ccc(F)cc1CNC(=O)C1(C)COc2c(oc3cc(C(=O)NC4(C)COC4)ccc23)C(=O)N1[C@H]1C[C@@H](OC)C1] | 1H NMR (400 MHZ, DMSO) δ 9.03 (s, 1H), 8.56 (s, 1H), 8.06 (s, 1H), 7.94-7.71 (m, 2H), 7.17-6.81 (m, 3H), 4.74 (d, J = 6.3 Hz, 2H), 4.64 (d, J = 12.3 Hz, 1H), 4.45 (d, J = 12.7 Hz, 1H), 4.40 (d, J = 6.4 Hz, 2H), 4.38-4.29 (m, 1H), 4.27-4.15 (m, 1H), 3.78 (s, 3H), 3.56-3.41 (m, 1H), 3.31-3.26 (m, 1H), 3.11 (s, 3H), 2.92-2.66 (m, 2H), 2.39-2.22 (m, 2H), 1.63 (s, 3H), 1.47 (s, 3H). LC-MS: 596.20[M + 1]+ |
| 71 | 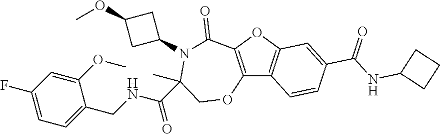 [SMILES: COc1cc(F)ccc1CNC(=O)C1(C)COc2c(oc3cc(C(=O)NC4CCC4)ccc23)C(=O)N1[C@H]1C[C@@H](OC)C1] | 1H NMR (400 MHZ, DMSO) δ 8.75 (d, J = 7.5 Hz, 1H), 8.55-8.45 (m, 1H), 8.06 (s, 1H), 7.83 (dd, J = 8.3, 1.2 Hz, 1H), 7.77 (d, J = 8.2 Hz, 1H), 7.25-7.10 (m, 1H), 6.89 (d, J = 10.7 Hz, 1H), 6.75-6.60 (m, 1H), 4.61 (d, J = 12.4 Hz, 1H), 4.52-4.37 (m, 2H), 4.35-4.15 (m, 2H), 3.80 (s, 3H), 3.45 (dd, J = 14.0, 7.1 Hz, 1H), 3.31-3.25 (m, 1H), 3.11 (s, 3H), 2.87-2.65 (m, 2H), 2.38-2.17 (m, 4H), 2.15- |
| | | 2.00 (m, 2H), 1.78-1.59 (m, 2H), 1.44 (s, 3H). |
| | | LC-MS: 580.20[M + 1]+ |
| 72 | 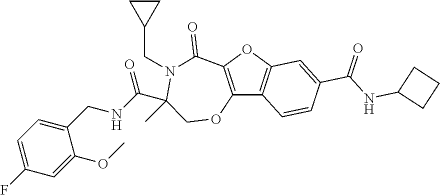 [SMILES: COc1cc(F)ccc1CNC(=O)C1(C)COc2c(oc3cc(C(=O)NC4CCC4)ccc23)C(=O)N1CC1CC1] | 1H NMR (400 MHZ, DMSO) δ 8.74 (d, J = 7.5 Hz, 1H), 8.26 (s, 1H), 8.05 (s, 1H), 7.83 (dd, J = 8.3, 1.2 Hz, 1H), 7.74 (d, J = 8.3 Hz, 1H), 6.96-6.66 (m, 2H), 6.22 (s, 1H), 4.91 (d, J = 12.0 Hz, 1H), 4.58-4.30 (m, 2H), 4.21-4.00 (m, 2H), 3.73 (s, 3H), 3.73- 3.69 (m, 1H), 3.23-2.98 (m, 1H), 2.34-2.18 (m, 2H), 2.18-1.97 (m, 2H), 1.87-1.52 (m, 5H), 1.15-0.93 (m, 1H), 0.52-0.38 (m, 2H), 0.37-0.15 (m, 2H). LC-MS: 550.20[M + 1]+ |
| 73 | 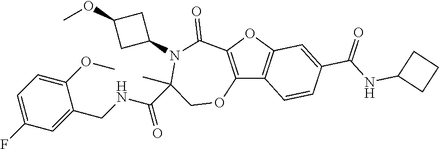 [SMILES: COc1ccc(F)cc1CNC(=O)C1(C)COc2c(oc3cc(C(=O)NC4CCC4)ccc23)C(=O)N1[C@H]1C[C@@H](OC)C1] | 1H NMR (400 MHZ, DMSO) δ 8.75 (d, J = 7.5 Hz, 1H), 8.57 (s, 1H), 8.06 (s, 1H), 7.83 (dd, J = 8.3, 1.2 Hz, 1H), 7.75 (d, J = 8.3 Hz, 1H), 7.15-6.90 (m, 3H), 4.64 (d, J = 12.0 Hz, 1H), 4.52-4.39 (m, 2H), 4.34 (dd, J = 15.4, 5.8 Hz, 1H), 4.21 (dd, J = 15.2, 5.0 Hz, 1H), 3.78 (s, 3H), 3.55-3.44 (m, 1H), 3.30-3.24 (m, 1H), 3.11 (s, 3H), 2.90-2.65 (m, 2H), 2.43-2.17 (m, 5H), 2.17-1.99 (m, 2H), 1.78-1.60 (m, 2H), 1.46 (s, 3H). LC-MS: 580.20[M + 1]+ |
| 74 | 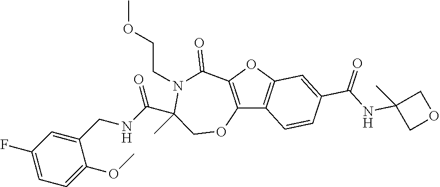 [SMILES: COCCN1C(=O)c2oc3cc(C(=O)NC4(C)COC4)ccc3c2OCC1(C)C(=O)NCc1cc(F)ccc1OC] | 1H NMR (400 MHZ, CDCl3) δ 7.79 (s, 1H), 7.62-7.54 (m, 2H), 7.51 (dd, J = 8.2, 1.3 Hz, 1H), 6.66-6.43 (m, 4H), 4.97-4.82 (m, 3H), 4.61 (d, J = 6.7 Hz, 2H), 4.32 (dd, J = 14.4, 7.3 Hz, 2H), 4.17-3.97 (m, 2H), 3.90- 3.79 (m, 1H), 3.76 (s, 3H), 3.66-3.51 (m, 2H), 3.32 (s, 3H), 1.81 (s, 3H), 1.80 (s, 3H). LC-MS: 570.20[M + 1]+ |
| 75 | 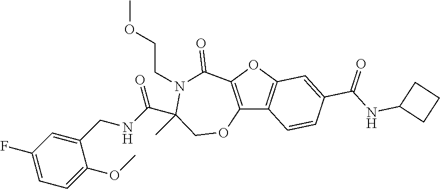 [SMILES: COCCN1C(=O)c2oc3cc(C(=O)NC4CCC4)ccc3c2OCC1(C)C(=O)NCc1cc(F)ccc1OC] | 1H NMR (400 MHz, CDCl3) δ 7.82 (s, 1H), 7.59-7.52 (m, 2H), 7.53-7.45 (m, 1H), 6.62 (d, J = 8.1 Hz, 1H), 6.52-6.42 (m, 2H), 6.28 (d, J = 7.4 Hz, 1H), 4.90 (d, J = 11.9 Hz, 1H), 4.74-4.55 (m, 1H), 4.44-4.26 (m, 2H), 4.18- 3.94 (m, 2H), 3.86-3.78 (m, 1H), 3.75 (s, 3H), 3.73-3.52 (m, 2H), 3.32 (s, 3H), 2.55-2.36 (m, 2H), 2.14-1.92 (m, 2H), 1.88-1.80 (m, 2H), 1.79 (s, 3H). LC-MS: 554.20[M + 1]+ |
| 76 | 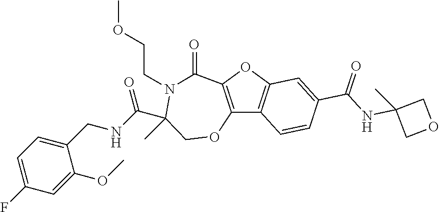 [SMILES: COCCN1C(=O)c2oc3cc(C(=O)NC4(C)COC4)ccc3c2OCC1(C)C(=O)NCc1ccc(F)cc1OC] | 1H NMR (400 MHZ, CDCl3) δ 7.73 (s, 1H), 7.62-7.47 (m, 4H), 6.86-6.75 (m, 1H), 6.58 (s, 1H), 6.27 (dd, J = 11.0, 2.2 Hz, 1H), 5.99 (t, J = 7.4 Hz, 1H), 4.95-4.90 (m, 2H), 4.61 (d, J = 6.7 Hz, 2H), 4.42-4.30 (m, 2H), 4.08 (d, J = 11.8 Hz, 1H), 3.99-3.89 (m, 1H), 3.85-3.73 (m, 5H), 3.63-3.53 (m, 2H), 3.36 (s, 3H), 1.81 (s, 3H), 1.79 (s, 3H). LC-MS: 570.20[M + 1]+ |
| 77 | 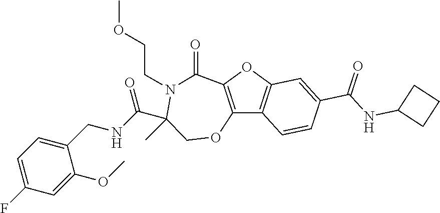 [SMILES: COCCN1C(=O)c2oc3cc(C(=O)NC4CCC4)ccc3c2OCC1(C)C(=O)NCc1ccc(F)cc1OC] | 1H NMR (400 MHz, CDCl3) δ 7.73 (s, 1H), 7.61-7.43 (m, 3H), 6.81 (t, J = 7.5 Hz, 1H), 6.35 (d, J = 7.0 Hz, 1H), 6.26 (dd, J = 11.0, 2.0 Hz, 1H), 6.02 (t, J = 7.5 Hz, 1H), 4.88 (d, J = 11.8 Hz, 1H), 4.69-4.54 (m, 1H), 4.36 (dd, J = 14.1, 7.8 Hz, 2H), 4.07 (d, J = 11.9 Hz, 1H), 3.94 (dd, J = 13.8, 3.9 Hz, 1H), 3.85-3.72 (m, 4H), 3.66-3.51 (m, 2H), 3.35 (m, 3H), 2.56-2.39 (m, 2H), 2.15-1.94 (m, 2H), 1.87-1.80 (m, 2H), 1.79 (s, 3H). LC-MS: 554.20[M + 1]+ |
| 78 | 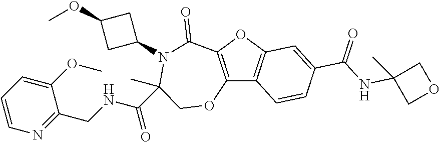 [SMILES: COc1cccnc1CNC(=O)C1(C)COc2c(oc3cc(C(=O)NC4(C)COC4)ccc23)C(=O)N1[C@H]1C[C@@H](OC)C1] | 1H NMR (400 MHZ, DMSO) δ 9.03 (s, 1H), 8.48 (s, 1H), 8.11-8.02 (m, 2H), 7.87-7.74 (m, 2H), 7.41 (d, J = 8.3 Hz, 1H), 7.29 (dd, J = 8.3, 4.7 Hz, 1H), 4.74 (d, J = 6.3 Hz, 2H), 4.60 (d, J = 12.7 Hz, 1H), 4.51-4.32 (m, 5H), 3.84 (s, 3H), 3.56-3.42 (m, 2H), 3.11 (s, 3H), 2.90-2.72 (m, 2H), 2.45-2.40 (m, 1H), 2.32- 2.20 (m, 1H), 1.63 (s, 3H), 1.44 (s, 3H). LC-MS: 579.20[M + 1]+ |
| 79 | 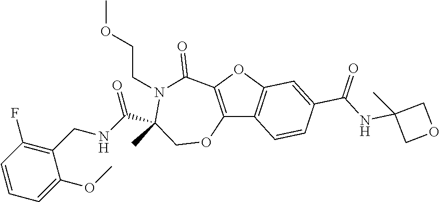 [SMILES: COCCN1C(=O)c2oc3cc(C(=O)NC4(C)COC4)ccc3c2OC[C@]1(C)C(=O)NCc1c(F)cccc1OC] | 1H NMR (400 MHz, CDCl3) δ 7.83 (s, 1H), 7.61 (d, J = 8.2 Hz, 1H), 7.57-7.49 (m, 1H), 7.35 (t, J = 5.6 Hz, 1H), 6.93-6.81 (m, 1H), 6.48 (s, 1H), 6.39 (d, J = 8.5 Hz, 1H), 6.34 (t, J = 8.4 Hz, 1H), 4.96-4.83 (m, 3H), 4.65-4.52 (m, 3H), 4.36-4.19 (m, 1H), 4.16- 4.02 (m, 2H), 3.85-3.75 (m, 1H), 3.73 (s, 3H), 3.63-3.49 (m, 2H), 3.22 (s, 3H), 1.81 (s, 3H), 1.77 (s, 3H). LC-MS: 570.20[M + 1]+ |
| 80 | 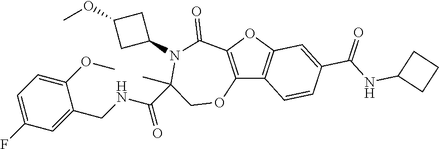 [SMILES: COc1ccc(F)cc1CNC(=O)C1(C)COc2c(oc3cc(C(=O)NC4CCC4)ccc23)C(=O)N1[C@H]1C[C@H](OC)C1] | 1H NMR (400 MHZ, DMSO) δ 8.75 (d, J = 7.3 Hz, 1H), 8.59-8.41 (m, 1H), 8.05 (s, 1H), 7.83 (d, J = 8.3 Hz, 1H), 7.76 (d, J = 8.2 Hz, 1H), 7.15-6.87 (m, 3H), 4.70-4.59 (m, 1H), 4.51-4.38 (m, 2H), 4.27 (d, J = 5.4 Hz, 2H), 4.05-3.96 (m, 1H), 3.85 (s, 1H), 3.78 (s, 3H), 3.13 (d, J = 30.2 Hz, 3H), 3.03-2.84 (m, 2H), 2.27-2.17 (m, 2H), 2.14-2.03 (m, 3H), 2.00- 1.87 (m, 1H), 1.75-1.61 (m, 2H), 1.41 (s, 3H). LC-MS: 580.20[M + 1]+ |
| 81 | 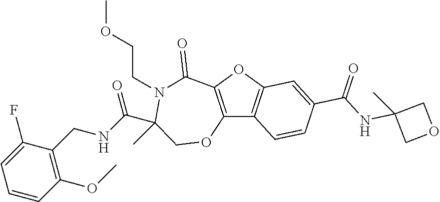 [SMILES: COCCN1C(=O)c2oc3cc(C(=O)NC4(C)COC4)ccc3c2OCC1(C)C(=O)NCc1c(F)cccc1OC] | 1H NMR (400 MHZ, DMSO) δ 9.01 (s, 1H), 8.09-7.93 (m, 2H), 7.79 (dd, J = 8.3, 1.2 Hz, 1H), 7.68 (d, J = 8.1 Hz, 1H), 7.25-7.08 (m, 1H), 6.75 (d, J = 8.3 Hz, 1H), 6.67-6.51 (m, 1H), 4.82 (d, J = 11.2 Hz, 1H), 4.74 (d, J = 6.4 Hz, 2H), 4.40 (d, J = 6.4 Hz, 2H), 4.36-4.11 (m, 3H), 3.72 (s, 3H), 3.68-3.58 (m, 2H), 3.54- 3.42 (m, 2H), 3.19 (s, 3H), 1.64 (s, 3H), 1.59 (s, 3H). LC-MS: 570.20[M + 1]+ |
| 82 | 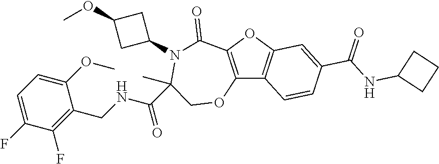 | 1H NMR (400 MHZ, DMSO) δ 8.74 (d, J = 7.5 Hz, 1H), 8.39 (s, 1H), 8.05 (s, 1H), 7.82 (dd, J = 8.3, 1.3 Hz, 1H), 7.75 (d, J = 8.3 Hz, 1H), 7.35 (q, J = 9.6 Hz, 1H), 6.84 (d, J = 8.0 Hz, 1H), 4.52 (d, J = 12.6 Hz, 1H), 4.49- 4.23 (m, 4H), 3.80 (s, 3H), 3.52-3.41 (m, 1H), 3.30-3.22 (m, 1H), 3.10 (s, 3H), 2.87-2.64 (m, 2H), 2.32-2.17 (m, 4H), 2.17-2.01 (m, 2H), 1.78-1.62 (m, 2H), 1.40 (s, 3H). LC-MS: 298.20[M + 1]+ |
| 83 | 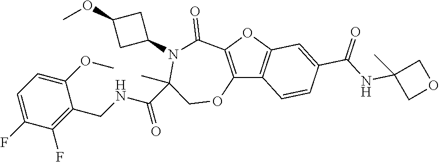 [SMILES: COc1ccc(F)c(F)c1CNC(=O)C1(C)COc2c(oc3cc(C(=O)NC4(C)COC4)ccc23)C(=O)N1[C@H]1C[C@@H](OC)C1] | 1H NMR (400 MHZ, DMSO) δ 9.02 (s, 1H), 8.39 (s, 1H), 8.05 (s, 1H), 7.82 (dd, J = 8.3, 1.2 Hz, 1H), 7.77 (d, J = 8.2 Hz, 1H), 7.35 (dd, J = 19.0, 9.4 Hz, 1H), 6.84 (d, J = 7.4 Hz, 1H), 4.73 (d, J = 6.2 Hz, 2H), 4.52 (d, J = 12.4 Hz, 1H), 4.45-4.24 (m, 5H), 3.80 (s, 3H), 3.53-3.42 (m, 1H), 3.28-3.19 (m, 1H), 3.10 (s, J = 7.6 Hz, 3H), 2.87-2.64 (m, 2H), 2.34-2.15 (m, 2H), 1.63 (s, 3H), 1.41 (s, 3H). LC-MS: 614.20[M + 1]+ |
| 84 | 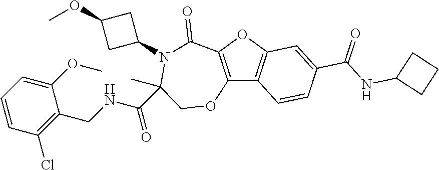 [SMILES: COc1cccc(Cl)c1CNC(=O)C1(C)COc2c(oc3cc(C(=O)NC4CCC4)ccc23)C(=O)N1[C@H]1C[C@@H](OC)C1] | 1H NMR (400 MHZ, DMSO) δ 8.74 (d, J = 7.5 Hz, 1H), 8.22 (s, 1H), 8.04 (s, 1H), 7.86-7.72 (m, 2H), 7.31 (t, J = 8.2 Hz, 1H), 7.03 (dd, J = 10.4, 8.4 Hz, 2H), 4.57 (d, J = 12.7 Hz, 1H), 4.50-4.37 (m, 3H), 4.33 (d, J = 12.7 Hz, 1H), 3.81 (s, 3H), 3.57-3.48 (m, 1H), 3.30-3.23 (m, 1H), 3.12 (s, 3H), 2.89-2.71 (m, 2H), 2.39-2.19 (m, 4H), 2.15-2.02 (m, 2H), 1.74-1.64 (m, 2H), 1.41 (s, 3H). LC-MS: 597.2[M + 1]+ |
| 85 | 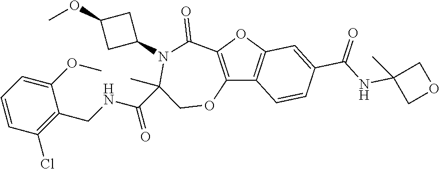 [SMILES: COc1cccc(Cl)c1CNC(=O)C1(C)COc2c(oc3cc(C(=O)NC4(C)COC4)ccc23)C(=O)N1[C@H]1C[C@@H](OC)C1] | 1H NMR (400 MHZ, DMSO) δ 9.02 (s, 1H), 8.22 (s, 1H), 8.05 (s, 1H), 7.85-7.72 (m, 2H), 7.31 (t, J = 8.2 Hz, 1H), 7.04 (dd, J = 10.0, 8.5 Hz, 2H), 4.73 (d, J = 6.2 Hz, 2H), 4.58 (d, J = 12.6 Hz, 1H), 4.48-4.30 (m, 5H), 3.82 (s, 3H), 3.56-3.47 (m, 1H), 3.29-3.24 (m, 1H), 3.12 (s, 3H), 2.91-2.73 (m, 2H), 2.40-2.19 (m, 2H), 1.63 (s, 3H), 1.41 (s, 3H). LC-MS: 612.2. [M + 1]+ |
| 86 | 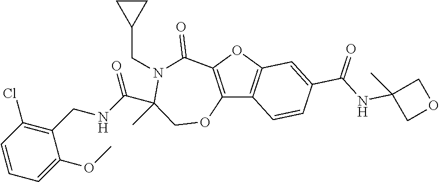 [SMILES: COc1cccc(Cl)c1CNC(=O)C1(C)COc2c(oc3cc(C(=O)NC4(C)COC4)ccc23)C(=O)N1CC1CC1] | 1H NMR (400 MHZ, CDCl3) δ 7.83 (s, 1H), 7.61-7.50 (m, 2H), 6.81 (t, J = 8.1 Hz, 2H), 6.59 (d, J = 9.1 Hz, 2H), 6.49 (d, J = 8.3 Hz, 1H), 4.96-4.86 (m, 3H), 4.76 (dd, J = 13.9, 7.1 Hz, 1H), 4.61 (d, J = 6.5 Hz, 2H), 4.21 (dd, J = 13.8, 4.2 Hz, 1H), 4.07 (d, J = 11.9 Hz, 1H), 3.88-3.76 (m, 1H), 3.72 (s, 3H), 3.53 (dd, J = 14.9, 5.4 Hz, 1H), 1.86 (s, 3H), 1.81 (s, 3H), 1.10-0.98 (m, 1H), 0.62-0.52 (m, 2H), 0.51-0.42 (m, 1H), 0.42-0.34 (m, 1H). LC-MS: 582.2[M + 1]+ |
| 87 | 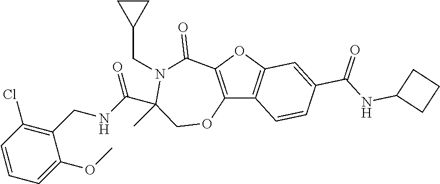 [SMILES: COc1cccc(Cl)c1CNC(=O)C1(C)COc2c(oc3cc(C(=O)NC4CCC4)ccc23)C(=O)N1CC1CC1] | 1H NMR (400 MHZ, CDCl3) δ 7.82 (s, 1H), 7.53 (dt, J = 8.2, 4.7 Hz, 2H), 6.86-6.73 (m, 2H), 6.57 (d, J = 8.1 Hz, 1H), 6.47 (d, J = 8.3 Hz, 1H), 6.29 (d, J = 7.6 Hz, 1H), 4.91 (d, J = 11.8 Hz, 1H), 4.80 (dd, J = 13.8, 7.3 Hz, 1H), 4.68-4.55 (m, 1H), 4.17 (dd, J = 13.9, 4.0 Hz, 1H), 4.06 (d, J = 11.9 Hz, 1H), 3.91-3.78 (m, 1H), 3.71 (s, 3H), 3.52 (dd, J = 14.7, 5.3 Hz, 1H), 2.54-2.42 (m, 2H), 2.07-1.95 (m, 2H), 1.89-1.78 (m, 5H), 1.10-1.00 (m, 1H), 0.64-0.52 (m, 2H), 0.50-0.36 (m, 2H). LC-MS: 566.2[M + 1]+ |
| 88 | 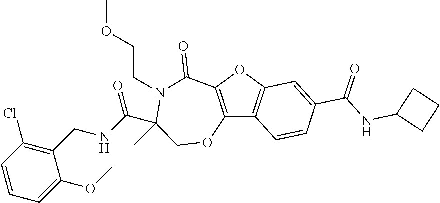 [SMILES: COCCN1C(=O)c2oc3cc(C(=O)NC4CCC4)ccc3c2OCC1(C)C(=O)NCc1c(Cl)cccc1OC] | 1H NMR (400 MHZ, CDCl3) δ 7.82 (s, 1H), 7.58 (d, J = 8.2 Hz, 1H), 7.52 (d, J = 4 Hz, 1H), 7.31 (s, 1H), 6.84 (t, J = 8.1 Hz, 1H), 6.62 (d, J = 8.0 Hz, 1H), 6.51 (d, J = 8.3 Hz, 1H), 6.27 (d, J = 7.2 Hz, 1H), 4.88 (d, J = 11.7 Hz, 1H), 4.74 (dd, J = 13.8, 6.9 Hz, 1H), 4.62 (dd, J = 16.2, 8.3 Hz, 1H), 4.36-4.01 (m, 3H), 3.78 (dt, J = 11.3, 3.9 Hz, 1H), 3.74 (d, J = 8.4 Hz, 3H), 3.55 (dq, J = 9.7, 3.8 Hz, 2H), 3.20 (s, 3H), 2.59-2.36 (m, 2H), 2.12-1.89 (m, 2H), 1.82-1.80 (m, 2H), 1.78 (s, 3H). LC-MS: 570.2[M + 1]+ |
| 89 | 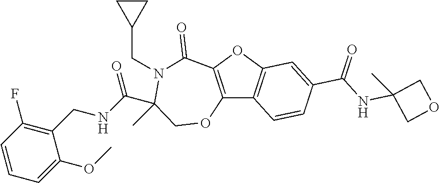 [SMILES: COc1cccc(F)c1CNC(=O)C1(C)COc2c(oc3cc(C(=O)NC4(C)COC4)ccc23)C(=O)N1CC1CC1] | 1H NMR (400 MHZ, CDCl3) δ 7.84 (s, 1H), 7.54 (tt, J = 4.6, 2.4 Hz, 2H), 6.88 (s, 1H), 6.82 (dd, J = 15.2, 8.1 Hz, 1H), 6.61 (s, 1H), 6.35 (d, J = 8.4 Hz, 1H), 6.30 (t, J = 8.5 Hz, 1H), 4.92-4.89 (m, 3H), 4.61 (d, J = 6.5 Hz, 2H), 4.60-4.51 (m, 1H), 4.13-4.06 (m, 2H), 3.86-3.75 (m, 1H), 3.74 (s, 3H), 3.56 (dd, J = 14.9, 5.5 Hz, 1H), 1.85 (s, 3H), 1.81 (s, 3H), 1.09-0.96 (m, 1H), 0.64-0.36 (m, 4H). LC-MS: 566.2[M + 1]+ |
| 90 | 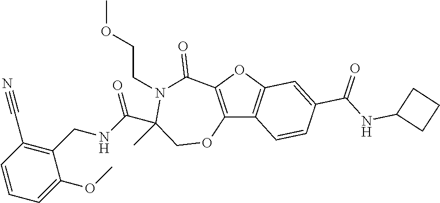 [SMILES: COCCN1C(=O)c2oc3cc(C(=O)NC4CCC4)ccc3c2OCC1(C)C(=O)NCc1c(C#N)cccc1OC] | 1H NMR (400 MHZ, CDCl3) δ 7.83 (s, 1H), 7.64 (t, J = 8 Hz, 1H), 7.52 (d, J = 8.2 Hz, 1H), 7.46 (d, J = 8.2 Hz, 1H), 6.86 (t, J = 7.5 Hz, 1H), 6.79 (d, J = 7.3 Hz, 1H), 6.70 (d, J = 8.2 Hz, 1H), 6.26 (d, J = 7.3 Hz, 1H), 4.89 (d, J = 11.7 Hz, 1H), 4.79 (dd, J = 14.1, 7.3 Hz, 1H), 4.63 (dd, J = 16.3, 8.2 Hz, 1H), 4.42-4.33 (m, 1H), 4.07 (t, J = 11.8 Hz, 2H), 3.85-3.81 (m, 1H), 3.76 (s, 3H), 3.68-3.48 (m, 2H), 3.34 (s, 3H), 2.58-2.34 (m, 2H), 2.18-1.93 (m, 2H), 1.85-1.81 (m, 2H), 1.79 (s, 3H). LC-MS: 561.2[M + 1]+ |
| 91 | 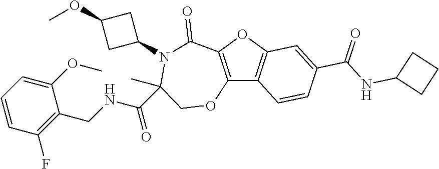 [SMILES: COc1cccc(F)c1CNC(=O)C1(C)COc2c(oc3cc(C(=O)NC4CCC4)ccc23)C(=O)N1[C@H]1C[C@@H](OC)C1] | 1H NMR (400 MHZ, DMSO) δ 8.74 (d, J = 7.5 Hz, 1H), 8.32 (s, 1H), 8.04 (s, 1H), 7.82 (dd, J = 8.3, 1.2 Hz, 1H), 7.75 (d, J = 8.3 Hz, 1H), 7.32 (dd, J = 15.4, 8.2 Hz, 1H), 6.87 (d, J = 8.4 Hz, 1H), 6.80 (t, J = 8.8 Hz, 1H), 4.55 (d, J = 12.7 Hz, 1H), 4.50-4.36 (m, 2H), 4.33 (d, J = 12.7 Hz, 1H), 4.29-4.21 (m, 1H), 3.82 (s, 3H), 3.55-3.42 (m, 1H), 3.28-3.23 (m, 1H), 3.11 (s, 3H), 2.88-2.63 (m, 2H), 2.37-2.17 (m, 4H), 2.17-1.99 (m, 2H), 1.80-1.61 (m, 2H), 1.40 (s, 3H). LC-MS:580.20[M + 1]+ |
| 92 | 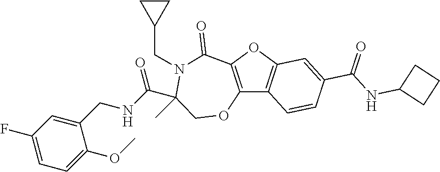 [SMILES: COc1ccc(F)cc1CNC(=O)C1(C)COc2c(oc3cc(C(=O)NC4CCC4)ccc23)C(=O)N1CC1CC1] | 1H NMR (400 MHZ, CDCl3) δ 7.83 (s, 1H), 7.52 (d, J = 8.2 Hz, 1H), 7.50-7.46 (m, 1H), 7.13-7.05 (m, 1H), 6.70-6.61 (m, 1H), 6.45- 6.37 (m, 2H), 6.29 (d, J = 7.5 Hz, 1H), 4.90 (d, J = 11.9 Hz, 1H), 4.70-4.57 (m, 1H), 4.40- 4.30 (m, 1H), 4.11-3.99 (m, 2H), 3.96-3.78 (m, 1H), 3.75 (s, 3H), 3.56 (dd, J = 14.9, 5.5 Hz, 1H), 2.59-2.40 (m, 2H), 2.13-1.94 (m, 2H), 1.86 (s, 3H), 1.85-1.72 (m, 2H), 1.13-0.99 (m, 1H), 0.71-0.55 (m, 2H), 0.54-0.43 (m, 2H). LC-MS: 550.20[M + 1]+ |
| 93 | 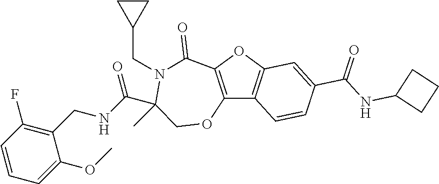 [SMILES: COc1cccc(F)c1CNC(=O)C1(C)COc2c(oc3cc(C(=O)NC4CCC4)ccc23)C(=O)N1CC1CC1] | 1H NMR (400 MHZ, CDCl3) δ 7.84 (s, 1H), 7.55 (d, J = 8.1 Hz, 1H), 7.51 (dd, J = 8.2, 1.1 Hz, 1H), 6.87 (s, 1H), 6.79 (dd, J = 15.2, 8.2 Hz, 1H), 6.37-6.28 (m, 2H), 6.28-6.19 (m, 1H), 4.90 (d, J = 11.9 Hz, 1H), 4.71-4.54 (m, 2H), 4.15-3.98 (m, 2H), 3.88-3.76 (m, 1H), 3.74 (s, 3H), 3.55 (dd, J = 14.9, 5.4 Hz, 1H), 2.56-2.37 (m, 2H), 2.10-1.93 (m, 2H), 1.93-1.74 (m, 6H), 1.13-0.98 (m, 1H), 0.71-0.53 (m, 2H), 0.53- 0.32 (m, 2H). LC-MS: 550.20[M + 1]+ |
| 94 | 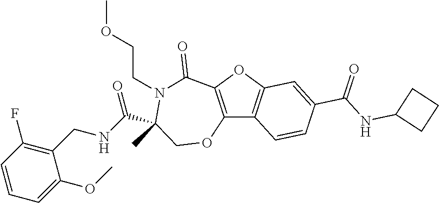 [SMILES: COCCN1C(=O)c2oc3cc(C(=O)NC4CCC4)ccc3c2OC[C@]1(C)C(=O)NCc1c(F)cccc1OC] | 1H NMR (400 MHZ, CDCl3) δ 7.83 (s, 1H), 7.57 (d, J = 8.2 Hz, 1H), 7.52 (dd, J = 8.2, 1.1 Hz, 1H), 7.40-7.32 (m, 1H), 6.84 (dd, J = 15.1, 8.0 Hz, 1H), 6.41-6.33 (m, 1H), 6.33-6.23 (m, 2H), 4.87 (d, J = 2H), 4.37-4.22 (m, 1H), 4.15- 4.01 (m, 2H), 3.83-3.74 11.8 Hz, 1H), 4.70-4.52 (m, (m, 1H), 3.72 (s, 3H), 3.63- 3.49 (m, 2H), 3.24 (s, 3H), 2.56-2.39 (m, 2H), 2.10- 1.93 (m, 2H), 1.88-1.72 (m, 5H). LC-MS: 554.20[M + 1]+ |

### Intermediate BB-6

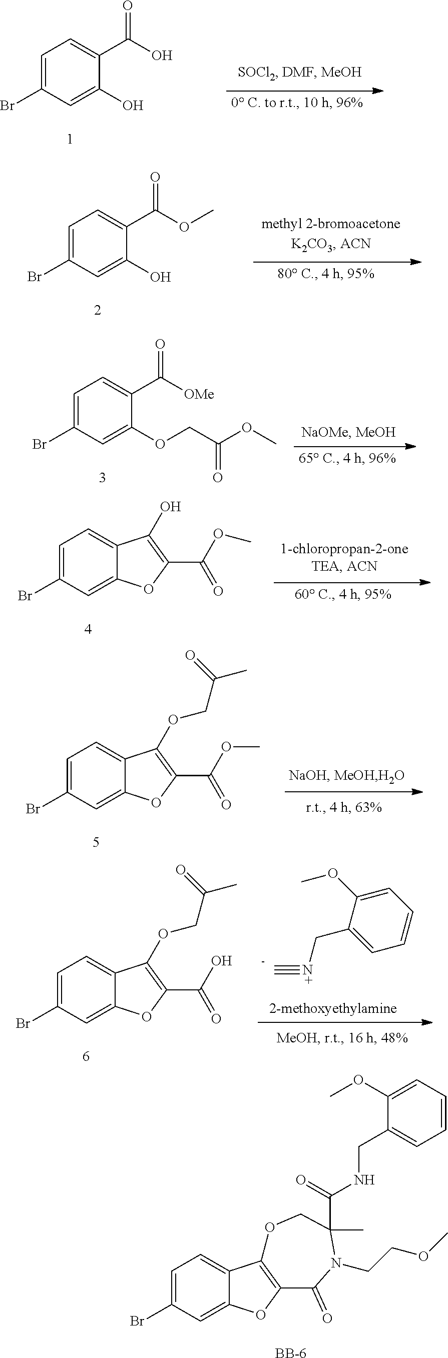

A mixture of compound 4-bromo-2-hydroxybenzoic acid (200 g, 0.92 mol, 1.0 eq), SOCl₂ (550 g, 4.60 mol, 5.0 eq), and DMF (15 mL) in MeOH (500 mL) was heated at 65° C. and stirred for 10 h. The reaction mixture was cooled to room temperature and concentrated under reduced pressure. The residue was washed with sat. NaOH, extracted with EA, the combined organic layers were dried over anhydrous Na₂SO₄, filtered, and concentrated under reduced pressure to give crude compound methyl 4-bromo-2-hydroxybenzoate (205 g, 96%) as a light yellow solid. TLC: PE/EA=3:1, UV; R_{f} (compound 1)=0.01; R_{f} (compound 2)=0.80; LC-MS: 231.0 [M+1]; 1H NMR (400 MHz, CDCl3) δ 10.82 (s, 1H), 7.67 (d, J=8.5 Hz, 1H), 7.17 (d, J=1.9 Hz, 1H), 7.01 (dd, J=8.5, 1.9 Hz, 1H), 3.94 (s, 3H).

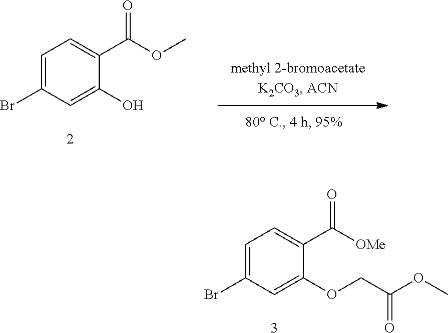

Methyl bromoacetate (164 g, 1.07 mol, 1.2 eq) was added dropwise over 30 min to a stirred mixture of compound methyl 4-bromo-2-hydroxybenzoate (205 g, 0.89 mol, 1.0 eq) and potassium carbonate (192 g, 1.33 mol, 1.5 eq) in MeCN (2 L). The mixture was heated and stirred under reflux for 4 h. The reaction was monitored by LCMS. The reaction was cooled, and the salts were separated by filtration and washed with acetone. The filtrate was concentrated to give a residue, which was diluted with EA (2 L), and the solution was washed successively with water, aqueous sodium carbonate, water, and brine. The crude product was distilled under reduced pressure to give compound methyl 4-bromo-2-(2-methoxy-2-oxoethoxy)benzoate (255 g, 95%) as a brown solid. TLC: PE/EA=3:1, UV; R_{f}(compound 2)=0.80; R_{f} (compound 3)=0.50; LC-MS: 303.0 [M+1]⁺. 1H NMR (400 MHz, DMSO) δ 7.61 (d, J=8.3 Hz, 1H), 7.34 (d, J=1.7 Hz, 1H), 7.27 (dd, J=8.3, 1.7 Hz, 1H), 4.97 (s, 2H), 3.80 (s, 3H), 3.71 (s, 3H).

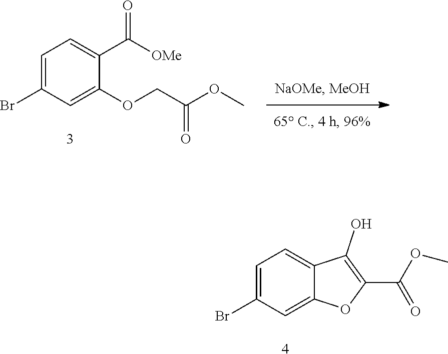

A mixture of compound methyl 4-bromo-2-(2-methoxy-2-oxoethoxy)benzoate (255 g, 0.84 mol, 1.0 eq) in MeOH (1 L) was added NaOMe (30% in methanol, 182 mL, 1.01 mol, 1.2 eq) and the mixture was stirred at 60° C. for 4 h. The reaction was monitored by LCMS. The resulting mixture was cooled, poured into ice water and acidified with 5% hydrochloric acid to pH 2. The solid was collected by filtration, washed with water and recrystallized from methanol to give compound methyl 6-bromo-3-hydroxybenzofuran-2-carboxylate (220 g, 96%) as yellow solid. TLC: PE/EA=3:1, UV; R_{f}(compound 3)=0.50; R_{f} (compound 4)=0.45; LC-MS: 271.1 [M+1]⁺. 1H NMR (400 MHz, DMSO) δ 11.04 (s, 1H), 7.92 (d, J=1.5 Hz, 1H), 7.85 (d, J=8.5 Hz, 1H), 7.49 (dd, J=8.5, 1.6 Hz, 1H), 3.84 (s, 3H).

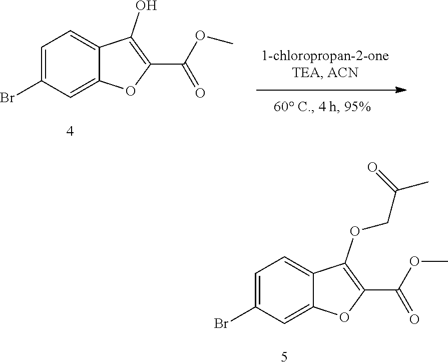

A mixture of compound methyl 6-bromo-3-hydroxybenzofuran-2-carboxylate (200 g, 0.74 mol, 1.0 eq) in acetonitrile (1 L) was added TEA (89 g, 0.89 mol, 1.2 eq), chloroacetone (75 g, 0.81 mmol, 1.1 eq), the mixture was refluxed for 4 h. The reaction was monitored by LCMS. The resulting mixture was filtered, concentrated, extracted with ethyl acetate to give crude compound methyl 6-bromo-3-(2-oxopropoxy)benzofuran-2-carboxylate (23 g, 95%) as brown solid. TLC: PE/EA=3:1, UV; R_{f}(compound 4)=0.45; R_{f} (compound 5)=0.6; LC-MS: 327.1 [M+1]⁺.

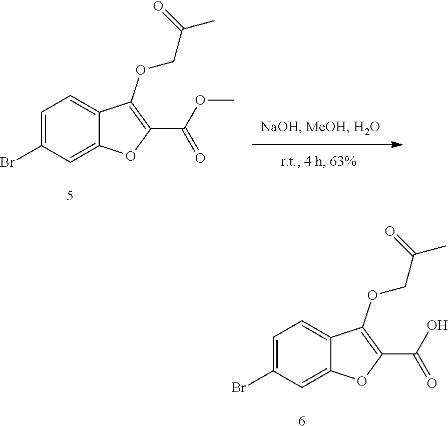

To a solution of crude compound methyl 6-bromo-3-(2-oxopropoxy)benzofuran-2-carboxylate (100 g, 0.31 mol, 1.0 eq) in MeOH (1 L) and 20% NaOH in water (400 mL) was stirred at rt for 4 h. The reaction was monitored by LCMS. The resulting mixture was concentrated, added 1 N HCl to adjust to pH 2, extracted with ethyl acetate. The combined organic layers were washed with brine, dried over Na₂SO₄. The solvent was removed under reduced pressure and the residue was purified by flash chromatography on silica (DCM:MeOH=10:1) to give a crude compound 6-bromo-3-(2-oxopropoxy)benzofuran-2-carboxylic acid (60 g, 63%) as yellow solid. TLC: DCM:MeOH=10:1, UV; R_{f}(compound 5)=0.9; R_{f} (compound 6)=0.2; LC-MS: 313.0 [M+1]⁺. 1H NMR (400 MHz, DMSO) δ 13.49 (s, 1H), 7.98 (d, J=1.4 Hz, 1H), 7.77 (d, J=8.5 Hz, 1H), 7.52 (dd, J=8.5, 1.5 Hz, 1H), 5.20 (s, 2H), 2.16 (s, 3H).

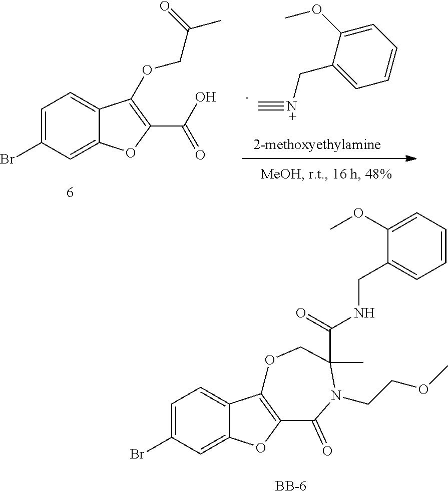

A solution of acid 6-bromo-3-(2-oxopropoxy)benzofuran-2-carboxylic acid (50 g, 0.16 mmol, 1.0 eq), 2-methoxyethylamine (18 g, 2.4 mmol, 1.5 eq) and isonitrile (30 g, 0.21 mmol, 1.3 eq) in methanol (500 mL) was stirred at r.t. for 16 h. The conversion of the reagents was followed by TLC. On completion, the reaction mixture was cooled to rt, the residue was purified by flash column chromatography on silica gel (PE:EA=3:1), triturated with ethyl acetate, filtered to give 8-bromo-N-(2-methoxybenzyl)-4-(2-methoxyethyl)-3-methyl-5-oxo-2,3,4,5-tetrahydrobenzofuro[2,3-f][1,4]oxazepine-3-carboxamide (40 g, 48%) as light-yellow solid. TLC: PE:EA=1:3, UV; R_{f}(compound 6)=0.01; R_{f} (compound BB-6)=0.2; LC-MS: 517.2 [M+1]⁺; 1H NMR (400 MHz, DMSO) δ 8.30 (s, 1H), 7.97 (s, 1H), 7.63 (d, J=8.4 Hz, 1H), 7.50 (d, J=8.3 Hz, 1H), 7.07 (t, J=7.3 Hz, 1H), 6.85 (d, J=8.1 Hz, 1H), 6.74 (s, 1H), 6.40 (s, 1H), 4.95 (d, J=11.8 Hz, 1H), 4.37 (d, J=12.3 Hz, 1H), 4.18 (d, J=3.8 Hz, 2H), 3.78-3.74 (m, 2H), 3.72 (s, 3H), 3.50 (t, J=5.6 Hz, 2H), 3.23 (s, 3H), 1.72 (s, 3H).

### Example 95

### N-(2-methoxybenzyl)-4-(2-methoxyethyl)-3,8-dimethyl-5-oxo-2,3,4,5-tetrahydrobenzofuro[2,3-f][1,4]oxazepine-3-carboxamide

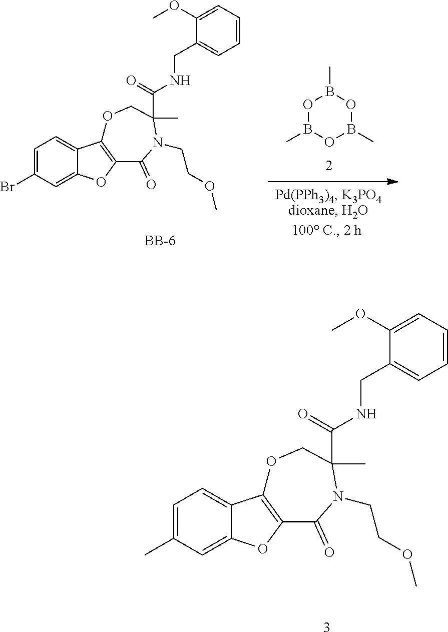

A mixture of Pd(PPh₃)₄ (134 mg, 0.011 mmol, 0.12 eq), K₃PO₄ (308 mg, 1.45 mmol), intermediate 8-bromo-N-(2-methoxybenzyl)-4-(2-methoxyethyl)-3-methyl-5-oxo-2,3,4,5-tetrahydrobenzofuro[2,3-f][1,4]oxazepine-3-carboxamide (500 mg, 0.10 mmol, 1.0 eq) and 2,4,6-trimethyl-1,3,5,2,4,6-trioxatriborinane (182 mg, 1.45 mmol, 1.5 eq) in dioxane (3 ml) and H₂O (0.3 ml) was degassed with argon for 2 mins, the resulting mixture stirred at 90° C. under argon for 3 h. The reaction mixture was allowed to cool and diluted with ethyl acetate.

This solution was washed with water. The organic layer was separated, dried over sodium sulfate and evaporated under reduced pressure, the residue was purified by flash column chromatography on silica gel (PE:EA=3:1), triturated with ethyl acetate, filtered to give N-(2-methoxybenzyl)-4-(2-methoxyethyl)-3,8-dimethyl-5-oxo-2,3,4,5-tetrahydrobenzofuro[2,3-f][1,4]oxazepine-3-carboxamide (150 mg, 34%) as light-yellow solid. TLC: PE:EA=1:1, UV; R_{f}(compound BB-6)=0.2; R_{f} (compound 3)=0.5; LC-MS: 453.2 [M+1]⁺; 1H NMR (400 MHz, CDCl3) δ 7.41 (d, J=8.1 Hz, 1H), 7.36 (s, 1H), 7.20 (s, 1H), 7.02 (d, J=8.1 Hz, 1H), 6.92 (dd, J=13.3, 7.1 Hz, 2H), 6.59 (d, J=8.1 Hz, 1H), 6.50 (t, J=7.1 Hz, 1H), 4.84 (d, J=12.0 Hz, 1H), 4.36 (dd, J=14.4, 6.7 Hz, 1H), 4.27-4.00 (m, 3H), 3.84-3.76 (m, 1H), 3.75 (s, 3H), 3.65-3.53 (m, 2H), 3.23 (s, 3H), 2.48 (s, 3H), 1.76 (s, 3H).

Using the above procedures and intermediates, the following compounds were prepared:

| Example | Structure | Analytical Data |
|---|---|---|
| 96 | 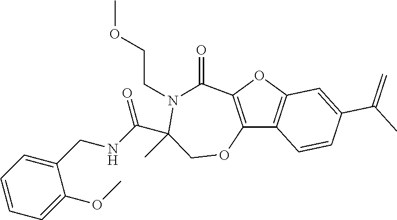 [SMILES: C=C(C)c1ccc2c3c(oc2c1)C(=O)N(CCOC)C(C)(C(=O)NCc1ccccc1OC)CO3] | 1H NMR (400 MHz, CDCl3) δ 7.47-7.45 (m, 2H), 7.42-7.37 (m, 1H), 7.34 (dd, J = 8.2, 1.5 Hz, 1H), 6.90-6.86 (m, 2H), 6.57 (d, J = 8.0 Hz, 1H), 6.47 (t, J = 7.2 Hz, 1H), 5.45 (s, 1H), 5.31-4.96 (m, 1H), 4.86 (d, J = 11.9 Hz, 1H), 4.37 (dd, J = 14.3, 6.9 Hz, 1H), 4.25-4.21 (m, 1H), 4.20-3.99 (m, 2H), 3.82- 3.77 (m, 1H), 3.76 (s, 3H), 3.63- 3.51 (m, 2H), 3.25 (s, 3H), 2.21 (s, 3H), 1.77 (s, 3H). LC-MS: 479.2 [M + 1]+ |
| 97 | 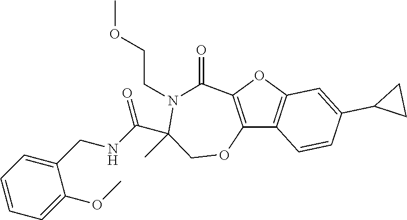 [SMILES: COCCN1C(=O)c2oc3cc(C4CC4)ccc3c2OCC1(C)C(=O)NCc1ccccc1OC] | 1H NMR (400 MHz, CDCl3) δ 7.40-7.36 (m, 2H), 7.08 (s, 1H), 6.99- 6.81 (m, 3H), 6.58 (d, J = 8.1 Hz, 1H), 6.48 (t, J = 7.3 Hz, 1H), 4.84 (d, J = 12.0 Hz, 1H), 4.36 (dd, J = 14.3, 6.7 Hz, 1H), 4.29-3.98 (m, 3H), 3.87-3.75 (m, 1H), 3.75 (s, 3H), 3.60-3.54 (m, 2H), 3.23 (s, 3H), 2.09-1.90 (m, 1H), 1.75 (s, 3H), 1.17-0.90 (m, 2H), 0.78-0.74 (m, 2H). LC-MS: 479.2 [M + 1]+ |

### Intermediate BB-7

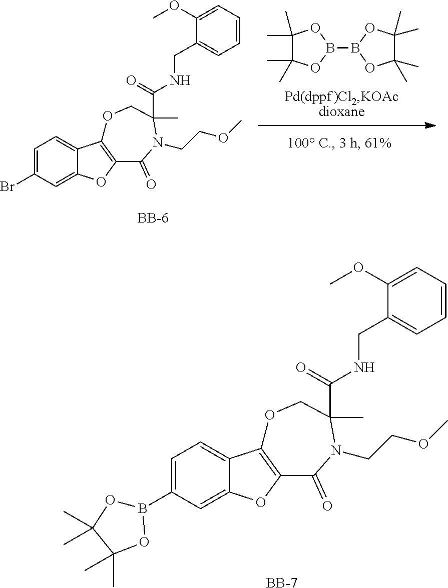

A mixture of (1,1′-bis(diphenylphosphino)ferrocene)palladium(11) chloride (200 mg, 0.27 mmol, 0.03 eq), potassium acetate (2.64 g, 26.9 mmol), Intermediate 8-bromo-N-(2-methoxybenzyl)-4-(2-methoxyethyl)-3-methyl-5-oxo-2,3,4,5-tetrahydrobenzofuro[2,3-f][1,4]oxazepine-3-carboxamide (4.2 g, 8.2 mmol, 1.1 eq) and bis(pinacolato)diboron (2.3 g, 9.1 mmol, 1.1 eq) in dioxane (60 ml) was degassed with argon for 5 mins, the resulting mixture stirred at 100° C. under argon for 3 h. The reaction mixture was allowed to cool and diluted with ethyl acetate. This solution was washed with water. The organic layer was separated, dried over sodium sulfate and evaporated under reduced pressure, the residue was purified by flash column chromatography on silica gel (PE:EA=3:1), triturated with ethyl acetate, filtered to give N-(2-methoxybenzyl)-4-(2-methoxyethyl)-3-methyl-5-oxo-8-(4,4,5,5-tetramethyl-1,3,2-dioxaborolan-2-yl)-2,3,4,5-tetrahydrobenzofuro[2,3-f][1,4]oxazepine-3-carboxamide (2.8 g, 61%) as light-yellow solid. LC-MS: 565.2 [M+1]⁺; TLC: PE:EA=3:1, UV; R_{f}(compound BB-6)=0.4; R_{f} (compound BB-7)=0.8.

Using the above procedures and intermediates, the following compounds were prepared:

| Example | Structure | Analytical Data |
|---|---|---|
| 99 | 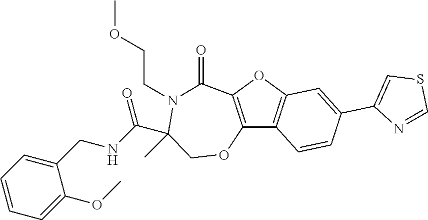 [SMILES: COCCN1C(=O)c2oc3cc(-c4cscn4)ccc3c2OCC1(C)C(=O)NCc1ccccc1OC] | 1H NMR (400 MHz, CDCl3) δ 8.93 (d, J = 1.9 Hz, 1H), 7.99 (s, 1H), 7.77 (dd, J = 8.3, 1.2 Hz, 1H), 7.62 (d, J = 1.9 Hz, 1H), 7.56 (d, J = 8.3 Hz, 1H), 7.42 (t, J = 5.7 Hz, 1H), 6.91 (d, J = 7.2 Hz, 1H), 6.85 (t, J = 7.4 Hz, 1H), 6.58 (d, J = 8.2 Hz, 1H), 6.47 (t, J = 7.3 Hz, 1H), 4.88 (d, J = 11.9 Hz, 1H), 4.39 (dd, J = 14.3, 7.0 Hz, 1H), 4.29-4.27 (m, 1H), 4.19- 4.03 (m, 2H), 3.84-3.77 (m, 1H), 3.77 (s, 3H), 3.65-3.54 |
| | | (m, 2H), 3.26 (s, 3H), 1.78 (s, |
| | | 3H). LC-MS: 522.20 [M + 1]+ |
| 100 | 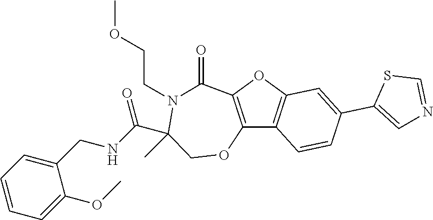 [SMILES: COCCN1C(=O)c2oc3cc(-c4cncs4)ccc3c2OCC1(C)C(=O)NCc1ccccc1OC] | 1H NMR (400 MHz, CDCl3) δ 8.83 (s, 1H), 8.15 (s, 1H), 7.57- 7.55 (m, 2H), 7.45-7.41 (m, 2H), 6.87 (dd, J = 14.6, 7.4 Hz, 2H), 6.59 (d, J = 8.1 Hz, 1H), 6.45 (t, J = 7.4 Hz, 1H), 4.89 (d, J = 11.9 Hz, 1H), 4.38 (dd, J = 14.3, 7.1 Hz, 1H), 4.30-4.28 (m, 1H), 4.17- 4.03 (m, 2H), 3.81-3.80 (m, 1H), 3.79 (s, 3H), 3.72-3.53 (m, 2H), 3.26 (s, 3H), 1.79 (s, 3H). LC-MS: 522.20 [M + 1]+ |
| 101 | 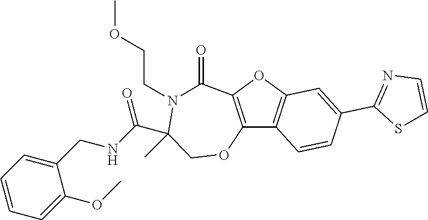 [SMILES: COCCN1C(=O)c2oc3cc(-c4nccs4)ccc3c2OCC1(C)C(=O)NCc1ccccc1OC] | 1H NMR (400 MHz, CDCl3) δ 8.00 (s, 1H), 7.92 (d, J = 3.2 Hz, 1H), 7.80 (dd, J = 8.3, 1.3 Hz, 1H), 7.56 (d, J = 8.3 Hz, 1H), 7.49-7.41 (m, 1H), 7.39 (d, J = 3.2 Hz, 1H), 6.90 (d, J = 7.6 Hz, 1H), 6.84 (t, J = 7.7 Hz, 1H), 6.58 (d, J = 8.1 Hz, 1H), 6.44 (t, J = 7.2 Hz, 1H), 4.89 (d, J = 11.8 Hz, 1H), 4.39 (dd, J = 14.2, 7.2 Hz, 1H), 4.30-4.28 (m, 1H), 4.15- 4.03 (m, 2H), 3.84-3.79 (m, 1H), 3.78 (s, 3H), 3.64-3.53 |
| | | (m, 2H), 3.28 (s, 3H), 1.79 (s, |
| | | 3H). LC-MS: 522.20 [M + 1]+ |

### Intermediate BB-8

### 5-chloro-3-(2-oxopropoxy)benzofuran-2-carboxylic acid

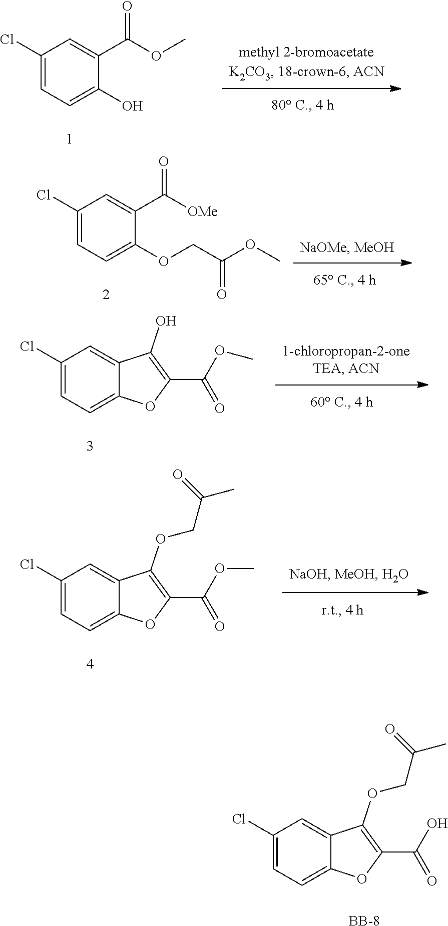

### methyl 5-chloro-2-(2-methoxy-2-oxoethoxy)benzoate

Methyl bromoacetate (90 g, 0.589 mol, 1.1 eq) was added dropwise over 30 min to a stirred mixture of compound methyl 5-chloro-2-hydroxybenzoate (100 g, 0.536 mol, 1.0 eq), 18-crown-6 (3 g, 0.011 mol, 0.02 eq) and potassium carbonate (111 g, 0.804 mol, 1.5 eq) in MeCN (2 L). The mixture was heated and stirred under reflux for 4 h. The reaction was monitored by LCMS. The reaction was cooled, and the salts were separated by filtration and washed with acetone. The filtrate was concentrated to give a residue, which was diluted with EA (2 L), and the solution was washed successively with water, aqueous sodium carbonate, water, and brine. The crude product was distilled under reduced pressure to give the product (138.7 g) as a brown solid. The crude product used in the next step directly. TLC: PE/EA=5:1, UV; R_{f} Starting Material=0.8, Product=0.5. LC-MS: Calculated Exact Mass=258.0, Found [M+H]⁺=259.0.

### methyl 5-chloro-3-hydroxybenzofuran-2-carboxylate

A mixture of compound methyl 5-chloro-2-(2-methoxy-2-oxoethoxy)benzoate (138.7 g, 0.537 mol, 1.0 eq) in MeOH (1 L) was added NaOMe (30% in methanol, 5 M, 128.8 mL, 0.644 mol, 1.2 eq) and the mixture was stirred at 60° C. for 4 hours. The reaction was monitored by LCMS. The resulting mixture was cooled, poured into ice water and acidified with 5% hydrochloric acid to pH 2. The solid was collected by filtration, washed with water and recrystallized from methanol to give the product (116 g, 96%) as yellow solid. TLC: PE/EA=1:1, UV; R_{f} Starting Material=0.5, Product=0.4. LC-MS: Calculated Exact Mass=226.0, Found [M+H]⁺=227.1.

### methyl 5-chloro-3-(2-oxopropoxy)benzofuran-2-carboxylate

A mixture of compound methyl 5-chloro-3-hydroxybenzofuran-2-carboxylate (115 g, 0.508 mol, 1.0 eq) in acetonitrile (1 L) was added TEA (103 g, 1.017 mol, 2 eq), chloroacetone (47 g, 0.508 mmol, 1.0 eq), the mixture was refluxed for 4 hours. The reaction was monitored by LCMS. The resulting mixture was filtered, concentrated, extracted with ethyl acetate to give the product (108 g, 75%) as brown solid. TLC: PE/EA=1:1, UV; R_{f} Starting Material=0.4, Product=0.8. LC-MS: Calculated Exact Mass=282.0, Found [M+H]⁺=283.0.

### 5-chloro-3-(2-oxopropoxy)benzofuran-2-carboxylic acid

To a solution of crude compound methyl 5-chloro-3-(2-oxopropoxy)benzofuran-2-carboxylate (90 g, 0.318 mol, 1.0 eq) in MeOH (1 L) and 10% NaOH in water (400 mL) was stirred at rt for 4 hours. The reaction was monitored by LCMS. The resulting mixture was concentrated, added 1 N HCl to adjust to pH 2, extracted with ethyl acetate. The combined organic layers were washed with brine, dried over Na₂SO₄. The solvent was removed under reduced pressure and the residue was purified by flash column chromatography on silica (DCM:MeOH=10:1) to give the product (26 g, 50%) as yellow solid. TLC: DCM:MeOH=10:1, UV; R_{f} Starting Material=0.9, Product=0.2. LC-MS: Calculated Exact Mass=268.0, Found [M+H]⁺=269.1. ¹H NMR (400 MHz, DMSO) δ 13.55 (s, 1H), 7.88 (d, J=2.1 Hz, 1H), 7.68 (d, J=8.9 Hz, 1H), 7.56 (dd, J=8.9, 2.2 Hz, 1H), 5.17 (s, 2H), 2.15 (s, 3H).

### Example 102

### (R)-4-(2-(bis(methyl-d3)amino)-2-oxoethyl)-9-chloro-N-(2-fluoro-6-methoxybenzyl)-3-methyl-5-oxo-2,3,4,5-tetrahydrobenzofuro[2,3-f][1,4]oxazepine-3-carboxamide

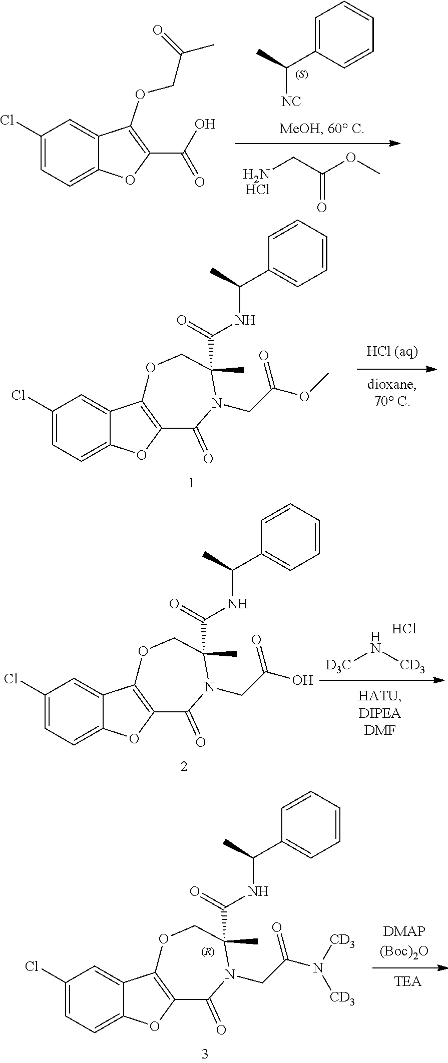

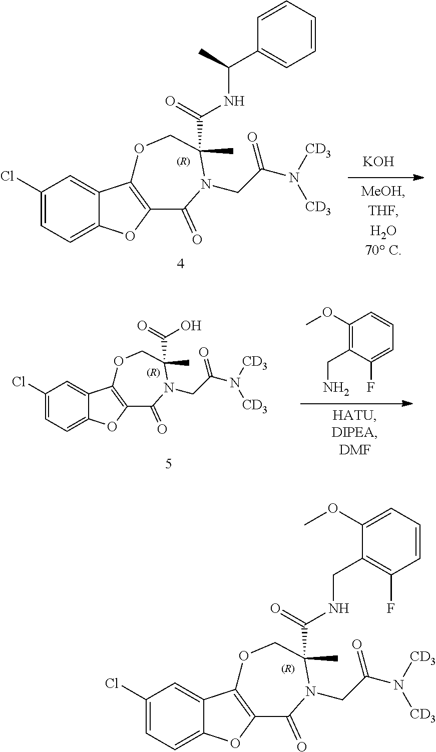

### methyl 2-((R)-9-chloro-3-methyl-5-oxo-3-(((S)-1-phenylethyl)carbamoyl)-2,3-dihydrobenzofuro[2,3-f][1,4]oxazepin-4(5H)-yl)acetate

A mixture of 5-chloro-3-(2-oxopropoxy)benzofuran-2-carboxylic acid (4 g, 14.89 mmol, 1.0 eq), methyl glycinate hydrochloride (2.8 g, 22.34 mmol, 1.5 eq), TEA (4.5 g, 44.68 mmol, 3.0 eq) and (S)-(1-isocyanoethyl)benzene (2.15 g, 16.38 mmol, 1.1 eq) in MeOH (20 mL) was stirred at 50° C. for 16 h. The mixture was concentrated under reduced pressure. The crude product was purified by flash chromatography on silica gel (PE:EA=1:1) to give the product (2 g, 28.6%) as a yellow solid. TLC: PE/EA=1:1, UV; R_{f} Starting Material=0.0, Product=0.4, isomer=0.6; LC-MS: Calculated Exact Mass=470.1, Found [M+H]⁺=471.1.

### 2-((R)-9-chloro-3-methyl-5-oxo-3-(((S)-1-phenylethyl)carbamoyl)-2,3-dihydrobenzofuro[2,3-f][1,4]oxazepin-4(5H)-yl)acetic acid

A mixture of methyl 2-((R)-9-chloro-3-methyl-5-oxo-3-(((S)-1-phenylethyl)carbamoyl)-2,3-dihydrobenzofuro[2,3-f][1,4]oxazepin-4(5H)-yl)acetate (2 g, 4.2 mmol, 1.0 eq), 6 M HCl (40 mL) in dioxane (80 mL) was stirred at 80° C. for 16 h. The mixture was concentrated under reduced pressure. The solution was extracted with EA, the solvent was removed under reduced pressure to give the product (1. g, 92.8%) as a yellow solid. TLC: PE/EA=1:1, UV; R_{f} Starting Material=0.4, Product=0.0. LC-MS: Calculated Exact Mass=456.1, Found [M+H]⁺=457.1.

### (R)-4-(2-(bis(methyl-d3)amino)-2-oxoethyl)-9-chloro-3-methyl-5-oxo-N—((S)-1-phenylethyl)-2,3,4,5-tetrahydrobenzofuro[2,3-f][1,4]oxazepine-3-carboxamide

To a solution of 2-((R)-9-chloro-3-methyl-5-oxo-3-(((S)-1-phenylethyl)carbamoyl)-2,3-dihydrobenzofuro[2,3-f][1,4]oxazepin-4(5H)-yl)acetic acid (1 g, 2.19 mmol, 1.0 eq) in DMF (30 mL) was added HATU (1.25 g, 3.29 mmol, 1.5 eq), bis(methyl-d3)amine hydrochloride (383 mg, 4.38 mmol, 2.0 eq) and DIPEA (1.4 g, 10.95 mmol, 5.0 eq) were added. The reaction mixture was stirred at room temperature for 2 h. The reaction mixture was poured into H₂O (30 mL) and extracted with EtOAc (3×30 mL). The combined organic layers were washed with brine, dried over Na₂SO₄, filtered, and concentrated under reduced pressure. The crude product was purified by flash chromatography on silica gel (DCM:MeOH=5:1) to give the product (800 mg, 74.7%) as a white solid. TLC: DCM/MeOH=5:1, UV; R_{f} Starting Material=0.15, Product=0.6. LC-MS: Calculated Exact Mass=565.2, Found [M+H]⁺=566.2.

### tert-butyl ((R)-4-(2-(bis(methyl-d3)amino)-2-oxoethyl)-9-chloro-3-methyl-5-oxo-2,3,4,5-tetrahydrobenzofuro[2,3-f][1,4]oxazepine-3-carbonyl)((S)-1-phenylethyl)carbamate

To a solution of (R)-4-(2-(bis(methyl-d3)amino)-2-oxoethyl)-9-chloro-3-methyl-5-oxo-N—((S)-1-phenylethyl)-2,3,4,5-tetrahydrobenzofuro[2,3-f][1,4]oxazepine-3-carboxamide (750 mg, 1.53 mmol, 1.0 eq) in TEA (20 mL) and DMA (2 mL) was added (Boc)₂O (6.5 g, 30.6 mmol, 20.0 eq), DMAP (238 mg, 1.95 mmol, 1.5 eq). The mixture was stirred at 100° C. for 3 hours. Water (50 mL) was added. The aqueous phase was extracted with EA (3×50 mL). The combined organic solution was washed with brine (50 mL), dried over anhydrous Na₂SO₄, and the solution was concentrated under reduced pressure. The residue was purified by silica gel chromatography (PE:EA=1:2) to give the product (750 mg, 83%) as brown solid. TLC: PE/EA=1:2, UV; R_{f} Starting Material=0.15, Product=0.6. LC-MS: Calculated Exact Mass=589.2, Found [M+H]⁺=590.2.

### (R)-4-(2-(bis(methyl-d3)amino)-2-oxoethyl)-9-chloro-3-methyl-5-oxo-2,3,4,5-tetrahydrobenzofuro[2,3-f][1,4]oxazepine-3-carboxylic acid

To a solution of tert-butyl ((R)-4-(2-(bis(methyl-d3)amino)-2-oxoethyl)-9-chloro-3-methyl-5-oxo-2,3,4,5-tetrahydrobenzofuro[2,3-f][1,4]oxazepine-3-carbonyl)((S)-1-phenylethyl)carbamate (750 mg, 1.27 mmol, 1.0 eq) in THE (5 mL), MeOH (6 mL), H₂O (3 mL) was added KOH (1.4 g, 25.4 mmol, 20.0 eq). The mixture was stirred at 70° C. for 3 hours. The reaction mixture was concentrated under reduced pressure and then acidified to pH 4-5 with 1 N HCl. The solid was filtered to give the product (410 mg, 83.6%) as white solid. The crude product used in the next step directly. LC-MS: Calculated Exact Mass=386.1, Found [M+H]⁺=387.1.

### (R)-4-(2-(bis(methyl-d3)amino)-2-oxoethyl)-9-chloro-N-(2-fluoro-6-methoxybenzyl)-3-methyl-5-oxo-2,3,4,5-tetrahydrobenzofuro[2,3-f][1,4]oxazepine-3-carboxamide

To a solution of (R)-4-(2-(bis(methyl-d3)amino)-2-oxoethyl)-9-chloro-3-methyl-5-oxo-2,3,4,5-tetrahydrobenzofuro[2,3-f][1,4]oxazepine-3-carboxylic acid (150 mg, 0.389 mmol, 1.0 eq) in DMF (2 mL) was added HATU (222 mg, 0.584 mmol, 1.5 eq). The mixture was stirred at room temperature for 10 min. (2-fluoro-6-methoxyphenyl)methanamine (72.3 mg, 0.466 mmol, 1.2 eq) and DIPEA (251 mg, 1.95 mmol, 5.0 eq) were added. The reaction mixture was stirred at room temperature for 2 h. The reaction mixture was poured into H₂O (30 mL) and extracted with EtOAc (3×30 mL). The combined organic layers were washed with brine, dried over Na₂SO₄, filtered, and concentrated under reduced pressure. The crude product was purified by prep-HPLC to give the product (85 mg, 41.8%) as a white solid. LC-MS: Calculated Exact Mass=523.2, Found [M+H]⁺=524.2. ¹H NMR (400 MHz, DMSO) δ 9.55 (s, 1H), 7.58 (d, J=13.5 Hz, 1H), 7.55 (d, J=8.9 Hz, 1H), 7.49 (dd, J=8.9, 2.1 Hz, 1H), 7.04 (dd, J=15.2, 7.8 Hz, 1H), 6.61 (d, J=8.3 Hz, 1H), 6.38 (s, 1H), 4.84 (d, J=11.8 Hz, 1H), 4.49 (s, 1H), 4.38-4.22 (m, 3H), 3.92 (s, 1H), 3.63 (s, 3H), 1.53 (s, 3H).

The following examples were prepared using a similar procedure as above

| Example | Structure | Analytical Data |
|---|---|---|
| 103 | 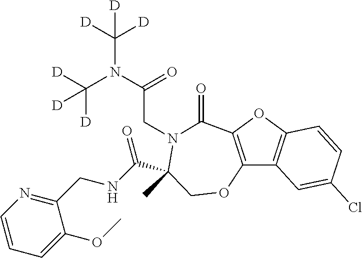 [SMILES: [2H]C([2H])([2H])N(C(=O)CN1C(=O)c2oc3ccc(Cl)cc3c2OC[C@]1(C)C(=O)NCc1ncccc1OC)C([2H])([2H])[2H]] | ¹H NMR (400 MHz, DMSO) δ 9.47 (s, 1H), 7.65 (s, 1H), 7.62 (d, J = 8.9 Hz, 1H), 7.52 (dd, J = 8.9, 2.1 Hz, 2H), 7.22 (d, J = 8.0 Hz, 1H), 7.06 (s, 1H), 4.86 (d, J = 10.4 Hz, 1H), 4.48 (s, 2H), 4.40-4.25 (m, 2H), 4.13 (s, 1H), 3.69 (s, 3H), 1.58 (s, 3H) LC-MS: Found [M + H]⁺ = 507.2 |
| 104 | 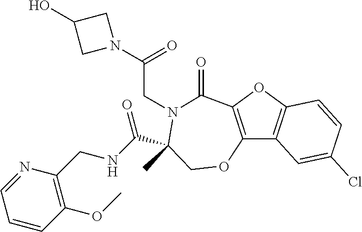 [SMILES: COc1cccnc1CNC(=O)[C@@]1(C)COc2c(oc3ccc(Cl)cc23)C(=O)N1CC(=O)N1CC(O)C1] | ¹H NMR (400 MHz, DMSO) δ 9.27 (s, 1H), 7.65 (s, 1H), 7.63 (d, J = 8.9 Hz, 1H), 7.57 (s, 1H), 7.52 (dd, J = 8.9, 2.2 Hz, 1H), 7.22 (d, J = 8.0 Hz, 1H), 7.07 (s, 1H), 5.82 (t, J = 5.4 Hz, 1H), 4.84 (d, J = 11.8 Hz, 1H), 4.53 (s, 1H), 4.45-3.95 (m, 8H), 3.69 (s, 3H), 3.63-3.61 (m, 1H), 1.58 (s, 3H). LC-MS: Found [M + H]⁺ = 529.2 |
| 105 | 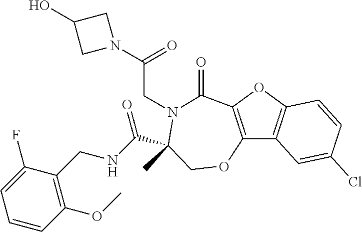 [SMILES: COc1cccc(F)c1CNC(=O)[C@@]1(C)COc2c(oc3ccc(Cl)cc23)C(=O)N1CC(=O)N1CC(O)C1] | ¹H NMR (400 MHz, DMSO) δ 9.31 (s, 1H), 7.64-7.53 (m, 2H), 7.49 (dd, J = 8.9, 2.1 Hz, 1H), 7.02 (d, J = 6.2 Hz, 1H), 6.59 (d, J = 7.7 Hz, 1H), 6.38 (s, 1H), 5.82 (dd, J = 7.8, 6.2 Hz, 1H), 4.82 (d, J = 11.8 Hz, 1H), 4.59-4.46 (m, 1H), 4.40 (dd, J = 18.1, 9.4 Hz, 1H), 4.35- 4.03 (m, 5H), 4.03-3.83 (m, 2H), 3.67-365 (m, 1H), 3.62 (s, 3H), 1.54 (s, 3H). LC-MS: Found [M + H]⁺ = 546.2 |

### Examples 106 and 107

### 9-chloro-4-(2-(dimethylamino)-2-oxoethyl)-N-(2-fluoro-6-methoxybenzyl)-3-methyl-5-oxo-2,3,4,5-tetrahydrobenzofuro[2,3-f][1,4]oxazepine-3-carboxamide and (S)-9-chloro-4-(2-(dimethylamino)-2-oxoethyl)-N-(2-fluoro-6-methoxybenzyl)-3-methyl-5-oxo-2,3,4,5-tetrahydrobenzofuro[2,3-f][1,4]oxazepine-3-carboxamide

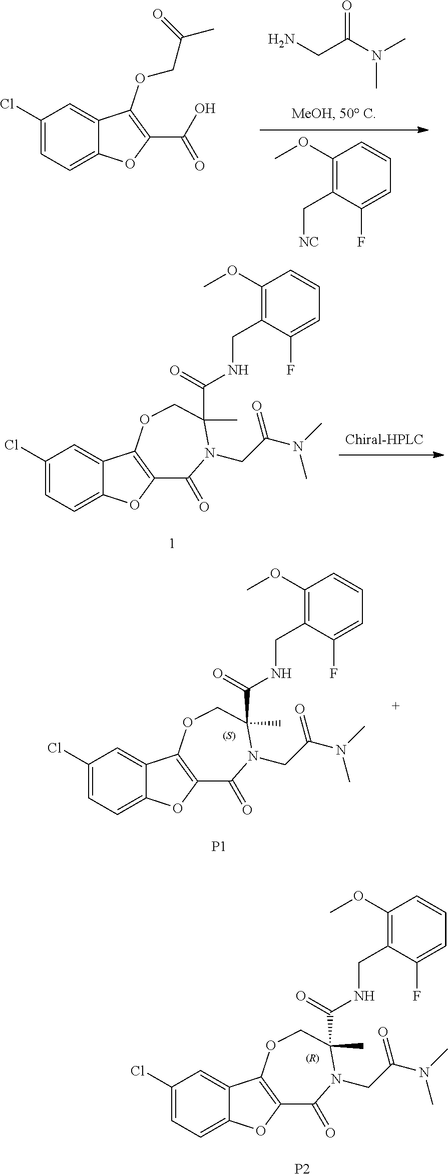

### 9-chloro-4-(2-(dimethylamino)-2-oxoethyl)-N-(2-fluoro-6-methoxybenzyl)-3-methyl-5-oxo-2,3,4,5-tetrahydrobenzofuro[2,3-f][1,4]oxazepine-3-carboxamide

A mixture of 5-chloro-3-(2-oxopropoxy)benzofuran-2-carboxylic acid (7 g, 26.1 mmol, 1.0 eq), 2-amino-N,N-dimethylacetamide (3.2 g, 31.32 mmol, 1.2 eq), and 1-fluoro-2-(isocyanomethyl)-3-methoxybenzene (4.3 g, 26.1 mmol, 1.0 eq) in MeOH (20 mL) was stirred at 50° C. for 16 h. The mixture was concentrated under reduced pressure. The crude product was purified by flash chromatography on silica gel (PE:EA=1:1) to give the product (7 g, 51.8%) as a yellow solid.

TLC: PE/EA=1:1, UV; R_{f} Starting Material=0.0, Product=0.4. LC-MS: Calculated Exact Mass=517.2, Found [M+H]⁺=518.2. ¹H NMR (400 MHz, DMSO) δ 9.54 (s, 1H), 7.60 (s, 1H), 7.55 (d, J=8.9 Hz, 1H), 7.49 (dd, J=8.9, 2.1 Hz, 1H), 7.04 (dd, J=15.0, 7.7 Hz, 1H), 6.61 (d, J=8.2 Hz, 1H), 6.38 (s, 1H), 4.84 (d, J=11.8 Hz, 1H), 4.49-4.47 (m, 1H), 4.38-4.20 (m, 3H), 4.01-3.92 (m, 1H), 3.63 (s, 3H), 3.03 (s, 3H), 2.86 (s, 3H), 1.53 (s, 3H).

### (S)-9-chloro-4-(2-(dimethylamino)-2-oxoethyl)-N-(2-fluoro-6-methoxybenzyl)-3-methyl-5-oxo-2,3,4,5-tetrahydrobenzofuro[2,3-f][1,4]oxazepine-3-carboxamide

Chiral HPLC: Chiral AS-H column (0.46 cm ID×15 cm L), CO₂:ethanol (0.1% DEA) (70:30); flow rate at 2.5 mL/min, monitored at 254 nm. Peak 1=3.397 min, 100% ee, 3.2 g. LC-MS: Calculated Exact Mass=517.2, Found [M+H]⁺=518.2. ¹H NMR (400 MHz, DMSO) δ 9.57 (s, 1H), 7.60 (s, 1H), 7.56 (d, J=8.9 Hz, 1H), 7.49 (dd, J=8.9, 2.2 Hz, 1H), 7.15-6.93 (m, 1H), 6.61 (d, J=8.2 Hz, 1H), 6.38 (s, 1H), 4.84 (d, J=11.8 Hz, 1H), 4.50 (m, 1H), 4.38-4.18 (m, 3H), 4.01-3.92 (m, 1H), 3.63 (s, 3H), 3.03 (s, 3H), 2.87 (s, 3H), 1.53 (s, 3H).

### (R)-9-chloro-4-(2-(dimethylamino)-2-oxoethyl)-N-(2-fluoro-6-methoxybenzyl)-3-methyl-5-oxo-2,3,4,5-tetrahydrobenzofuro[2,3-f][1,4]oxazepine-3-carboxamide

Chiral HPLC: Chiral AS-H column (0.46 cm ID×15 cm L), CO₂:ethanol (0.1% DEA) (70:30); flow rate at 2.5 mL/min, monitored at 254 nm. Peak 2=3.661 min, 98.05% ee, 3.1 g. LC-MS: Calculated Exact Mass=517.2, Found [M+H]⁺=518.2. ¹H NMR (400 MHz, DMSO) δ 9.57 (s, 1H), 7.60 (s, 1H), 7.56 (d, J=8.9 Hz, 1H), 7.49 (dd, J=8.9, 2.2 Hz, 1H), 7.15-6.93 (m, 1H), 6.61 (d, J=8.2 Hz, 1H), 6.38 (s, 1H), 4.84 (d, J=11.8 Hz, 1H), 4.50 (m, 1H), 4.38-4.18 (m, 3H), 4.01-3.92 (m, 1H), 3.63 (s, 3H), 3.03 (s, 3H), 2.87 (s, 3H), 1.53 (s, 3H).

### Example 108

### 9-chloro-N-(2-fluoro-6-methoxybenzyl)-3-methyl-4-((1-methyl-11H-1,2,4-triazol-5-yl)methyl)-5-oxo-2,3,4,5-tetrahydrobenzofuro[2,3-f][1,4]oxazepine-3-carboxamide

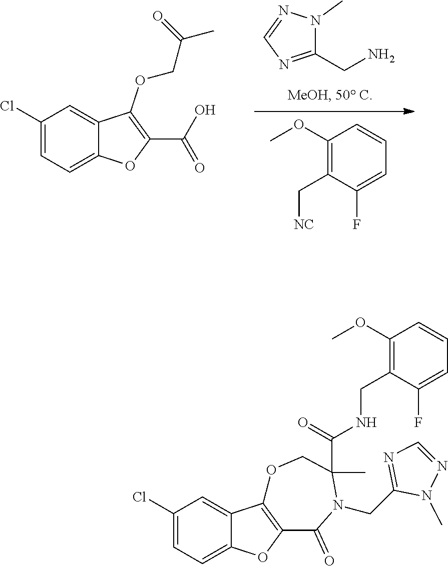

### 9-chloro-N-(2-fluoro-6-methoxybenzyl)-3-methyl-4-((1-methyl-11H-1,2,4-triazol-5-yl)methyl)-5-oxo-2,3,4,5-tetrahydrobenzofuro[2,3-f][1,4]oxazepine-3-carboxamide

A mixture of 5-chloro-3-(2-oxopropoxy)benzofuran-2-carboxylic acid (80 mg, 0.298 mmol, 1.0 eq), (1-methyl-1H-1,2,4-triazol-5-yl)methanamine (40 mg, 0.358 mmol, 1.2 eq), and 1-fluoro-2-(isocyanomethyl)-3-methoxybenzene (59 mg, 0.358 mmol, 1.02 eq) in MeOH (20 mL) was stirred at 50° C. for 16 h. The mixture was concentrated under reduced pressure. The crude product was purified by flash chromatography on silica gel (PE:EA=1:1) to give the product (70 mg, 44.5%) as a white solid. TLC: DCM/MeOH=10:1, UV; R_{f} Starting Material=0.1, Product=0.4. LC-MS: Calculated Exact Mass=527.2, Found [M+H]⁺=528.2. ¹H NMR (400 MHz, DMSO) δ 9.30 (s, 1H), 7.66 (d, J=2.0 Hz, 1H), 7.60 (d, J=8.9 Hz, 1H), 7.52 (dd, J=8.9, 2.2 Hz, 1H), 7.50 (s, 1H), 7.16 (dd, J=15.3, 8.3 Hz, 1H), 6.70 (d, J=8.4 Hz, 1H), 6.54 (t, J=8.7 Hz, 1H), 4.93 (d, J=12.1 Hz, 1H), 4.87 (s, 2H), 4.40 (d, J=12.1 Hz, 1H), 4.20 (dd, J=13.7, 5.1 Hz, 1H), 4.13-4.03 (m, 1H), 3.86 (s, 3H), 3.60 (s, 3H), 1.66 (s, 3H).

### Example 109

### 4-(2-(bis(methyl-d3)amino)-2-oxoethyl)-9-chloro-N-((3-ethoxypyridin-2-yl)methyl)-3-methyl-5-oxo-2,3,4,5-tetrahydrobenzofuro[2,3-f][1,4]oxazepine-3-carboxamide

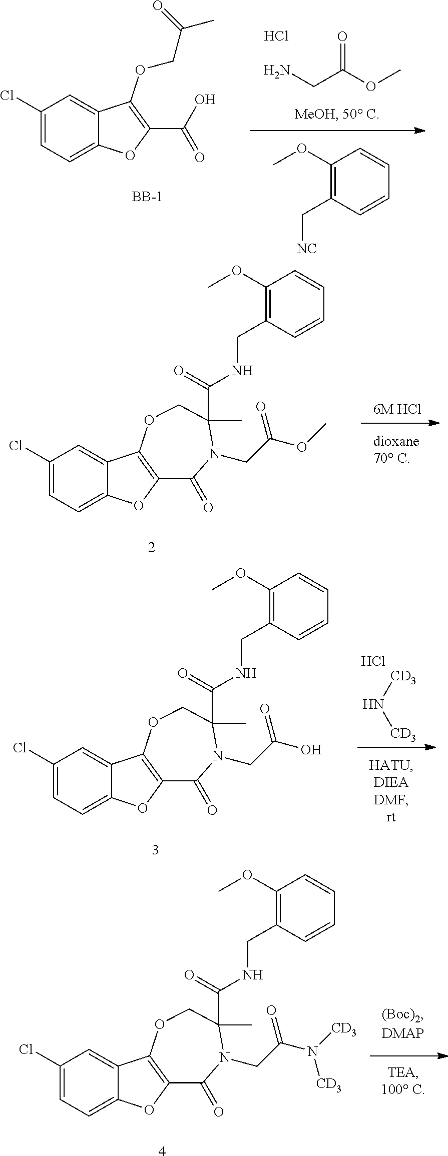

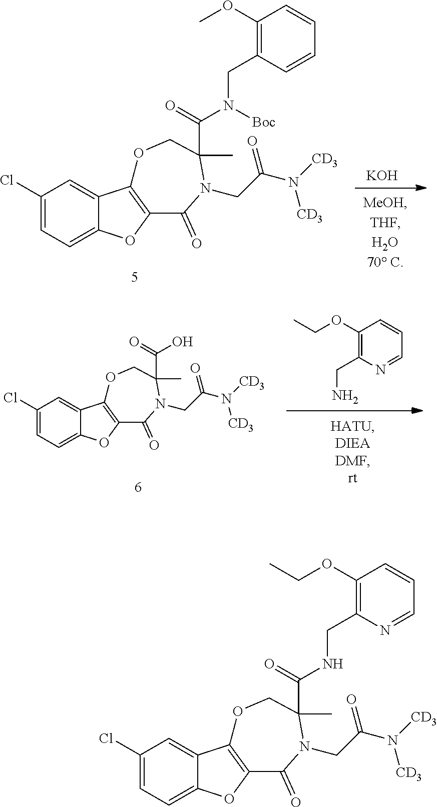

### methyl 2-(9-chloro-3-((2-methoxybenzyl)carbamoyl)-3-methyl-5-oxo-2,3-dihydrobenzofuro[2,3-f][1,4]oxazepin-4(5H)-yl)acetate

A mixture of 5-chloro-3-(2-oxopropoxy)benzofuran-2-carboxylic acid (2.4 g, 8.9 mmol, 1.0 eq), methyl glycinate hydrochloride (1.68 g, 13.4 mmol, 1.5 eq), TEA (2.71 g, 26.8 mmol, 3.0 eq) and 1-(isocyanomethyl)-2-methoxybenzene (2.63 g, 16.38 mmol, 1.2 eq) in MeOH (20 mL) was stirred at 50° C. for 16 h. The mixture was concentrated under reduced pressure. The crude product was purified by flash chromatography on silica gel (PE:EA=1:1) to give the product (2.6 g, 59.7%) as a yellow solid. TLC: PE/EA=1:1, UV; R_{f} Starting Material=0.0, Product=0.4. LC-MS: Calculated Exact Mass=486.1, Found [M+H]⁺=487.1.

### 2-(9-chloro-3-((2-methoxybenzyl)carbamoyl)-3-methyl-5-oxo-2,3-dihydrobenzofuro[2,3-f][1,4]oxazepin-4(5H)-yl)acetic acid

A mixture of methyl 2-(9-chloro-3-((2-methoxybenzyl)carbamoyl)-3-methyl-5-oxo-2,3-dihydrobenzofuro[2,3-f][1,4]oxazepin-4(5H)-yl)acetate (2.5 g, 5.13 mmol, 1.0 eq), 6 M HCl (40 mL) in dioxane (80 mL) was stirred at 70° C. for 16 h. The mixture was concentrated under reduced pressure. The solution was extracted with EA, the solvent was removed under reduced pressure to give the product (2.3 g, 95%) as a yellow solid. LC-MS: Calculated Exact Mass=472.0, Found [M+H]⁺=473.1.

### 4-(2-(bis(methyl-d3)amino)-2-oxoethyl)-9-chloro-N-(2-methoxybenzyl)-3-methyl-5-oxo-2,3,4,5-tetrahydrobenzofuro[2,3-f][1,4]oxazepine-3-carboxamide

To a solution of 2-(9-chloro-3-((2-methoxybenzyl)carbamoyl)-3-methyl-5-oxo-2,3-dihydrobenzofuro[2,3-f][1,4]oxazepin-4(5H)-yl)acetic acid (1 g, 2.1 mmol, 1.0 eq) in DMF (30 mL) was added HATU (1.2 g, 3.1 mmol, 1.5 eq), bis(methyl-d3)amine hydrochloride (370 mg, 4.2 mmol, 2.0 eq) and DIPEA (1.36 g, 10.5 mmol, 5.0 eq) were added. The reaction mixture was stirred at room temperature for 2 h. The reaction mixture was poured into H₂O (30 mL) and extracted with EtOAc (3×30 mL). The combined organic layers were washed with brine, dried over Na₂SO₄, filtered, and concentrated under reduced pressure. The crude product was purified by flash chromatography on silica gel (DCM:MeOH=5:1) to give the product (900 mg, 85%) as a white solid. TLC: DCM/MeOH=5:1, UV; R_{f} Starting Material=0.15, Product=0.6. LC-MS: Calculated Exact Mass=505.1, Found [M+H]⁺=506.2.

### tert-butyl (4-(2-(bis(methyl-d3)amino)-2-oxoethyl)-9-chloro-3-methyl-5-oxo-2,3,4,5-tetrahydrobenzofuro[2,3-f][1,4]oxazepine-3-carbonyl)(2-methoxybenzyl)carbamate

To a solution of 4-(2-(bis(methyl-d3)amino)-2-oxoethyl)-9-chloro-N-(2-methoxybenzyl)-3-methyl-5-oxo-2,3,4,5-tetrahydrobenzofuro[2,3-f][1,4]oxazepine-3-carboxamide (900 mg, 1.78 mmol, 1.0 eq) in TEA (20 mL) and DMA (2 mL) was added (Boc)₂O (7.7 g, 35.6 mmol, 20.0 eq), DMAP (238 mg, 1.95 mmol, 1.5 eq). The mixture was stirred at 100° C. for 3 hours. Water (50 mL) was added. The aqueous phase was extracted with EA (3×50 mL). The combined organic solution was washed with brine (50 mL), dried over anhydrous Na₂SO₄, and the solution was concentrated under reduced pressure. The residue was purified by silica gel chromatography (PE:EA=1:2) to give the product (650 mg, 60%) as brown solid. TLC: PE/EA=2:1, UV; R_{f} Starting Material=0.15, Product=0.6. LC-MS: Calculated Exact Mass=605.1, Found [M+H]⁺=606.1.

### 4-(2-(bis(methyl-d3)amino)-2-oxoethyl)-9-chloro-3-methyl-5-oxo-2,3,4,5-tetrahydrobenzofuro[2,3-f][1,4]oxazepine-3-carboxylic acid

To a solution of tert-butyl (4-(2-(bis(methyl-d3)amino)-2-oxoethyl)-9-chloro-3-methyl-5-oxo-2,3,4,5-tetrahydrobenzofuro[2,3-f][1,4]oxazepine-3-carbonyl)(2-methoxybenzyl)carbamate (500 mg, 0.826 mmol, 1.0 eq) in THE (5 mL), MeOH (6 mL), H₂O (3 mL) was added KOH (0.925 g, 16.5 mmol, 20.0 eq). The mixture was stirred at 70° C. for 3 hours. The reaction mixture was concentrated under reduced pressure and then acidified to pH 4-5 with 1 N HCl. The solid was filtered to give the product (300 mg, 94%) as white solid. The crude product used in the next step directly. LC-MS: Calculated Exact Mass=386.1, Found [M+H]⁺=387.1.

### 4-(2-(bis(methyl-d3)amino)-2-oxoethyl)-9-chloro-N-((3-ethoxypyridin-2-yl)methyl)-3-methyl-5-oxo-2,3,4,5-tetrahydrobenzofuro[2,3-f][1,4]oxazepine-3-carboxamide

To a solution of 4-(2-(bis(methyl-d3)amino)-2-oxoethyl)-9-chloro-3-methyl-5-oxo-2,3,4,5-tetrahydrobenzofuro[2,3-f][1,4]oxazepine-3-carboxylic acid (100 mg, 0.077 mmol, 1.0 eq) in DMF (2 mL) was added HATU (141 mg, 0.116 mmol, 1.5 eq). (3-ethoxypyridin-2-yl)methanamine (43.3 mg, 0.085 mmol, 1.1 eq) and DIPEA (167 mg, 0.388 mmol, 5.0 eq) were added. The reaction mixture was stirred at room temperature for 2 h. The reaction mixture was poured into H₂O (30 mL) and extracted with EtOAc (3×30 mL). The combined organic layers were washed with brine, dried over Na₂SO₄, filtered, and concentrated under reduced pressure. The crude product was purified by prep-HPLC to give the product (80 mg, 59.7%) as a white solid. LC-MS: Calculated Exact Mass=521.2, Found [M+H]⁺=522.2. ¹H NMR (400 MHz, DMSO) δ 9.42 (s, 1H), 7.63-7.61 (m, 2H), 7.52 (dd, J=8.9, 2.2 Hz, 2H), 7.18 (d, J=7.6 Hz, 1H), 7.02 (s, 1H), 4.85 (d, J=11.2 Hz, 1H), 4.48 (s, 2H), 4.40-4.23 (m, 2H), 4.21-3.80 (m, 3H), 1.58 (s, 3H), 1.27 (t, J=6.9 Hz, 3H).

The following examples were prepared using a similar procedure as above

| Example | Structure | Analytical Data |
|---|---|---|
| 110 | 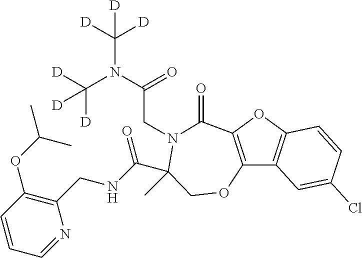 [SMILES: [2H]C([2H])([2H])N(C(=O)CN1C(=O)c2oc3ccc(Cl)cc3c2OCC1(C)C(=O)NCc1ncccc1OC(C)C)C([2H])([2H])[2H]] | ¹H NMR (400 MHz, DMSO) δ 9.31 (s, 1H), 7.66 (s, 1H), 7.63 (d, J = 8.9 Hz, 1H), 7.52 (dd, J = 8.9, 2.2 Hz, 2H), 7.23 (d, J = 8.1 Hz, 1H), 7.04 (s, 1H), 4.86 (d, J = 10.0 Hz, 1H), 4.65-4.44 (m, 3H), 4.37 (d, J = 12.3 Hz, 1H), 4.26 (dd, J = 15.8, 6.0 Hz, 1H), 4.15-4.13 (m, 1H), 1.58 (s, 3H), 1.21 (dd, J = 9.6, 6.0 Hz, 6H). LC-MS: Found [M + H]⁺ = 535.2 |

### Intermediate BB-9

### 5-fluoro-3-(2-oxopropoxy)benzofuran-2-carboxylic acid

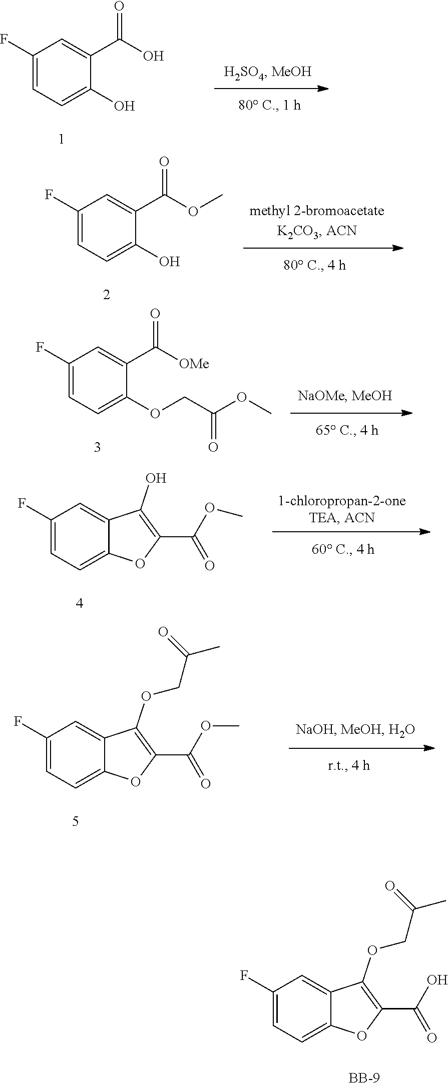

### methyl 5-fluoro-2-hydroxybenzoate

To a solution of 5-fluoro-2-hydroxybenzoic acid (190 g, 1.218 mol, 1.0 eq) in MeOH (2 L) was added H₂SO₄ (98%, 100 mL), the mixture was heated and stirred under reflux for 1 h. The reaction was monitored by LCMS. The crude product was distilled under reduced pressure, washed with H₂O, extracted with ethyl acetate, the organic phase was distilled under reduced pressure to give the product (192 g, 94%) as brown solid, TLC: PE/EA=5:1, UV; R_{f} Starting Material=0.0, Product=0.5. LC-MS: Calculated Exact Mass=170.0, Found [M+H]⁺=169.0.

### methyl 5-fluoro-2-(2-methoxy-2-oxoethoxy)benzoate

Methyl bromoacetate (205 g, 1.34 mol, 1.2 eq) was added dropwise over 30 min to a stirred mixture of compound methyl 5-fluoro-2-hydroxybenzoate (190 g, 1.12 mol, 1.0 eq), 18-crown-6 (3 g, 0.011 mol, 0.02 eq) and potassium carbonate (232 g, 1.68 mol, 1.5 eq) in MeCN (1 L). The mixture was heated and stirred under reflux for 4 h. The reaction was monitored by LCMS. The reaction was cooled, and the salts were separated by filtration and washed with acetone. The filtrate was concentrated to give a residue, which was diluted with EA (2 L), and the solution was washed successively with water, aqueous sodium carbonate, water, and brine. The crude product was distilled under reduced pressure to give the product (270 g, 87%) as a brown solid. The crude product used in the next step directly. TLC: PE/EA=4:1, UV; R_{f} Starting Material=0.7, Product=0.4. LC-MS: Calculated Exact Mass=242.0, Found [M+H]⁺=243.0.

### methyl 6-bromo-3-hydroxyfuro[3,2-b]pyridine-2-carboxylate

A mixture of compound methyl 5-fluoro-2-(2-methoxy-2-oxoethoxy)benzoate (250 g, 1.1 mol, 1.0 eq) in MeOH (3 L) was added NaOMe (30% in methanol, 5 M, 264 mL, 1.32 mol, 1.2 eq) and the mixture was stirred at 60° C. for 4 hours. The reaction was monitored by LCMS. The resulting mixture was cooled, poured into ice water and acidified with 5% hydrochloric acid to pH 2. The solid was collected by filtration, washed with water and recrystallized from methanol to give the product (190 g, 80%) as yellow solid. TLC: PE/EA=1:1, UV; R_{f} Starting Material=0.5, Product=0.4. LC-MS: Calculated Exact Mass=210.0, Found [M+H]⁺=211.1.

### methyl 5-fluoro-3-(2-oxopropoxy)benzofuran-2-carboxylate

A mixture of compound methyl 5-fluoro-3-hydroxybenzofuran-2-carboxylate (190 g, 0.905 mol, 1.0 eq) in acetonitrile (1 L) was added TEA (110 g, 1.086 mol, 1.2 eq), chloroacetone (92 g, 0.995 mmol, 1.1 eq), the mixture was refluxed for 4 hours. The reaction was monitored by LCMS. The resulting mixture was filtered, concentrated, extracted with ethyl acetate to give the product (132 g, 73%) as brown solid. TLC: PE/EA=1:1, UV; R_{f} Starting Material=0.4, Product=0.8. LC-MS: Calculated Exact Mass=266.0, Found [M+H]⁺=267.0.

### 5-fluoro-3-(2-oxopropoxy)benzofuran-2-carboxylic acid (BB-9)

To a solution of crude compound methyl 5-fluoro-3-(2-oxopropoxy)benzofuran-2-carboxylate (55 g, 0.207 mol, 1.0 eq) in MeOH (1.2 L) and 10% NaOH in water (600 mL) was stirred at rt for 4 hours. The reaction was monitored by LCMS. The resulting mixture was concentrated, added 1 N HCl to adjust to pH 2, extracted with ethyl acetate. The combined organic layers were washed with brine, dried over Na₂SO₄. The solvent was removed under reduced pressure and the residue was purified by flash column chromatography on silica (DCM:MeOH=10:1) to give the product (27 g, 31%) as yellow solid. TLC: DCM:MeOH=5:1, UV; R_{f} Starting Material=0.9, Product=0.2. LC-MS: Calculated Exact Mass=252.0, Found [M+H]⁺=253.0. ¹H NMR (400 MHz, DMSO) δ 13.51 (s, 1H), 7.69 (dd, J=9.2, 4.0 Hz, 1H), 7.63 (dd, J=8.5, 2.6 Hz, 1H), 7.41 (td, J=9.2, 2.7 Hz, 1H), 2.17 (s, 3H).

### Example 111

### 9-fluoro-N-(2-fluoro-6-methoxybenzyl)-4-(2-((2-hydroxyethyl)(methyl)amino)-2-oxoethyl)-3-methyl-5-oxo-2,3,4,5-tetrahydrobenzofuro[2,3-f][1,4]oxazepine-3-carboxamide

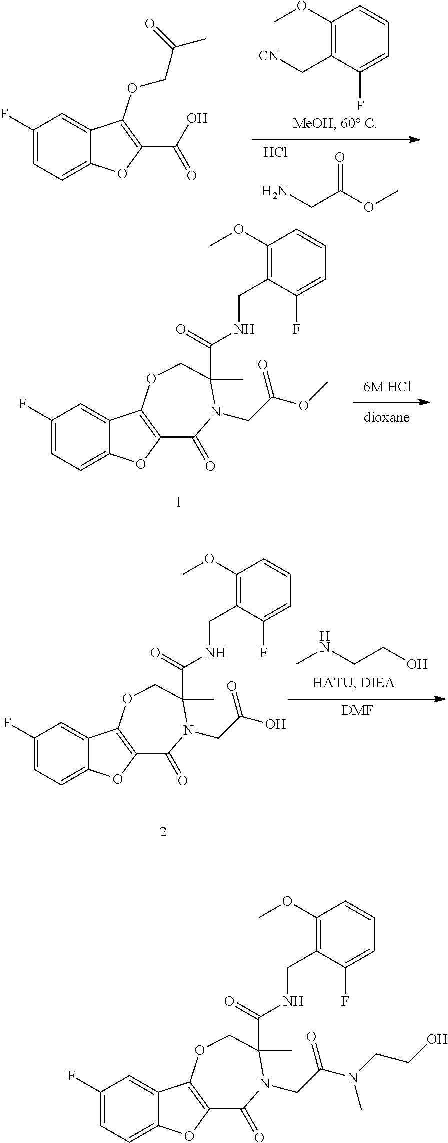

### methyl 2-(9-fluoro-3-((2-fluoro-6-methoxybenzyl)carbamoyl)-3-methyl-5-oxo-2,3-dihydrobenzofuro[2,3-f][1,4]oxazepin-4(5H)-yl)acetate

A mixture of 5-fluoro-3-(2-oxopropoxy)benzofuran-2-carboxylic acid (900 mg, 3.57 mmol, 1.0 eq), methyl glycinate hydrochloride (669 mg, 5.36 mmol, 1.5 eq), and 1-fluoro-2-(isocyanomethyl)-3-methoxybenzene (707 mg, 4.285 mmol, 1.2 eq) in MeOH (20 mL) was stirred at 50° C. for 16 h. The mixture was concentrated under reduced pressure. The crude product was purified by flash chromatography on silica gel (PE:EA=1:1) to give the product (1 g, 57.4%) as a yellow solid. TLC: PE/EA=1:1, UV; R_{f} Starting Material=0.0, Product=0.4. LC-MS: Calculated Exact Mass=488.1, Found [M+H]⁺=489.1.

### 2-(9-fluoro-3-((2-fluoro-6-methoxybenzyl)carbamoyl)-3-methyl-5-oxo-2,3-dihydrobenzofuro[2,3-f][1,4]oxazepin-4(5H)-yl)acetic acid

A mixture of methyl 2-(9-fluoro-3-((2-fluoro-6-methoxybenzyl)carbamoyl)-3-methyl-5-oxo-2,3-dihydrobenzofuro[2,3-f][1,4]oxazepin-4(5H)-yl)acetate (820 mg, 1.68 mmol, 1.0 eq), 6 M HCl (40 mL) in dioxane (80 mL) was stirred at 70° C. for 16 h. The mixture was concentrated under reduced pressure. The solution was extracted with EA, the solvent was removed under reduced pressure to give the product (750 mg, 94%) as a yellow solid. LC-MS: Calculated Exact Mass=474.1, Found [M+H]⁺=475.1.

### 9-fluoro-N-(2-fluoro-6-methoxybenzyl)-4-(2-((2-hydroxyethyl)(methyl)amino)-2-oxoethyl)-3-methyl-5-oxo-2,3,4,5-tetrahydrobenzofuro[2,3-f][1,4]oxazepine-3-carboxamide

To a solution of 2-(9-fluoro-3-((2-fluoro-6-methoxybenzyl)carbamoyl)-3-methyl-5-oxo-2,3-dihydrobenzofuro[2,3-f][1,4]oxazepin-4(5H)-yl)acetic acid (100 mg, 0.21 mmol, 1.0 eq) in DMF (2 mL) was added HATU (120 mg, 0.420 mmol, 1.5 eq). 2-(methylamino)ethan-1-ol (31 mg, 0.42 mmol, 2.0 eq) and DIPEA (136 mg, 1.05 mmol, 5.0 eq) were added. The reaction mixture was stirred at room temperature for 2 h. The reaction mixture was poured into H₂O (30 mL) and extracted with EtOAc (3×30 mL). The combined organic layers were washed with brine, dried over Na₂SO₄, filtered, and concentrated under reduced pressure. The crude product was purified by prep-HPLC to give the product (70 mg, 62.5%) as a white solid. LC-MS: Calculated Exact Mass=531.2, Found [M+H]⁺=532.2. ¹H NMR (400 MHz, DMSO) δ 9.54 (s, 1H), 7.54 (dt, J=7.6, 3.7 Hz, 1H), 7.45-7.21 (m, 2H), 7.02 (dd, J=15.2, 7.7 Hz, 1H), 6.61 (d, J=8.3 Hz, 1H), 6.38 (s, 1H), 4.99 (s, 1H), 4.82 (d, J=11.6 Hz, 1H), 4.36 (ddd, J=80.0, 54.0, 18.7 Hz, 5H), 3.89 (d, J=10.8 Hz, 1H), 3.63 (s, 3H), 3.61-3.43 (m, 3H), 3.08 (s, 1H), 2.88 (s, 2H), 1.53 (s, 1H), 1.50 (s, 2H).

### Example 112

### 4-(2-(dimethylamino)-2-oxoethyl)-9-fluoro-N-(2-fluoro-6-methoxybenzyl)-3-methyl-5-oxo-2,3,4,5-tetrahydrobenzofuro[2,3-f][1,4]oxazepine-3-carboxamide

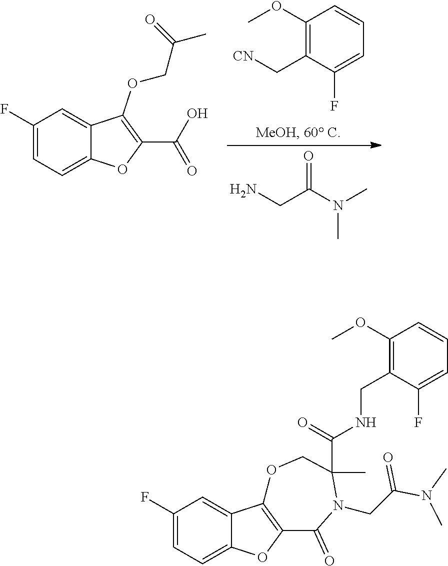

### 4-(2-(dimethylamino)-2-oxoethyl)-9-fluoro-N-(2-fluoro-6-methoxybenzyl)-3-methyl-5-oxo-2,3,4,5-tetrahydrobenzofuro[2,3-f][1,4]oxazepine-3-carboxamide

A mixture of 5-fluoro-3-(2-oxopropoxy)benzofuran-2-carboxylic acid (100 mg, 0.59 mmol, 1.0 eq), 2-amino-N,N-dimethylacetamide (91 mg, 1.5 mmol, 1.5 eq), and 1-fluoro-2-(isocyanomethyl)-3-methoxybenzene (147 mg, 0.89 mmol, 1.5 eq) in MeOH (20 mL) was stirred at 50° C. for 16 h. The mixture was concentrated under reduced pressure. The crude product was purified by flash chromatography on silica gel (PE:EA=1:1) to give the product (58 mg, 29%) as a yellow solid. LC-MS: Calculated Exact Mass=502.2, Found [M+H]⁺=501.2. ¹H NMR (400 MHz, DMSO) δ 9.54 (s, 1H), 7.55 (dd, J=9.0, 3.9 Hz, 1H), 7.41-7.27 (m, 2H), 7.03 (dd, J=15.2, 7.8 Hz, 1H), 6.61 (d, J=8.3 Hz, 1H), 6.40 (s, 1H), 4.83 (d, J=11.7 Hz, 1H), 4.64-4.21 (m, 4H), 3.93-3.91 (m, 1H), 3.63 (s, 3H), 3.03 (s, 3H), 2.86 (s, 3H), 1.53 (s, 3H).

### Example 113

### 4-(2-cyclopropyl-2-oxoethyl)-9-fluoro-N-(2-methoxybenzyl)-3-methyl-5-oxo-2,3,4,5-tetrahydrobenzofuro[2,3-f][1,4]oxazepine-3-carboxamide

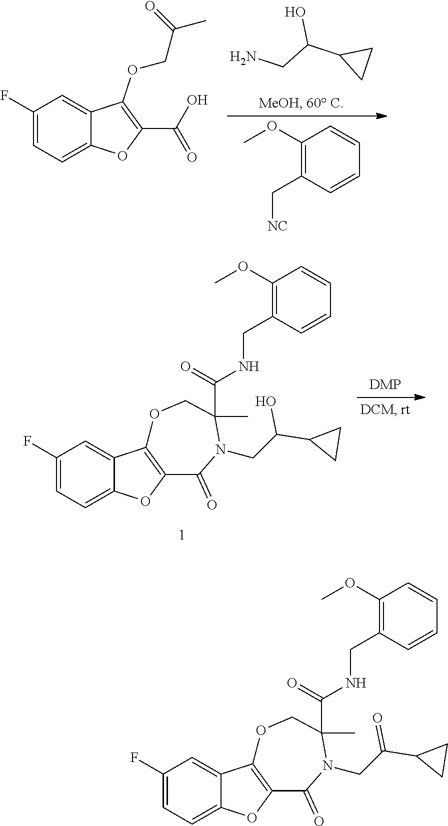

### 4-(2-cyclopropyl-2-hydroxyethyl)-9-fluoro-N-(2-methoxybenzyl)-3-methyl-5-oxo-2,3,4,5-tetrahydrobenzofuro[2,3-f][1,4]oxazepine-3-carboxamide

A mixture of 5-fluoro-3-(2-oxopropoxy)benzofuran-2-carboxylic acid (998 mg, 3.96 mmol, 1.0 eq), 2-amino-1-cyclopropylethan-1-ol (600 mg, 5.94 mmol, 1.5 eq), and 1-fluoro-2-(isocyanomethyl)-3-methoxybenzene (698 mg, 4.75 mmol, 1.2 eq) in MeOH (20 mL) was stirred at 50° C. for 16 h. The mixture was concentrated under reduced pressure. The crude product was purified by flash chromatography on silica gel (PE:EA=1:1) to give the product (1.1 g, 58%) as a yellow solid. TLC: PE/EA=1:1, UV; R_{f} Starting Material=0.0, Product=0.4. LC-MS: Calculated Exact Mass=482.2, Found [M+H]⁺=483.2.

### 4-(2-cyclopropyl-2-oxoethyl)-9-fluoro-N-(2-methoxybenzyl)-3-methyl-5-oxo-2,3,4,5-tetrahydrobenzofuro[2,3-f][1,4]oxazepine-3-carboxamide

A mixture of 4-(2-cyclopropyl-2-hydroxyethyl)-9-fluoro-N-(2-methoxybenzyl)-3-methyl-5-oxo-2,3,4,5-tetrahydrobenzofuro[2,3-f][1,4]oxazepine-3-carboxamide (100 mg, 0.207 mmol, 1.0 eq) and Dess-Martin Periodinane (131 mg, 0.31 mmol, 1.5 eq) in DCM (20 mL) was stirred at 25° C. for 16 h. The mixture was washed with water, extracted with DCM, the organic phase were concentrated under reduced pressure. The crude product was purified by Prep-HPLC to give the product (25 mg, 25%) as a yellow solid. LC-MS: Calculated Exact Mass=480.2, Found [M+H]⁺=481.2. ¹H NMR (400 MHz, DMSO) δ 8.75 (s, 1H), 7.67 (dd, J=9.1, 3.9 Hz, 1H), 7.49 (dd, J=8.1, 2.6 Hz, 1H), 7.41 (td, J=9.3, 2.7 Hz, 1H), 7.03 (t, J=7.4 Hz, 1H), 6.82 (d, J=8.2 Hz, 1H), 6.53 (s, 1H), 6.23 (s, 1H), 4.94 (d, J=12.2 Hz, 1H), 4.84 (d, J=18.3 Hz, 1H), 4.55-4.51 (m, 1H), 4.41 (d, J=12.3 Hz, 1H), 4.25 (dd, J=15.9, 6.4 Hz, 1H), 4.12-3.95 (m, 1H), 3.70 (s, 3H), 2.19-2.17 (m, 1H), 1.56 (s, 3H), 1.06-0.73 (m, 4H).

### Example 114

### (R)-4-(2-(bis(methyl-d3)amino)-2-oxoethyl)-9-fluoro-N-(2-fluoro-6-methoxybenzyl)-3-methyl-5-oxo-2,3,4,5-tetrahydrobenzofuro[2,3-f][1,4]oxazepine-3-carboxamide

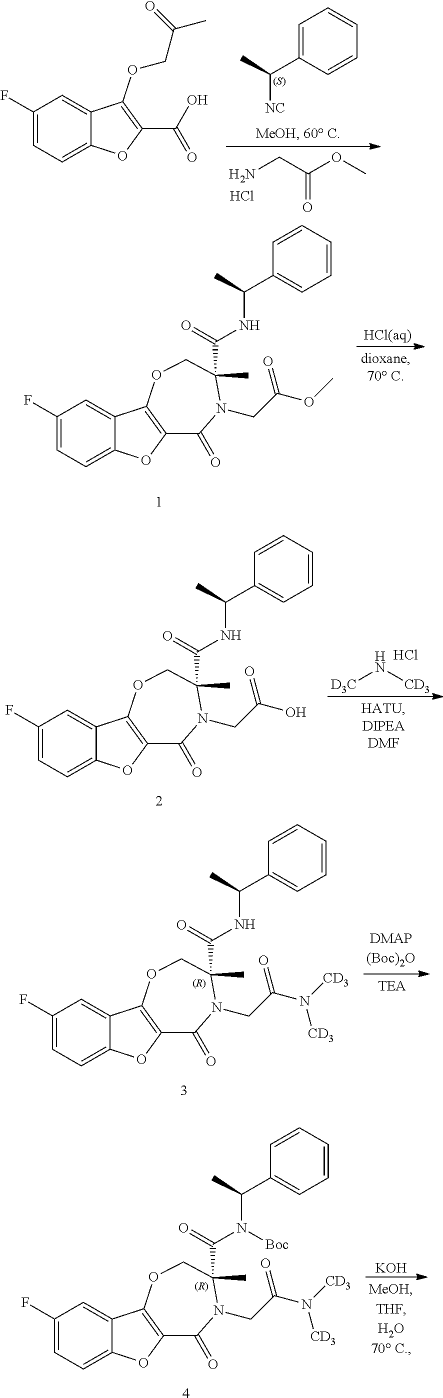

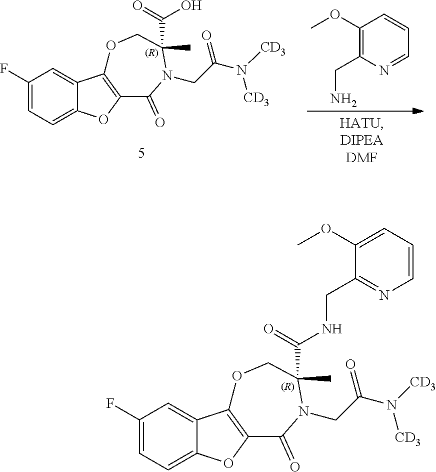

### methyl 2-((R)-9-fluoro-3-methyl-5-oxo-3-(((S)-1-phenylethyl)carbamoyl)-2,3-dihydrobenzofuro[2,3-f][1,4]oxazepin-4(5H)-yl)acetate

A mixture of 5-fluoro-3-(2-oxopropoxy)benzofuran-2-carboxylic acid (1.3 g, 5.155 mmol, 1.0 eq), methyl glycinate hydrochloride (1.4 g, 10.31 mmol, 2.0 eq), TEA (1.03 g, 10.31 mmol, 2.0 eq) and (S)-(1-isocyanoethyl)benzene (0.9 g, 10.31 mmol, 2.0 eq) in MeOH (20 mL) was stirred at 50° C. for 16 h. The mixture was concentrated under reduced pressure. The crude product was purified by flash chromatography on silica gel (PE:EA=1:1) to give the product (550 mg, 23.5%) as a yellow solid. TLC: PE/EA=1:1, UV; R_{f}Starting Material=0.0, Product=0.4, isomer=0.6; LC-MS: Calculated Exact Mass=454.1, Found [M+H]⁺=455.1.

### 2-((R)-9-fluoro-3-methyl-5-oxo-3-(((S)-1-phenylethyl)carbamoyl)-2,3-dihydrobenzofuro[2,3-f][1,4]oxazepin-4(5H)-yl)acetic acid

A mixture of methyl 2-((R)-9-fluoro-3-methyl-5-oxo-3-(((S)-1-phenylethyl)carbamoyl)-2,3-dihydrobenzofuro[2,3-f][1,4]oxazepin-4(5H)-yl)acetate (500 mg, 1.1 mmol, 1.0 eq), 6 M HCl (8 mL) in dioxane (6 mL) was stirred at 70° C. for 16 h. The mixture was concentrated under reduced pressure. The solution was extracted with EA, the solvent was removed under reduced pressure to give the product (436 mg, 90%) as a yellow solid. TLC: PE/EA=1:1, UV; R_{f} Starting Material=0.4, Product=0.0. LC-MS: Calculated Exact Mass=440.1, Found [M+H]⁺=441.1.

### (R)-4-(2-(bis(methyl-d3)amino)-2-oxoethyl)-9-fluoro-3-methyl-5-oxo-N—((S)-1-phenylethyl)-2,3,4,5-tetrahydrobenzofuro[2,3-f][1,4]oxazepine-3-carboxamide

To a solution of 2-((R)-9-fluoro-3-methyl-5-oxo-3-(((S)-1-phenylethyl)carbamoyl)-2,3-dihydrobenzofuro[2,3-f][1,4]oxazepin-4(5H)-yl)acetic acid (400 mg, 0.908 mmol, 1.0 eq) in DMF (30 mL) was added HATU (690 mg, 1.816 mmol, 2.0 eq), bis(methyl-d3)amine hydrochloride (92 mg, 1.816 mmol, 2.0 eq) and DIPEA (234 mg, 1.816 mmol, 2.0 eq) were added. The reaction mixture was stirred at room temperature for 2 h. The reaction mixture was poured into H₂O (30 mL) and extracted with EtOAc (3×30 mL). The combined organic layers were washed with brine, dried over Na₂SO₄, filtered, and concentrated under reduced pressure. The crude product was purified by flash chromatography on silica gel (DCM:MeOH=5:1) to give the product (200 mg, 46.7%) as a white solid. LC-MS: Calculated Exact Mass=473.2, Found [M+H]⁺=474.2.

### tert-butyl ((R)-4-(2-(bis(methyl-d3)amino)-2-oxoethyl)-9-fluoro-3-methyl-5-oxo-2,3,4,5-tetrahydrobenzofuro[2,3-f][1,4]oxazepine-3-carbonyl)((S)-1-phenylethyl)carbamate

To a solution of (R)-4-(2-(bis(methyl-d3)amino)-2-oxoethyl)-9-fluoro-3-methyl-5-oxo-N—((S)-1-phenylethyl)-2,3,4,5-tetrahydrobenzofuro[2,3-f][1,4]oxazepine-3-carboxamide (150 mg, 0.317 mmol, 1.0 eq) in TEA (50 mL) was added (Boc)₂O (10 g, 45.8 mmol, 144.0 eq), DMAP (18 mg, 0.158 mmol, 0.5 eq). The mixture was stirred at 100° C. for 3 hours. Water (50 mL) was added. The aqueous phase was extracted with EA (3×50 mL). The combined organic solution was washed with brine (50 mL), dried over anhydrous Na₂SO₄, and the solution was concentrated under reduced pressure. The residue was purified by silica gel chromatography (PE:EA=1:2) to give the product (210 mg, 100%) as brown solid. TLC: PE/EA=1:2, UV; R_{f} Starting Material=0.15, Product=0.6. LC-MS: Calculated Exact Mass=589.2, Found [M+H]⁺=590.2.

### (R)-4-(2-(bis(methyl-d3)amino)-2-oxoethyl)-9-fluoro-3-methyl-5-oxo-2,3,4,5-tetrahydrobenzofuro[2,3-f][1,4]oxazepine-3-carboxylic acid

To a solution of tert-butyl ((R)-4-(2-(bis(methyl-d3)amino)-2-oxoethyl)-9-fluoro-3-methyl-5-oxo-2,3,4,5-tetrahydrobenzofuro[2,3-f][1,4]oxazepine-3-carbonyl)((S)-1-phenylethyl)carbamate (150 mg, 0.261 mmol, 1.0 eq) in THE (5 mL) and MeOH (10 mL) was added KOH (10%, 10 mL). The mixture was stirred at 70° C. for 3 hours. The reaction mixture was concentrated under reduced pressure and then acidified to pH 4-5 with 1 N HCl. The solid was filtered to give the product (120 mg, 89%) as white solid. The crude product used in the next step directly. LC-MS: Calculated Exact Mass=370.1, Found [M+H]⁺=371.1.

### (R)-4-(2-(bis(methyl-d3)amino)-2-oxoethyl)-9-fluoro-N-((3-methoxypyridin-2-yl)methyl)-3-methyl-5-oxo-2,3,4,5-tetrahydrobenzofuro[2,3-f][1,4]oxazepine-3-carboxamide

To a solution of (R)-4-(2-(bis(methyl-d3)amino)-2-oxoethyl)-9-fluoro-3-methyl-5-oxo-2,3,4,5-tetrahydrobenzofuro[2,3-f][1,4]oxazepine-3-carboxylic acid (100 mg, 0.27 mmol, 1.0 eq) in DMF (2 mL) was added HATU (205 mg, 0.54 mmol, 2.0 eq). The mixture was stirred at room temperature for 10 min. (3-methoxypyridin-2-yl)methanamine (75 mg, 0.54 mmol, 2.0 eq) and DIPEA (70 mg, 0.54 mmol, 2.0 eq) were added. The reaction mixture was stirred at room temperature for 2 h. The reaction mixture was poured into H₂O (30 mL) and extracted with EtOAc (3×30 mL). The combined organic layers were washed with brine, dried over Na₂SO₄, filtered, and concentrated under reduced pressure. The crude product was purified by prep-HPLC to give the product (20 mg, 15%) as a white solid. LC-MS: Calculated Exact Mass=579 Found [M+H]⁺=580.2; ¹H NMR (400 MHz, DMSO) δ 9.41 (s, 1H), 7.62 (dd, J=9.1, 3.9 Hz, 1H), 7.56 (s, 1H), 7.41 (d, J=7.5 Hz, 1H), 7.35 (dt, J=9.2, 4.6 Hz, 1H), 7.22 (d, J=8.2 Hz, 1H), 7.06 (s, 1H), 4.84 (d, J=11.4 Hz, 1H), 4.47 (s, 2H), 4.41-4.25 (m, 2H), 4.15 (s, 1H), 3.69 (s, 3H), 1.57 (s, 3H).

The following examples were prepared using a similar procedure as above:

| Example | Structure | Analytical Data |
|---|---|---|
| 115 | 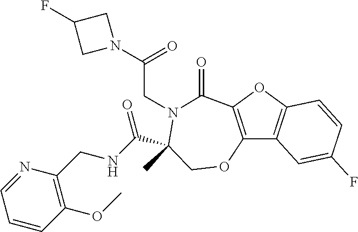 [SMILES: COc1cccnc1CNC(=O)[C@@]1(C)COc2c(oc3ccc(F)cc23)C(=O)N1CC(=O)N1CC(F)C1] | ¹H NMR (400 MHz, DMSO) δ 9.13 (s, 1H), 7.63 (dd, J = 9.1, 3.9 Hz, 1H), 7.60 (s, 1H), 7.43 (d, J = 7.9 Hz, 1H), 7.37 (td, J = 9.3, 2.7 Hz, 1H), 7.23 (d, J = 8.1 Hz, 1H), 7.08 (s, 1H), 5.62-5.32 (m, 1H), 4.84 (d, J = 11.7 Hz, 1H), 4.67-4.53 (m, 1H), 4.46- 4.03 (m, 7H), 4.02-3.85 (m, 1H), 3.70 (s, 3H), 1.59 (s, 3H). LC-MS: Found [M + H]⁺ = 515.2 |
| 116 | 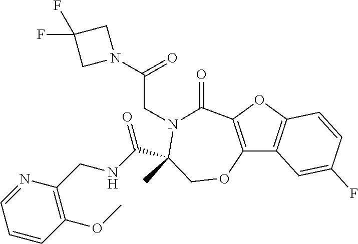 [SMILES: COc1cccnc1CNC(=O)[C@@]1(C)COc2c(oc3ccc(F)cc23)C(=O)N1CC(=O)N1CC(F)(F)C1] | ¹H NMR (400 MHz, DMSO) δ 8.98 (s, 1H), 7.64 (dd, J = 9.1, 3.9 Hz, 2H), 7.48-7.42 (m, 1H), 7.37 (td, J = 9.3, 2.7 Hz, 1H), 7.23 (d, J = 8.1 Hz, 1H), 7.09 (d, J = 4.7 Hz, 1H), 4.85 (d, J = 12.2 Hz, 1H), 4.77 (t, J = 12.2 Hz, 2H), 4.47-4.28 (m, 5H), 4.18 (s, 2H), 3.71 (s, 3H), 1.61 (s, 3H). LC-MS: Found [M + H]⁺ = 533.2 |
| 117 | 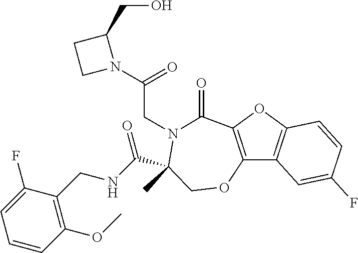 [SMILES: COc1cccc(F)c1CNC(=O)[C@@]1(C)COc2c(oc3ccc(F)cc23)C(=O)N1CC(=O)N1CC[C@H]1CO] | ¹H NMR (400 MHz, DMSO) δ 9.57 (s, 0.5H), 9.21 (s, 0.5H), 7.55 (td, J = 8.6, 3.9 Hz, 1H), 7.33 (tt, J = 6.8, 4.6 Hz, 2H), 7.01 (dd, J = 15.5, 7.1 Hz, 1H), 6.58 (dd, J = 19.2, 7.8 Hz, 1H), 6.37 (s, 1H), 5.25-4.72 (m, 2H), 4.66-4.06 (m, 7H), 3.97-3.70 (m, 3H), 3.62 (d, J = 7.8 Hz, 3H), 2.34-2.33 (m, 1H), 2.17-2.13 (m, 0.5H), 2.03-2.00 (m, 0.5H), 1.54 (s, 3H). LC-MS: Found [M + H]⁺ = 544.2 |
| 118 | 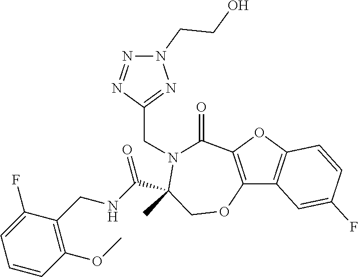 [SMILES: COc1cccc(F)c1CNC(=O)[C@@]1(C)COc2c(oc3ccc(F)cc23)C(=O)N1Cc1nnn(CCO)n1] | ¹H NMR (400 MHz, DMSO) δ 8.31 (s, 1H), 7.61 (dd, J = 9.0, 3.9 Hz, 1H), 7.37 (ddd, J = 11.8, 8.4, 3.7 Hz, 2H), 7.14 (d, J = 8.0 Hz, 1H), 6.70 (d, J = 8.2 Hz, 1H), 6.53 (s, 1H), 5.23 (d, J = 16.6 Hz, 1H), 5.03 (t, J = 5.5 Hz, 1H), 4.91 (d, J = 12.7 Hz, 1H), 4.68- 4.60 (m, 3H), 4.39 (d, J = 12.5 Hz, 1H), 4.27 (dd, J = 14.0, 4.9 Hz, 1H), 4.17-4.13 (m, 1H), 3.87 (dd, J = 10.6, 5.4 Hz, 2H), 3.70 (d, J = 14.7 Hz, 4H), 1.63 (s, 3H). LC-MS: Found [M + H]⁺ = 543.2 |
| 119 | 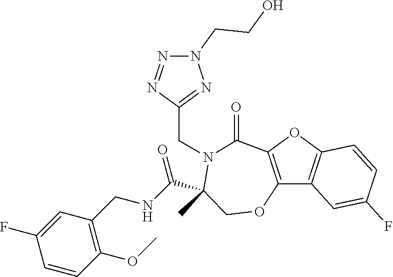 [SMILES: COc1ccc(F)cc1CNC(=O)[C@@]1(C)COc2c(oc3ccc(F)cc23)C(=O)N1Cc1nnn(CCO)n1] | ¹H NMR (400 MHz, DMSO) δ 8.64 (t, J = 5.9 Hz, 1H), 7.64 (dd, J = 9.1, 3.9 Hz, 1H), 7.43 (dd, J = 8.1, 2.6 Hz, 1H), 7.37 (td, J = 9.3, 2.7 Hz, 1H), 6.86 (d, J = 4.6 Hz, 2H), 6.54 (s, 1H), 5.38 (d, J = 17.1 Hz, 1H), 5.05 (dd, J = 11.0, 5.5 Hz, 2H), 4.80 (d, J = 16.7 Hz, 1H), 4.70-4.60 (m, 2H), 4.46 (d, J = 12.4 Hz, 1H), 4.17 (ddd, J = 36.6, 16.0, 5.6 Hz, 2H), 3.88 (dd, J = 10.7, 5.3 Hz, 2H), 3.71 (s, 3H), 1.77 (s, 3H). LC-MS: Found [M + H]⁺ = 543.2 |

### Example 120

### (R)-9-fluoro-4-((1-(2-hydroxyethyl)-1H-1,2,3-triazol-4-yl)methyl)-N-((3-methoxypyridin-2-yl)methyl)-3-methyl-5-oxo-2,3,4,5-tetrahydrobenzofuro[2,3-f][1,4]oxazepine-3-carboxamide

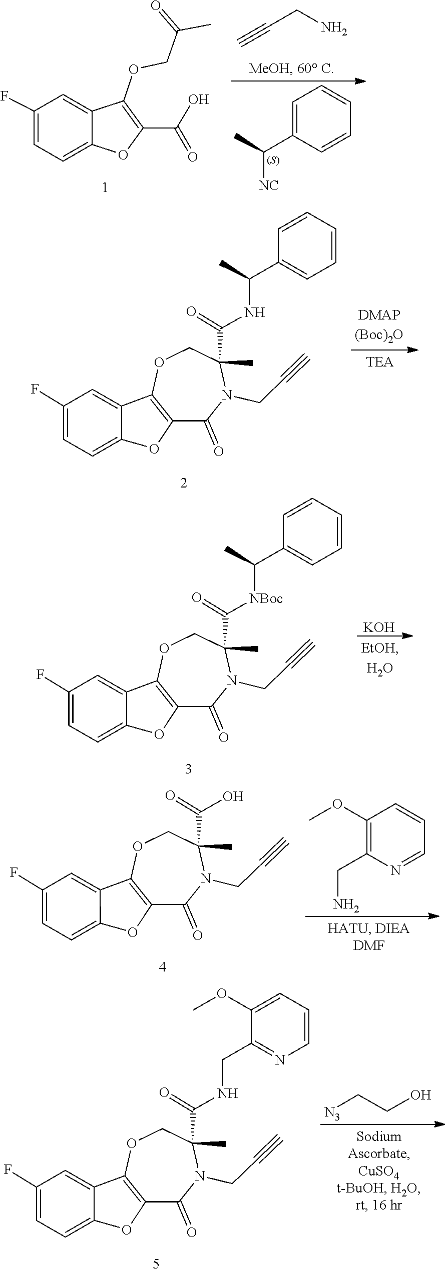

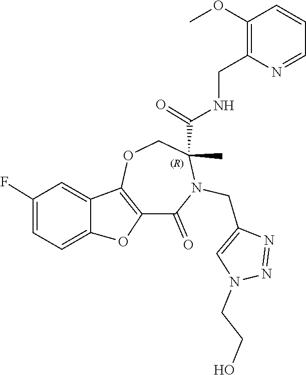

### (R)-9-fluoro-3-methyl-5-oxo-N—((S)-1-phenylethyl)-4-(prop-2-yn-1-yl)-2,3,4,5-tetrahydrobenzofuro[2,3-f][1,4]oxazepine-3-carboxamide

A mixture of 5-fluoro-3-(2-oxopropoxy)benzofuran-2-carboxylic acid (4 g, 15.86 mmol, 1.0 eq), prop-2-yn-1-amine (1.3 g, 23.79 mmol, 1.5 eq) and 1-(isocyanomethyl)-2-methoxybenzene (2.3 g, 17.45 mmol, 1.1 eq) in MeOH (20 mL) was stirred at 50° C. for 16 h. The mixture was concentrated under reduced pressure. The crude product was purified by flash chromatography on silica gel (PE:EA=1:1) to give the product (2.7 g, contained 30% isomer) as a yellow solid. TLC: PE/EA=1:1, UV; R_{f} Starting Material=0.0, Product=0.4. isomer=0.5; LC-MS: Calculated Exact Mass=420.1, Found [M+H]⁺=421.1.

### tert-butyl ((R)-9-fluoro-3-methyl-5-oxo-4-(prop-2-yn-1-yl)-2,3,4,5-tetrahydrobenzofuro[2,3-f][1,4]oxazepine-3-carbonyl)((S)-1-phenylethyl)carbamate

To a solution of (R)-9-fluoro-3-methyl-5-oxo-N—((S)-1-phenylethyl)-4-(prop-2-yn-1-yl)-2,3,4,5-tetrahydrobenzofuro[2,3-f][1,4]oxazepine-3-carboxamide (2.7 g, 6.43 mmol, 1.0 eq) in TEA (20 mL) was added (Boc)₂O (20 mL), DMAP (1.18 g, 9.64 mmol, 1.5 eq). The mixture was stirred at 100° C. for 3 hours. Water (50 mL) was added. The aqueous phase was extracted with EA (3×50 mL). The combined organic solution was washed with brine (50 mL), dried over anhydrous Na₂SO₄, and the solution was concentrated under reduced pressure. The residue was purified by silica gel chromatography (PE:EA=5:1) to give the product (1.05 g) as brown solid. TLC: PE/EA=5:1, UV; R_{f} Starting Material=0.15, Product=0.5; LC-MS: Calculated Exact Mass=520.1, Found [M+H]⁺=521.1.

### (R)-9-fluoro-3-methyl-5-oxo-4-(prop-2-yn-1-yl)-2,3,4,5-tetrahydrobenzofuro[2,3-f][1,4]oxazepine-3-carboxylic acid

To a solution of tert-butyl ((R)-9-fluoro-3-methyl-5-oxo-4-(prop-2-yn-1-yl)-2,3,4,5-tetrahydrobenzofuro[2,3-f][1,4]oxazepine-3-carbonyl)((S)-1-phenylethyl)carbamate (1.05 g, 2.0 mmol, 1.0 eq) in THE (10 mL) and MeOH (20 mL) was added KOH (10%, 20 mL). The mixture was stirred at 70° C. for 3 hours. The reaction mixture was concentrated under reduced pressure and then acidified to pH 4-5 with 1 N HCl. The solid was filtered to give the product (640 mg, 100%) as white solid. The crude product used in the next step directly. LC-MS: Calculated Exact Mass=317.1, Found [M+H]⁺=318.1.

### (R)-9-fluoro-N-((3-methoxypyridin-2-yl)methyl)-3-methyl-5-oxo-4-(prop-2-yn-1-yl)-2,3,4,5-tetrahydrobenzofuro[2,3-f][1,4]oxazepine-3-carboxamide

To a solution of (R)-9-fluoro-3-methyl-5-oxo-4-(prop-2-yn-1-yl)-2,3,4,5-tetrahydrobenzofuro[2,3-f][1,4]oxazepine-3-carboxylic acid (600 mg, 1.89 mmol, 1.0 eq) in DMF (15 mL) was added HATU (1.07 g, 2.83 mmol, 1.5 eq). The mixture was stirred at room temperature for 10 min. (3-methoxypyridin-2-yl)methanamine (522 mg, 3.78 mmol, 2.0 eq) and DIPEA (1.5 g, 11.34 mmol, 6.0 eq) were added. The reaction mixture was stirred at room temperature for 2 h. The reaction mixture was poured into H₂O (30 mL) and extracted with EtOAc (3×30 mL). The combined organic layers were washed with brine, dried over Na₂SO₄, filtered, and concentrated under reduced pressure. The crude product was purified by prep-HPLC to give the product (650 mg, 78.6%) as a white solid. LC-MS: Calculated Exact Mass=437.1, Found [M+H]⁺=438.1.

### (R)-9-fluoro-4-((1-(2-hydroxyethyl)-1H-1,2,3-triazol-4-yl)methyl)-N-((3-methoxypyridin-2-yl)methyl)-3-methyl-5-oxo-2,3,4,5-tetrahydrobenzofuro[2,3-f][1,4]oxazepine-3-carboxamide

To a solution of 4-(2-(dimethylamino)-2-oxoethyl)-3-((2-methoxybenzyl)carbamoyl)-3-methyl-5-oxo-2,3,4,5-tetrahydropyrido[2′,3′:4,5]furo[2,3-f][1,4]oxazepine-8-carboxylic acid (150 mg, 0.34 mmol, 1.0 eq) in t-BuOH (3 mL) and H₂O (3 mL) was added 2-azidoethan-1-ol (33 mg, 0.34 mmol, 1.0 eq), CuSO₄·5H₂O (21 mg, 0.085 mmol, 0.25 eq), TEA (34 mg, 0.34 mmol, 1.0 eq) and sodium ascorbate (34 mg, 0.17 mmol, 0.5 eq), The reaction mixture was stirred at room temperature for 16 h. The reaction mixture was poured into H₂O (30 mL) and extracted with EtOAc (3×30 mL). The combined organic layers were washed with brine, dried over Na₂SO₄, filtered, and concentrated under reduced pressure. The crude product was purified by prep-HPLC to give the product (22 mg, 12%) as a white solid. LC-MS: Calculated Exact Mass=524.2, Found [M+H]⁺=525.3. ¹H NMR (400 MHz, DMSO) δ 8.34 (s, 1H), 7.94 (s, 1H), 7.77 (s, 1H), 7.66 (dd, J=9.1, 3.9 Hz, 1H), 7.46 (dd, J=8.1, 2.5 Hz, 1H), 7.37 (td, J=9.3, 2.7 Hz, 1H), 7.30 (d, J=7.7 Hz, 1H), 7.17 (dd, J=8.2, 4.7 Hz, 1H), 5.05 (d, J=16.1 Hz, 1H), 4.99 (t, J=5.3 Hz, 1H), 4.87 (d, J=12.4 Hz, 1H), 4.56 (d, J=16.1 Hz, 1H), 4.41 (d, J=12.6 Hz, 1H), 4.37-4.14 (m, 4H), 3.75 (s, 3H), 3.75-3.71 (m, 2H), 1.65 (s, 3H).

### Intermediate BB-10.

### 6-bromo-3-(2-oxopropoxy)furo[3,2-b]pyridine-2-carboxylic acid

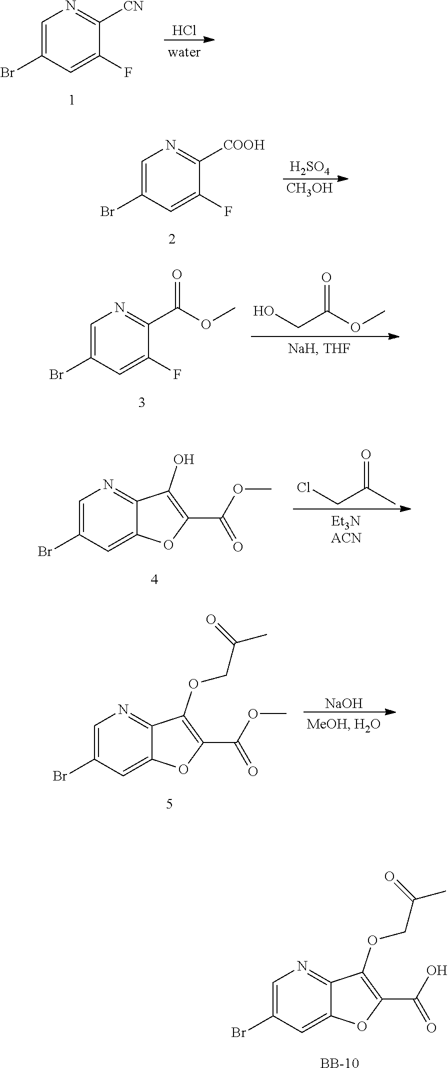

### 5-bromo-3-fluoropicolinic acid

5-bromo-3-fluoropicolinonitrile (45 g, 0.223 mol, 1.0 eq) in conc. HCl (36%) (500 mL) was stirred at 60° C. for 16 h. The reaction was monitored by LCMS. The resulting mixture was cooled, poured into ice water. The solid was collected by filtration, washed with water and recrystallized from methanol to give product (49 g, 99.4%) as yellow solid, TLC: DCM/MeOH=1:1, UV; R_{f} Starting Material=0.9, Product=0.15.

### methyl 5-bromo-3-fluoropicolinate

To a solution of 5-bromo-3-fluoropicolinic acid (49 g, 0.222 mol, 1.0 eq) in MeOH (0.7 L) was added H₂SO₄ (98%, 50 mL), the mixture was heated and stirred under reflux for 1 h. The reaction was monitored by LCMS. The crude product was distilled under reduced pressure, washed with H₂O, extracted with ethyl acetate, the organic phase was distilled under reduced pressure to give the product (48.3 g, 92.7%) as brown solid, TLC: PE/EA=1:1, UV; R_{f} Starting Material=0.0, Product=0.5.

### methyl 5-bromo-3-fluoropicolinate

To a solution of methyl 5-bromo-3-fluoropicolinate (48.3 g, 0.208 mol, 1.0 eq) in THE (1.5 L) was added NaH (25 g, 0.619 mol, 3.0 eq) at 0° C., the mixture was stirred for 30 min, then methyl 2-hydroxyacetate (28 g, 0.309 mol, 1.5 eq), The reaction was monitored by LCMS. The resulting mixture was cooled, poured into ice water and acidified with 5% hydrochloric acid to pH 2. The solid was collected by filtration, washed with water and recrystallized from methanol to give product (49 g, 87.2%) as brown solid, TLC: PE/EA=1:1, UV; R_{f} Starting Material=0.5, Product=0.1. LC-MS: Calculated Exact Mass=270.9, Found [M+H]⁺=271.9.

### methyl 6-bromo-3-(2-oxopropoxy)furo[3,2-b]pyridine-2-carboxylate

A mixture of compound methyl 6-bromo-3-hydroxyfuro[3,2-b]pyridine-2-carboxylate (49 g, 0.18 mol, 1.0 eq) in acetonitrile (1 L) was added TEA (54 g, 0.54 mol, 3.0 eq), chloroacetone (33.3 g, 0.36 mmol, 2.0 eq), the mixture was refluxed for 4 hours. The reaction was monitored by LCMS. The resulting mixture was filtered, concentrated, extracted with ethyl acetate to give the product (42 g, 87%) as brown solid. TLC: PE/EA=2:1, UV; R_{f} Starting Material=0.2, Product=0.5. LC-MS: Calculated Exact Mass=326.9, Found [M+H]⁺=327.9.

### 6-bromo-3-(2-oxopropoxy)furo[3,2-b]pyridine-2-carboxylic acid (BB-3, V2616-041)

To a solution of methyl 6-bromo-3-(2-oxopropoxy)furo[3,2-b]pyridine-2-carboxylate (42 g, 0.128 mol, 1.0 eq) in MeOH (300 L) and 10% NaOH in water (100 mL) was stirred at rt for 4 hours. The reaction was monitored by LCMS. The resulting mixture was concentrated, added 1 N HCl to adjust to pH 5, extracted with ethyl acetate. The combined organic layers were washed with brine, dried over Na₂SO₄. The solvent was removed under reduced pressure and the residue was purified by flash column chromatography on silica (DCM:MeOH=10:1) to give the product (28 g, 69.7%) as yellow solid. TLC: DCM:MeOH=10:1, UV; R_{f} Starting Material=0.9, Product=0.1. LC-MS: Calculated Exact Mass=312.9, Found [M+H]⁺=313.9.

### Example 121

### 4-(2-(dimethylamino)-2-oxoethyl)-N₃-(2-methoxybenzyl)-3-methyl-N₈-(3-methyloxetan-3-yl)-5-oxo-2,3,4,5-tetrahydropyrido[2′,3′:4,5]furo[2,3-f][1,4]oxazepine-3,8-dicarboxamide

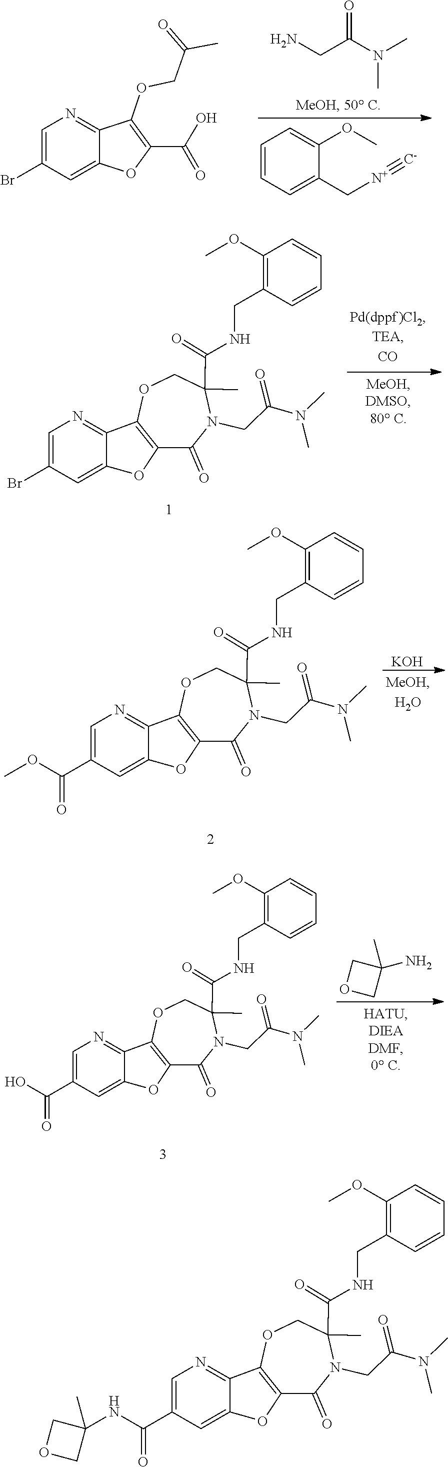

### 8-bromo-4-(2-(dimethylamino)-2-oxoethyl)-N-(2-methoxybenzyl)-3-methyl-5-oxo-2,3,4,5-tetrahydropyrido[2′,3′:4,5]furo[2,3-f][1,4]oxazepine-3-carboxamide

A mixture of 6-bromo-3-(2-oxopropoxy)furo[3,2-b]pyridine-2-carboxylic acid (3 g, 9.55 mmol, 1.0 eq), 2-amino-N,N-dimethylacetamide (1.4 g, 14.33 mmol, 1.5 eq), and 1-(isocyanomethyl)-2-methoxybenzene (1.3 g, 12.42 mmol, 1.3 eq) in MeOH (20 mL) was stirred at 50° C. for 16 h. The mixture was concentrated under reduced pressure. The crude product was purified by flash chromatography on silica gel (DCM:MeOH=20:1) to give the product (2.4 g, 46%) as a yellow solid. TLC: DCM/MeOH=20:1, UV; R_{f} Starting Material=0.0, Product=0.4.

### methyl 4-(2-(dimethylamino)-2-oxoethyl)-3-((2-methoxybenzyl)carbamoyl)-3-methyl-5-oxo-2,3,4,5-tetrahydropyrido[2′,3′:4,5]furo[2,3-f][1,4]oxazepine-8-carboxylate

A mixture of 8-bromo-4-(2-(dimethylamino)-2-oxoethyl)-N-(2-methoxybenzyl)-3-methyl-5-oxo-2,3,4,5-tetrahydropyrido[2′,3′:4,5]furo[2,3-f][1,4]oxazepine-3-carboxamide (500 mg, 0.92 mmol, 1.0 eq), TEA (464 mg, 4.58 mmol, 5 eq), and Pd(dppf)Cl2 (67 mg, 0.092 mmol, 0.1 eq) in MeOH (20 mL) and DMSO (5 mL) was stirred at 80° C. for 16 h under CO. The mixture was concentrated under reduced pressure. The crude product was purified by flash chromatography on silica gel (PE:EA=1:1) to give the product (330 mg, 60%) as a yellow solid. TLC: DCM/MeOH=20:1, UV; R_{f} Starting Material=0.4, Product=0.5.

### 4-(2-(dimethylamino)-2-oxoethyl)-3-((2-methoxybenzyl)carbamoyl)-3-methyl-5-oxo-2,3,4,5-tetrahydropyrido[2′,3′:4,5]furo[2,3-f][1,4]oxazepine-8-carboxylic acid

To a solution of methyl 4-(2-(dimethylamino)-2-oxoethyl)-3-((2-methoxybenzyl)carbamoyl)-3-methyl-5-oxo-2,3,4,5-tetrahydropyrido[2′,3′:4,5]furo[2,3-f][1,4]oxazepine-8-carboxylate (330 mg, 0.629 mmol, 1.0 eq) in THE (10 mL) and MeOH (20 mL) was added KOH (10%, 20 mL). The mixture was stirred at 25° C. for 3 hours. The reaction mixture was concentrated under reduced pressure and then acidified to pH 4-5 with 1 N HCl. The solid was filtered to give the product (310 mg, 96.5%) as white solid. The crude product used in the next step directly.

### 4-(2-(dimethylamino)-2-oxoethyl)-N₃-(2-methoxybenzyl)-3-methyl-N₈-(3-methyloxetan-3-yl)-5-oxo-2,3,4,5-tetrahydropyrido[2′,3′:4,5]furo[2,3-f][1,4]oxazepine-3,8-dicarboxamide

To a solution of (R)-9-fluoro-N-((3-methoxypyridin-2-yl)methyl)-3-methyl-5-oxo-4-(prop-2-yn-1-yl)-2,3,4,5-tetrahydrobenzofuro[2,3-f][1,4]oxazepine-3-carboxamide (380 mg, 0.744 mmol, 1.0 eq) in DMF (2 mL) was added HATU (425 mg, 1.17 mmol, 1.5 eq), 3-methyloxetan-3-amine (92 mg, 1.17 mmol, 1.5 eq) and DIPEA (141 mg, 1.17 mmol, 1.5 eq). The reaction mixture was stirred at room temperature for 2 h. The reaction mixture was poured into H₂O (30 mL) and extracted with EtOAc (3×30 mL). The combined organic layers were washed with brine, dried over Na₂SO₄, filtered, and concentrated under reduced pressure. The crude product was purified by prep-HPLC to give the product (150 mg, 42%) as a white solid. LC-MS: Calculated Exact Mass=490.2, Found [M+H]⁺=491.3. ¹H NMR (400 MHz, DMSO) δ 9.71 (s, 1H), 9.20 (s, 1H), 9.04 (d, J=1.7 Hz, 1H), 8.43 (d, J=1.7 Hz, 1H), 6.97 (t, J=7.6 Hz, 1H), 6.81 (d, J=8.1 Hz, 1H), 6.47 (s, 1H), 6.19 (s, 1H), 4.99 (d, J=11.9 Hz, 1H), 4.78 (d, J=6.3 Hz, 2H), 4.54-4.42 (m, 5H), 4.22 (dd, J=15.7, 6.6 Hz, 1H), 3.95 (dd, J=15.4, 4.8 Hz, 1H), 3.69 (s, 3H), 3.07 (s, 3H), 2.89 (s, 3H), 1.67 (s, 3H), 1.64 (s, 2H).

The following examples were prepared using a similar procedure as above:

| Example | Structure | Analytical Data |
|---|---|---|
| 122 | 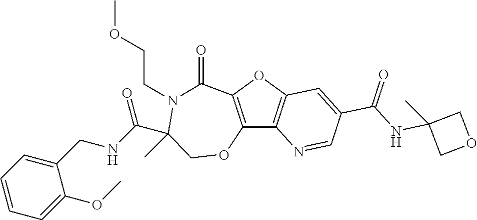 [SMILES: COCCN1C(=O)c2oc3cc(C(=O)NC4(C)COC4)cnc3c2OCC1(C)C(=O)NCc1ccccc1OC] | ¹H NMR (400 MHz, CDCl3) δ 8.86 (d, J = 1.6 Hz, 1H), 8.08 (d, J = 1.6 Hz, 1H), 7.40 (d, J = 5.3 Hz, 1H), 7.26 (s, 1H), 7.04 (s, 1H), 6.94 (dd, J = 17.9, 7.9 Hz, 2H), 6.66 (d, J = 8.1 Hz, 1H), 6.50 (s, 1H), 4.93 (d, J = 6.5 Hz, 2H), 4.90 (s, 1H), 4.61 (d, J = 6.6 Hz, 2H), 4.33 (dd, J = 14.2, 6.7 Hz, 1H), 4.27-4.07 (m, 3H), 3.84-3.81 (m, 1H), 3.79 (s, 3H), 3.69-3.52 (m, 2H), 1.82 (s, 3H), 1.77 (s, 3H). LC-MS: Found [M + H]⁺ = 553 |
| 123 | 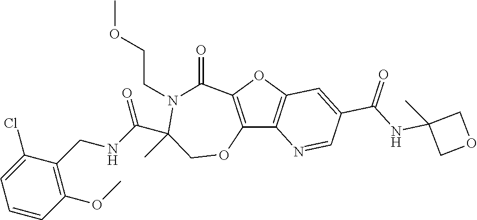 [SMILES: COCCN1C(=O)c2oc3cc(C(=O)NC4(C)COC4)cnc3c2OCC1(C)C(=O)NCc1c(Cl)cccc1OC] | ¹H NMR (400 MHz, DMSO) δ 9.29 (s, 1H), 9.03 (d, J = 1.6 Hz, 1H), 8.45 (d, J = 1.6 Hz, 1H), 7.94 (s, 1H), 7.19 (t, J = 8.1 Hz, 1H), 6.97-6.83 (m, 2H), 4.90 (d, J = 11.1 Hz, 1H), 4.78 (d, J = 6.3 Hz, 2H), 4.42 (d, J = 6.4 Hz, 2H), 4.36 (dd, J = 13.4, 4.8 Hz, 2H), 4.26 (d, J = 12.3 Hz, 1H), 3.75 (s, 3H), 3.66 (s, 2H), 3.51-3.48 (m, 2H), 3.20 (s, 3H), 1.66 (s, 3H), 1.64 (s, 3H). LC-MS: 587.2 [M + H]⁺ |

### Example 124

### 4-(2-(dimethylamino)-2-oxoethyl)-N-(2-fluoro-6-methoxybenzyl)-3-methyl-8-(oxazol-2-yl)-5-oxo-2,3,4,5-tetrahydropyrido[2′,3′:4,5]furo[2,3-f][1,4]oxazepine-3-carboxamide

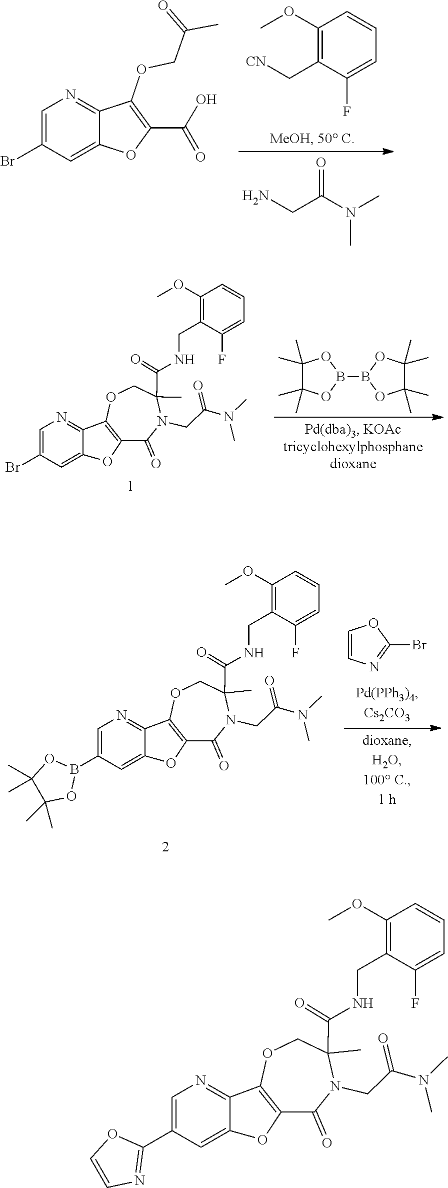

### 8-bromo-4-(2-(dimethylamino)-2-oxoethyl)-N-(2-methoxybenzyl)-3-methyl-5-oxo-2,3,4,5-tetrahydropyrido[2′,3′:4,5]furo[2,3-f][1,4]oxazepine-3-carboxamide

A mixture of 6-bromo-3-(2-oxopropoxy)furo[3,2-b]pyridine-2-carboxylic acid (500 mg, 1.59 mmol, 1.0 eq), 2-amino-N,N-dimethylacetamide (146 m g, 1.43 mmol, 0.9 eq), and 1-fluoro-2-(isocyanomethyl)-3-methoxybenzene (289 mg, 1.75 mmol, 1.1 eq) in MeOH (20 mL) was stirred at 50° C. for 16 h. The mixture was concentrated under reduced pressure. The crude product was purified by flash chromatography on silica gel (DCM:MeOH=20:1) to give the product (320 mg, 35%) as a yellow solid. TLC: DCM/MeOH=20:1, UV; R_{f} Starting Material=0.0, Product=0.4.

### 4-(2-(dimethylamino)-2-oxoethyl)-N-(2-fluoro-6-methoxybenzyl)-3-methyl-5-oxo-8-(4,4,5,5-tetramethyl-1,3,2-dioxaborolan-2-yl)-2,3,4,5-tetrahydropyrido[2′,3′:4,5]furo[2,3-f][1,4]oxazepine-3-carboxamide

A mixture of 8-bromo-4-(2-(dimethylamino)-2-oxoethyl)-N-(2-fluoro-6-methoxybenzyl)-3-methyl-5-oxo-2,3,4,5-tetrahydropyrido[2′,3′:4,5]furo[2,3-f][1,4]oxazepine-3-carboxamide (300 mg, 0.53 mmol, 1.0 eq), 4,4,4′,4′,5,5,5′,5′-octamethyl-2,2′-bi(1,3,2-dioxaborolane) (2700 mg, 10.6 mmol, 20 eq), KOAc (156 mg, 1.6 mmol, 3.0 eq), Pd₂(dba)₃ (48 mg, 0.053 mmol, 0.1 eq) and tricyclohexylphosphane (29 mg, 0.106 mmol, 0.2 eq) in dioxane (20 mL) was stirred at 50° C. for 16 h. The mixture was concentrated under reduced pressure. The crude product was purified by flash chromatography on silica gel (PE:EA=1:1) to give the product (200 mg, 61%) as a yellow solid.

### 4-(2-(dimethylamino)-2-oxoethyl)-N-(2-fluoro-6-methoxybenzyl)-3-methyl-8-(oxazol-2-yl)-5-oxo-2,3,4,5-tetrahydropyrido[2′,3′:4,5]furo[2,3-f][1,4]oxazepine-3-carboxamide

A mixture of Pd(PPh₃)₄ (34 mg, 0.029 mmol, 0.1 eq), Cs₂CO₃ (288 mg, 0.88 mmol, 3.0 eq), intermediate 4-(2-(dimethylamino)-2-oxoethyl)-N-(2-fluoro-6-methoxybenzyl)-3-methyl-5-oxo-8-(4,4,5,5-tetramethyl-1,3,2-dioxaborolan-2-yl)-2,3,4,5-tetrahydropyrido[2′,3′:4,5]furo[2,3-f][1,4]oxazepine-3-carboxamide (180 mg, 0.294 mmol, 1.0 eq) and 2-bromooxazole (87 mg, 0.589 mmol, 2.0 eq) in dioxane (3 ml) and H₂O (0.3 ml) was degassed with argon for 2 mins, the resulting mixture stirred at 100° C. under argon for 3 hours. The reaction mixture was allowed to cool and diluted with ethyl acetate. This solution was washed with water. The organic layer was separated, dried over sodium sulfate and evaporated under reduced pressure, the residue was purified by flash column chromatography on silica gel (DCM:MeOH=20:1) to give the product (28 mg, 17%) as white solid. LC-MS: Calculated Exact Mass=551.2, Found [M+H]⁺=552.2.

¹H NMR (400 MHz, DMSO) δ 9.62 (s, 1H), 9.12 (d, J=1.6 Hz, 1H), 8.46 (d, J=1.5 Hz, 1H), 8.39 (d, J=0.7 Hz, 1H), 7.52 (d, J=0.7 Hz, 1H), 6.93-6.91 (m, 1H), 6.64 (d, J=8.1 Hz, 1H), 6.33-6.31 (m, 1H), 4.90 (d, J=11.9 Hz, 1H), 4.55-4.51 (m, 1H), 4.43-4.26 (m, 3H), 3.89-3.84 (m, 1H), 3.66 (s, 3H), 3.05 (s, 3H), 2.89 (s, 3H), 1.57 (s, 3H).

### Intermediate BB-11

### 6-bromo-3-(2-oxopropoxy)benzofuran-2-carboxylic acid

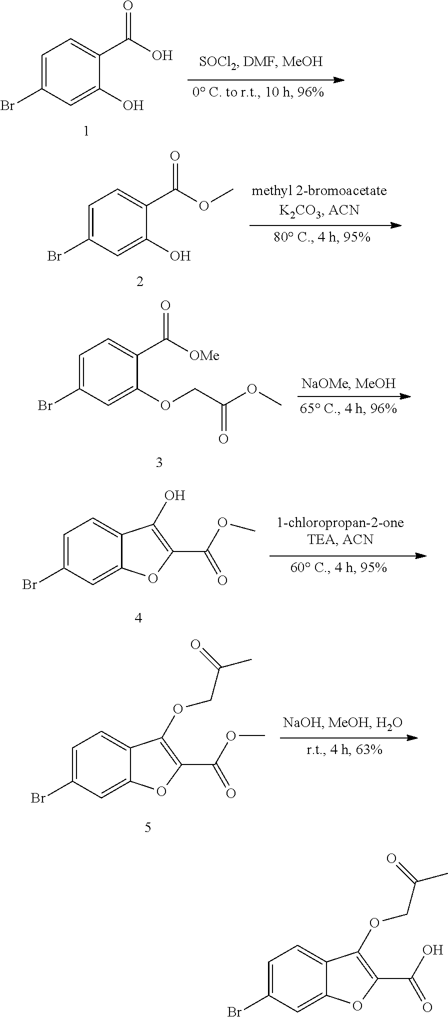

### methyl 4-bromo-2-hydroxybenzoate

A mixture of compound 4-bromo-2-hydroxybenzoic acid (200 g, 0.92 mol, 1.0 eq), SOCl₂ (550 g, 4.60 mol, 5.0 eq), and DMF (15 mL) in MeOH (500 mL) was heated at 65° C. and stirred for 10 h. The reaction mixture was cooled to room temperature and concentrated under reduced pressure. The residue was washed with sat. NaOH, extracted with EA, the combined organic layers were dried over anhydrous Na₂SO₄, filtered, and concentrated under reduced pressure to give the crude product (205 g, 96%) as a light yellow solid. TLC: PE/EA=3:1, UV; R_{f} Starting Material=0.0, Product=0.8. LC-MS: Calculated Exact Mass=230.0, Found [M+H]⁺=231.0. ¹H NMR (400 MHz, CDCl₃) δ (ppm): 10.82 (s, 1H), 7.67 (d, J=8.5 Hz, 1H), 7.17 (d, J=1.9 Hz, 1H), 7.01 (dd, J=8.5, 1.9 Hz, 1H), 3.94 (s, 3H).

### methyl 4-bromo-2-(2-methoxy-2-oxoethoxy)benzoate

Methyl bromoacetate (164 g, 1.07 mol, 1.2 eq) was added dropwise over 30 min to a stirred mixture of compound methyl 4-bromo-2-hydroxybenzoate (205 g, 0.89 mol, 1.0 eq) and potassium carbonate (192 g, 1.33 mol, 1.5 eq) in MeCN (2 L). The mixture was heated and stirred under reflux for 4 h. The reaction was monitored by LCMS. The reaction was cooled, and the salts were separated by filtration and washed with acetone. The filtrate was concentrated to give a residue, which was diluted with EA (2 L), and the solution was washed successively with water, aqueous sodium carbonate, water, and brine. The crude product was distilled under reduced pressure to give the product (255 g, 95%) as a brown solid. TLC: PE/EA=3:1, UV; R_{f} Starting Material=0.8, Product=0.5. LC-MS: Calculated Exact Mass=302.0, Found [M+H]⁺=303.0. ¹H NMR (400 MHz, DMSO-d₆) δ (ppm): 7.61 (d, J=8.3 Hz, 1H), 7.34 (d, J=1.7 Hz, 1H), 7.27 (dd, J=8.3, 1.7 Hz, 1H), 4.97 (s, 2H), 3.80 (s, 3H), 3.71 (s, 3H).

### methyl 6-bromo-3-hydroxybenzofuran-2-carboxylate

A mixture of compound methyl 4-bromo-2-(2-methoxy-2-oxoethoxy)benzoate (255 g, 0.84 mol, 1.0 eq) in MeOH (1 L) was added NaOMe (30% in methanol, 182 mL, 1.01 mol, 1.2 eq) and the mixture was stirred at 60° C. for 4 hours. The reaction was monitored by LCMS. The resulting mixture was cooled, poured into ice water and acidified with 5% hydrochloric acid to pH 2. The solid was collected by filtration, washed with water and recrystallized from methanol to give the product (220 g, 96%) as yellow solid. TLC: PE/EA=3:1, UV; R_{f} Starting Material=0.5, Product=0.4. LC-MS: Calculated Exact Mass=270.0, Found [M+H]⁺=271.1. ¹H NMR (400 MHz, DMSO-d₆) δ (ppm): 11.04 (s, 1H), 7.92 (d, J=1.5 Hz, 1H), 7.85 (d, J=8.5 Hz, 1H), 7.49 (dd, J=8.5, 1.6 Hz, 1H), 3.84 (s, 3H).

### methyl 6-bromo-3-(2-oxopropoxy)benzofuran-2-carboxylate

A mixture of compound methyl 6-bromo-3-hydroxybenzofuran-2-carboxylate (200 g, 0.74 mol, 1.0 eq) in acetonitrile (1 L) was added TEA (89 g, 0.89 mol, 1.2 eq), chloroacetone (75 g, 0.81 mmol, 1.1 eq), the mixture was refluxed for 4 hours. The reaction was monitored by LCMS. The resulting mixture was filtered, concentrated, extracted with ethyl acetate to give the product (23 g, 95%) as brown solid. TLC: PE/EA=3:1, UV; R_{f} Starting Material=0.4, Product=0.6. LC-MS: Calculated Exact Mass=326.0, Found [M+H]⁺=327.1.

### 6-bromo-3-(2-oxopropoxy)benzofuran-2-carboxylic acid

To a solution of crude compound methyl 6-bromo-3-(2-oxopropoxy)benzofuran-2-carboxylate (100 g, 0.31 mol, 1.0 eq) in MeOH (1 L) and 20% NaOH in water (400 mL) was stirred at rt for 4 hours. The reaction was monitored by LCMS. The resulting mixture was concentrated, added 1 N HCl to adjust to pH 2, extracted with ethyl acetate. The combined organic layers were washed with brine, dried over Na₂SO₄. The solvent was removed under reduced pressure and the residue was purified by flash column chromatography on silica (DCM:MeOH=10:1) to give the product (60 g, 63%) as yellow solid. TLC: DCM:MeOH=10:1, UV; R_{f} Starting Material=0.9, Product=0.2. LC-MS: Calculated Exact Mass=312.0, Found [M+H]⁺=313.0. ¹H NMR (400 MHz, DMSO-d₆) δ (ppm): 13.49 (s, 1H), 7.98 (d, J=1.4 Hz, 1H), 7.77 (d, J=8.5 Hz, 1H), 7.52 (dd, J=8.5, 1.5 Hz, 1H), 5.20 (s, 2H), 2.16 (s, 3H).

### Example 125

### (R)-4-(2-(dimethylamino)-2-oxoethyl)-N-((3-methoxypyridin-2-yl)methyl)-3-methyl-8-(oxazol-2-yl)-5-oxo-2,3,4,5-tetrahydrobenzofuro[2,3-f][1,4]oxazepine-3-carboxamide

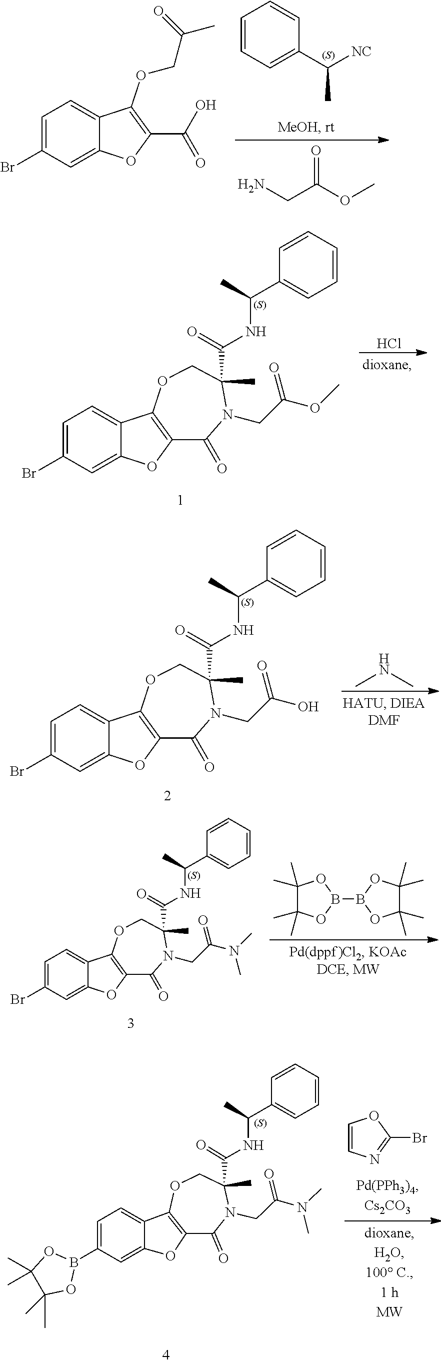

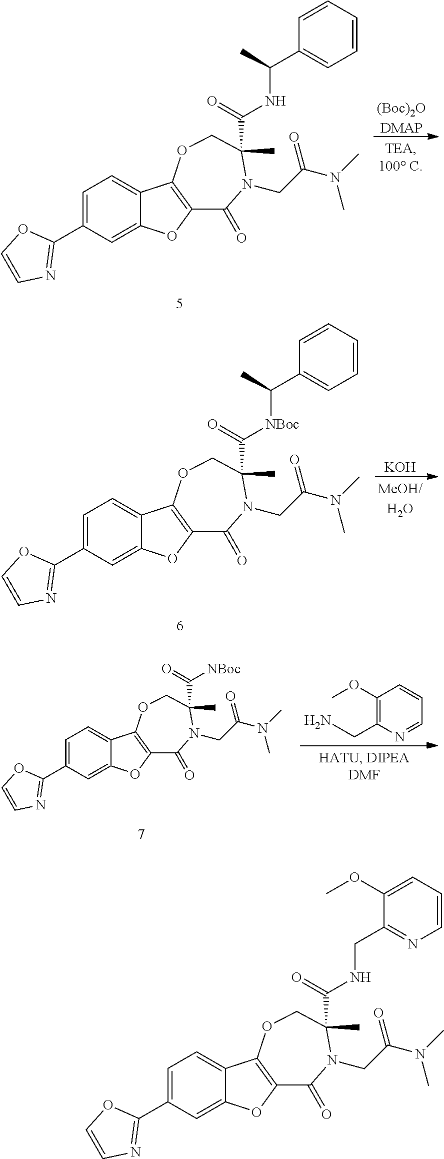

### methyl 2-((R)-8-bromo-3-methyl-5-oxo-3-(((S)-1-phenylethyl)carbamoyl)-2,3-dihydrobenzofuro[2,3-f][1,4]oxazepin-4(5H)-yl)acetate

A mixture of 6-bromo-3-(2-oxopropoxy)benzofuran-2-carboxylic acid (55 g, 0.176 mol, 1.0 eq), methyl glycinate (24 g, 0.263 mol, 1.5 eq) and (S)-(1-isocyanoethyl)benzene (24 g, 0.263 mol, 1.5 eq) in MeOH (20 mL) was stirred at 50° C. for 16 h. The mixture was concentrated under reduced pressure. The crude product was purified by flash chromatography on silica gel (PE:EA=1:1) to give the product (40 g, 43.9%) as a yellow solid. TLC: PE/EA=1:1, UV; R_{f} Starting Material=0.0, Product=0.4, isomer=0.6.

### 2-((R)-8-bromo-3-methyl-5-oxo-3-(((S)-1-phenylethyl)carbamoyl)-2,3-dihydrobenzofuro[2,3-f][1,4]oxazepin-4(5H)-yl)acetic acid

A mixture of methyl 2-((R)-8-bromo-3-methyl-5-oxo-3-(((S)-1-phenylethyl)carbamoyl)-2,3-dihydrobenzofuro[2,3-f][1,4]oxazepin-4(5H)-yl)acetate (40 g, 77.62 mmol, 1.0 eq), 6M HCl (600 mL) in dioxane (800 mL) was stirred at 80° C. for 16 h. The mixture was concentrated under reduced pressure. The solution was extracted with EA, the solvent was removed under reduced pressure to give the product (35 g, 92%) as a yellow solid.

### (R)-8-bromo-4-(2-(dimethylamino)-2-oxoethyl)-3-methyl-5-oxo-N—((S)-1-phenylethyl)-2,3,4,5-tetrahydrobenzofuro[2,3-f][1,4]oxazepine-3-carboxamide

To a solution of 2-((R)-8-bromo-3-methyl-5-oxo-3-(((S)-1-phenylethyl)carbamoyl)-2,3-dihydrobenzofuro[2,3-f][1,4]oxazepin-4(5H)-yl)acetic acid (27 g, 53.86 mmol, 1.0 eq) in DMF (30 mL) was added HATU (41 g, 107.7 mmol, 2.0 eq), dimethylamine (50 mL, 2M, 107.7 mmol, 2.0 eq) and DIPEA (45 mL, 269.3 mmol, 5.0 eq) were added. The reaction mixture was stirred at room temperature for 2 h. The reaction mixture was poured into H₂O (30 mL) and extracted with EtOAc (3×30 mL). The combined organic layers were washed with brine, dried over Na₂SO₄, filtered, and concentrated under reduced pressure. The crude product was purified by flash chromatography on silica gel (DCM:MeOH=5:1) to give the product (25 g, 89%) as a white solid.

### (R)-4-(2-(dimethylamino)-2-oxoethyl)-3-methyl-5-oxo-N—((S)-1-phenylethyl)-8-(4,4,5,5-tetramethyl-1,3,2-dioxaborolan-2-yl)-2,3,4,5-tetrahydrobenzofuro[2,3-f][1,4]oxazepine-3-carboxamide

A mixture of (1,1′-bis(diphenylphosphino)ferrocene)palladium(11) chloride (3.5 g, 4.73 mmol, 0.1 eq), potassium acetate (23 g, 236.6 mmol, 5.0 eq), (R)-8-bromo-4-(2-(dimethylamino)-2-oxoethyl)-3-methyl-5-oxo-N—((S)-1-phenylethyl)-2,3,4,5-tetrahydrobenzofuro[2,3-f][1,4]oxazepine-3-carboxamide (25 g, 47.31 mmol, 1.0 eq) and bis(pinacolato)diboron (60 g, 236.6 mmol, 5.0 eq) in dioxane (600 ml) was degassed with argon for 5 min, the resulting mixture stirred at 100° C. under argon for 3 hours. The reaction mixture was allowed to cool and diluted with ethyl acetate. This solution was washed with water. The organic layer was separated, dried over sodium sulfate and evaporated under reduced pressure, the residue was purified by flash column chromatography on silica gel (PE:EA=3:1) to give the product (12 g, 45%) as light-yellow solid.

### (R)-4-(2-(dimethylamino)-2-oxoethyl)-3-methyl-8-(oxazol-2-yl)-5-oxo-N—((S)-1-phenylethyl)-2,3,4,5-tetrahydrobenzofuro[2,3-f][1,4]oxazepine-3-carboxamide

A mixture of Pd(PPh₃)₄ (1.2 g, 1.04 mmol, 0.1 eq), Cs₂CO₃ (6.8 g, 20.86 mmol, 2.0 eq), intermediate (R)-4-(2-(dimethylamino)-2-oxoethyl)-3-methyl-5-oxo-N—((S)-1-phenylethyl)-8-(4,4,5,5-tetramethyl-1,3,2-dioxaborolan-2-yl)-2,3,4,5-tetrahydrobenzofuro[2,3-f][1,4]oxazepine-3-carboxamide (6 g, 10.43 mmol, 2.0 eq) and 2-bromooxazole (3 g, 20.86 mmol, 2.0 eq) in dioxane (100 ml) and H₂O (10 ml) was degassed with argon for 2 mins, the resulting mixture stirred at 100° C. under argon for 16 hours. The reaction mixture was allowed to cool and diluted with ethyl acetate. This solution was washed with water. The organic layer was separated, dried over sodium sulfate and evaporated under reduced pressure, the residue was purified by flash column chromatography on silica gel (DCM:MeOH=20:1) to give the product (3.8 g, 72%) as light-yellow solid.

### tert-butyl ((R)-4-(2-(dimethylamino)-2-oxoethyl)-3-methyl-8-(oxazol-2-yl)-5-oxo-2,3,4,5-tetrahydrobenzofuro[2,3-f][1,4]oxazepine-3-carbonyl)((S)-1-phenylethyl)carbamate

To a solution of ((R)-4-(2-(dimethylamino)-2-oxoethyl)-3-methyl-8-(oxazol-2-yl)-5-oxo-N—((S)-1-phenylethyl)-2,3,4,5-tetrahydrobenzofuro[2,3-f][1,4]oxazepine-3-carboxamide (4 g, 7.74 mmol, 1.0 eq) in TEA (100 mL) was added (Boc)₂O (17 g, 77.44 mmol, 10.0 eq), DMAP (0.95 g, 7.74 mmol, 1.0 eq). The mixture was stirred at 100° C. for 16 hours. Water (150 mL) was added. The aqueous phase was extracted with EA (3×50 mL). The combined organic solution was washed with brine (50 mL), dried over anhydrous Na₂SO₄, and the solution was concentrated under reduced pressure. The residue was purified by silica gel chromatography (PE:EA=1:2) to give the product (4 g, 75%) as brown solid.

### (R)-4-(2-(dimethylamino)-2-oxoethyl)-3-methyl-8-(oxazol-2-yl)-5-oxo-2,3,4,5-tetrahydrobenzofuro[2,3-f][1,4]oxazepine-3-carboxylic acid

To a solution of tert-butyl ((R)-4-(2-(dimethylamino)-2-oxoethyl)-3-methyl-8-(oxazol-2-yl)-5-oxo-2,3,4,5-tetrahydrobenzofuro[2,3-f][1,4]oxazepine-3-carbonyl)((S)-1-phenylethyl)carbamate (3.5 g, 5.676 mmol, 1.0 eq) in THF (25 mL), MeOH (50 mL was added KOH (10%, 50 mL), the mixture was stirred at 70° C. for 3 hours. The reaction mixture was concentrated under reduced pressure and then acidified to pH 4-5 with 1 N HCl. The solid was filtered to give the product (2.3 g, 82%) as white solid. The crude product used in the next step directly.

### (R)-4-(2-(dimethylamino)-2-oxoethyl)-N-((3-methoxypyridin-2-yl)methyl)-3-methyl-8-(oxazol-2-yl)-5-oxo-2,3,4,5-tetrahydrobenzofuro[2,3-f][1,4]oxazepine-3-carboxamide

To a solution of (R)-4-(2-(dimethylamino)-2-oxoethyl)-3-methyl-8-(oxazol-2-yl)-5-oxo-2,3,4,5-tetrahydrobenzofuro[2,3-f][1,4]oxazepine-3-carboxylic acid (2.3 g, 5.564 mmol, 1.0 eq) in DMF (20 mL) was added HATU (4.2 g, 11.14 mmol, 2.0 eq), (3-methoxypyridin-2-yl)methanamine (1.5 g, 11.13 mmol, 2.0 eq) and DIPEA (3.6 g, 27.82 mmol, 5.0 eq). The reaction mixture was stirred at room temperature for 2 h. The reaction mixture was poured into H₂O (130 mL) and extracted with EtOAc (3×130 mL). The combined organic layers were washed with brine, dried over Na₂SO₄, filtered, and concentrated under reduced pressure. The crude product was purified by prep-HPLC to give the product (2 g, 70%) as a white solid. LC-MS: Calculated Exact Mass=533.2, Found [M+H]⁺=534.2. ¹H NMR (400 MHz, DMSO) δ 9.45 (s, 1H), 8.30 (d, J=0.6 Hz, 1H), 8.09 (s, 1H), 7.92 (dd, J=8.3, 1.0 Hz, 1H), 7.76 (d, J=8.2 Hz, 1H), 7.55 (s, 1H), 7.46 (d, J=0.7 Hz, 1H), 7.22 (d, J=8.1 Hz, 1H), 7.02 (s, 1H), 4.88 (d, J=11.9 Hz, 1H), 4.50 (s, 2H), 4.38 (d, J=11.4 Hz, 1H), 4.31 (dd, J=15.6, 6.2 Hz, 1H), 4.14 (s, 1H), 3.69 (s, 3H), 3.06 (s, 3H), 2.86 (s, 3H), 1.59 (s, 3H).

The following examples were prepared using a similar procedure as above:

| Example | Structure | Analytical Data |
|---|---|---|
| 126 | 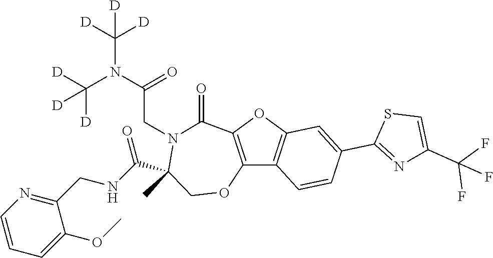 [SMILES: [2H]C([2H])([2H])N(C(=O)CN1C(=O)c2oc3cc(-c4nc(C(F)(F)F)cs4)ccc3c2OC[C@]1(C)C(=O)NCc1ncccc1OC)C([2H])([2H])[2H]] | ¹H NMR (400 MHz, DMSO) δ 9.46 (s, 1H), 8.63 (s, 1H), 8.19 (s, 1H), 7.93 (dd, J = 8.3, 1.1 Hz, 1H), 7.76 (d, J = 8.2 Hz, 1H), 7.56 (s, 1H), 7.23 (d, J = 8.1 Hz, 1H), 7.02 (s, 1H), 4.88 (d, J = 10.7 Hz, 1H), 4.49 (s, 2H), 4.39 (d, J = 12.2 Hz, 1H), 4.33 (d, J = 6.2 Hz, 1H), 4.14 (s, 1H), 3.69 (s, 3H), 1.59 (s, 3H); LC-MS: Found [M + H]⁺ = 624.2 |
| 127 | 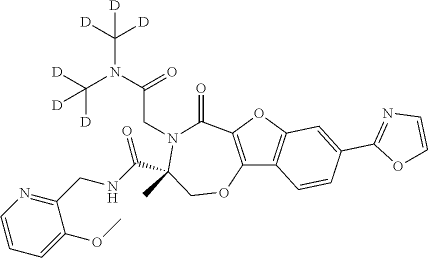 [SMILES: [2H]C([2H])([2H])N(C(=O)CN1C(=O)c2oc3cc(-c4ncco4)ccc3c2OC[C@]1(C)C(=O)NCc1ncccc1OC)C([2H])([2H])[2H]] | ¹H NMR (400 MHz, DMSO) δ 9.46 (s, 1H), 8.30 (d, J = 0.7 Hz, 1H), 8.09 (s, 1H), 7.92 (dd, J = 8.3, 1.1 Hz, 1H), 7.76 (d, J = 8.2 Hz, 1H), 7.54 (s, 1H), 7.46 (d, J = 0.7 Hz, 1H), 7.22 (d, J = 7.9 Hz, 1H), 7.02 (s, 1H), 4.88 (d, J = 11.9 Hz, 1H), 4.49 (s, 2H), 4.38 (d, J = 11.9 Hz, 1H), 4.31 (dd, J = 15.5, 6.2 Hz, 1H), 4.14 (s, 1H), 3.68 (s, 3H), 1.59 (s, 3H). LC-MS: Found [M + H]⁺ = 540.2 |
| 128 | 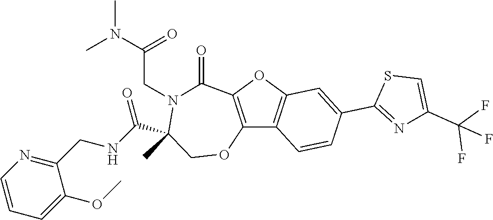 [SMILES: COc1cccnc1CNC(=O)[C@@]1(C)COc2c(oc3cc(-c4nc(C(F)(F)F)cs4)ccc23)C(=O)N1CC(=O)N(C)C] | ¹H NMR (400 MHz, DMSO) δ 9.45 (s, 1H), 8.63 (d, J = 0.8 Hz, 1H), 8.19 (s, 1H), 7.93 (dd, J = 8.3, 1.2 Hz, 1H), 7.76 (d, J = 8.2 Hz, 1H), 7.56 (s, 1H), 7.23 (d, J = 8.0 Hz, 1H), 7.02 (s, 1H), 4.89 (d, J = 11.3 Hz, 1H), 4.50 (s, 2H), 4.39 (d, J = 11.6 Hz, 1H), 4.31 (dd, J = 15.5, 6.2 Hz, 1H), 4.15 (s, 1H), 3.70 (s, 3H), 3.07 (s, 3H), 2.86 (s, 3H), 1.60 (s, 3H). LC-MS: Found [M + H]⁺ = 618.2 |

### Example 129

### (R)-8-(4-cyanooxazol-2-yl)-4-(2-(dimethylamino)-2-oxoethyl)-N-((3-methoxypyridin-2-yl)methyl)-3-methyl-5-oxo-2,3,4,5-tetrahydrobenzofuro[2,3-f][1,4]oxazepine-3-carboxamide

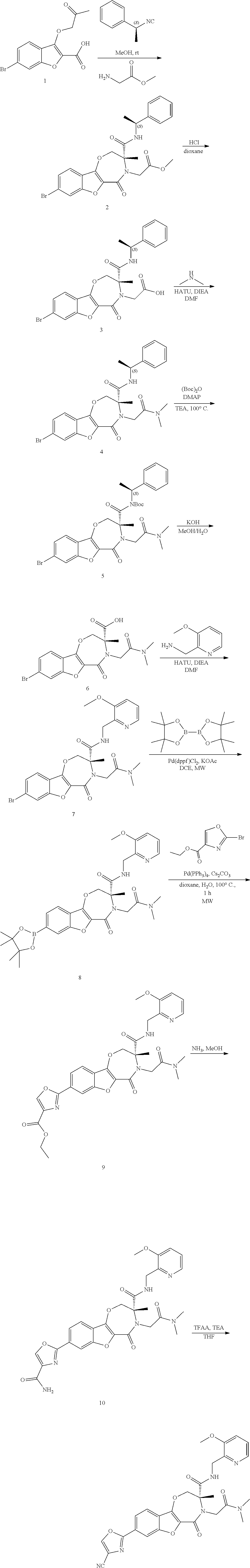

### methyl 2-((R)-8-bromo-3-methyl-5-oxo-3-(((S)-1-phenylethyl)carbamoyl)-2,3-dihydrobenzofuro[2,3-f][1,4]oxazepin-4(5H)-yl)acetate

A mixture of 6-bromo-3-(2-oxopropoxy)benzofuran-2-carboxylic acid (55 g, 0.176 mol, 1.0 eq), methyl glycinate (24 g, 0.263 mol, 1.5 eq) and (S)-(1-isocyanoethyl)benzene (24 g, 0.263 mol, 1.5 eq) in MeOH (20 mL) was stirred at 50° C. for 16 h. The mixture was concentrated under reduced pressure. The crude product was purified by flash chromatography on silica gel (PE:EA=1:1) to give the product (40 g, 43.9%) as a yellow solid. TLC: PE/EA=1:1, UV; R_{f} Starting Material=0.0, Product=0.4, isomer=0.6

### 2-((R)-8-bromo-3-methyl-5-oxo-3-(((S)-1-phenylethyl)carbamoyl)-2,3-dihydrobenzofuro[2,3-f][1,4]oxazepin-4(5H)-yl)acetic acid

A mixture of methyl 2-((R)-8-bromo-3-methyl-5-oxo-3-(((S)-1-phenylethyl)carbamoyl)-2,3-dihydrobenzofuro[2,3-f][1,4]oxazepin-4(5H)-yl)acetate (40 g, 77.62 mmol, 1.0 eq), 6M HCl (600 mL) in dioxane (800 mL) was stirred at 80° C. for 16 h. The mixture was concentrated under reduced pressure. The solution was extracted with EA, the solvent was removed under reduced pressure to give the product (35 g, 92%) as a yellow solid.

### (R)-8-bromo-4-(2-(dimethylamino)-2-oxoethyl)-3-methyl-5-oxo-N—((S)-1-phenylethyl)-2,3,4,5-tetrahydrobenzofuro[2,3-f][1,4]oxazepine-3-carboxamide

To a solution of 2-((R)-8-bromo-3-methyl-5-oxo-3-(((S)-1-phenylethyl)carbamoyl)-2,3-dihydrobenzofuro[2,3-f][1,4]oxazepin-4(5H)-yl)acetic acid (27 g, 53.86 mmol, 1.0 eq) in DMF (30 mL) was added HATU (41 g, 107.7 mmol, 2.0 eq), dimethylamine (50 mL, 2M, 107.7 mmol, 2.0 eq) and DIPEA (45 mL, 269.3 mmol, 5.0 eq) were added. The reaction mixture was stirred at room temperature for 2 h. The reaction mixture was poured into H₂O (30 mL) and extracted with EtOAc (3×30 mL). The combined organic layers were washed with brine, dried over Na₂SO₄, filtered, and concentrated under reduced pressure. The crude product was purified by flash chromatography on silica gel (DCM:MeOH=5:1) to give the product (25 g, 89%) as a white solid.

### tert-butyl ((R)-8-bromo-4-(2-(dimethylamino)-2-oxoethyl)-3-methyl-5-oxo-2,3,4,5-tetrahydrobenzofuro[2,3-f][1,4]oxazepine-3-carbonyl)((S)-1-phenylethyl)carbamate

To a solution of (R)-8-bromo-4-(2-(dimethylamino)-2-oxoethyl)-3-methyl-5-oxo-N—((S)-1-phenylethyl)-2,3,4,5-tetrahydrobenzofuro[2,3-f][1,4]oxazepine-3-carboxamide (22 g, 41.7 mmol, 1.0 eq) in TEA (200 mL) and DMA (90 mL) was added (Boc)₂O (150 mL), DMAP (2.8 g, 22.7 mmol, 1.0 eq). The mixture was stirred at 100° C. for 16 hours. Water (150 mL) was added. The aqueous phase was extracted with EA (3×250 mL). The combined organic solution was washed with brine (250 mL), dried over anhydrous Na₂SO₄, and the solution was concentrated under reduced pressure. The residue was purified by silica gel chromatography (PE:EA=1:2) to give the product (15 g, 57.6%) as brown solid.

### (R)-8-bromo-4-(2-(dimethylamino)-2-oxoethyl)-3-methyl-5-oxo-2,3,4,5-tetrahydrobenzofuro[2,3-f][1,4]oxazepine-3-carboxylic acid

To a solution of tert-butyl ((R)-8-bromo-4-(2-(dimethylamino)-2-oxoethyl)-3-methyl-5-oxo-2,3,4,5-tetrahydrobenzofuro[2,3-f][1,4]oxazepine-3-carbonyl)((S)-1-phenylethyl)carbamate (15 g, 23.9 mmol, 1.0 eq) in THE (170 mL), MeOH (170 mL) and H₂O (100 mL) was added KOH (26.7 g, 478 mmol, 20 eq), the mixture was stirred at 70° C. for 3 hours. The reaction mixture was concentrated under reduced pressure and then acidified to pH 4-5 with 1 N HCl. The solid was filtered to give the product (8 g, 80%) as white solid. The crude product used in the next step directly.

### (R)-8-bromo-4-(2-(dimethylamino)-2-oxoethyl)-N-((3-methoxypyridin-2-yl)methyl)-3-methyl-5-oxo-2,3,4,5-tetrahydrobenzofuro[2,3-f][1,4]oxazepine-3-carboxamide

To a solution of ((R)-8-bromo-4-(2-(dimethylamino)-2-oxoethyl)-3-methyl-5-oxo-2,3,4,5-tetrahydrobenzofuro[2,3-f][1,4]oxazepine-3-carboxylic acid (8 g, 18.86 mmol, 1.0 eq) in DMF (20 mL) was added HATU (37.7 g, 18.86 mmol, 1.0 eq), (3-methoxypyridin-2-yl)methanamine (3.8 g, 27.82 mmol, 2.0 eq) and DIPEA (3.6 g, 27.82 mmol, 2.0 eq). The reaction mixture was stirred at room temperature for 2 h. The reaction mixture was poured into H₂O (130 mL) and extracted with EtOAc (3×130 mL). The combined organic layers were washed with brine, dried over Na₂SO₄, filtered, and concentrated under reduced pressure. The crude product was purified by prep-HPLC to give the product (8 g, 77.9%) as a white solid.

### (R)-4-(2-(dimethylamino)-2-oxoethyl)-N-((3-methoxypyridin-2-yl)methyl)-3-methyl-5-oxo-8-(4,4,5,5-tetramethyl-1,3,2-dioxaborolan-2-yl)-2,3,4,5-tetrahydrobenzofuro[2,3-f][1,4]oxazepine-3-carboxamide

A mixture of (1,1′-bis(diphenylphosphino)ferrocene)palladium(11) chloride (670 mg, 0.917 mmol, 0.1 eq), potassium acetate (4.5 g, 45.85 mmol, 5.0 eq), (R)-8-bromo-4-(2-(dimethylamino)-2-oxoethyl)-N-((3-methoxypyridin-2-yl)methyl)-3-methyl-5-oxo-2,3,4,5-tetrahydrobenzofuro[2,3-f][1,4]oxazepine-3-carboxamide (25 g, 9.17 mmol, 1.0 eq) and bis(pinacolato)diboron (11.6 g, 45.85 mmol, 5.0 eq) in dioxane (100 ml) was degassed with argon for 5 min, the resulting mixture stirred at 100° C. under argon for 3 hours. The reaction mixture was allowed to cool and diluted with ethyl acetate. This solution was washed with water. The organic layer was separated, dried over sodium sulfate and evaporated under reduced pressure, the residue was purified by flash column chromatography on silica gel (PE:EA=3:1) to give the product (3.3 g, 60.5%) as light-yellow solid.

### ethyl (R)-2-(4-(2-(dimethylamino)-2-oxoethyl)-3-(((3-methoxypyridin-2-yl)methyl)carbamoyl)-3-methyl-5-oxo-2,3,4,5-tetrahydrobenzofuro[2,3-f][1,4]oxazepin-8-yl)oxazole-4-carboxylate

A mixture of Pd(PPh₃)₄ (643 mg, 0.557 mmol, 0.1 eq), Cs₂CO₃ (3.6 g, 11.18 mmol, 2.0 eq), intermediate (R)-4-(2-(dimethylamino)-2-oxoethyl)-N-((3-methoxypyridin-2-yl)methyl)-3-methyl-5-oxo-8-(4,4,5,5-tetramethyl-1,3,2-dioxaborolan-2-yl)-2,3,4,5-tetrahydrobenzofuro[2,3-f][1,4]oxazepine-3-carboxamide (3.3 g, 5.57 mmol, 1.0 eq) and ethyl 2-bromooxazole-4-carboxylate (1.6 g, 7.2 mmol, 1.3 eq) in dioxane (100 ml) and H₂O (10 ml) was degassed with argon for 2 mins, the resulting mixture stirred at 100° C. under argon for 16 hours. The reaction mixture was allowed to cool and diluted with ethyl acetate. This solution was washed with water. The organic layer was separated, dried over sodium sulfate and evaporated under reduced pressure, the residue was purified by flash column chromatography on silica gel (DCM:MeOH=20:1) to give the product (2.6 g, 77%) as light-yellow solid.

### (R)-8-(4-carbamoyloxazol-2-yl)-4-(2-(dimethylamino)-2-oxoethyl)-N-((3-methoxypyridin-2-yl)methyl)-3-methyl-5-oxo-2,3,4,5-tetrahydrobenzofuro[2,3-f][1,4]oxazepine-3-carboxamide

A mixture of ethyl (R)-2-(4-(2-(dimethylamino)-2-oxoethyl)-3-(((3-methoxypyridin-2-yl)methyl)carbamoyl)-3-methyl-5-oxo-2,3,4,5-tetrahydrobenzofuro[2,3-f][1,4]oxazepin-8-yl)oxazole-4-carboxylate (2.6 g, 4.29 mmol, 1.0 eq) in NH MeOH (10 mL, 7N) was stirred at 80° C. for 2 h in MW. The solvent was removed under reduced pressure to give the product (2.2 g, 89%) as a yellow solid.

### (R)-8-(4-cyanooxazol-2-yl)-4-(2-(dimethylamino)-2-oxoethyl)-N-((3-methoxypyridin-2-yl)methyl)-3-methyl-5-oxo-2,3,4,5-tetrahydrobenzofuro[2,3-f][1,4]oxazepine-3-carboxamide

A mixture of (R)-8-(4-carbamoyloxazol-2-yl)-4-(2-(dimethylamino)-2-oxoethyl)-N-((3-methoxypyridin-2-yl)methyl)-3-methyl-5-oxo-2,3,4,5-tetrahydrobenzofuro[2,3-f][1,4]oxazepine-3-carboxamide (2.2 g, 3.81 mol, 1.0 eq), TEA (1.54 g, 15.24 mol, 4.0 eq) and TFAA (1.6 g, 7.6 mol, 2 eq) in DCM (50 mL) was stirred at 0° C. for 2 h. The mixture was concentrated under reduced pressure. The crude product was purified by flash chromatography on silica gel (PE:EA=1:1) to give the product (1.15 g, 52%) as a white solid.

LC-MS: Calculated Exact Mass=558.2, Found [M+H]⁺=559.2.

¹H NMR (400 MHz, DMSO) δ 9.47 (s, 1H), 9.28 (s, 1H), 8.18 (s, 1H), 7.92 (dd, J=8.3, 1.0 Hz, 1H), 7.80 (d, J=8.2 Hz, 1H), 7.51 (s, 1H), 7.21 (d, J=8.1 Hz, 1H), 7.01 (s, 1H), 4.89 (d, J=11.8 Hz, 1H), 4.50 (s, 2H), 4.39 (d, J=11.7 Hz, 1H), 4.32 (dd, J=15.5, 6.3 Hz, 1H), 4.19-4.06 (m, 1H), 3.68 (s, 3H), 3.07 (s, 3H), 2.86 (s, 3H), 1.60 (s, 3H).

The following example was prepared in racemic form using similar procedures as above:

| Example | Structure graphic | Analytical Data |
|---|---|---|
| 130 | 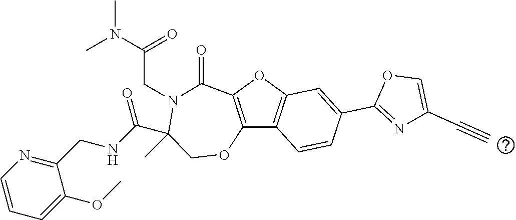 [SMILES: C#Cc1coc(-c2ccc3c4c(oc3c2)C(=O)N(CC(=O)N(C)C)C(C)(C(=O)NCc2ncccc2OC)CO4)n1] | ¹H NMR (400 MHz, DMSO) δ 9.46 (s, 1H), 9.28 (s, 1H), 8.19 (s, 1H), 7.93 (dd, J = 8.3, 1.1 Hz, 1H), 7.80 (d, J = 8.2 Hz, 1H), 7.51 (s, 1H), 7.21 (d, J = 8.0 Hz, 1H), 7.01 (s, 1H), 4.89 (d, J = 11.1 Hz, 1H), 4.50 (s, 2H), 4.39 (d, J = 11.8 Hz, 1H), 4.32 (dd, J = 15.5, 6.3 Hz, 1H), 4.13 (s, 1H), 3.68 (s, 3H), 3.07 (s, 3H), 2.86 (s, 3H), 1.59 (s, 3H). LC-MS: Found [M + H]⁺ = 559.2 |

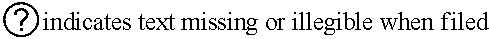

### Intermediate BB-12

### 5-trifluoromethyl-3-(2-oxopropoxy)benzofuran-2-carboxylic acid

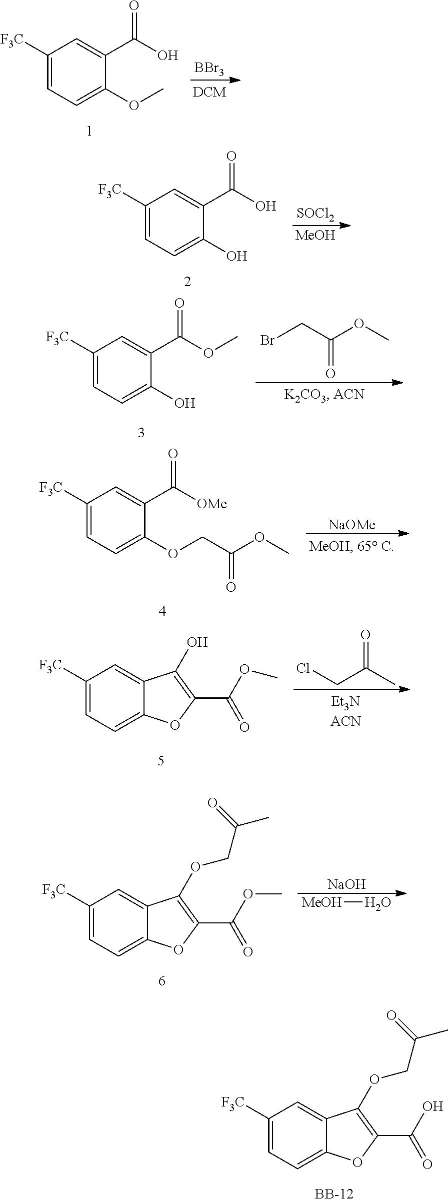

### 2-hydroxy-5-(trifluoromethyl)benzoic acid

BBr₃ (1M in DCM) (89 mL, 89 mmol, 2.0 eq) was added dropwise over 30 min to a stirred mixture of compound 2-methoxy-5-(trifluoromethyl)benzoic acid (9.8 g, 44.5 mmol, 1.0 eq), in DCM (100 mL) at −20° C., the mixture stirred at −20° C. for 4 h. The reaction was monitored by LCMS. The reaction was cooled, poured into ice water, extracted with DCM, the solution was washed successively with water, aqueous sodium carbonate, water, and brine. The crude product was purified by flash chromatography on silica gel (PE:EA=1:1) to give the product (9 g, 98%) as a yellow solid. TLC: PE/EA=5:1, UV; R_{f} Starting Material=0.7, Product=0.5.

### methyl 2-hydroxy-5-(trifluoromethyl)benzoate

SOCl₂ (10 mL) was added dropwise over 5 min to a stirred mixture of compound 2-hydroxy-5-(trifluoromethyl)benzoic acid (8.5 g, 41.24 mol, 1.0 eq) in MeOH (85 mL). The mixture was heated and stirred under reflux for 4 h. The reaction was monitored by LCMS. The crude product was distilled under reduced pressure, washed with H₂O, extracted with ethyl acetate, the organic phase was distilled under reduced pressure to give the product (8 g, 95%) as brown solid. The crude product used in the next step directly. TLC: PE/EA=5:1, UV; R_{f} Starting Material=0.3, Product=0.8.

### methyl 2-(2-methoxy-2-oxoethoxy)-5-(trifluoromethyl)benzoate

Methyl bromoacetate (6.11 g, 39.97 mol, 1.1 eq) was added dropwise over 5 min to a stirred mixture of compound methyl 2-hydroxy-5-(trifluoromethyl)benzoate (8 g, 36.34 mol, 1.0 eq) and potassium carbonate (15.07 g, 109 mol, 3.0 eq) in MeCN (80 mL). The mixture was heated and stirred under reflux for 4 h. The reaction was monitored by LCMS. The reaction was cooled, and the salts were separated by filtration and washed with acetone. The filtrate was concentrated to give a residue, which was diluted with EA (0.25 L), and the solution was washed successively with water, aqueous sodium carbonate, water, and brine. The crude product was distilled under reduced pressure to give the product (10 g, 94%) as a brown solid. The crude product used in the next step directly.

### methyl 3-hydroxy-5-(trifluoromethyl)benzofuran-2-carboxylate

A mixture of compound methyl 2-(2-methoxy-2-oxoethoxy)-5-(trifluoromethyl)benzoate (10 g, 34.22 mmol, 1.0 eq) in MeOH (100 mL) was added NaOMe (30% in methanol, 5M, 13.6 mL, 68.44 mmol, 1.2 eq) and the mixture was stirred at 60° C. for 4 hours. The reaction was monitored by LCMS. The resulting mixture was cooled, poured into ice water and acidified with 5% hydrochloric acid to pH 2. The solid was collected by filtration, washed with water and recrystallized from methanol to give the product (8.7 g, 97%) as yellow solid. LC-MS: Calculated Exact Mass=260.0, Found [M+H]⁺=261.0.

### methyl 3-(2-oxopropoxy)-5-(trifluoromethyl)benzofuran-2-carboxylate

A mixture of compound methyl 3-hydroxy-5-(trifluoromethyl)benzofuran-2-carboxylate (8.7 g, 33.44 mmol, 1.0 eq) in acetonitrile (90 mL) was added TEA (9.32 mL, 66.88 mol, 2 eq), chloroacetone (3.7 g, 40.13 mmol, 1.2 eq), the mixture was refluxed for 4 hours. The reaction was monitored by LCMS. The resulting mixture was filtered, concentrated, extracted with ethyl acetate to give the product (9 g, 85%) as brown solid. LC-MS: Calculated Exact Mass=316.0, Found [M+H]⁺=317.0.

### 5-trifluoromethyl-3-(2-oxopropoxy)benzofuran-2-carboxylic acid (BB-5)

To a solution of crude compound methyl 3-(2-oxopropoxy)-5-(trifluoromethyl)benzofuran-2-carboxylate (8 g, 25.3 mmol, 1.0 eq) in MeOH (100 mL) and 5% NaOH in water (40 mL) was stirred at rt for 4 hours. The reaction was monitored by LCMS. The resulting mixture was concentrated, added 1 N HCl to adjust to pH 2, extracted with ethyl acetate. The combined organic layers were washed with brine, dried over Na₂SO₄. The solvent was removed under reduced pressure and the residue was purified by flash column chromatography on silica (DCM:MeOH=10:1) to give the product (3.8 g, 49%) as yellow solid. LC-MS: Calculated Exact Mass=302.0, Found [M+H]⁺=303.0.

### Example 131

### 4-(2-(dimethylamino)-2-oxoethyl)-N-(2-fluoro-6-methoxybenzyl)-3-methyl-5-oxo-9-(trifluoromethyl)-2,3,4,5-tetrahydrobenzofuro[2,3-f][1,4]oxazepine-3-carboxamide

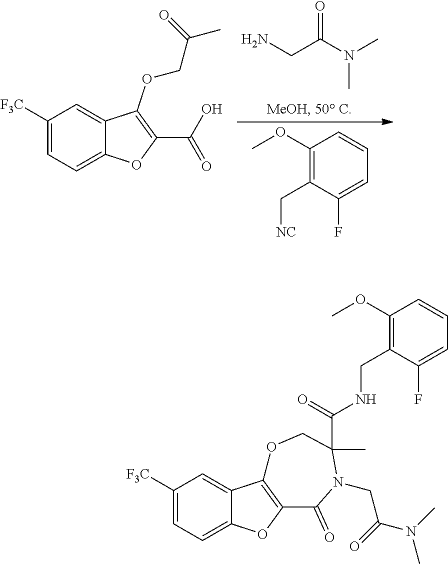

### 4-(2-(dimethylamino)-2-oxoethyl)-N-(2-fluoro-6-methoxybenzyl)-3-methyl-5-oxo-9-(trifluoromethyl)-2,3,4,5-tetrahydrobenzofuro[2,3-f][1,4]oxazepine-3-carboxamide

3-(2-oxopropoxy)-5-(trifluoromethyl)benzofuran-2-carboxylic acid (100 mg, 0.33 mmol, 1.0 eq), 2-amino-N,N-dimethylacetamide (40.5 mg, 0.397 mmol, 1.2 eq), and 1-fluoro-2-(isocyanomethyl)-3-methoxybenzene (65 mg, 0.397 mmol, 1.2 eq) in MeOH (10 mL) was stirred at 50° C. for 16 h. The mixture was concentrated under reduced pressure. The crude product was purified by flash chromatography on silica gel (PE:EA=1:1) to give the product (90 mg, 30%) as a white solid. LC-MS: Calculated Exact Mass=551.2, Found [M+H]⁺=552.2. ¹H NMR (400 MHz, DMSO) δ 9.59 (s, 1H), 7.90 (s, 1H), 7.80 (dd, J=8.9, 1.6 Hz, 1H), 7.74 (d, J=8.8 Hz, 1H), 6.97 (d, J=7.7 Hz, 1H), 6.57 (d, J=8.3 Hz, 1H), 6.29 (s, 1H), 4.87 (d, J=12.0 Hz, 1H), 4.52 (s, 1H), 4.31 (dt, J=13.7, 11.3 Hz, 3H), 3.89 (s, 1H), 3.62 (s, 3H), 3.04 (s, 3H), 2.88 (s, 3H), 1.55 (s, 3H).

Using similar procedures, the following examples were prepared:

| Example | Structure graphic | Analytical Data |
|---|---|---|
| 132 | 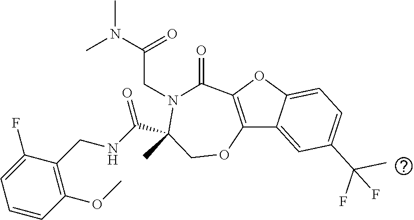 | ¹H NMR (400 MHz, DMSO) δ 9.59 (s, 1H), 7.90 (s, 1H), 7.80 (dd, J = 8.9, 1.6 Hz, 1H), 7.74 (d, J = 8.8 Hz, 1H), 6.97 (d, J = 7.7 Hz, 1H), 6.57 (d, J = 8.3 Hz, 1H), 6.29 (s, 1H), 4.87 (d, J = 12.0 Hz, 1H), 4.52 (s, 1H), 4.31 (dt, J = 13.7, 11.3 Hz, 3H), 3.89 (s, 1H), 3.62 (s, 3H), 3.04 (s, 3H), 2.88 (s, 3H), 1.55 (s, 3H) LC-MS: Found [M + H]⁺ = 552.2 |

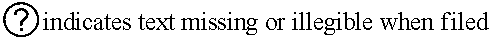

### Example 133

### 4-(2-(dimethylamino)-2-oxoethyl)-N-(2-fluoro-6-methoxybenzyl)-3,9-dimethyl-5-oxo-2,3,4,5-tetrahydrobenzofuro[2,3-f][1,4]oxazepine-3-carboxamide

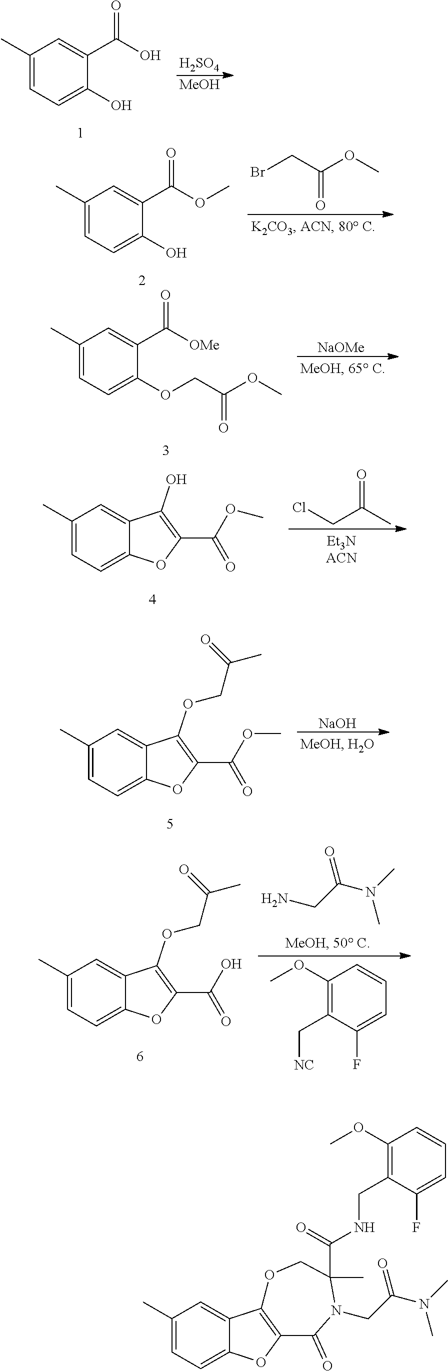

5-methyl-3-(2-oxopropoxy)benzofuran-2-carboxylic acid was prepared using a similar procedure as above

| Example | Structure | Analytical Data |
|---|---|---|
| 133 | 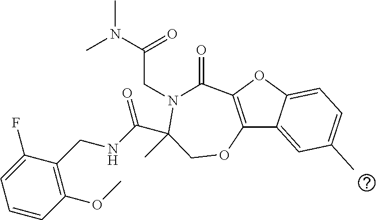 [SMILES: COc1cccc(F)c1CNC(=O)C1(C)COc2c(oc3ccc(C)cc23)C(=O)N1CC(=O)N(C)C] | ¹H NMR (400 MHz, DMSO) δ 9.45 (s, 1H), 7.38 (d, J = 8.5 Hz, 1H), 7.35 (s, 1H), 7.29 (d, J = 8.6 Hz, 1H), 7.08 (dd, J = 15.3, 7.7 Hz, 1H), 6.63 (d, J = 8.4 Hz, 1H), 6.45 (s, 1H), 4.82 (d, J = 11.7 Hz, 1H), 4.45 (s, 1H), 4.35-4.15 (m, 3H), 4.00 (s, 1H), 3.61 (s, 3H), 3.02 (s, 3H), 2.84 (s, 3H), 2.40 (s, 3H), 1.51 (s, 3H) LC-MS: Found [M + H]⁺ = 498.2 |

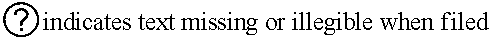

### Example 134

### (R)-4-(2-(dimethylamino)-2-oxoethyl)-N-(2-fluoro-6-methoxybenzyl)-3-methyl-5-oxo-8-(trifluoromethyl)-2,3,4,5-tetrahydropyrido[2′,3′:4,5]furo[2,3-f][1,4]oxazepine-3-carboxamide

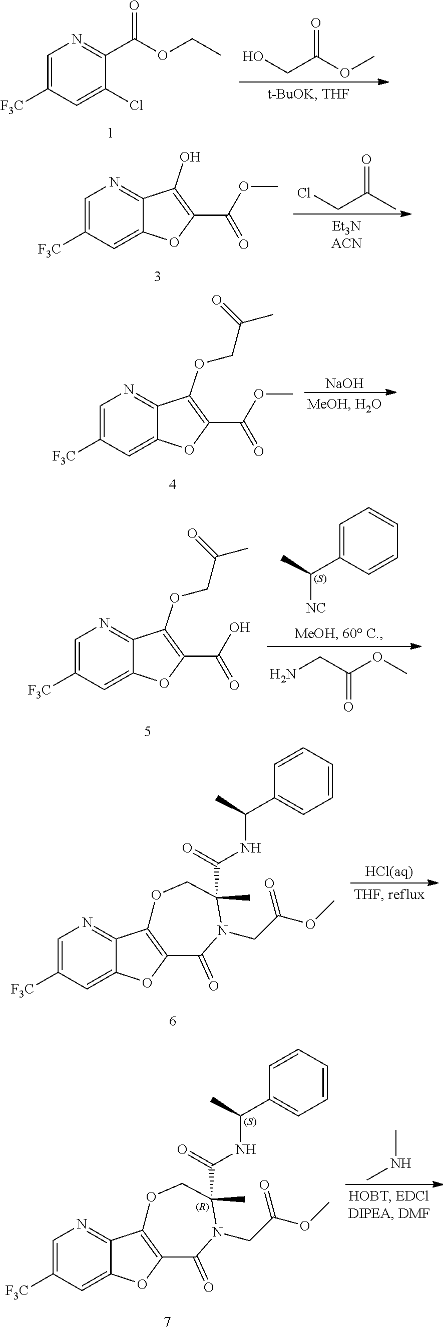

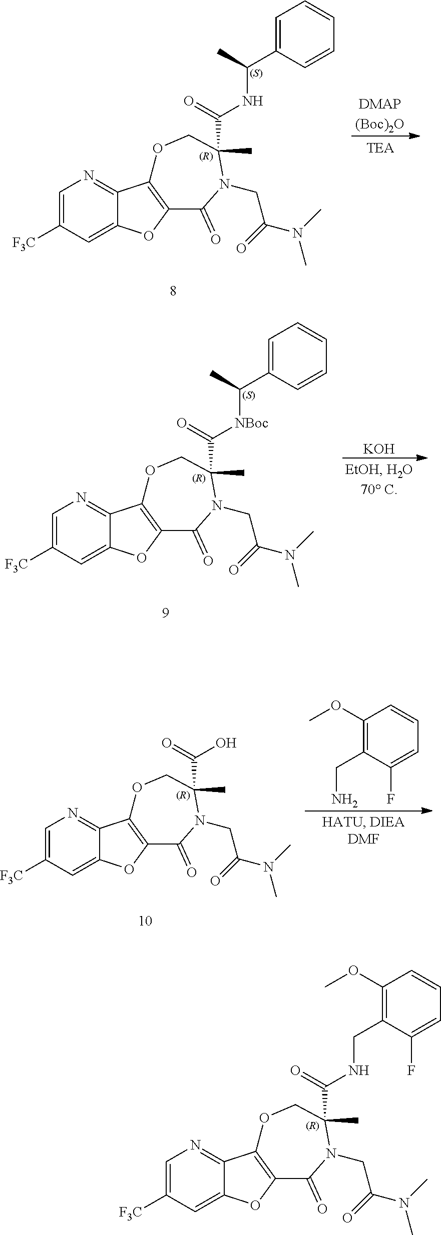

3-(2-oxopropoxy)-6-(trifluoromethyl)furo[3,2-b]pyridine-2-carboxylic acid was prepared using a similar procedure as above

| Example | Structure | Analytical Data |
|---|---|---|
| 134 | 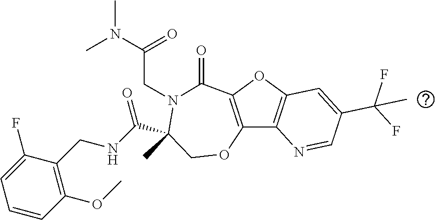 | ¹H NMR (400 MHz, DMSO) δ 9.71 (s, 1H), 8.89 (s, 1H), 8.57 (s, 1H), 6.87 (d, J = 7.0 Hz, 1H), 6.61 (d, J = 8.2 Hz, 1H), 6.24 (s, 1H), 4.89 (d, J = 11.8 Hz, 1H), 4.58 (s, 1H), 4.47-4.31 (m, 3H), 3.79 (d, J = 6.5 Hz, 1H), 3.69 (d, J = 6.8 Hz, 3H), 3.06 (s, 3H), 2.91 (s, 3H), 1.57 (s, 3H). LC-MS: Found [M + H]⁺ = 553.2 |

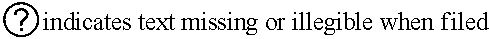

### Examples 135-138

| Example | Structure | Analytical Data | BB |
|---|---|---|---|
| 135 | 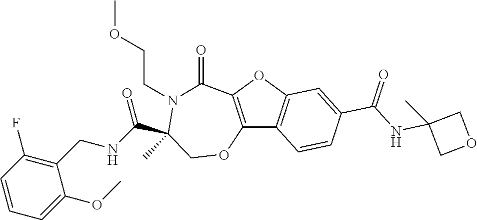 [SMILES: COCCN1C(=O)c2oc3cc(C(=O)NC4(C)COC4)ccc3c2OC[C@@]1(C)C(=O)NCc1c(F)cccc1OC] | 1H NMR (400 MHz, CDCl3) δ 7.83 (s, 1H), 7.61 (d, J = 8.2 Hz, 1H), 7.53 (d, J = 7.9 Hz, 1H), 7.39-7.30 (m, 1H), 6.94- 6.79 (m, 1H), 6.48 (s, 1H), 6.44-6.27 (m, 2H), 4.96-4.82 (m, 3H), 4.66-4.50 (m, 3H), 4.33-4.19 (m, 1H), 4.17-4.02 (m, 2H), 3.85-3.75 (m, 1H), 3.73 (s, 3H), 3.63- 3.49 (m, 2H), 3.23 (s, 3H), 1.81 (s, 3H), 1.77 (s, 3H). LC-MS: 570.20 [M + 1]+ | BB-4 |
| 136 | 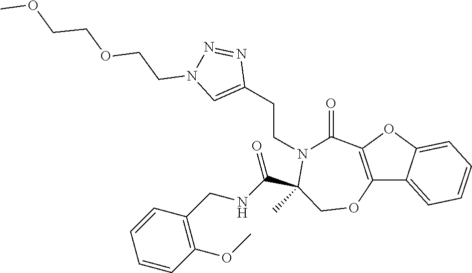 [SMILES: COCCOCCn1cc(CCN2C(=O)c3oc4ccccc4c3OC[C@@]2(C)C(=O)NCc2ccccc2OC)nn1] | 1H NMR (400 MHz, CDCl3) δ 7.61 (s, 1H), 7.51 (d, J = 7.9 Hz, 1H), 7.46-7.37 (m, 2H), 7.20 (ddd, J = 8.0, 5.1, 3.0 Hz, 1H), 7.04 (t, J = 5.8 Hz, 1H), 6.98 (d, J = 7.2 Hz, 1H), 6.93 (t, J = 7.7 Hz, 1H), 6.54 (dd, J = 12.2, 7.8 Hz, 2H), 4.81 (d, J = 12.1 Hz, 1H), 4.62-4.45 (m, 2H), 4.40 (dd, J = 14.3, 6.8 Hz, 1H), 4.21 (dd, J = 14.2, 5.0 Hz, 1H), 4.08 (d, J = 12.1 Hz, 1H), 4.05- 3.94 (m, 1H), 3.94-3.78 (m, 3H), 3.70 (s, 3H), 3.64-3.55 (m, 2H), 3.52-3.48 (m, 2H), 3.36 (s, 3H), 3.23-3.16 (m, 1H), 3.11- 3.01 (m, 1H), 1.79 (s, 3H). LC-MS: 478.2 [M + 1]+ | BB-1 |
| 137 | 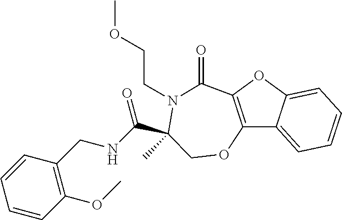 | 1H NMR (400 MHz, DMSO) δ 7.60 (d, J = 8.4 Hz, 1H), 7.51 (t, J = 7.8 Hz, 1H), 7.29 (t, J = 7.4 Hz, 1H), 7.17-7.15 (m, 4H), 4.96-4.95 (m, 1H), 4.79 (d, J = 15.5 Hz, 1H), 4.57-4.54 (m, 2H), 3.92 (d, J = 12.0 Hz, 2H), 3.47-3.42 (m, 3H), 3.19-3.18 (m, 4H), 2.76-2.75 (m, 2H), 1.58 (s, 3H). LC-MS: 435.04 [M + 1]+ | BB-2 |
| 138 | 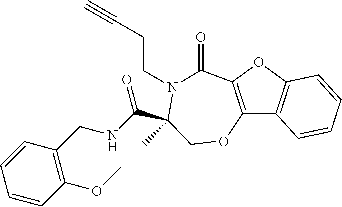 [SMILES: C#CCCN1C(=O)c2oc3ccccc3c2OC[C@@]1(C)C(=O)NCc1ccccc1OC] | 1H NMR (400 MHz, CDCl3) δ 7.52 (d, J = 7.9 Hz, 1H), 7.45-7.40 (m, 2H), 7.21 (ddd, J = 8.0, 5.2, 2.9 Hz, 1H), 6.99-6.95 (m, 2H), 6.72 (t, J = 4 Hz, 1H), 6.5-6.57 (m, 2H), 4.80 (d, J = 11.9 Hz, 1H), 4.37 (dd, J = 14.2, 6.8 Hz, 1H), 4.23 (dd, J = 14.3, 5.1 Hz, 1H), 4.17 (d, J = 12.2 Hz, 1H), 3.86-3.84 (m, 1H), 3.72 (s, 3H), 3.73- 3.58 (m, 1H), 2.73-3.69 (m, 1H), 2.66- 2.49 (m, 1H), 2.00 (t, J = 2.7 Hz, 1H), 1.79 (s, 3H). LC-MS: 433.2 [M + 1]+ | BB-1 |

### Conditions for KLF2 Induction Cell Based Bioassay

In endothelial cell culture media, a stock of the highest working concentration was made at no more than 1% DMSO vehicle. The working stock was then serially diluted 1:2 to make an 11-point concentration response curve. Included in the 96-well plate were 2 reference compounds with known potencies. A DMSO column was included in the 96-well plate at the highest percent vehicle of the working stocks. The 96-well cell culture plate of primary human endothelial cells expressing luciferase under the control of the KLF2 promoter was dosed with 200 μL per well of the dilution series as well as the DMSO control column. The assay plate was incubated at 37° C. for 24 hours. The supernatant was collected at 24 hours.

20 μL of the supernatant was then placed in a 384-well plate with optical duplicates. The corners of the plate received 20 μL of the Gaussia Luciferase Enzyme positive control, as well as 20 μL of the 50 μM Coelenterizine substrate. The plate read is then calibrated using the control, and read using a Molecular Devices SpectraMax iD5, providing luminescence in relative light units (RLU). The plate reader injected 20 μL of the 50 μM Coelenterizine substrate immediately before taking the RLU measurement for each well.

The sample signals were then normalized to the average signal of the DMSO column, as well as to the maximum signal of one of the reference compounds, as shown in the equations below:

$$\frac{Mean of sample optical replicates ( RLU )}{Mean of all DMSO controls ( RLU )} = Vehicle Normalization \\ \frac{Vehicle Normalization - 1}{Reference Compound Maximum - 1} = Scaled to Maximum Signal$$

The EC50 of each compound is then defined as when the concentration response curve passes 0.5.

The Activity Range is defined as follows:

| Category | EC₅₀ (nM) |
|---|---|
| A | Between 501 and 5000 |
| B | Between 51 and 500 |
| C | Less than or equal to 50 |

Using these activity definitions, the biological activity of the examples is shown below:

| Example | Activity Range | Example | Activity Range |
|---|---|---|---|
| 2 | A | 68 | B |
| 3 | B | 69 | B |
| 4 | A | 70 | B |
| 5 | A | 71 | B |
| 5 | C | 72 | B |
| 6 | A | 73 | B |
| 7 | B | 74 | A |
| 9 | A | 75 | B |
| 10 | A | 76 | B |
| 11 | A | 77 | B |
| 12 | A | 78 | B |
| 13 | A | 79 | B |
| 14 | B | 80 | A |
| 15 | A | 81 | B |
| 16 | A | 82 | B |
| 17 | A | 83 | B |
| 18 | A | 84 | C |
| 19 | A | 85 | C |
| 20 | A | 86 | C |
| 21 | A | 87 | C |
| 22 | A | 88 | C |
| 23 | A | 89 | C |
| 24 | A | 90 | C |
| 25 | A | 91 | C |
| 26 | A | 92 | C |
| 27 | A | 93 | C |
| 28 | A | 94 | C |
| 29 | A | 95 | A |
| 30 | A | 96 | A |
| 31 | A | 97 | A |
| 32 | A | 99 | A |
| 33 | A | 100 | B |
| 34 | A | 101 | B |
| 35 | A | 102 | B |
| 36 | A | 103 | B |
| 37 | A | 104 | B |
| 38 | A | 105 | B |
| 39 | A | 106 | B |
| 40 | A | 107 | B |
| 41 | A | 108 | B |
| 42 | B | 109 | B |
| 43 | A | 110 | A |
| 44 | A | 111 | B |
| 45 | A | 112 | B |
| 46 | A | 113 | B |
| 47 | A | 114 | B |
| 48 | A | 115 | B |
| 49 | A | 116 | B |
| 50 | B | 117 | B |
| 52 | B | 118 | B |
| 53 | B | 119 | B |
| 54 | B | 120 | B |
| 55 | B | 121 | B |
| 56 | B | 122 | B |
| 57 | B | 123 | B |
| 58 | B | 124 | B |
| 59 | B | 125 | C |
| 60 | B | 126 | C |
| 61 | B | 127 | C |
| 62 | B | 128 | C |
| 63 | B | 129 | C |
| 64 | B | 130 | C |
| 65 | B | 131 | B |
| 66 | A | 132 | B |
| 67 | B | 133 | B |
| 51 | C | 134 | B |

### Confirmation of Stereochemistry of Example 129

Crystal obtained from dichloromethane/methanol/ethylacetate/n-hexane in a 1:1:1:1 ratio after 8 days at room temperature (15-23° C.) Instrument parameters (Bruker D8 Venture):

| | |
|---|---|
| Light source: Cu target | X-ray: Cu-Kα (=1.54178 Å) |
| Detector: CMOS surface detector | Resolution: 0.88 Å |
| Current and voltage: 50 kV, 1.2 mA | Exposure time: 3 s |
| Distance from surface detector to sample: | Test temperature: 170(2)K |
| 40 mm | |

### Structural Analysis and Refining Process:

After the integral reduction of the diffraction data by the SAINT program, empirical absorption correction of data is done by using the SADABS program, analysis of monocrystal structure with the direct method using SHEXLT2014, and the structure is refined by least squares method. The hydrogen atom refinement process is obtained by isotropic calculation, hydrogen atoms on 0 and N are obtained by residual electron density, hydrogen atoms on C—H are Obtained by calculating hydrogenation, and refined by adopting a riding model. The flack constant is −0.03 (10) through the structure diagram, and we can determine the absolute configuration. The C10 configuration in the structure is S.

Crystal data

| | |
|---|---|
| C₂₈H₂₆N₆O₇ | F(000) = 584 |
| Mᵣ = 558.55 | Dₓ = 1.404 Mg m⁻³ |
| Monoclinic, P2₁ | Cu Kα radiation, λ = 1.54184 Å |
| a = 6.3656 (3) Å | Cell parameters from 7553 reflections |
| b = 13.1446 (7) Å | θ = 2.8-74.4° |
| c = 15.7883 (9) Å | μ = 0.86 mm⁻¹ |
| β = 89.949 (3)° | T = 170 K |
| V = 1321.06 (12) Å³ | Block, colourless |
| Z = 2 | 0.08 × 0.05 × 0.04 mm |

Data collection

| | |
|---|---|
| D8 VENTURE | 4717 reflections with I > |
| diffractometer | 2σ(I) |
| ϕ and ω scans | Rᵢₙₜ = 0.043 |
| Absorption correction: multi-scan | θₘₐₓ = 74.4°, θₘᵢₙ = 2.8° |
| SADABS2016/2 (Bruker, 2016/2) was used | |
| for absorption correction. wR2(int) was | |
| 0.1698 before and 0.0611 after correction. | |
| The Ratio of minimum to maximum | |
| transmission is 0.8191. The λ/2 correction | |
| factor is Not present. | |
| Tₘᵢₙ = 0.617, Tₘₐₓ = 0.754 | h = −7 ®7 |
| 11589 measured reflections | k = −16 ®15 |
| 4863 independent reflections | l = −19 ®16 |

Refinement

| | |
|---|---|
| Refinement on F² | Hydrogen site location: inferred from |
| | neighbouring sites |
| Least-squares matrix: full | H-atom parameters constrained |
| R[F² > 2σ(F²)] = 0.043 | w = 1/[σ²(Fₒ²) + (0.0646P)² + 0.1991P] |
| | where P = (Fₒ² + 2F_{c}²)/3 |
| wR(F²) = 0.110 | (Δ/σ)ₘₐₓ < 0.001 |
| S = 1.06 | Δ_{ñmax} = 0.49 e Å⁻³ |
| 4863 reflections | Δ_{ñmin} = −0.21 e Å⁻³ |
| 375 parameters | Absolute structure: Flack x determined |
| | using 1956 quotients [(I+) − (I−)]/[(I+) + (I−)] |
| | (Parsons, Flack and Wagner, Acta Cryst. |
| | B69 (2013) 249-259). |
| 1 restraint | Absolute structure parameter: −0.03 (10) |

### Special Details:

Geometry. All esds (except the esd in the dihedral angle between two 1.s. planes) are estimated using the full covariance matrix. The cell esds are taken into account individually in the estimation of esds in distances, angles and torsion angles; correlations between esds in cell parameters are only used when they are defined by crystal symmetry. An approximate (isotropic) treatment of cell esds is used for estimating esds involving 1.s. planes.

Refinement. Refined as a 2-component twin.

Fractional atomic coordinates and isotropic or equivalent isotropic
displacement parameters (Å²) for (cu_20221392_0m)

| | x | | y | | z | | Uᵢₛₒ*/U_{eq} | |
|---|---|---|---|---|---|---|---|---|
| O3 | 0.5519 | (5) | 0.2648 | (2) | 0.7687 | (2) | 0.0287 | (6) |
| O2 | 0.9176 | (6) | 0.5833 | (3) | 0.8086 | (2) | 0.0388 | (8) |
| C13 | 0.6397 | (7) | 0.4484 | (3) | 0.5830 | (2) | 0.0219 | (8) |
| O4 | 0.6585 | (5) | 0.2615 | (2) | 0.59000 | (18) | 0.0253 | (6) |
| O7 | −0.1727 | (5) | 0.5640 | (2) | 0.3728 | (2) | 0.0310 | (7) |
| O6 | 0.8678 | (5) | 0.5839 | (2) | 0.6054 | (2) | 0.0294 | (7) |
| C10 | 0.8667 | (7) | 0.3393 | (3) | 0.7116 | (2) | 0.0217 | (8) |
| O5 | 0.5025 | (5) | 0.5178 | (2) | 0.54468 | (19) | 0.0232 | (6) |
| O1 | 0.3515 | (8) | 0.5777 | (4) | 0.9929 | (3) | 0.0534 | (10) |
| C8 | 0.6611 | (6) | 0.3424 | (3) | 0.7645 | (2) | 0.0206 | (7) |
| N2 | 0.6300 | (6) | 0.4279 | (3) | 0.8085 | (2) | 0.0276 | (7) |
| H2 | 0.696509 | | 0.483661 | | 0.793301 | | 0.033* | |
| C26 | −0.3533 | (8) | 0.5738 | (4) | 0.3275 | (3) | 0.0327 | (9) |
| H26 | −0.421457 | | 0.635543 | | 0.312575 | | 0.039* | |
| N1 | 0.8012 | (8) | 0.4135 | (5) | 0.9724 | (3) | 0.0507 | (13) |
| N4 | 1.2270 | (7) | 0.6547 | (3) | 0.7743 | (3) | 0.0346 | (9) |
| C16 | 1.0826 | (7) | 0.5820 | (3) | 0.7671 | (3) | 0.0282 | (8) |
| C15 | 1.1276 | (6) | 0.4931 | (3) | 0.7065 | (3) | 0.0243 | (8) |
| H15A | 1.200230 | | 0.519208 | | 0.655510 | | 0.029* | |
| H15B | 1.222450 | | 0.443988 | | 0.734751 | | 0.029* | |
| C11 | 0.8494 | (7) | 0.2626 | (3) | 0.6384 | (3) | 0.0249 | (8) |
| H11A | 0.967142 | | 0.275554 | | 0.598860 | | 0.030* | |
| H11B | 0.870268 | | 0.193600 | | 0.662164 | | 0.030* | |
| C19 | 0.3531 | (7) | 0.4594 | (3) | 0.5048 | (2) | 0.0209 | (8) |
| C27 | −0.4162 | (7) | 0.4794 | (4) | 0.3079 | (3) | 0.0286 | (9) |
| C24 | 0.1803 | (7) | 0.4993 | (3) | 0.4618 | (3) | 0.0237 | (8) |
| H24 | 0.154411 | | 0.570306 | | 0.458215 | | 0.028* | |
| C25 | −0.1390 | (7) | 0.4618 | (3) | 0.3790 | (3) | 0.0253 | (8) |
| C23 | 0.0490 | (7) | 0.4281 | (4) | 0.4249 | (3) | 0.0239 | (8) |
| C20 | 0.3921 | (7) | 0.3566 | (3) | 0.5138 | (3) | 0.0221 | (8) |
| N3 | 0.9340 | (5) | 0.4405 | (3) | 0.6808 | (2) | 0.0206 | (7) |
| N5 | −0.2784 | (7) | 0.4087 | (3) | 0.3411 | (3) | 0.0305 | (8) |
| C28 | −0.5985 | (9) | 0.4518 | (5) | 0.2598 | (3) | 0.0407 | (11) |
| C2 | 0.5380 | (11) | 0.5369 | (5) | 1.0144 | (4) | 0.0476 | (13) |
| C21 | 0.2619 | (7) | 0.2853 | (3) | 0.4737 | (3) | 0.0240 | (8) |
| H21 | 0.290126 | | 0.214405 | | 0.476865 | | 0.029* | |
| C1 | 0.2748 | (15) | 0.6559 | (7) | 1.0473 | (5) | 0.069 | (2) |
| H1A | 0.135086 | | 0.677283 | | 1.028362 | | 0.104* | |
| H1B | 0.370820 | | 0.714144 | | 1.045570 | | 0.104* | |
| H1C | 0.265899 | | 0.629981 | | 1.105397 | | 0.104* | |
| C12 | 0.5774 | (7) | 0.3506 | (3) | 0.5657 | (3) | 0.0229 | (8) |
| C5 | 0.9095 | (11) | 0.4405 | (6) | 1.0388 | (5) | 0.0607 | (17) |
| H5 | 1.040729 | | 0.408573 | | 1.049512 | | 0.073* | |
| C9 | 1.0295 | (7) | 0.2954 | (3) | 0.7728 | (3) | 0.0283 | (9) |
| H9A | 1.036197 | | 0.337823 | | 0.823747 | | 0.043* | |
| H9B | 1.167509 | | 0.294186 | | 0.745269 | | 0.043* | |
| H9C | 0.988988 | | 0.225969 | | 0.788553 | | 0.043* | |
| C3 | 0.6585 | (12) | 0.5673 | (6) | 1.0845 | (4) | 0.0585 | (17) |
| H3 | 0.616722 | | 0.620320 | | 1.121792 | | 0.070* | |
| C7 | 0.4883 | (7) | 0.4308 | (4) | 0.8812 | (3) | 0.0305 | (9) |
| H7A | 0.428834 | | 0.362301 | | 0.891280 | | 0.037* | |
| H7B | 0.370775 | | 0.478152 | | 0.869589 | | 0.037* | |
| N6 | −0.7444 | (9) | 0.4333 | (6) | 0.2191 | (4) | 0.0627 | (16) |
| C17 | 1.4234 | (8) | 0.6586 | (4) | 0.7263 | (4) | 0.0408 | (11) |
| H17A | 1.450935 | | 0.591851 | | 0.700870 | | 0.061* | |
| H17B | 1.539056 | | 0.676665 | | 0.764289 | | 0.061* | |
| H17C | 1.411688 | | 0.709811 | | 0.681415 | | 0.061* | |
| C4 | 0.8377 | (14) | 0.5149 | (7) | 1.0942 | (5) | 0.0662 | (19) |
| H4 | 0.921462 | | 0.529666 | | 1.142477 | | 0.079* | |
| C14 | 0.8200 | (6) | 0.4966 | (3) | 0.6241 | (2) | 0.0220 | (8) |
| C22 | 0.0903 | (7) | 0.3222 | (3) | 0.4292 | (3) | 0.0245 | (8) |
| H22 | −0.000811 | | 0.275873 | | 0.401233 | | 0.029* | |
| C6 | 0.6072 | (9) | 0.4658 | (4) | 0.9587 | (3) | 0.0376 | (11) |
| C18 | 1.1860 | (11) | 0.7403 | (4) | 0.8303 | (4) | 0.0455 | (13) |

| | | | | |
|---|---|---|---|---|
| H18A | 1.132766 | 0.797822 | 0.797132 | 0.068* |
| H18B | 1.316419 | 0.760205 | 0.858796 | 0.068* |
| H18C | 1.081134 | 0.720683 | 0.872757 | 0.068* |

Atomic displacement parameters (Å²) for (cu_20221392_0 m)

| | U¹¹ | U²² | U³³ | U¹² | U¹³ | U²³ |
|---|---|---|---|---|---|---|
| O3 | 0.0277 (14) | 0.0267 (15) | 0.0317 (15) | −0.0066 (13) | 0.0016 (13) | −0.0018 (13) |
| O2 | 0.0398 (18) | 0.0347 (18) | 0.0417 (17) | −0.0114 (15) | 0.0064 (15) | −0.0164 (15) |
| C13 | 0.0234 (19) | 0.0206 (19) | 0.0218 (17) | 0.0054 (15) | −0.0037 (15) | 0.0048 (15) |
| O4 | 0.0293 (14) | 0.0171 (12) | 0.0294 (14) | 0.0038 (11) | −0.0063 (12) | −0.0025 (11) |
| O7 | 0.0313 (16) | 0.0240 (15) | 0.0376 (17) | 0.0001 (12) | −0.0104 (14) | 0.0053 (13) |
| O6 | 0.0277 (15) | 0.0223 (15) | 0.0382 (16) | −0.0018 (12) | −0.0066 (13) | 0.0062 (12) |
| C10 | 0.0239 (19) | 0.0177 (17) | 0.0236 (18) | 0.0039 (15) | −0.0013 (16) | 0.0004 (14) |
| O5 | 0.0240 (14) | 0.0162 (14) | 0.0293 (15) | 0.0000 (11) | −0.0085 (12) | 0.0000 (12) |
| O1 | 0.063 (3) | 0.051 (2) | 0.046 (2) | 0.000 (2) | −0.0123 (19) | −0.0057 (18) |
| C8 | 0.0206 (17) | 0.0231 (19) | 0.0181 (16) | 0.0018 (14) | −0.0041 (15) | −0.0013 (14) |
| N2 | 0.0238 (17) | 0.0285 (18) | 0.0305 (17) | −0.0012 (14) | −0.0001 (14) | −0.0030 (14) |
| C26 | 0.026 (2) | 0.032 (2) | 0.040 (2) | −0.0015 (19) | −0.0054 (18) | 0.0085 (19) |
| N1 | 0.040 (3) | 0.069 (3) | 0.043 (3) | −0.021 (2) | −0.008 (2) | 0.005 (2) |
| N4 | 0.036 (2) | 0.029 (2) | 0.039 (2) | −0.0047 (16) | −0.0051 (18) | −0.0051 (17) |
| C16 | 0.031 (2) | 0.026 (2) | 0.0284 (19) | −0.0031 (18) | −0.0072 (17) | −0.0020 (18) |
| C15 | 0.0177 (17) | 0.0232 (19) | 0.032 (2) | −0.0009 (15) | −0.0045 (16) | −0.0032 (15) |
| C11 | 0.0253 (18) | 0.0188 (17) | 0.031 (2) | 0.0049 (15) | −0.0055 (18) | −0.0013 (16) |
| C19 | 0.0191 (18) | 0.0213 (18) | 0.0223 (17) | 0.0009 (15) | −0.0027 (14) | −0.0010 (15) |
| C27 | 0.024 (2) | 0.035 (2) | 0.027 (2) | −0.0038 (17) | −0.0037 (17) | 0.0026 (17) |
| C24 | 0.023 (2) | 0.0220 (19) | 0.0262 (19) | 0.0017 (16) | −0.0033 (16) | −0.0001 (17) |
| C25 | 0.0250 (19) | 0.0241 (19) | 0.0268 (19) | 0.0050 (16) | −0.0029 (17) | 0.0025 (16) |
| C23 | 0.024 (2) | 0.025 (2) | 0.0233 (18) | 0.0029 (16) | −0.0066 (15) | −0.0007 (16) |
| C20 | 0.025 (2) | 0.0183 (17) | 0.0230 (18) | 0.0006 (15) | −0.0032 (15) | −0.0008 (15) |
| N3 | 0.0191 (15) | 0.0177 (15) | 0.0250 (16) | 0.0008 (13) | −0.0044 (13) | 0.0005 (12) |
| N5 | 0.0294 (19) | 0.031 (2) | 0.0307 (19) | −0.0033 (15) | −0.0091 (15) | 0.0017 (16) |
| C28 | 0.035 (3) | 0.047 (3) | 0.040 (2) | −0.007 (2) | −0.012 (2) | 0.005 (2) |
| C2 | 0.053 (4) | 0.051 (3) | 0.039 (3) | −0.005 (3) | −0.002 (2) | 0.001 (2) |
| C21 | 0.026 (2) | 0.0201 (19) | 0.026 (2) | −0.0006 (15) | −0.0047 (16) | −0.0015 (16) |
| C1 | 0.081 (5) | 0.068 (5) | 0.059 (4) | 0.023 (4) | 0.010 (4) | −0.014 (4) |
| C12 | 0.0239 (19) | 0.0225 (19) | 0.0222 (19) | −0.0012 (16) | −0.0035 (16) | −0.0014 (15) |
| C5 | 0.048 (3) | 0.067 (4) | 0.068 (4) | −0.002 (3) | −0.021 (3) | 0.015 (3) |
| C9 | 0.027 (2) | 0.027 (2) | 0.031 (2) | 0.0007 (15) | −0.0062 (19) | 0.0024 (17) |
| C3 | 0.060 (4) | 0.079 (5) | 0.036 (3) | −0.013 (4) | −0.008 (3) | −0.001 (3) |
| C7 | 0.0236 (19) | 0.039 (2) | 0.029 (2) | 0.0033 (18) | −0.0010 (16) | −0.008 (2) |
| N6 | 0.047 (3) | 0.084 (4) | 0.057 (3) | −0.020 (3) | −0.029 (3) | 0.015 (3) |
| C17 | 0.029 (2) | 0.031 (2) | 0.062 (3) | −0.0083 (19) | −0.001 (2) | −0.007 (2) |
| C4 | 0.072 (5) | 0.068 (4) | 0.059 (4) | −0.017 (4) | −0.004 (4) | −0.009 (3) |
| C14 | 0.0234 (19) | 0.0183 (19) | 0.0242 (18) | 0.0010 (14) | 0.0002 (15) | −0.0006 (16) |
| C22 | 0.024 (2) | 0.026 (2) | 0.0239 (19) | −0.0024 (17) | −0.0058 (17) | −0.0032 (16) |
| C6 | 0.046 (3) | 0.041 (3) | 0.026 (2) | −0.016 (2) | 0.001 (2) | 0.0069 (19) |
| C18 | 0.061 (4) | 0.034 (3) | 0.042 (3) | −0.006 (2) | 0.001 (2) | −0.013 (2) |

Geometric parameters (Å, °) for (cu_20221392_0m)

| O3—C8 | 1.236 (5) | C27—C28 | 1.434 | (7) |
|---|---|---|---|---|
| O2—C16 | 1.238 (6) | C24—H24 | 0.9500 | |
| C13—O5 | 1.401 (5) | C24—C23 | 1.384 | (6) |
| C13—C12 | 1.373 (6) | C25—C23 | 1.468 | (5) |
| C13—C14 | 1.463 (6) | C25—N5 | 1.278 | (6) |
| O4—C11 | 1.436 (5) | C23—C22 | 1.419 | (6) |
| O4—C12 | 1.337 (5) | C20—C21 | 1.403 | (6) |
| O7—C26 | 1.361 (6) | C20—C12 | 1.439 | (6) |
| O7—C25 | 1.364 (5) | N3—C14 | 1.369 | (5) |
| O6—C14 | 1.222 (5) | C28—N6 | 1.155 | (8) |
| C10—C8 | 1.552 (6) | C2—C3 | 1.405 | (9) |
| C10—C11 | 1.538 (6) | C2—C6 | 1.357 | (9) |
| C10—N3 | 1.479 (5) | C21—H21 | 0.9500 | |
| C10—C9 | 1.530 (6) | C21—C22 | 1.386 | (6) |
| O5—C19 | 1.375 (5) | C1—H1A | 0.9800 | |
| O1—C2 | 1.347 (9) | C1—H1B | 0.9800 | |
| O1—C1 | 1.426 (9) | C1—H1C | 0.9800 | |
| C8—N2 | 1.335 (6) | C5—H5 | 0.9500 | |
| N2—H2 | 0.8800 | C5—C4 | 1.389 | (13) |
| N2—C7 | 1.460 (6) | C9—H9A | 0.9800 | |
| C26—H26 | 0.9500 | C9—H9B | 0.9800 | |
| C26—C27 | 1.340 (7) | C9—H9C | 0.9800 | |
| N1—C5 | 1.304 (8) | C3—H3 | 0.9500 | |
| N1—C6 | 1.430 (9) | C3—C4 | 1.342 | (13) |
| N4—C16 | 1.331 (6) | C7—H7A | 0.9900 | |
| N4—C17 | 1.462 (7) | C7—H7B | 0.9900 | |
| N4—C18 | 1.456 (7) | C7—C6 | 1.511 | (6) |
| C16—C15 | 1.536 (6) | C17—H17A | 0.9800 | |
| C15—H15A | 0.9900 | C17—H17B | 0.9800 | |
| C15—H15B | 0.9900 | C17—H17C | 0.9800 | |
| C15—N3 | 1.471 (5) | C4—H4 | 0.9500 | |
| C11—H11A | 0.9900 | C22—H22 | 0.9500 | |
| C11—H11B | 0.9900 | C18—H18A | 0.9800 | |
| C19—C24 | 1.395 (6) | C18—H18B | 0.9800 | |
| C19—C20 | 1.382 (6) | C18—H18C | 0.9800 | |
| C27—N5 | 1.381 (6) | | | |
| O5—C13—C14 | 113.5 (3) | C14—N3—C15 | 111.8 | (4) |
| C12—C13—O5 | 110.1 (4) | C25—N5—C27 | 104.6 | (4) |
| C12—C13—C14 | 136.1 (4) | N6—C28—C27 | 177.2 | (7) |
| C12—O4—C11 | 118.1 (3) | O1—C2—C3 | 124.6 | (7) |
| C26—O7—C25 | 105.3 (4) | O1—C2—C6 | 113.3 | (5) |
| C11—C10—C8 | 111.1 (3) | C6—C2—C3 | 122.0 | (7) |
| N3—C10—C8 | 113.4 (3) | C20—C21—H21 | 121.3 | |
| N3—C10—C11 | 111.3 (3) | C22—C21—C20 | 117.4 | (4) |
| N3—C10—C9 | 110.5 (3) | C22—C21—H21 | 121.3 | |
| C9—C10—C8 | 104.0 (3) | O1—C1—H1A | 109.5 | |
| C9—C10—C11 | 106.0 (3) | O1—C1—H1B | 109.5 | |
| C19—O5—C13 | 105.4 (3) | O1—C1—H1C | 109.5 | |
| C2—O1—C1 | 115.9 (5) | H1A—C1—H1B | 109.5 | |
| O3—C8—C10 | 118.7 (3) | H1A—C1—H1C | 109.5 | |
| O3—C8—N2 | 125.7 (4) | H1B—C1—H1C | 109.5 | |
| N2—C8—C10 | 115.3 (4) | C13—C12—C20 | 107.4 | (4) |
| C8—N2—H2 | 119.2 | O4—C12—C13 | 130.7 | (4) |
| C8—N2—C7 | 121.5 (4) | O4—C12—C20 | 121.9 | (3) |
| C7—N2—H2 | 119.2 | N1—C5—H5 | 119.2 | |
| O7—C26—H26 | 126.7 | N1—C5—C4 | 121.6 | (7) |
| C27—C26—O7 | 106.6 (4) | C4—C5—H5 | 119.2 | |
| C27—C26—H26 | 126.7 | C10—C9—H9A | 109.5 | |
| C5—N1—C6 | 116.6 (6) | C10—C9—H9B | 109.5 | |
| C16—N4—C17 | 124.8 (4) | C10—C9—H9C | 109.5 | |
| C16—N4—C18 | 118.9 (5) | H9A—C9—H9B | 109.5 | |
| C18—N4—C17 | 116.2 (4) | H9A—C9—H9C | 109.5 | |
| O2—C16—N4 | 122.0 (4) | H9B—C9—H9C | 109.5 | |
| O2—C16—C15 | 119.9 (4) | C2—C3—H3 | 122.9 | |
| N4—C16—C15 | 118.1 (4) | C4—C3—C2 | 114.1 | (7) |
| C16—C15—H15A | 109.2 | C4—C3—H3 | 122.9 | |
| C16—C15—H15B | 109.2 | N2—C7—H7A | 109.8 | |
| H15A—C15—H15B | 107.9 | N2—C7—H7B | 109.8 | |
| N3—C15—C16 | 111.9 (3) | N2—C7—C6 | 109.6 | (4) |
| N3—C15—H15A | 109.2 | H7A—C7—H7B | 108.2 | |
| N3—C15—H15B | 109.2 | C6—C7—H7A | 109.8 | |
| O4—C11—C10 | 117.9 (3) | C6—C7—H7B | 109.8 | |
| O4—C11—H11A | 107.8 | N4—C17—H17A | 109.5 | |
| O4—C11—H11B | 107.8 | N4—C17—H17B | 109.5 | |
| C10—C11—H11A | 107.8 | N4—C17—H17C | 109.5 | |
| C10—C11—H11B | 107.8 | H17A—C17—H17B | 109.5 | |
| H11A—C11—H11B | 107.2 | H17A—C17—H17C | 109.5 | |
| O5—C19—C24 | 124.0 (4) | H17B—C17—H17C | 109.5 | |
| O5—C19—C20 | 112.0 (4) | C5—C4—H4 | 117.7 | |
| C20—C19—C24 | 124.0 (4) | C3—C4—C5 | 124.7 | (7) |
| C26—C27—N5 | 110.2 (4) | C3—C4—H4 | 117.7 | |
| C26—C27—C28 | 126.7 (5) | O6—C14—C13 | 119.7 | (4) |
| N5—C27—C28 | 123.1 (4) | O6—C14—N3 | 122.1 | (4) |
| C19—C24—H24 | 122.3 | N3—C14—C13 | 118.2 | (4) |
| C23—C24—C19 | 115.4 (4) | C23—C22—H22 | 119.5 | |
| C23—C24—H24 | 122.3 | C21—C22—C23 | 120.9 | (4) |
| O7—C25—C23 | 117.5 (4) | C21—C22—H22 | 119.5 | |
| N5—C25—O7 | 113.2 (4) | N1—C6—C7 | 114.2 | (5) |
| N5—C25—C23 | 129.3 (4) | C2—C6—N1 | 120.8 | (5) |
| C24—C23—C25 | 119.8 (4) | C2—C6—C7 | 124.9 | (5) |
| C24—C23—C22 | 122.1 (4) | N4—C18—H18A | 109.5 | |
| C22—C23—C25 | 118.1 (4) | N4—C18—H18B | 109.5 | |
| C19—C20—C21 | 120.1 (4) | N4—C18—H18C | 109.5 | |
| C19—C20—C12 | 105.0 (3) | H18A—C18—H18B | 109.5 | |
| C21—C20—C12 | 134.9 (4) | H18A—C18—H18C | 109.5 | |
| C15—N3—C10 | 125.1 (3) | H18B—C18—H18C | 109.5 | |
| C14—N3—C10 | 123.1 (3) | | | |

Bioactivity of steroisomers

| Example ID | | | Bioactivity Bucket | | |
|---|---|---|---|---|---|
| Racemate | S | R | Racemate | S | R |
| 130 | | 129 | C | inactive | C |
| | 49 | 50 | B | A | C |
| 81 | 135 | 79 | B | inactive | B |
| 106 | | 107 | B | inactive | B |
| not made | | 102 | n/a | inactive | B |
| 15 | | | A | inactive | B |
| | 136 | 7 | A | inactive | B |
| 15 | | | A | inactive | A |
| 8 | 137 | 40 | A | inactive | A |
| 5 | 138 | 6 | A | inactive | A |

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A compound represented by formula I: 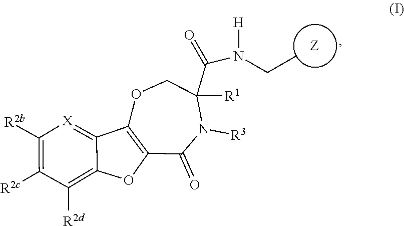
or a pharmaceutically acceptable salt thereof, wherein:
R¹ represents lower alkyl;
X represents C—R^{2a} or N;
R^{2a}, R^{2b}, R^{2c}, and R^{2d} each independently represent hydrogen, alkyl, alkenyl, alkynyl, halo, aryl, heteroaryl, cycloalkyl, heterocyclyl, cyano, acyl, carboxy, ester, or amido;
R³ represents alkyl, alkenyl, alkynyl, aryl, heteroaryl, cycloalkyl, heterocyclyl, arylalkyl, heteroaralkyl, (cycloalkyl)alkyl, heterocyclylalkyl, amidoalkyl, alkoxyalkyl, or acylalkyl; and
Z represents a substituted or unsubstituted aryl or heteroaryl ring, e.g., optionally substituted with one or more groups chosen from alkyl, alkenyl, alkynyl, cyano, acyl, carboxy, ester, amido, alkoxy, and halo,
provided the compound is not:

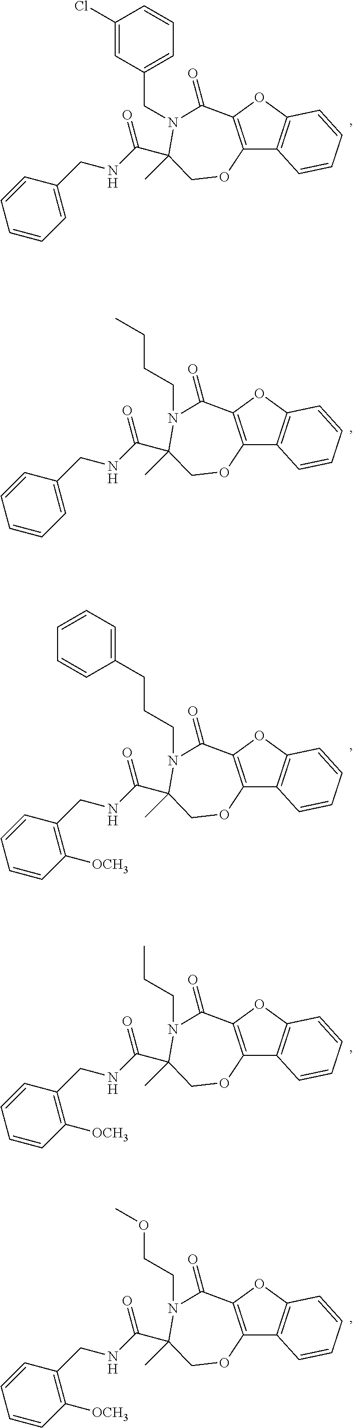 [SMILES: CC1(C(=O)NCc2ccccc2)COc2c(oc3ccccc23)C(=O)N1Cc1cccc(Cl)c1 ; CCCCN1C(=O)c2oc3ccccc3c2OCC1(C)C(=O)NCc1ccccc1 ; COc1ccccc1CNC(=O)C1(C)COc2c(oc3ccccc23)C(=O)N1CCCc1ccccc1 ; CCCN1C(=O)c2oc3ccccc3c2OCC1(C)C(=O)NCc1ccccc1OC ; COCCN1C(=O)c2oc3ccccc3c2OCC1(C)C(=O)NCc1ccccc1OC]

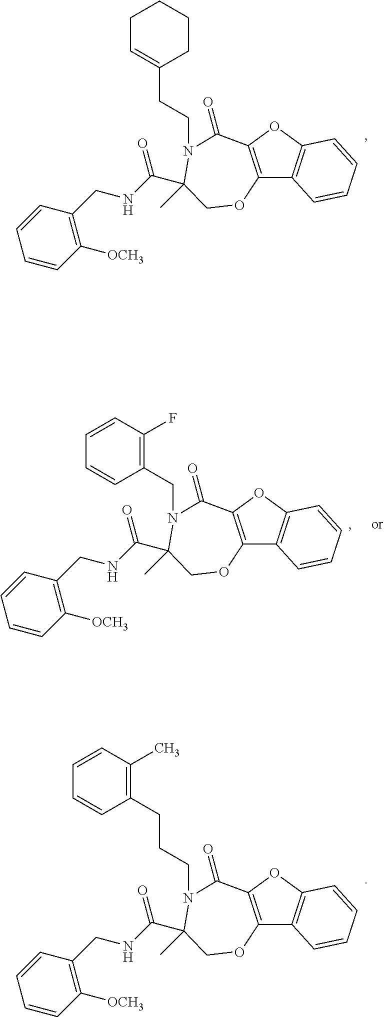 [SMILES: COc1ccccc1CNC(=O)C1(C)COc2c(oc3ccccc23)C(=O)N1CCC1=CCCCC1 ; COc1ccccc1CNC(=O)C1(C)COc2c(oc3ccccc23)C(=O)N1Cc1ccccc1F ; COc1ccccc1CNC(=O)C1(C)COc2c(oc3ccccc23)C(=O)N1CCCc1ccccc1C]

2. The compound of claim 1, wherein R¹ is methyl.

3. The compound of claim 1 or 2, wherein X is N.

4. The compound of claim 1 or 2, wherein X is C—R^{2a}.

5. The compound of any one of claims 1 to 4, wherein R^{2a}, R^{2b}, R^{2c}, and R^{2d} independently represent hydrogen, methyl, propenyl, chloro, fluoro, haloalkyl (e.g., trifluoromethyl), a five-membered heteroaryl, cyclopropyl, or amido having the structure: 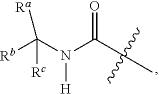
wherein R^{a} is hydrogen or alkyl, and R^{b} and R^{c} taken together form a cycloalkyl or heterocyclyl.

6. The compound of claim 5, wherein R^{b} and R^{c} taken together form cyclobutyl.

7. The compound of claim 5 or 6, wherein R^{a} is hydrogen or methyl.

8. The compound of claim 5, wherein R^{b} and R^{c} taken together form oxetane.

9. The compound of claim 8, wherein R^{a} is methyl.

10. The compound of claim 5, wherein at least one of R^{2a}, R^{2b}, R^{2c}, and R^{2d} is 5-membered heteroaryl.

11. The compound of claim 10, wherein the 5-membered heteroaryl is thiazolyl or oxazolyl, optionally substituted with trifluoromethyl, chloro, or cyano.

12. The compound of claim 10, wherein the 5-membered heteroaryl is oxazol-2-yl.

13. The compound of claim 10, wherein the 5-membered heteroaryl is 4-cyanooxazol-2-yl.

14. The compound of any one of claims 1 to 13, wherein R^{2a} is hydrogen.

15. The compound of claims 1 to 14, wherein R^{2b} and R^{2d} are each hydrogen.

16. The compound of claims 1 to 14, wherein R^{2c} and R^{2d} are each hydrogen.

17. The compound of any one of claims 1 to 14, wherein R^{2b}, R^{2c}, and R^{2d} are each hydrogen.

18. The compound of any one of claims 1 to 4, wherein R^{2a}, R^{2b}, R^{2c}, and R^{2d} are each hydrogen.

19. The compound of any one of claims 1 to 18, wherein R³ is amidoalkyl.

20. The compound of claim 19, wherein the amidoalkyl has the structure: 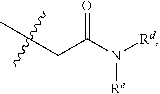
wherein R^{d} and R^{e} are independently chosen from alkyl or hydroxyalkyl, or R^{d} and R^{e} taken together form a heterocyclic ring.

21. The compound of claim 20, wherein R^{d} and R^{e} are each methyl.

22. The compound of claim 21, wherein R^{d} and R^{e} are independently substituted with one or more deuterium atoms.

23. The compound of claim 22, wherein R^{d} is methyl and R^{e} is —(CH₂)₂OH.

24. The compound of claim 23, wherein R^{d} and R^{e} taken together with the nitrogen to which they are attached form an azetidine optionally substituted with one or more halo, hydroxyl, or hydroxyalkyl.

25. The compound of claim 24, wherein the azetidine is:

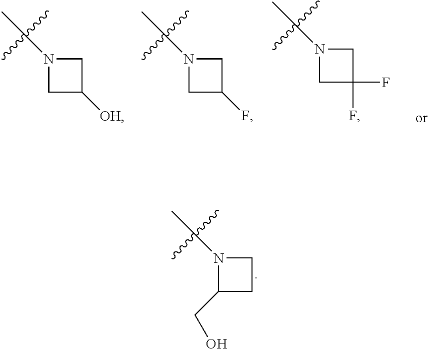 [SMILES: *N1CC(O)C1 ; *N1CC(F)C1 ; *N1CC(F)(F)C1 ; *N1CCC1CO]

26. The compound of any one of claims 1 to 18, wherein R³ is C₃-C₆ cycloalkyl.

27. The compound of claim 26, wherein R³ is:

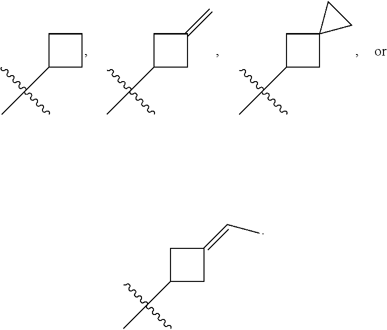

28. The compound of any one of claims 1 to 18, wherein R³ represents C₁-C₆ alkyl, C₂-C₆ alkenyl, or C₂-C₆ alkynyl, optionally substituted with alkoxy.

29. The compound of claim 28, wherein R³ is:

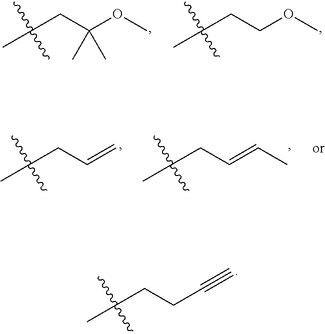

30. The compound of any one of claims 1 to 18, wherein R³ is —CH₂-cycloalkyl optionally substituted with halo, alkoxy, or hydroxyl.

31. The compound of claim 30, wherein R³ is:

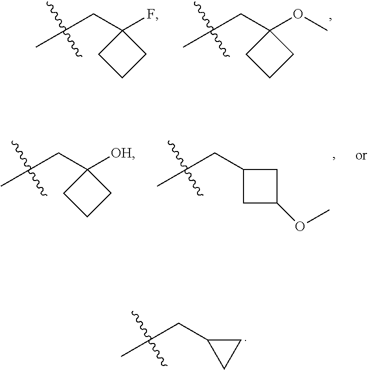

32. The compound of any one of claims 1 to 18, wherein R³ is acylalkyl having the structure: 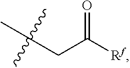
wherein R^{f} represents alkyl or cycloalkyl.

33. The compound of claim 32, wherein R^{f} is ethyl or cyclopropyl.

34. The compound of any one of claims 1 to 18, wherein R³ represents —(CH₂)₁₋₃-heteroaryl, optionally substituted with alkyl, hydroxyalkyl or alkoxyalkoxyalkyl.

35. The compound of claim 34, wherein the heteroaryl is tetrazole, 1,2,3-triazole, or 1,2,4-triazole.

36. The compound of claim 35, wherein R³ is:

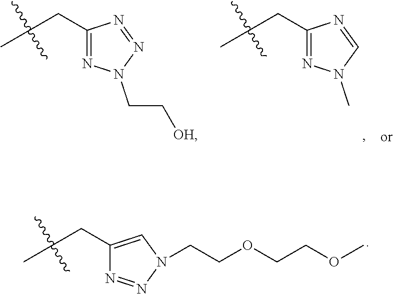

37. The compound of any one of claims 1 to 36, wherein Z represents phenyl, pyridinyl, naphthyl, isoquinolinyl, or quinolinyl, each of which is optionally substituted with one or more groups chosen from lower alkyl, lower alkoxy, halo, haloalkoxy, amido, and cyano.

38. The compound of claim 37, wherein Z is substituted with one or more groups chosen from methoxy, isopropyloxy, chloro, fluoro, trifluoromethoxy, cyano, and carbamoyl.

39. The compound of claim 37 or 38, wherein Z is mono-, di-, or trisubstituted.

40. The compound of claim 37 or 38, wherein Z is phenyl substituted with methoxy and at least one additional substitutent.

41. The compound of any one of claims 1 to 40, wherein Z represents:

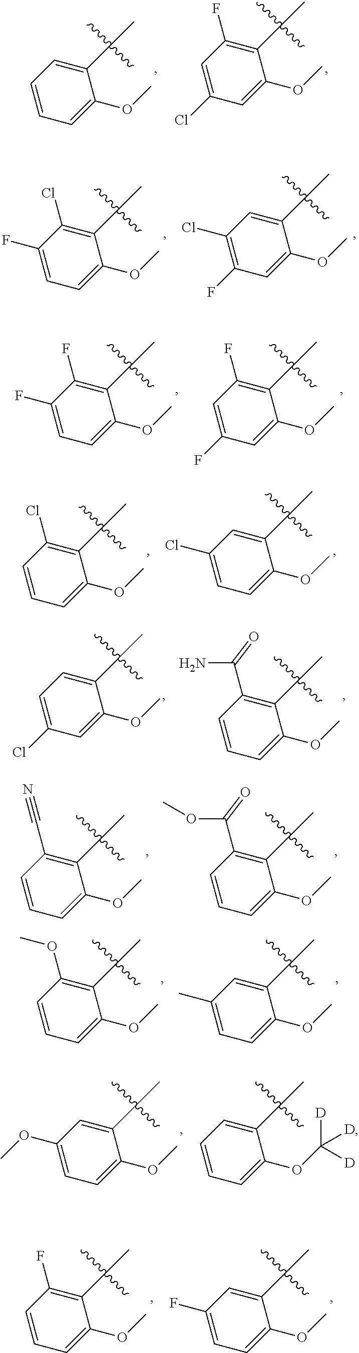

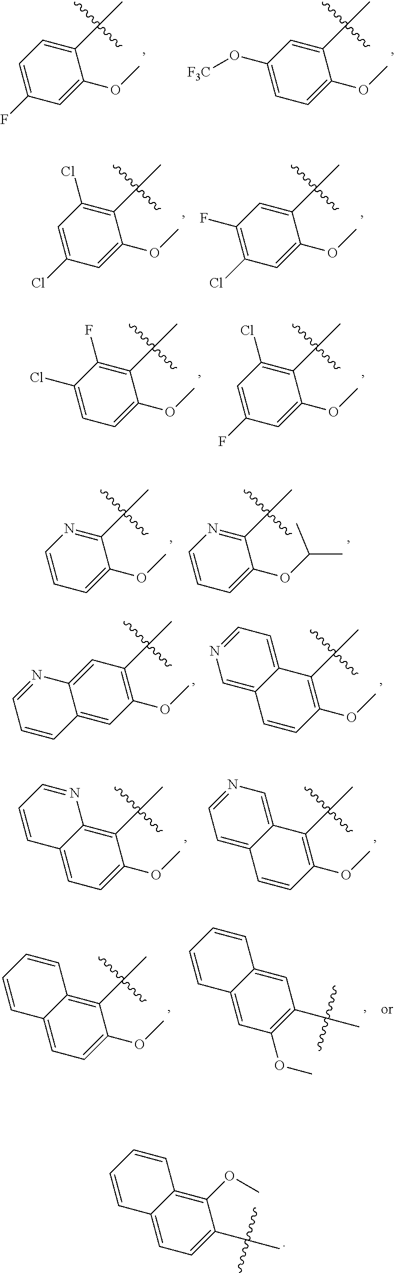

42. The compound of claim 1, wherein the compound is chosen from: 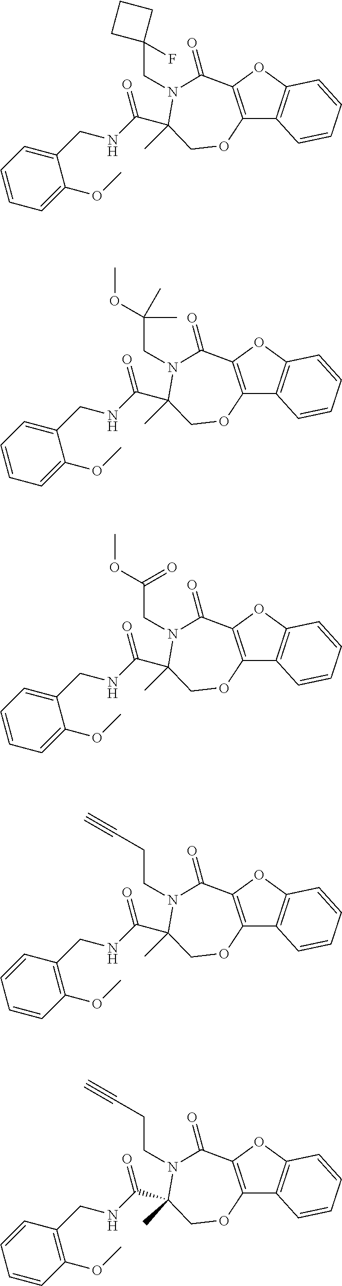 [SMILES: COc1ccccc1CNC(=O)C1(C)COc2c(oc3ccccc23)C(=O)N1CC1(F)CCC1 ; COc1ccccc1CNC(=O)C1(C)COc2c(oc3ccccc23)C(=O)N1CC(C)(C)OC ; COC(=O)CN1C(=O)c2oc3ccccc3c2OCC1(C)C(=O)NCc1ccccc1OC ; C#CCCN1C(=O)c2oc3ccccc3c2OCC1(C)C(=O)NCc1ccccc1OC ; C#CCCN1C(=O)c2oc3ccccc3c2OC[C@]1(C)C(=O)NCc1ccccc1OC] 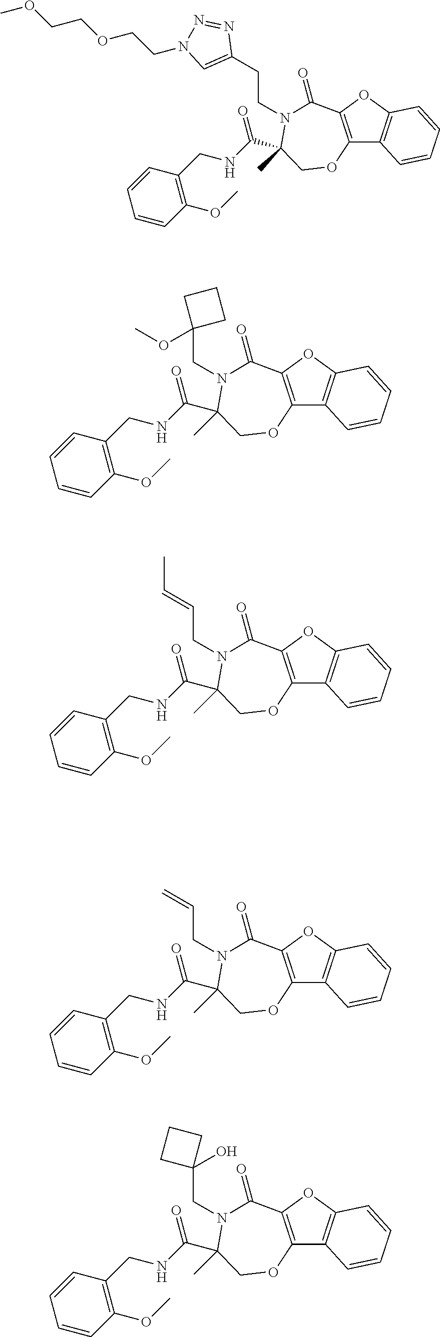 [SMILES: COCCOCCn1cc(CCN2C(=O)c3oc4ccccc4c3OC[C@]2(C)C(=O)NCc2ccccc2OC)nn1 ; COc1ccccc1CNC(=O)C1(C)COc2c(oc3ccccc23)C(=O)N1CC1(OC)CCC1 ; C/C=C/CN1C(=O)c2oc3ccccc3c2OCC1(C)C(=O)NCc1ccccc1OC ; C=CCN1C(=O)c2oc3ccccc3c2OCC1(C)C(=O)NCc1ccccc1OC ; COc1ccccc1CNC(=O)C1(C)COc2c(oc3ccccc23)C(=O)N1CC1(O)CCC1] 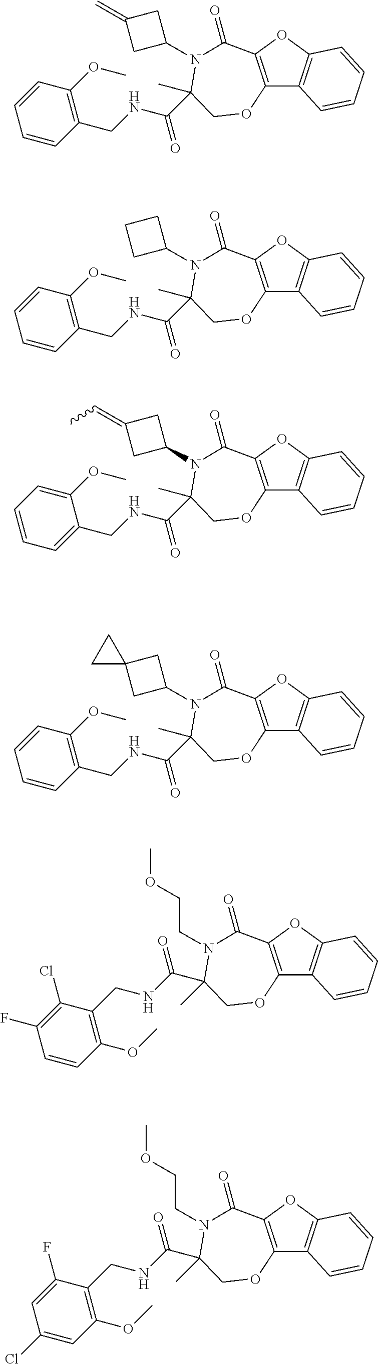 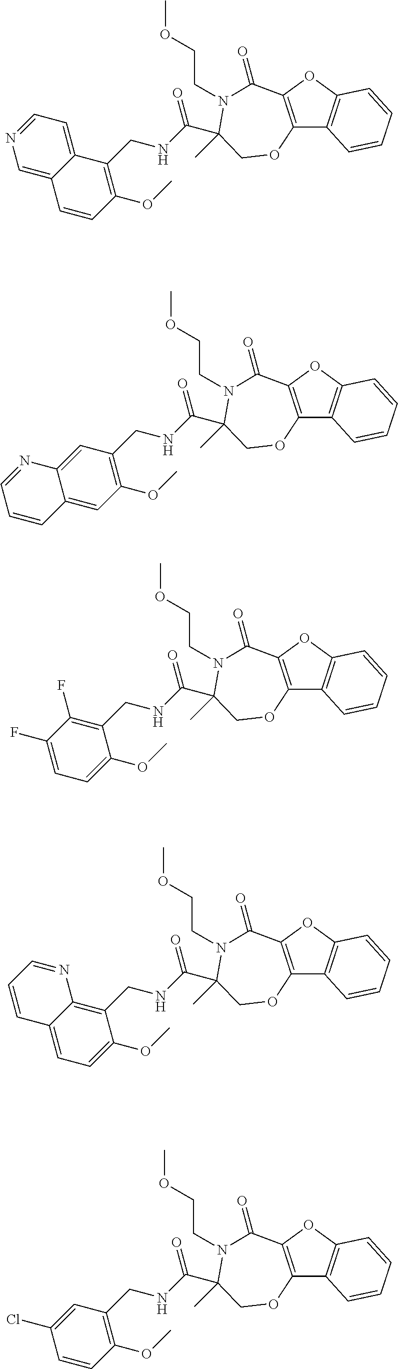 [SMILES: COCCN1C(=O)c2oc3ccccc3c2OCC1(C)C(=O)NCc1c(OC)ccc2cnccc12 ; COCCN1C(=O)c2oc3ccccc3c2OCC1(C)C(=O)NCc1cc2ncccc2cc1OC ; COCCN1C(=O)c2oc3ccccc3c2OCC1(C)C(=O)NCc1c(OC)ccc(F)c1F ; COCCN1C(=O)c2oc3ccccc3c2OCC1(C)C(=O)NCc1c(OC)ccc2cccnc12 ; COCCN1C(=O)c2oc3ccccc3c2OCC1(C)C(=O)NCc1cc(Cl)ccc1OC] 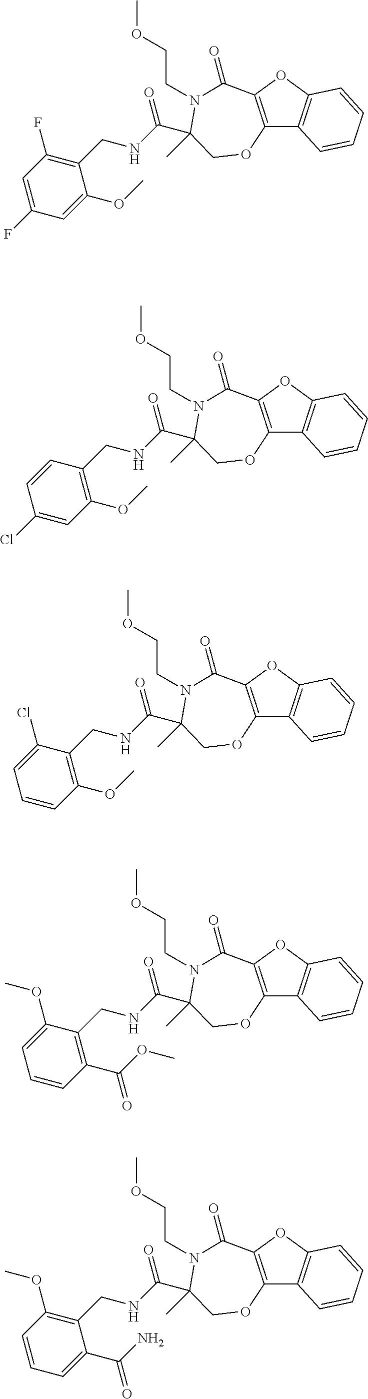 [SMILES: COCCN1C(=O)c2oc3ccccc3c2OCC1(C)C(=O)NCc1c(F)cc(F)cc1OC ; COCCN1C(=O)c2oc3ccccc3c2OCC1(C)C(=O)NCc1ccc(Cl)cc1OC ; COCCN1C(=O)c2oc3ccccc3c2OCC1(C)C(=O)NCc1c(Cl)cccc1OC ; COCCN1C(=O)c2oc3ccccc3c2OCC1(C)C(=O)NCc1c(OC)cccc1C(=O)OC ; COCCN1C(=O)c2oc3ccccc3c2OCC1(C)C(=O)NCc1c(OC)cccc1C(N)=O] 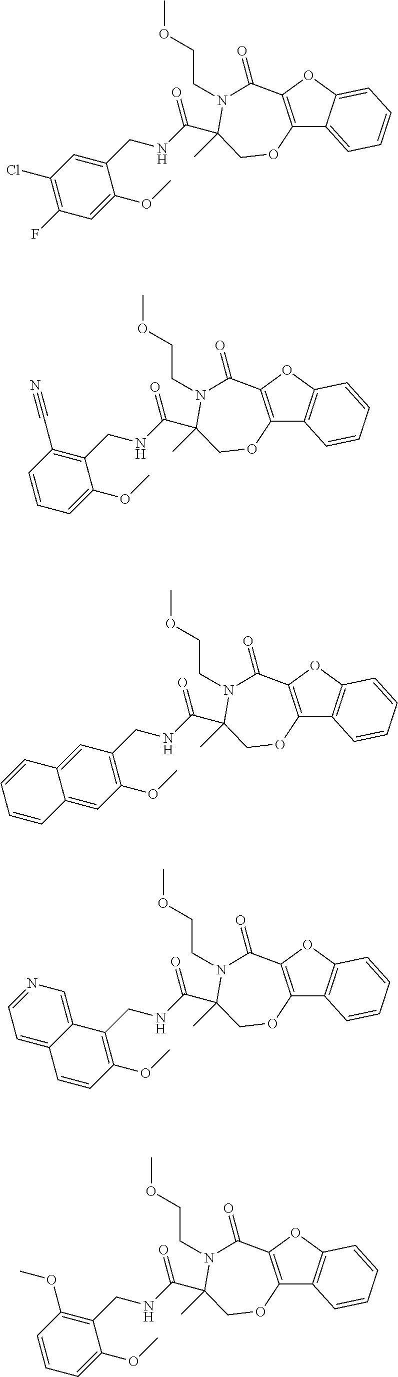 [SMILES: COCCN1C(=O)c2oc3ccccc3c2OCC1(C)C(=O)NCc1cc(Cl)c(F)cc1OC ; COCCN1C(=O)c2oc3ccccc3c2OCC1(C)C(=O)NCc1c(C#N)cccc1OC ; COCCN1C(=O)c2oc3ccccc3c2OCC1(C)C(=O)NCc1cc2ccccc2cc1OC ; COCCN1C(=O)c2oc3ccccc3c2OCC1(C)C(=O)NCc1c(OC)ccc2ccncc12 ; COCCN1C(=O)c2oc3ccccc3c2OCC1(C)C(=O)NCc1c(OC)cccc1OC] 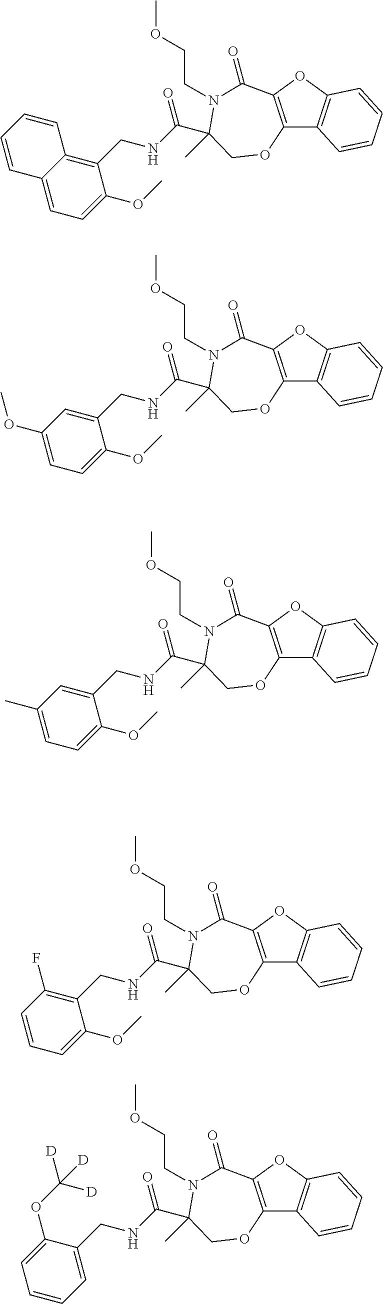 [SMILES: COCCN1C(=O)c2oc3ccccc3c2OCC1(C)C(=O)NCc1c(OC)ccc2ccccc12 ; COCCN1C(=O)c2oc3ccccc3c2OCC1(C)C(=O)NCc1cc(OC)ccc1OC ; COCCN1C(=O)c2oc3ccccc3c2OCC1(C)C(=O)NCc1cc(C)ccc1OC ; COCCN1C(=O)c2oc3ccccc3c2OCC1(C)C(=O)NCc1c(F)cccc1OC ; [2H]C([2H])([2H])Oc1ccccc1CNC(=O)C1(C)COc2c(oc3ccccc23)C(=O)N1CCOC] 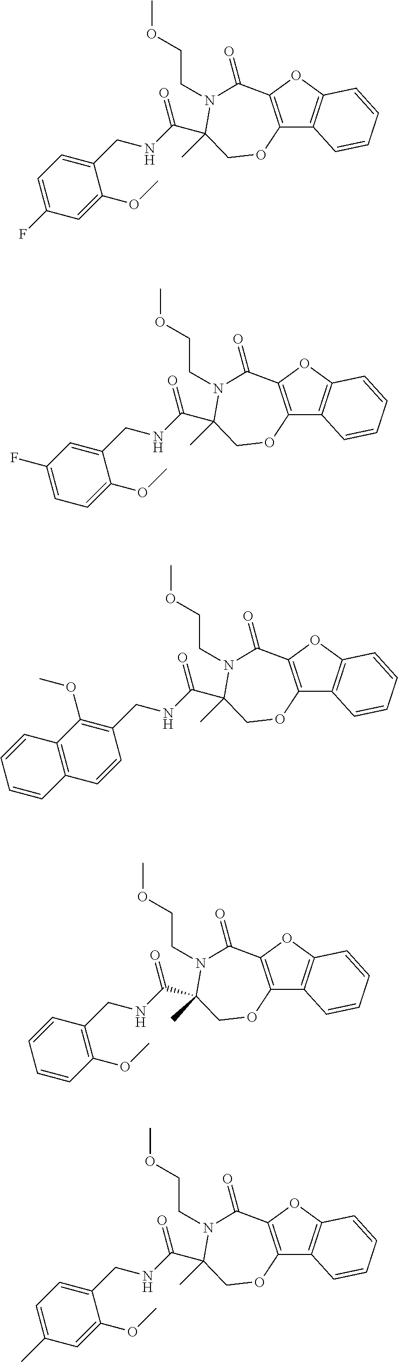 [SMILES: COCCN1C(=O)c2oc3ccccc3c2OCC1(C)C(=O)NCc1ccc(F)cc1OC ; COCCN1C(=O)c2oc3ccccc3c2OCC1(C)C(=O)NCc1cc(F)ccc1OC ; COCCN1C(=O)c2oc3ccccc3c2OCC1(C)C(=O)NCc1ccc2ccccc2c1OC ; COCCN1C(=O)c2oc3ccccc3c2OC[C@]1(C)C(=O)NCc1ccccc1OC ; COCCN1C(=O)c2oc3ccccc3c2OCC1(C)C(=O)NCc1ccc(C)cc1OC] 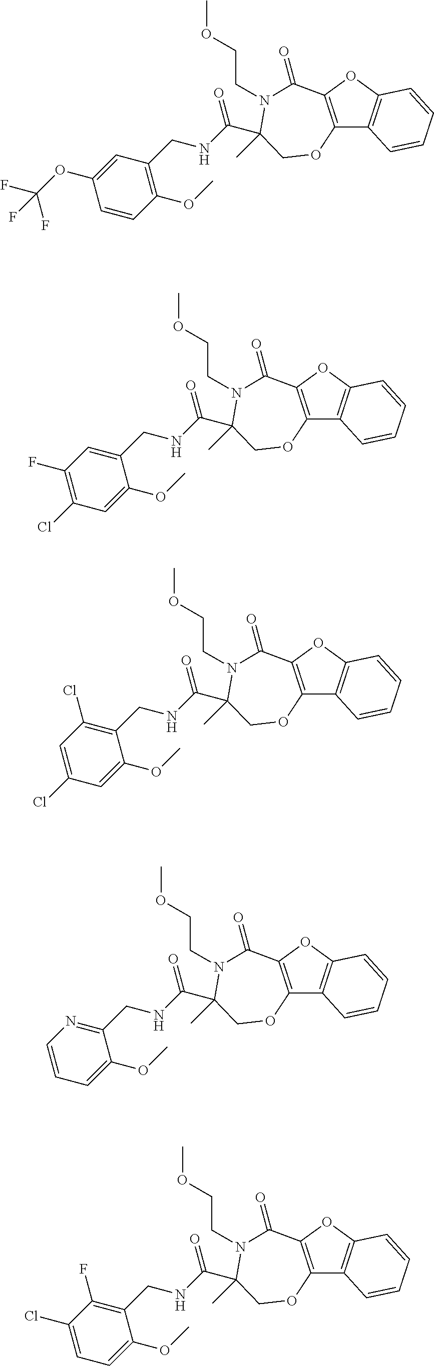 [SMILES: COCCN1C(=O)c2oc3ccccc3c2OCC1(C)C(=O)NCc1cc(OC(F)(F)F)ccc1OC ; COCCN1C(=O)c2oc3ccccc3c2OCC1(C)C(=O)NCc1cc(F)c(Cl)cc1OC ; COCCN1C(=O)c2oc3ccccc3c2OCC1(C)C(=O)NCc1c(Cl)cc(Cl)cc1OC ; COCCN1C(=O)c2oc3ccccc3c2OCC1(C)C(=O)NCc1ncccc1OC ; COCCN1C(=O)c2oc3ccccc3c2OCC1(C)C(=O)NCc1c(OC)ccc(Cl)c1F] 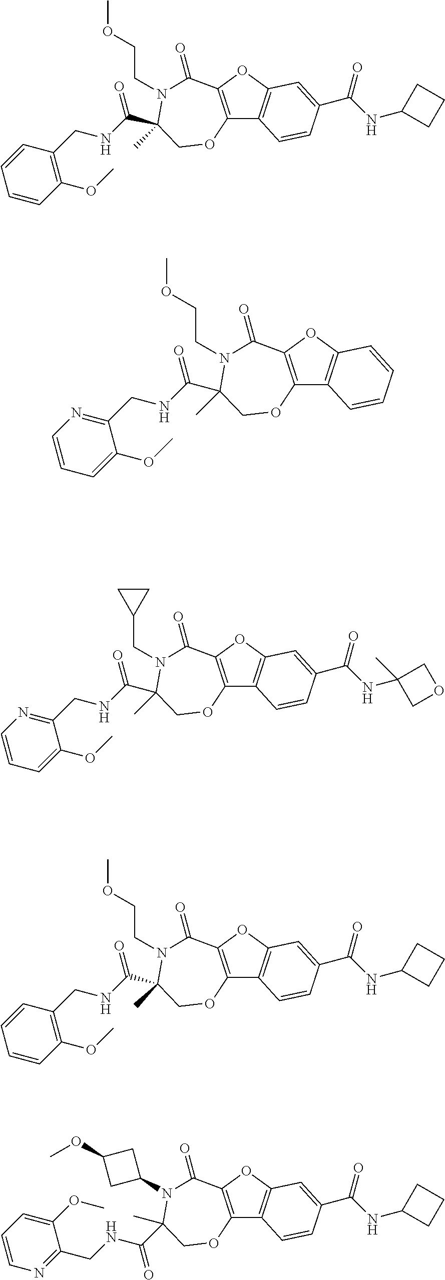 [SMILES: COCCN1C(=O)c2oc3cc(C(=O)NC4CCC4)ccc3c2OC[C@@]1(C)C(=O)NCc1ccccc1OC ; COCCN1C(=O)c2oc3ccccc3c2OCC1(C)C(=O)NCc1ncccc1OC ; COc1cccnc1CNC(=O)C1(C)COc2c(oc3cc(C(=O)NC4(C)COC4)ccc23)C(=O)N1CC1CC1 ; COCCN1C(=O)c2oc3cc(C(=O)NC4CCC4)ccc3c2OC[C@]1(C)C(=O)NCc1ccccc1OC ; COc1cccnc1CNC(=O)C1(C)COc2c(oc3cc(C(=O)NC4CCC4)ccc23)C(=O)N1[C@H]1C[C@@H](OC)C1] 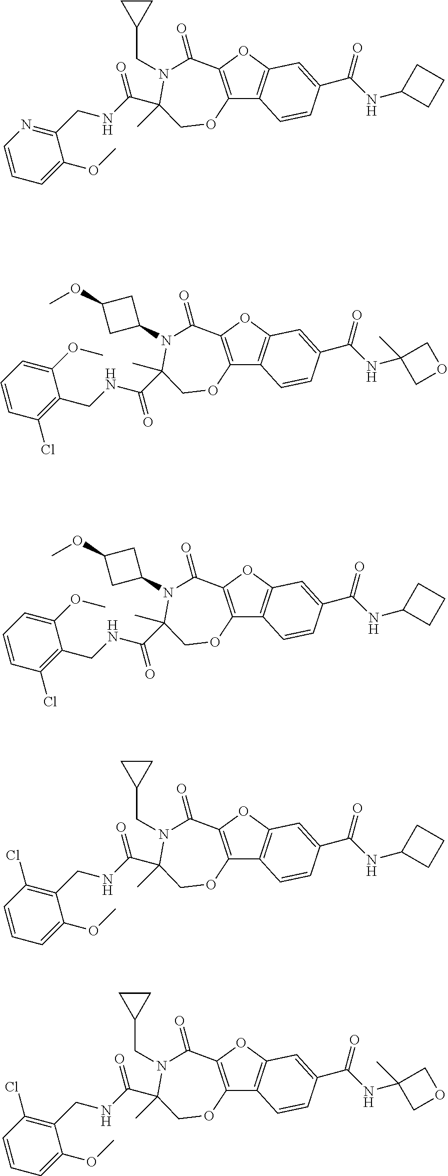 [SMILES: COc1cccnc1CNC(=O)C1(C)COc2c(oc3cc(C(=O)NC4CCC4)ccc23)C(=O)N1CC1CC1 ; COc1cccc(Cl)c1CNC(=O)C1(C)COc2c(oc3cc(C(=O)NC4(C)COC4)ccc23)C(=O)N1[C@H]1C[C@@H](OC)C1 ; COc1cccc(Cl)c1CNC(=O)C1(C)COc2c(oc3cc(C(=O)NC4CCC4)ccc23)C(=O)N1[C@H]1C[C@@H](OC)C1 ; COc1cccc(Cl)c1CNC(=O)C1(C)COc2c(oc3cc(C(=O)NC4CCC4)ccc23)C(=O)N1CC1CC1 ; COc1cccc(Cl)c1CNC(=O)C1(C)COc2c(oc3cc(C(=O)NC4(C)COC4)ccc23)C(=O)N1CC1CC1] 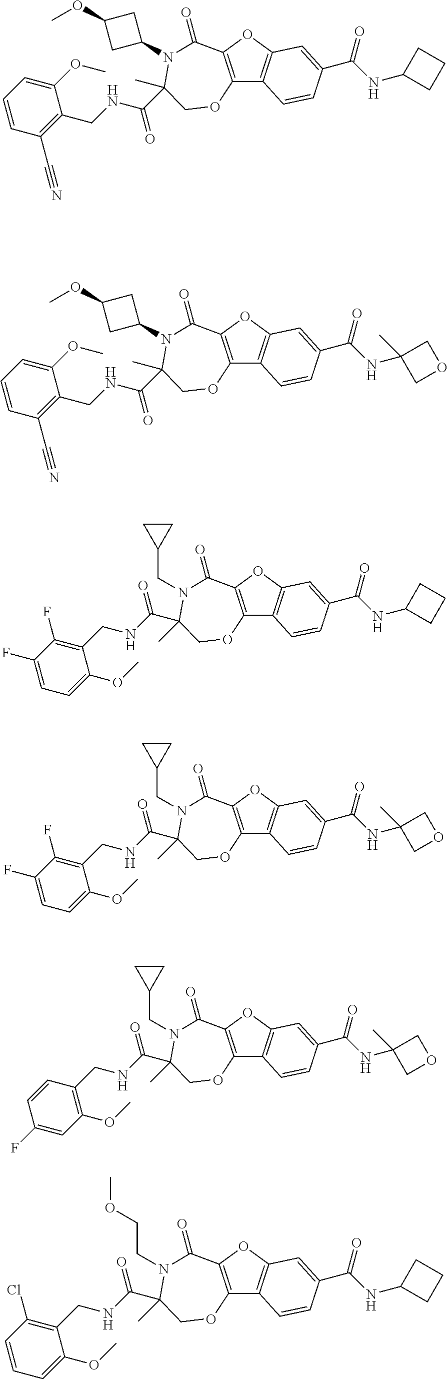 [SMILES: COc1cccc(C#N)c1CNC(=O)C1(C)COc2c(oc3cc(C(=O)NC4CCC4)ccc23)C(=O)N1[C@H]1C[C@@H](OC)C1 ; COc1cccc(C#N)c1CNC(=O)C1(C)COc2c(oc3cc(C(=O)NC4(C)COC4)ccc23)C(=O)N1[C@H]1C[C@@H](OC)C1 ; COc1ccc(F)c(F)c1CNC(=O)C1(C)COc2c(oc3cc(C(=O)NC4CCC4)ccc23)C(=O)N1CC1CC1 ; COc1ccc(F)c(F)c1CNC(=O)C1(C)COc2c(oc3cc(C(=O)NC4(C)COC4)ccc23)C(=O)N1CC1CC1 ; COc1cc(F)ccc1CNC(=O)C1(C)COc2c(oc3cc(C(=O)NC4(C)COC4)ccc23)C(=O)N1CC1CC1 ; COCCN1C(=O)c2oc3cc(C(=O)NC4CCC4)ccc3c2OCC1(C)C(=O)NCc1c(Cl)cccc1OC] 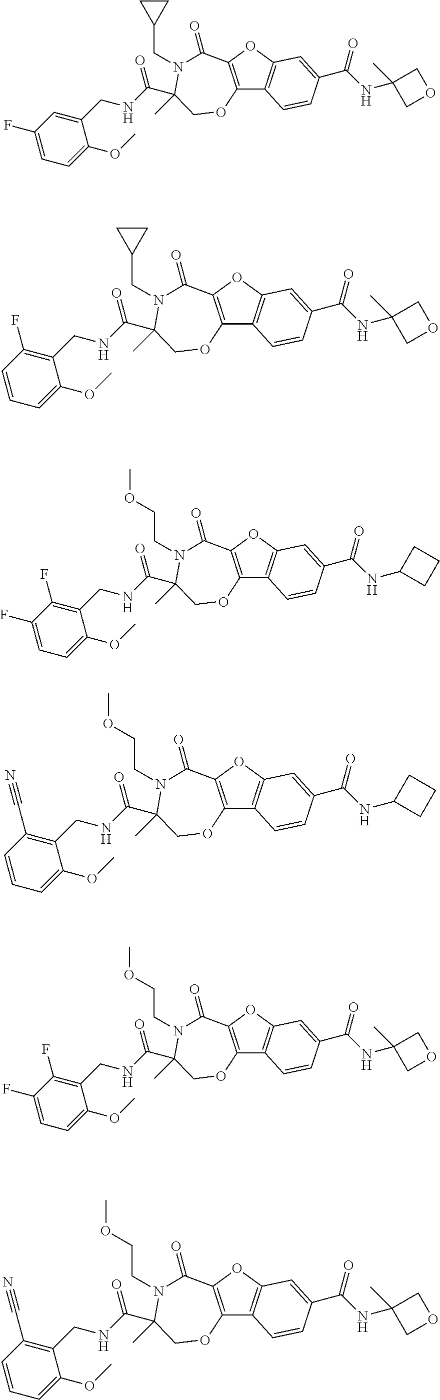 [SMILES: COc1ccc(F)cc1CNC(=O)C1(C)COc2c(oc3cc(C(=O)NC4(C)COC4)ccc23)C(=O)N1CC1CC1 ; COc1cccc(F)c1CNC(=O)C1(C)COc2c(oc3cc(C(=O)NC4(C)COC4)ccc23)C(=O)N1CC1CC1 ; COCCN1C(=O)c2oc3cc(C(=O)NC4CCC4)ccc3c2OCC1(C)C(=O)NCc1c(OC)ccc(F)c1F ; COCCN1C(=O)c2oc3cc(C(=O)NC4CCC4)ccc3c2OCC1(C)C(=O)NCc1c(C#N)cccc1OC ; COCCN1C(=O)c2oc3cc(C(=O)NC4(C)COC4)ccc3c2OCC1(C)C(=O)NCc1c(OC)ccc(F)c1F ; COCCN1C(=O)c2oc3cc(C(=O)NC4(C)COC4)ccc3c2OCC1(C)C(=O)NCc1c(C#N)cccc1OC] 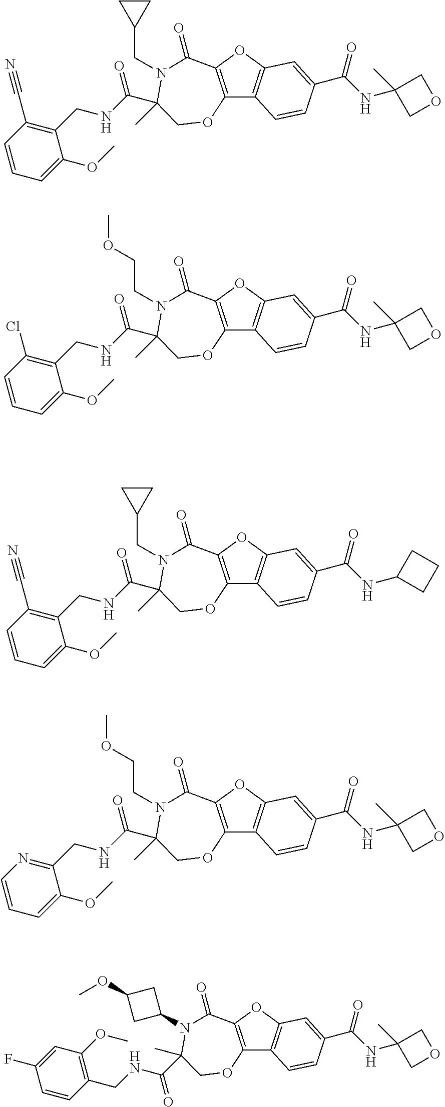 [SMILES: COc1cccc(C#N)c1CNC(=O)C1(C)COc2c(oc3cc(C(=O)NC4(C)COC4)ccc23)C(=O)N1CC1CC1 ; COCCN1C(=O)c2oc3cc(C(=O)NC4(C)COC4)ccc3c2OCC1(C)C(=O)NCc1c(Cl)cccc1OC ; COc1cccc(C#N)c1CNC(=O)C1(C)COc2c(oc3cc(C(=O)NC4CCC4)ccc23)C(=O)N1CC1CC1 ; COCCN1C(=O)c2oc3cc(C(=O)NC4(C)COC4)ccc3c2OCC1(C)C(=O)NCc1ncccc1OC ; COc1cc(F)ccc1CNC(=O)C1(C)COc2c(oc3cc(C(=O)NC4(C)COC4)ccc23)C(=O)N1[C@H]1C[C@@H](OC)C1] 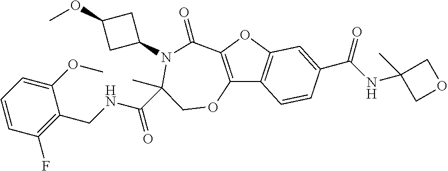 [SMILES: COc1cccc(F)c1CNC(=O)C1(C)COc2c(oc3cc(C(=O)NC4(C)COC4)ccc23)C(=O)N1[C@H]1C[C@@H](OC)C1] 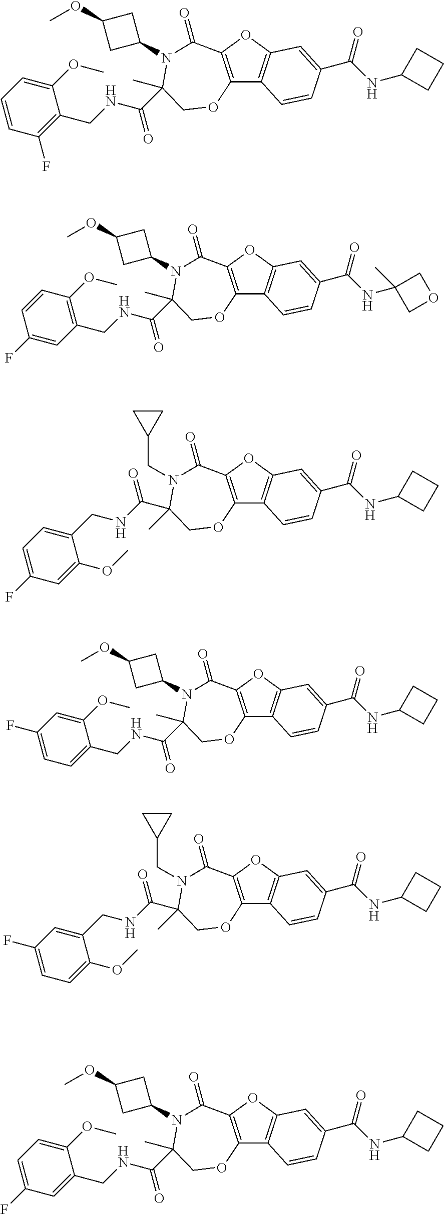 [SMILES: COc1cccc(F)c1CNC(=O)C1(C)COc2c(oc3cc(C(=O)NC4CCC4)ccc23)C(=O)N1[C@H]1C[C@@H](OC)C1 ; COc1ccc(F)cc1CNC(=O)C1(C)COc2c(oc3cc(C(=O)NC4(C)COC4)ccc23)C(=O)N1[C@H]1C[C@@H](OC)C1 ; COc1cc(F)ccc1CNC(=O)C1(C)COc2c(oc3cc(C(=O)NC4CCC4)ccc23)C(=O)N1CC1CC1 ; COc1cc(F)ccc1CNC(=O)C1(C)COc2c(oc3cc(C(=O)NC4CCC4)ccc23)C(=O)N1[C@H]1C[C@@H](OC)C1 ; COc1ccc(F)cc1CNC(=O)C1(C)COc2c(oc3cc(C(=O)NC4CCC4)ccc23)C(=O)N1CC1CC1 ; COc1ccc(F)cc1CNC(=O)C1(C)COc2c(oc3cc(C(=O)NC4CCC4)ccc23)C(=O)N1[C@H]1C[C@@H](OC)C1] 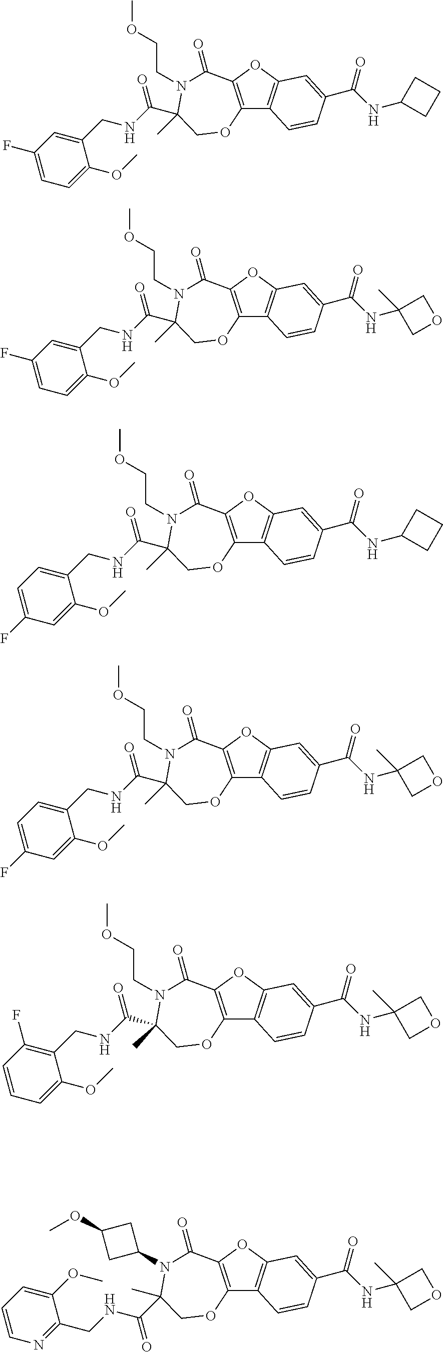 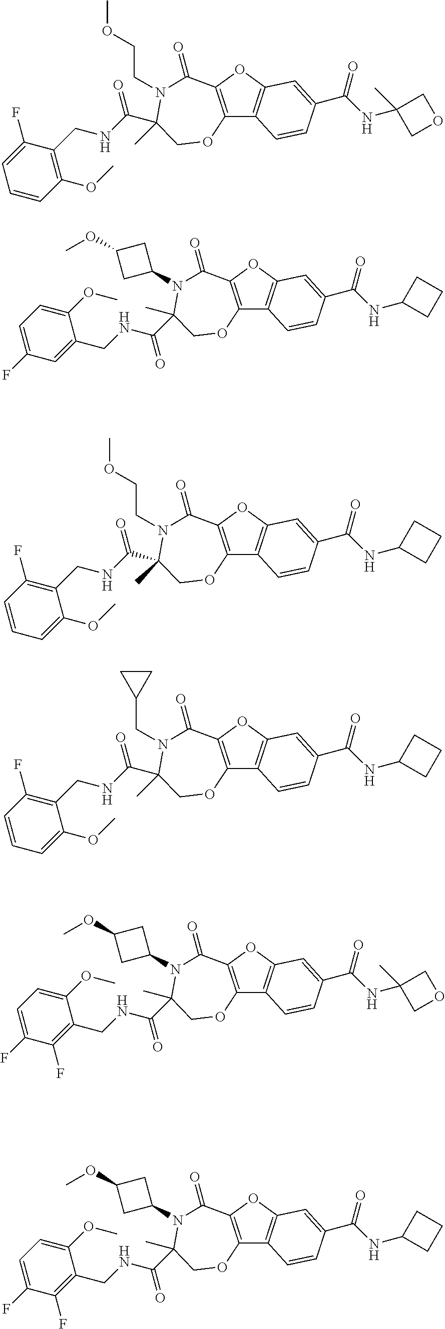 [SMILES: COCCN1C(=O)c2oc3cc(C(=O)NC4(C)COC4)ccc3c2OCC1(C)C(=O)NCc1c(F)cccc1OC ; COc1ccc(F)cc1CNC(=O)C1(C)COc2c(oc3cc(C(=O)NC4CCC4)ccc23)C(=O)N1[C@H]1C[C@H](OC)C1 ; COCCN1C(=O)c2oc3cc(C(=O)NC4CCC4)ccc3c2OC[C@]1(C)C(=O)NCc1c(F)cccc1OC ; COc1cccc(F)c1CNC(=O)C1(C)COc2c(oc3cc(C(=O)NC4CCC4)ccc23)C(=O)N1CC1CC1 ; COc1ccc(F)c(F)c1CNC(=O)C1(C)COc2c(oc3cc(C(=O)NC4(C)COC4)ccc23)C(=O)N1[C@H]1C[C@@H](OC)C1 ; COc1ccc(F)c(F)c1CNC(=O)C1(C)COc2c(oc3cc(C(=O)NC4CCC4)ccc23)C(=O)N1[C@H]1C[C@@H](OC)C1] 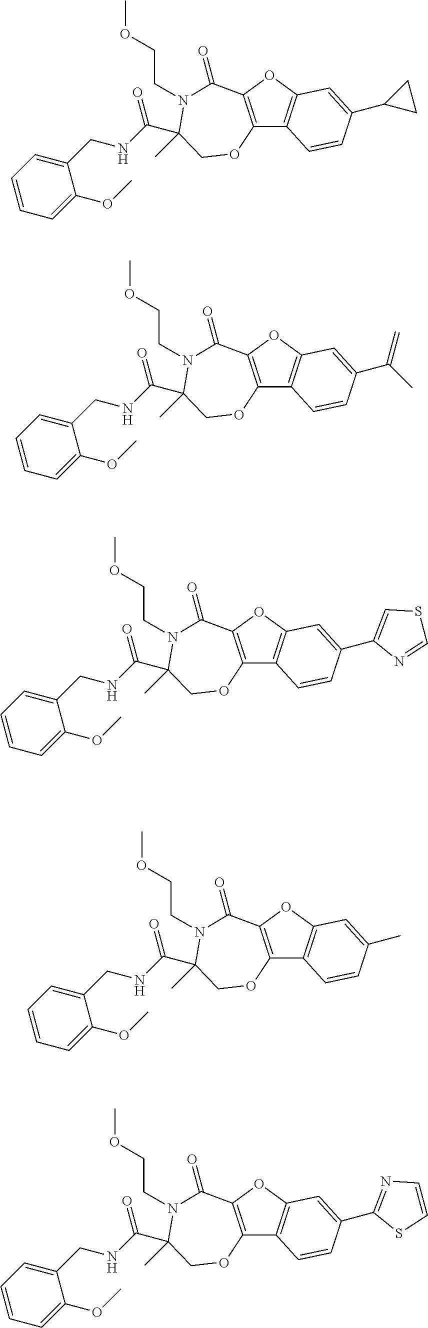 [SMILES: COCCN1C(=O)c2oc3cc(C4CC4)ccc3c2OCC1(C)C(=O)NCc1ccccc1OC ; C=C(C)c1ccc2c3c(oc2c1)C(=O)N(CCOC)C(C)(C(=O)NCc1ccccc1OC)CO3 ; COCCN1C(=O)c2oc3cc(-c4cscn4)ccc3c2OCC1(C)C(=O)NCc1ccccc1OC ; COCCN1C(=O)c2oc3cc(C)ccc3c2OCC1(C)C(=O)NCc1ccccc1OC ; COCCN1C(=O)c2oc3cc(-c4nccs4)ccc3c2OCC1(C)C(=O)NCc1ccccc1OC] 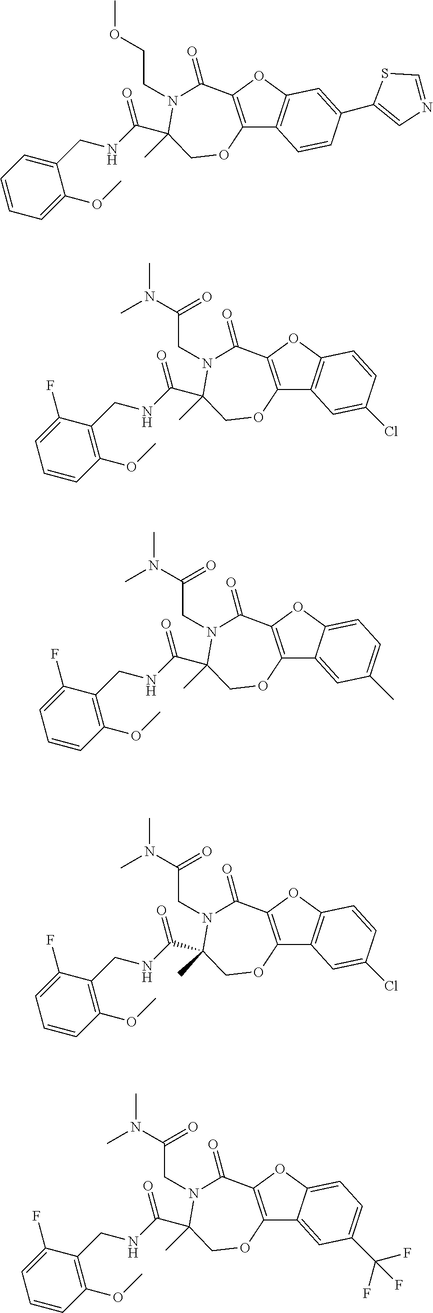 [SMILES: COCCN1C(=O)c2oc3cc(-c4cncs4)ccc3c2OCC1(C)C(=O)NCc1ccccc1OC ; COc1cccc(F)c1CNC(=O)C1(C)COc2c(oc3ccc(Cl)cc23)C(=O)N1CC(=O)N(C)C ; COc1cccc(F)c1CNC(=O)C1(C)COc2c(oc3ccc(C)cc23)C(=O)N1CC(=O)N(C)C ; COc1cccc(F)c1CNC(=O)[C@@]1(C)COc2c(oc3ccc(Cl)cc23)C(=O)N1CC(=O)N(C)C ; COc1cccc(F)c1CNC(=O)C1(C)COc2c(oc3ccc(C(F)(F)F)cc23)C(=O)N1CC(=O)N(C)C] 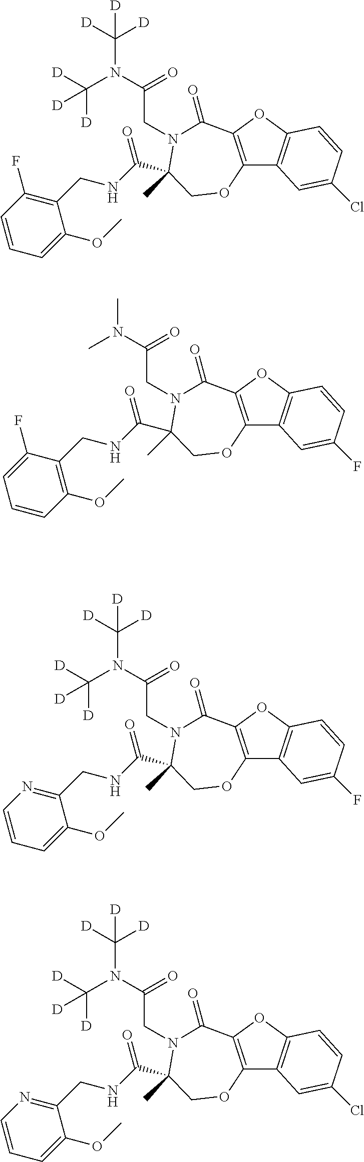 [SMILES: [2H]C([2H])([2H])N(C(=O)CN1C(=O)c2oc3ccc(Cl)cc3c2OC[C@]1(C)C(=O)NCc1c(F)cccc1OC)C([2H])([2H])[2H] ; COc1cccc(F)c1CNC(=O)C1(C)COc2c(oc3ccc(F)cc23)C(=O)N1CC(=O)N(C)C ; [2H]C([2H])([2H])N(C(=O)CN1C(=O)c2oc3ccc(F)cc3c2OC[C@]1(C)C(=O)NCc1ncccc1OC)C([2H])([2H])[2H] ; [2H]C([2H])([2H])N(C(=O)CN1C(=O)c2oc3ccc(Cl)cc3c2OC[C@]1(C)C(=O)NCc1ncccc1OC)C([2H])([2H])[2H]] 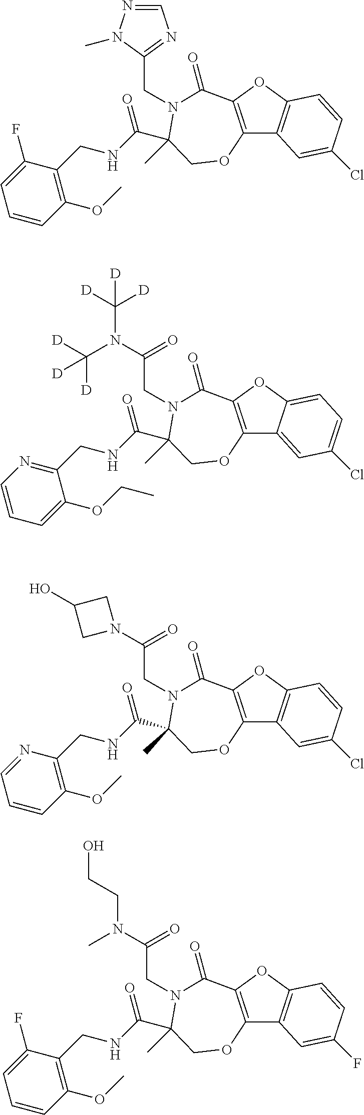 [SMILES: COc1cccc(F)c1CNC(=O)C1(C)COc2c(oc3ccc(Cl)cc23)C(=O)N1Cc1ncnn1C ; [2H]C([2H])([2H])N(C(=O)CN1C(=O)c2oc3ccc(Cl)cc3c2OCC1(C)C(=O)NCc1ncccc1OCC)C([2H])([2H])[2H] ; COc1cccnc1CNC(=O)[C@@]1(C)COc2c(oc3ccc(Cl)cc23)C(=O)N1CC(=O)N1CC(O)C1 ; COc1cccc(F)c1CNC(=O)C1(C)COc2c(oc3ccc(F)cc23)C(=O)N1CC(=O)N(C)CCO] 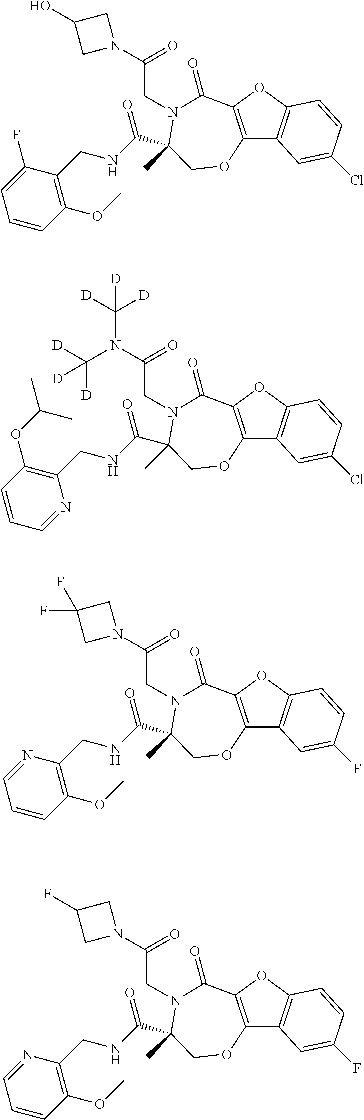 [SMILES: COc1cccc(F)c1CNC(=O)[C@@]1(C)COc2c(oc3ccc(Cl)cc23)C(=O)N1CC(=O)N1CC(O)C1 ; [2H]C([2H])([2H])N(C(=O)CN1C(=O)c2oc3ccc(Cl)cc3c2OCC1(C)C(=O)NCc1ncccc1OC(C)C)C([2H])([2H])[2H] ; COc1cccnc1CNC(=O)[C@@]1(C)COc2c(oc3ccc(F)cc23)C(=O)N1CC(=O)N1CC(F)(F)C1 ; COc1cccnc1CNC(=O)[C@@]1(C)COc2c(oc3ccc(F)cc23)C(=O)N1CC(=O)N1CC(F)C1] 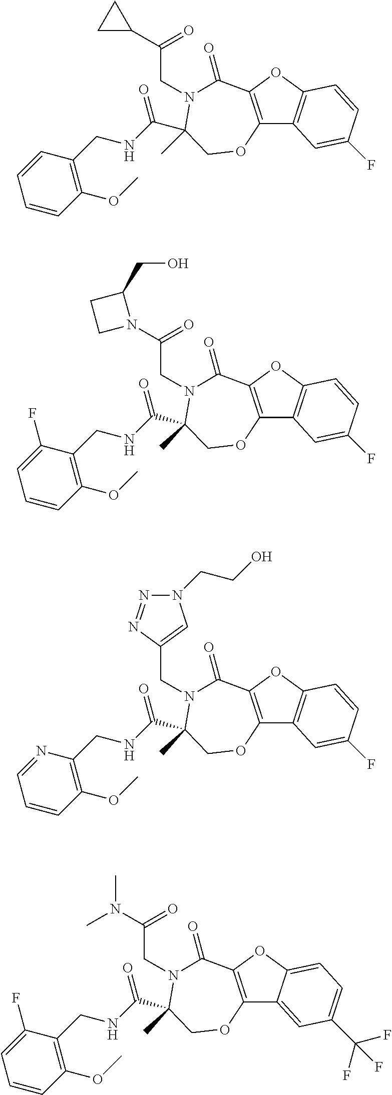 [SMILES: COc1ccccc1CNC(=O)C1(C)COc2c(oc3ccc(F)cc23)C(=O)N1CC(=O)C1CC1 ; COc1cccc(F)c1CNC(=O)[C@@]1(C)COc2c(oc3ccc(F)cc23)C(=O)N1CC(=O)N1CC[C@H]1CO ; COc1cccnc1CNC(=O)[C@@]1(C)COc2c(oc3ccc(F)cc23)C(=O)N1Cc1cn(CCO)nn1 ; COc1cccc(F)c1CNC(=O)[C@@]1(C)COc2c(oc3ccc(C(F)(F)F)cc23)C(=O)N1CC(=O)N(C)C] 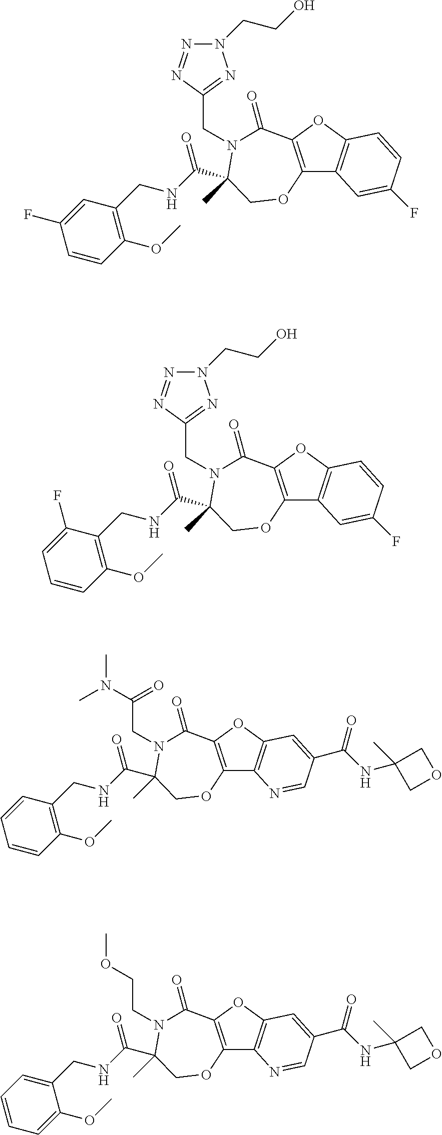 [SMILES: COc1ccc(F)cc1CNC(=O)[C@@]1(C)COc2c(oc3ccc(F)cc23)C(=O)N1Cc1nnn(CCO)n1 ; COc1cccc(F)c1CNC(=O)[C@@]1(C)COc2c(oc3ccc(F)cc23)C(=O)N1Cc1nnn(CCO)n1 ; COc1ccccc1CNC(=O)C1(C)COc2c(oc3cc(C(=O)NC4(C)COC4)cnc23)C(=O)N1CC(=O)N(C)C ; COCCN1C(=O)c2oc3cc(C(=O)NC4(C)COC4)cnc3c2OCC1(C)C(=O)NCc1ccccc1OC] 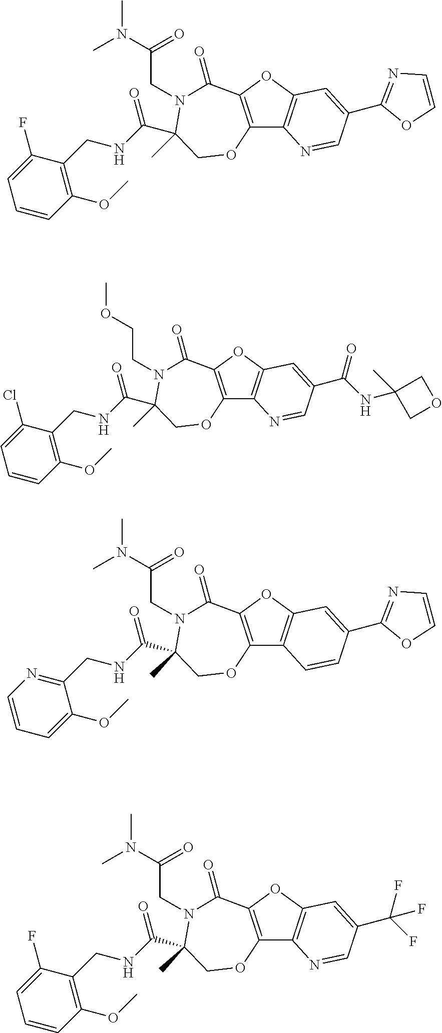 [SMILES: COc1cccc(F)c1CNC(=O)C1(C)COc2c(oc3cc(-c4ncco4)cnc23)C(=O)N1CC(=O)N(C)C ; COCCN1C(=O)c2oc3cc(C(=O)NC4(C)COC4)cnc3c2OCC1(C)C(=O)NCc1c(Cl)cccc1OC ; COc1cccnc1CNC(=O)[C@@]1(C)COc2c(oc3cc(-c4ncco4)ccc23)C(=O)N1CC(=O)N(C)C ; COc1cccc(F)c1CNC(=O)[C@@]1(C)COc2c(oc3cc(C(F)(F)F)cnc23)C(=O)N1CC(=O)N(C)C] 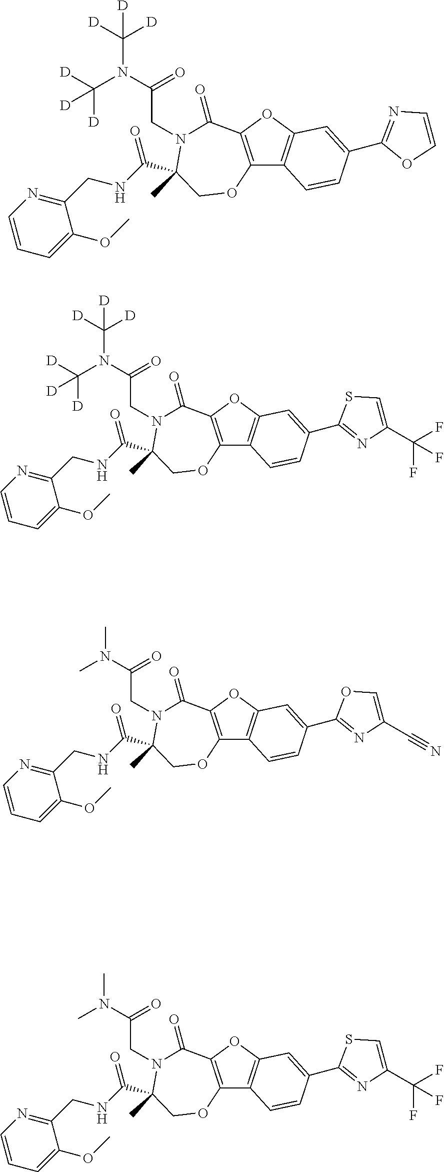 [SMILES: [2H]C([2H])([2H])N(C(=O)CN1C(=O)c2oc3cc(-c4ncco4)ccc3c2OC[C@]1(C)C(=O)NCc1ncccc1OC)C([2H])([2H])[2H] ; [2H]C([2H])([2H])N(C(=O)CN1C(=O)c2oc3cc(-c4nc(C(F)(F)F)cs4)ccc3c2OC[C@]1(C)C(=O)NCc1ncccc1OC)C([2H])([2H])[2H] ; COc1cccnc1CNC(=O)[C@@]1(C)COc2c(oc3cc(-c4nc(C#N)co4)ccc23)C(=O)N1CC(=O)N(C)C ; COc1cccnc1CNC(=O)[C@@]1(C)COc2c(oc3cc(-c4nc(C(F)(F)F)cs4)ccc23)C(=O)N1CC(=O)N(C)C] 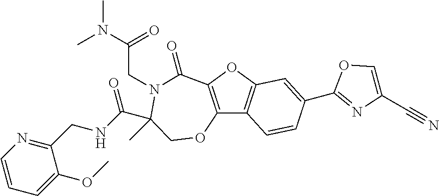 [SMILES: COc1cccnc1CNC(=O)C1(C)COc2c(oc3cc(-c4nc(C#N)co4)ccc23)C(=O)N1CC(=O)N(C)C]
or a pharmaceutically acceptable salt thereof.

43. A pharmaceutical composition comprising a compound of any one of claims 1 to 42, or a pharmaceutically acceptable salt thereof, and a pharmaceutically acceptable carrier.

44. A method of treating an inflammatory disease or endothelial dysfunction comprising administering a therapeutically effective amount of a compound of any one of claims 1 to 42, or a pharmaceutically acceptable salt thereof, or the composition of claim 43, to a subject in need thereof.

45. The method of claim 44, wherein the inflammatory disease or endothelial dysfunction is chosen from atherosclerosis, coronary artery disease, stroke, peripheral arterial disease, coronary microvascular diseases, angina, systemic hypertension, pulmonary arterial hypertension, heart failure, and diabetic microvascular diseases, such as diabetic nephropathy, diabetic retinopathy or diabetic neuropathy, or autoimmune, inflammatory or infectious diseases.
